(19) United States
(12) Patent Application Publication
(10) Pub. No.: US 2024/0116922 A1
(43) Pub. Date: Apr. 11, 2024

(54) IMIDAZO[1,2-A]PYRIDINE DERIVATIVES AS IRAK4 INHIBITORS AND THEIR USE IN THE TREATMENT OF DISEASE

(71) Applicant: BIOGEN MA INC., Cambridge, MA (US)

(72) Inventors: Emily Anne Peterson, Belmont, MA (US); Magnus Pfaffenbach, Chelsea, MA (US); Fang Gao, Somerville, MA (US); Philippe Bolduc, Weymouth, MA (US); Zhili Xin, Lexington, MA (US); Ryan Evans, Somerville, MA (US)

(21) Appl. No.: 18/269,073

(22) PCT Filed: Dec. 21, 2021
(86) PCT No.: PCT/US2021/064666

§ 371 Date: Jun. 22, 2023

Related U.S. Application Data
(60) Provisional application No. 63/128,964, filed on Dec. 22, 2020.

(51) Int. Cl. C07D 471/04; A61K 45/06; C07D 487/04

(52) U.S. Cl. CPC C07D 471/04; A61K 45/06; C07D 487/04

(57) ABSTRACT

This disclosure relates to imidazo[1,2-a]pyridinyl derivatives of formula (I), or pharmaceutically acceptable salts thereof, Formula (I) in which all of the variables are as defined in the specification, capable of modulating the activity of IRAK4. The disclosure further provides methods to their preparation, to their medical use, in particular to their use in the treatment and management of diseases or disorders including inflammatory disease, autoimmune disease, cancer, cardiovascular disease, a disease of the central nervous system, disease of the skin, an ophthalmic disease and condition, and a bone disease.

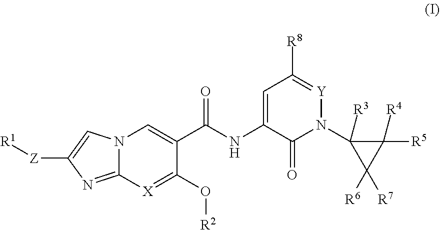

### RELATED APPLICATIONS

This application claims the benefit of and priority to the filing date under 35 U.S.C. § 119(e) of U.S. Provisional Application No. 63/128,964, filed Dec. 22, 2020, the entire contents of which are incorporated herein by reference.

### FIELD OF THE INVENTION

The present disclosure relates to imidazo[1,2-a]pyridinyl derivatives and pharmaceutically acceptable salts thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the imidazo[1,2-a]pyridinyl derivatives to a warm-blooded animal, especially a human.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the protein kinase family.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Kinase targets of particular interest are members of the IRAK family. The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs). IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2. IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572). Many diseases are associated with abnormal cellular responses triggered by kinase-mediated events.

Many diseases and/or disorders are associated with abnormal cellular responses triggered by kinase-mediated events. These diseases and/or disorders include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, epilepsy, ischemic stroke, ophthalmic diseases, ocular diseases, asthma, Alzheimer's disease, amyotrophic lateral sclerosis, Parkinson's disease, traumatic brain injury, chronic traumatic encephalopathy and hormone-related diseases.

In view of the above, IRAK4 inhibitors are considered to be of value in the treatment and/or prevention for multiple therapeutic indications over a wide range of unmet needs.

### SUMMARY OF THE INVENTION

Compounds of the present disclosure are potent and brain penetrant IRAK4 inhibitors. Specifically, including a cyclopropyl pyridone moiety in the compounds of the present disclosure surprisingly result in dramatic increase in potency against IRAK4 (e.g, picomolar potency in the IRAK4 biochemical assay as described in the Examples) while maintaining the solubility and brain penetration observed with compounds with more polar moieties (e.g., methyl pyrazole and methyl pyridone). The compounds of the present disclosure have the desirable potency, solubility and brain penetrating properties.

In a first aspect, the present disclosure relates to a compound of formula (I)

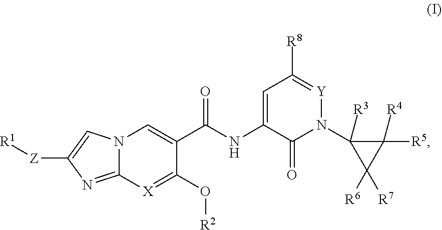

or a pharmaceutically acceptable salt thereof, wherein:
X is CH, CF or N;
Y is CH or N;
Z is ring A or —CH₂-ring A-*, wherein -* indicates the point of connection to R¹;
Ring A is

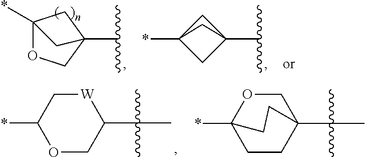

wherein n is 1 or 2; W is absent, CH₂ or O, and [symbol] indicates the point of connection to R¹;
R¹ is H, —CN, C₁₋₃alkoxy or C₁₋₃alkyl optionally substituted with 1 to 3 substituents independently selected from halo and C₁-C₃alkoxy;
R² is C₃₋₆cycloalkyl or C₁₋₄alkyl, wherein the C₃₋₆cycloalkyl or C₁₋₄alkyl is optionally substituted with 1 to 3 halo; and
R³, R⁴, R⁵, R⁶ and R⁷ are each independently selected from H, halo, CN, C₁₋₄alkyl, C₁₋₄haloalkyl, C₁₋₄alkoxy, and C₁₋₄alkoxyC₁₋₄alkyl, or any two of R³, R⁴, R⁵, R⁶ and R⁷ together with the carbon atoms from which they are attached form a C₃₋₆cycloalkyl or a 4 to 6 membered heterocyclyl containing one or two heteroatoms independently selected O, N, and S; and
R⁸ is H or halo.

Another aspect of the disclosure relates to pharmaceutical compositions comprising compounds of formula (I) or pharmaceutically acceptable salts thereof, and a pharmaceutical carrier. Such compositions can be administered in accordance with a method of the present disclosure, typically as part of a therapeutic regimen for the treatment or prevention of conditions and disorders related to interleukin-1 receptor-associated kinases activity. In certain embodiments, the pharmaceutical compositions may additionally comprise further one or more therapeutically active ingredients or therapeutic agent suitable for the use in combination with the compounds of the invention. In certain embodiments, the compounds or the pharmaceutical compositions of the present disclosure can be used in combination with one or more additional therapeutically active ingredients or therapeutic agents in a method of present disclosure. In some embodiments, the further or additional therapeutically active ingredient or therapeutic agent is an agent that can be used for the treatment of autoimmune diseases, inflammatory diseases, bone diseases, metabolic diseases, neurological and neurodegenerative diseases, cancer, cardiovascular diseases, allergies, asthma, Alzheimer's disease, and hormone-related diseases.

Another aspect of the present disclosure relates to the pharmaceutical combinations comprising compounds of the invention and other therapeutic agents for the use as a medicament in the treatment of patients having disorders related to interleukin-1 receptor-associated kinases activity. Such combinations can be administered in accordance with a method of the invention, typically as part of a therapeutic regiment for the treatment or prevention of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Also provided in the present disclosure are compounds or pharmaceutical compositions described herein for use in the treatment of patients having disorders related to interleukin-1 receptor-associated kinases activity. Uses of the compounds or pharmaceutical compositions described herein for the manufacture of a medicament for treating patients having disorders related to interleukin-1 receptor-associated kinases activity are also included in the present disclosure.

### DETAILED DESCRIPTION OF THE INVENTION

The present disclosure provides compounds and pharmaceutical compositions thereof that may be useful in the treatment or prevention of conditions and/or disorders through mediation of IRAK4 function. In some embodiments, the compounds of present disclosure are IRAK4 inhibitors.

In a first embodiment, the present disclosure provides a compound of formula (I):

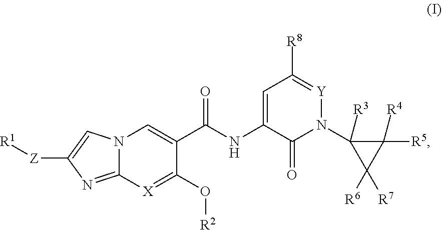

or a pharmaceutically acceptable salt thereof, wherein the variables in formula (I) are as defined in the first aspect above.

In a second embodiment, for the compound of formula (I) described in the first embodiment, or a pharmaceutically acceptable salt thereof, X is CH; and the remaining variables are as described in the first embodiment.

In a third embodiment, for the compound of formula (I) described in the first embodiment, or a pharmaceutically acceptable salt thereof, X is N; and the remaining variables are as described in the first embodiment.

In a fourth embodiment, for the compound of formula (I), or a pharmaceutically acceptable salt thereof, Y is CH; and the remaining variables are as described in the first, second or third embodiment.

In a fifth embodiment, for the compound of formula (I), or a pharmaceutically acceptable salt thereof, Y is N; and the remaining variables are as described in the first, second or third embodiment.

In a sixth embodiment, for the compound of formula (I), or a pharmaceutically acceptable salt thereof, Z is ring A, ring A is

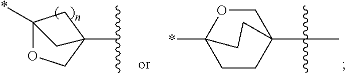

and the remaining variables are as described in the first, second, third, fourth or fifth embodiment.

In a seventh embodiment, for the compound of formula (I), or a pharmaceutically acceptable salt thereof, Z is ring A, ring A is

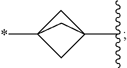 [SMILES: *C12CC(*)(C1)C2]

and the remaining variables are as described in the first, second, third, fourth or fifth embodiment.

In an eighth embodiment, for the compound of formula (I), or a pharmaceutically acceptable salt thereof, ring A is

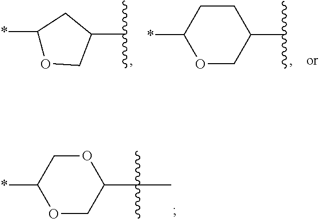

and the remaining variables are as described in the first, second, third, fourth or fifth embodiment. In some embodiments, for compounds of the eighth embodiment, Z is —CH₂-ring A-*. In some embodiments, for compounds of the eighth embodiment, Z is ring A.

In a ninth embodiment, the compound of the present disclosure is represented by Formula (II), (III), (IV) or (V):

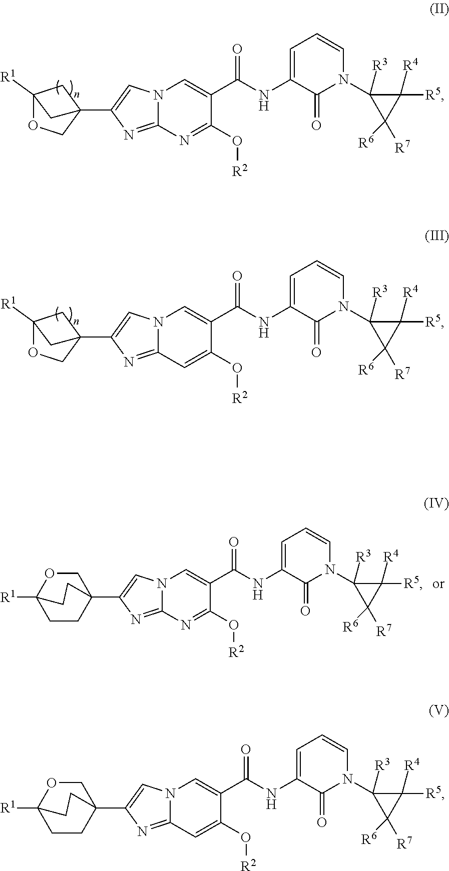

or a pharmaceutically acceptable salt thereof, wherein the variables R¹, R², R³, R⁴, R⁵, R⁶, R⁷ and n depicted in Formula (II), (III), (IV) or (V) are as described in the first embodiment.

In a tenth embodiment, the compound of the present disclosure is represented by Formula (IIA), (JIB), (IIIA), or (IIIB):

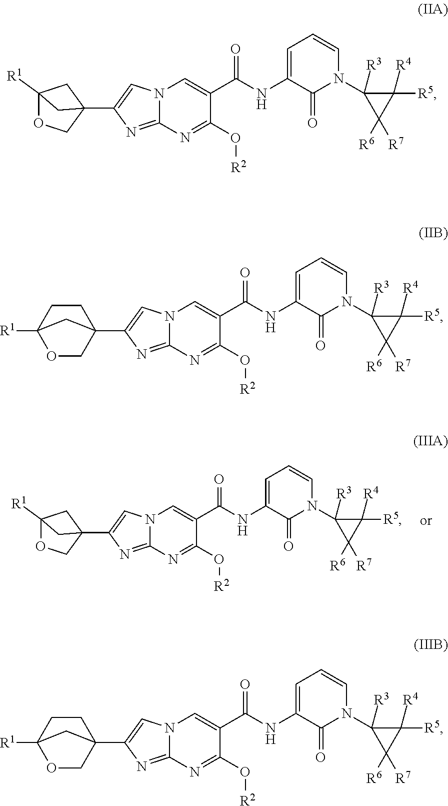

or a pharmaceutically acceptable salt thereof, the variables R¹, R², R³, R⁴, R⁵, R⁶ and R⁷ depicted in Formula (IIA), (IIB), (IIIA) or (IIIB) are as described in the first embodiment.

In an eleventh embodiment, for compounds of formula (I), (II), (III), (IV), (V), (IIA), (IIB), (IIIA) or (IIIB), or a pharmaceutically acceptable salt thereof, R¹ is H or C₁₋₃alkyl optionally substituted with 1 to 3 substituents independently selected from halo or C₁-C₃alkoxy; and the remaining variables are as described in the first, second, third, fourth, fifth, sixth, seventh, eighth, ninth or tenth embodiment.

In a twelfth embodiment, for compounds of formula (I), (II), (III), (IV), (V), (IIA), (IIB), (IIIA) or (IIIB), or a pharmaceutically acceptable salt thereof, R¹ is C₁₋₃alkyl; and the remaining variables are as described in the first, second, third, fourth, fifth, sixth, seventh, eighth, ninth or tenth embodiment.

In a thirteenth embodiment, for compounds of formula (I), (II), (III), (IV), (V), (IIA), (IIB), (IIIA) or (IIIB), or a pharmaceutically acceptable salt thereof, R¹ is C₁₋₃alkyl optionally substituted with 1 to 3 halo; and the remaining variables are as described in the first, second, third, fourth, fifth, sixth, seventh, eighth, ninth or tenth embodiment.

In a fourteenth embodiment, for compounds of formula (I), (II), (III), (IV), (V), (IIA), (IIB), (IIIA) or (IIIB), or a pharmaceutically acceptable salt thereof, R¹ is H, —CH₃, —CH₂F, —CH₂CH₃, —CH₂OCH₃, —OCH₃, or —CN; and the remaining variables are as described in the first, second, third, fourth, fifth, sixth, seventh, eighth, ninth or tenth embodiment.

In a fifteenth embodiment, for compounds of formula (I), (II), (III), (IV), (V), (IIA), (IIB), (IIIA) or (IIIB), or a pharmaceutically acceptable salt thereof, R¹ is —CH₃; and the remaining variables are as described in the first, second, third, fourth, fifth, sixth, seventh, eighth, ninth or tenth embodiment.

In a sixteenth embodiment, for compounds of formula (I), (II), (III), (IV), (V), (IIA), (IIB), (IIIA) or (IIIB), or a pharmaceutically acceptable salt thereof, R¹ is —CH₃ or —CH₂F; and the remaining variables are as described in the first, second, third, fourth, fifth, sixth, seventh, eighth, ninth or tenth embodiment.

In a seventeenth embodiment, for compounds of formula (I), (II), (III), (IV), (V), (IIA), (IIB), (IIIA) or (IIIB), or a pharmaceutically acceptable salt thereof, R² is C₃₋₄alkyl or C₃₋₄cycloalkyl, wherein the C₃₋₄alkyl is optionally substituted with 1 to 3 fluoro; and the remaining variables are as described in the first, second, third, fourth, fifth, sixth, seventh, eighth, ninth, tenth, eleventh, twelfth, thirteenth, fourteenth, fifteenth, or sixteenth embodiment.

In an eighteenth embodiment, for compounds of formula (I), (II), (III), (IV), (V), (IIA), (IIB), (IIIA) or (IIIB), or a pharmaceutically acceptable salt thereof, R² is C₃₋₄alkyl; and the remaining variables are as described in the first, second, third, fourth, fifth, sixth, seventh, eighth, ninth, tenth, eleventh, twelfth, thirteenth, fourteenth, fifteenth, or sixteenth embodiment.

In a nineteenth embodiment, for compounds of formula (I), (II), (III), (IV), (V), (IIA), (IIB), (IIIA) or (IIIB), or a pharmaceutically acceptable salt thereof, R² is —CH(CH₃)₂, —CH(CH₃)CH₂CH₃, —CH(CH₃)CH₂F, —CH(CH₃)CHF₂, cyclopropyl, or cyclobutyl; and the remaining variables are as described in the first, second, third, fourth, fifth, sixth, seventh, eighth, ninth, tenth, eleventh, twelfth, thirteenth, fourteenth, fifteenth, or sixteenth embodiment.

In a twentieth embodiment, for compounds of formula (I), (II), (III), (IV), (V), (IIA), (IIB), (IIIA) or (IIIB), or a pharmaceutically acceptable salt thereof, R² is —CH(CH₃)₂; and the remaining variables are as described in the first, second, third, fourth, fifth, sixth, seventh, eighth, ninth, tenth, eleventh, twelfth, thirteenth, fourteenth, fifteenth, or sixteenth embodiment.

In a twenty-first embodiment, for compounds of formula (I), (II), (III), (IV), (V), (IIA), (IIB), (IIIA) or (IIIB), or a pharmaceutically acceptable salt thereof, R² is —CH(CH₃)₂ or —CH(CH₃)CH₂CH₃; and the remaining variables are as described in the first, second, third, fourth, fifth, sixth, seventh, eighth, ninth, tenth, eleventh, twelfth, thirteenth, fourteenth, fifteenth, or sixteenth embodiment.

In a twenty-second embodiment, for compounds of formula (I), (II), (III), (IV), (V), (IIA), (IIB), (IIIA) or (IIIB), or a pharmaceutically acceptable salt thereof, R¹ is H or C₁₋₃alkyl optionally substituted with 1 to 3 substituents independently selected from halo or C₁-C₃alkoxy; R² is C₃₋₄alkyl; and the remaining variables are as described in the first, second, third, fourth, fifth, sixth, seventh, eighth, ninth or tenth embodiment.

In a twenty-third embodiment, for compounds of formula (I), (II), (III), (IV), (V), (IIA), (IIB), (IIIA) or (IIIB), or a pharmaceutically acceptable salt thereof, R¹ is H or C₁₋₃alkyl optionally substituted with 1 to 3 halo; R² is C₃₋₄alkyl; and the remaining variables are as described in the first, second, third, fourth, fifth, sixth, seventh, eighth, ninth or tenth embodiment.

In a twenty-fourth embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are each independently selected from H, halo and C₁₋₃alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are each independently selected from H, halo, C₁₋₃alkyl, C₁₋₃haloalkyl and C₁₋₃alkoxy, or any two of R³, R⁴, R⁵, R⁶ and R⁷ together with the carbon atoms from which they are attached form a C₃₋₆cycloalkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-sixth embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are each independently selected from H, F, and —CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-seventh embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are each independently selected from H, F, —CH₃, —CH₂CH₃, —CHF₂, and —OCH₃, or any two of R³, R⁴, R⁵, R⁶ and R⁷ together with the carbon atoms from which they are attached form a cyclopropyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-eighth embodiment, for compounds of formula (I), (II), (III), (IV), (V), (IIA), (IIB), (IIIA) or (IIIB), or a pharmaceutically acceptable salt thereof, R³, R⁴, R⁵, R⁶ and R⁷ are all H; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-ninth embodiment, for compounds of formula (I), (II), (III), (IV), (V), (IIA), (IIB), (IIIA) or (IIIB), or a pharmaceutically acceptable salt thereof, R³, R⁵, R⁶ and R⁷ are all H, and R⁴ is F or —CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a thirtieth embodiment, the compound of present disclosure is represented by the following formula:

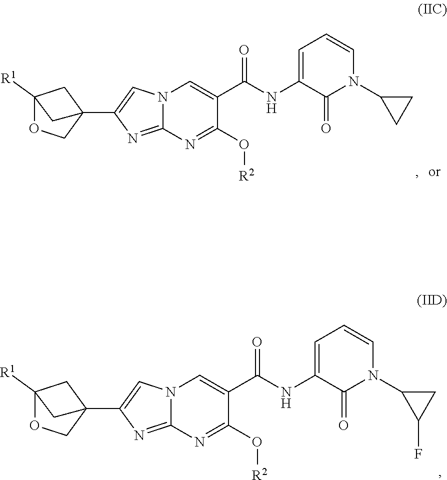

or a pharmaceutically acceptable salt thereof, wherein R¹ is C₁₋₃alkyl and R² is C₃₋₄alkyl.

In a thirty-first embodiment, the compound of present disclosure is represented by the following formula:

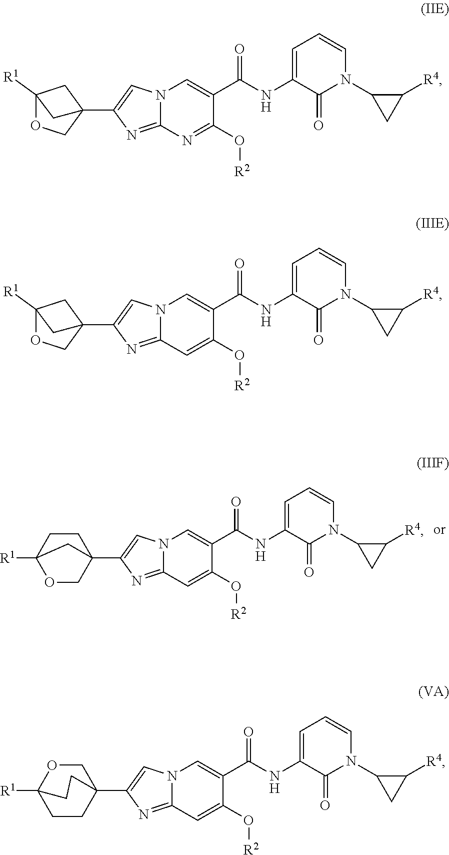

or a pharmaceutically acceptable salt thereof, wherein R¹ is C₁₋₃alkyl optionally substituted with 1 to 3 halo; R² is C₃₋₄alkyl; and R⁴ is H, halo or C₁₋₃alkyl.

In a thirty-second embodiment, for compounds of formula (IIE), (IIIE), (IIIF), or (VA), or a pharmaceutically acceptable salt thereof, R¹ is —CH₃ or —CH₂F; R² is —CH(CH₃)₂ or —CH(CH₃)CH₂CH₃; and R⁴ is H, F or —CH₃.

In a thirty-third embodiment, for compounds of formula (IIE), (IIIE), (IIIF), or (VA), or a pharmaceutically acceptable salt thereof, when R⁴ is not H, R⁴ and the pyridone group are in a cis orientation, and the remaining variables are as described in the thirty-first or thirty-second embodiment.

In a thirty-fourth embodiment, the present disclosure provides a compound described herein (e.g., a compound of any one Examples 1-192) or a pharmaceutically acceptable salt thereof.

In a thirty-fifth embodiment, the present disclosure provides a compound selected from the group consisting of:
7-cyclobutoxy-N-(1-cyclopropyl-2-oxo-1,2-dihydropyridin-3-yl)-2-(1-methyl-2-oxabicyclo[2.2.2]octan-4-yl)imidazo[1,2-a]pyridine-6-carboxamide,
N-(1-cyclopropyl-2-oxo-1,2-dihydropyridin-3-yl)-2-(1-ethyl-2-oxabicyclo[2.1.1]hexan-4-yl)-7-isopropoxyimidazo[1,2-a]pyridine-6-carboxamide,
N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyridine-6-carboxamide,
N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyridine-6-carboxamide,
N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyridine-6-carboxamide,
N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyridine-6-carboxamide,
N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide,
N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyridine-6-carboxamide,
N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide,
N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide,
(R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide,
(S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide,
7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide,
7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide,
N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
(S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide,
7-(sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide,
7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide,
7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide,
7-isopropoxy-2-(1-methyl-2-oxabicyclo[2.1.1]hexan-4-yl)-N-(1-(1-methylcyclopropyl)-2-oxo-1,2-dihydropyridin-3-yl)imidazo[1,2-a]pyrimidine-6-carboxamide,
7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide,
(S)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide,
(R)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide,
N-(1-cyclopropyl-2-oxo-1,2-dihydropyridin-3-yl)-7-(((S)-1-fluoropropan-2-yl)oxy)-2-(1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide,
7-cyclobutoxy-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide,
2-(2-oxabicyclo[2.1.1]hexan-4-yl)-N-(1-cyclopropyl-2-oxo-1,2-dihydropyridin-3-yl)-7-isopropoxyimidazo[1,2-a]pyridine-6-carboxamide,
N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide,
2-(2-oxabicyclo[2.1.1]hexan-4-yl)-7-cyclobutoxy-N-(1-cyclopropyl-2-oxo-1,2-dihydropyridin-3-yl)imidazo[1,2-a]pyrimidine-6-carboxamide,
2-(2-oxabicyclo[2.1.1]hexan-4-yl)-7-cyclobutoxy-N-(1-cyclopropyl-2-oxo-1,2-dihydropyridin-3-yl)imidazo[1,2-a]pyridine-6-carboxamide,
2-(2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide,
2-((1S,4R)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide,
2-((1R,4S)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide,
7-((R)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-((S)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-7-ethoxy-2-(1-methyl-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-2-(1-ethyl-2-oxabicyclo[2.2.1]heptan-4-yl)-7-isopropoxyimidazo[1,2-a]pyrimidine-6-carboxamide,
N-(1-cyclopropyl-2-oxo-1,2-dihydropyridin-3-yl)-2-(1-(fluoromethyl)-2-oxabicyclo[2.2.1]heptan-4-yl)-7-isopropoxyimidazo[1,2-a]pyrimidine-6-carboxamide,
N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyrimidine-6-carboxamide,
N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyrimidine-6-carboxamide,
7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide,
7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide,
7-((S)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide,
7-((R)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide,
N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide,
N-(1-cyclopropyl-2-oxo-1,2-dihydropyridin-3-yl)-7-isopropoxy-2-(1-(methoxymethyl)-2-oxabicyclo[2.2.1]heptan-4-yl)imidazo[1,2-a]pyrimidine-6-carboxamide,
2-(2-oxabicyclo[2.1.1]hexan-4-yl)-N-(1-cyclopropyl-2-oxo-1,2-dihydropyridin-3-yl)-7-isopropoxyimidazo[1,2-a]pyrimidine-6-carboxamide,
7-((R)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide,
7-((S)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide,
7-((R)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-((R)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-8-fluoro-7-isopropoxy-2-(1-methyl-2-oxabicyclo[2.1.1]hexan-4-yl)imidazo[1,2-a]pyridine-6-carboxamide,
N-(1-cyclopropyl-2-oxo-1,2-dihydropyridin-3-yl)-7-isopropoxy-2-(3-methoxybicyclo[1.1.1]pentan-1-yl)imidazo[1,2-a]pyridine-6-carboxamide,
(R)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide,
(S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide,
(R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide,
(S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-8-fluoro-7-isopropoxy-2-((1S,4R)-1-methyl-2-oxabicyclo[2.2.1]heptan-4-yl)imidazo[1,2-a]pyridine-6-carboxamide,
N-(1-cyclopropyl-2-oxo-1,2-dihydropyridin-3-yl)-8-fluoro-7-isopropoxy-2-((1R,4S)-1-methyl-2-oxabicyclo[2.2.1]heptan-4-yl)imidazo[1,2-a]pyridine-6-carboxamide,
(R)—N-(1-cyclopropyl-2-oxo-1,2-dihydropyridin-3-yl)-2-(1,4-dioxan-2-yl)-7-isopropoxyimidazo[1,2-a]pyridine-6-carboxamide,
(S)—N-(1-cyclopropyl-2-oxo-1,2-dihydropyridin-3-yl)-2-(1,4-dioxan-2-yl)-7-isopropoxyimidazo[1,2-a]pyridine-6-carboxamide,
N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide,
7-cyclobutoxy-N-(2-cyclopropyl-3-oxo-2,3-dihydropyridazin-4-yl)-2-(1-methyl-2-oxabicyclo[2.1.1]hexan-4-yl)imidazo[1,2-a]pyrimidine-6-carboxamide,
N-(2-cyclopropyl-3-oxo-2,3-dihydropyridazin-4-yl)-8-fluoro-7-isopropoxy-2-(1-methyl-2-oxabicyclo[2.1.1]hexan-4-yl)imidazo[1,2-a]pyridine-6-carboxamide,
N-(2-cyclopropyl-3-oxo-2,3-dihydropyridazin-4-yl)-7-isopropoxy-2-((1S,4R)-1-methyl-2-oxabicyclo[2.2.1]heptan-4-yl)imidazo[1,2-a]pyridine-6-carboxamide,
N-(2-cyclopropyl-3-oxo-2,3-dihydropyridazin-4-yl)-7-isopropoxy-2-((1R,4S)-1-methyl-2-oxabicyclo[2.2.1]heptan-4-yl)imidazo[1,2-a]pyridine-6-carboxamide,
N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyrimidine-6-carboxamide,
(R)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
(S)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
2-(3-cyanobicyclo[1.1.1]pentan-1-yl)-N-(1-cyclopropyl-2-oxo-1,2-dihydropyridin-3-yl)-7-isopropoxyimidazo[1,2-a]pyridine-6-carboxamide,
N-(1-trans-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
N-(1-(2,2-difluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
Cis-7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
Cis-2-(2-oxabicyclo[2.1.1]hexan-4-yl)-7-cyclobutoxy-N-(1-(2-fluorocyclopropyl)-2-oxo-1,2-dihydropyridin-3-yl)imidazo[1,2-a]pyrimidine-6-carboxamide,
Cis-2-(2-oxabicyclo[2.1.1]hexan-4-yl)-N-(1-(2-fluorocyclopropyl)-2-oxo-1,2-dihydropyridin-3-yl)-7-isopropoxyimidazo[1,2-a]pyrimidine-6-carboxamide,
2-(2-oxabicyclo[2.1.1]hexan-4-yl)-N-(1-((1R,2S)-2-fluorocyclopropyl)-2-oxo-1,2-dihydropyridin-3-yl)-7-isopropoxyimidazo[1,2-a]pyrimidine-6-carboxamide,
2-(2-oxabicyclo[2.1.1]hexan-4-yl)-N-(1-((1S,2R)-2-fluorocyclopropyl)-2-oxo-1,2-dihydropyridin-3-yl)-7-isopropoxyimidazo[1,2-a]pyrimidine-6-carboxamide,
Cis-N-(1-(2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide,
Cis-8-fluoro-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
8-fluoro-N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
8-fluoro-N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
Cis-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
trans-7-isopropoxy-2-(1-methyl-2-oxabicyclo[2.1.1]hexan-4-yl)-N-(1-(2-methylcyclopropyl)-2-oxo-1,2-dihydropyridin-3-yl)imidazo[1,2-a]pyrimidine-6-carboxamide,
7-((S)-sec-butoxy)-N-(1-cis-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
Cis-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
trans-7-isopropoxy-2-((1S,4R)-1-methyl-2-oxabicyclo[2.2.1]heptan-4-yl)-N-(1-(2-methylcyclopropyl)-2-oxo-1,2-dihydropyridin-3-yl)imidazo[1,2-a]pyridine-6-carboxamide,
N-(1-(2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-(2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
Cis-N-(1-(2-fluorocyclopropyl)-2-oxo-1,2-dihydropyridin-3-yl)-7-isopropoxy-2-(1-methyl-2-oxabicyclo[2.2.1]heptan-4-yl)imidazo[1,2-a]pyrimidine-6-carboxamide,
(S)—N-(1-cyclopropyl-2-oxo-1,2-dihydropyridin-3-yl)-7-((1,1-difluoropropan-2-yl)oxy)-2-(1-methyl-2-oxabicyclo[2.1.1]hexan-4-yl)imidazo[1,2-a]pyridine-6-carboxamide,
Cis-7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-cyclopropoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide,
Cis-7-cyclopropoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
Cis-7-cyclobutoxy-N-(1-(2-fluorocyclopropyl)-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide,
(R)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide,
(S)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide,
(R)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide, and
(S)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide,
N-(1-cyclopropyl-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide,
N-(1-cyclopropyl-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide,
7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
N-(1-cyclopropyl-2-oxo-1,2-dihydropyridin-3-yl)-7-isopropoxy-2-(4-methyl-2-oxabicyclo[2.1.1]hexan-1-yl)imidazo[1,2-a]pyrimidine-6-carboxamide,
N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-((1S,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-((1R,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
N-(1-((1S,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
N-(1-((1R,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide,
N-(1-((1S,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide,
N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide,
N-(1-((1S,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide,
8-fluoro-N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
8-fluoro-N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
8-fluoro-N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
8-fluoro-N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
7-cyclobutoxy-N-(1-((1S,2R)-2-fluorocyclopropyl)-2-oxo-1,2-dihydropyridin-3-yl)-2-(1-(methoxymethyl)-2-oxabicyclo[2.1.1]hexan-4-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide,
N-(1-((1S,2R)-2-fluorocyclopropyl)-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide,
N-(1-((1S,2R)-2-fluorocyclopropyl)-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide,
7-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyridine-6-carboxamide,
7-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyridine-6-carboxamide,
7-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyrimidine-6-carboxamide,
7-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyrimidine-6-carboxamide,
7-isopropoxy-N-(1-((1R,2R)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-isopropoxy-N-(1-((1S,2S)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide,
7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide,
7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide,
7-isopropoxy-2-((1R,4S)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide,
7-isopropoxy-2-((1R,4S)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide,
N-(1-((1R,2R)-2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide,
8-fluoro-7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide,
8-fluoro-7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide,
7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide,
7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide,
7-isopropoxy-2-((1S,4R)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide,
7-isopropoxy-2-((1S,4R)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide,
7-isopropoxy-2-((1R,4S)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide,
7-isopropoxy-2-((1R,4S)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide,
N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyrimidine-6-carboxamide,
7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
N-(1-((1R,2S)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide,
N-(1-((1S,2R)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide,
2-(2-oxabicyclo[2.1.1]hexan-4-yl)-7-cyclobutoxy-N-(1-((1R,2S)-2-fluorocyclopropyl)-2-oxo-1,2-dihydropyridin-3-yl)imidazo[1,2-a]pyrimidine-6-carboxamide,
2-(2-oxabicyclo[2.1.1]hexan-4-yl)-7-cyclobutoxy-N-(1-((1S,2R)-2-fluorocyclopropyl)-2-oxo-1,2-dihydropyridin-3-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide,
N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide,
7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-cyclopropoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-cyclopropoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide,
N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide,
N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide,
N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide,
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyridine-6-carboxamide trifluoroacetate,
7-isopropoxy-2-((1R,4S)-1-methyl-2-oxabicyclo[2.2.1]heptan-4-yl)-N-(1-((1R,2S)-2-methylcyclopropyl)-2-oxo-1,2-dihydropyridin-3-yl)imidazo[1,2-a]pyridine-6-carboxamide trifluoroacetate,
7-isopropoxy-2-((1S,4R)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyridine-6-carboxamide trifluoroacetate,
7-isopropoxy-2-((1S,4R)-1-methyl-2-oxabicyclo[2.2.1]heptan-4-yl)-N-(1-((1R,2S)-2-methylcyclopropyl)-2-oxo-1,2-dihydropyridin-3-yl)imidazo[1,2-a]pyridine-6-carboxamide trifluoroacetate,
7-(((S)-1,1-difluoropropan-2-yl)oxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-(((R)-1,1-difluoropropan-2-yl)oxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide,
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate,
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate,
7-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate,
7-cyclobutoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyrimidine-6-carboxamide trifluoroacetate,
7-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyridine-6-carboxamide trifluoroacetate,
7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyridine-6-carboxamide,
7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyridine-6-carboxamide, or a pharmaceutically acceptable salt thereof.

The present disclosure also provides a pharmaceutical composition comprising a compound according to any one of the preceding embodiments, or a pharmaceutically acceptable salt thereof, and one or more pharmaceutically acceptable carriers.

In certain embodiments, the pharmaceutical composition further comprises one or more additional pharmaceutical or therapeutic agent(s).

In certain embodiments, the present disclosure provides a method of treating an IRAK4 mediated disease in a subject in need of the treatment comprising administering to the subject a compound described herein (e.g., a compound described in any one of the first to thirty-fifth embodiments) or a pharmaceutically acceptable salt thereof or a pharmaceutical composition thereof.

In certain embodiments, the present disclosure provides the use of a compound described herein (e.g., a compound described in any one of the first to thirty-fifth embodiments) or a pharmaceutically acceptable salt thereof or a pharmaceutical composition comprising a compound described herein or a pharmaceutically acceptable salt thereof for the manufacture of a medicament for the treatment of a disorder or disease mediated by IRAK4 in a subject in need of the treatment.

In certain embodiments, the present disclosure provides the use of a compound described herein (e.g., a compound described in any one of the first to thirty-fifth embodiments) or a pharmaceutically acceptable salt thereof or a pharmaceutical composition comprising a compound described herein or a pharmaceutically acceptable salt thereof for the treatment of a disorder or disease mediated by IRAK4 in a subject in need of the treatment.

In certain embodiments, the IRAK4 mediated disease is selected from an autoimmune disease, an inflammatory disease, a bone disease, a metabolic disease, a neurological and neurodegenerative disease and/or disorder, cancer, a cardiovascular disease, allergies, asthma, Alzheimer's disease, a hormone-related disease, ischemic stroke, cerebral ischemia, hypoxia, TBI (Traumatic Brain Injury), CTE (Chronic Traumatic Encephalopathy), epilepsy, Parkinson's disease (PD), multiple Sclerosis (MS) and amyotrophic lateral sclerosis (ALS).

In some embodiments, the present disclosure provides a method of treating MS selected from relapsing-remitting MS (RRMS), secondary progressive MS (SPMS), non-relapsing SPMS, primary progressive MS (PPMS), and clinically isolated syndrome (CIS).

The method comprises administering to the subject a compound described herein (e.g., a compound described in any one of the first to thirty-fifth embodiments) or a pharmaceutically acceptable salt thereof or a pharmaceutical composition thereof. In certain embodiments, the present disclosure provides a method of treating a relapsing form of MS. The method comprises administering to the subject a compound described herein (e.g., a compound described in any one of the first to thirty-fifth embodiments) or a pharmaceutically acceptable salt thereof or a pharmaceutical composition thereof. As used herein, a “relapsing form of MS” includes clinically isolated syndrome (CIS), relapsing-remitting disease (RRMS), and active secondary progressive disease.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

RRMS, the most common disease course of MS, is characterized by clearly defined attacks of new or increasing neurologic symptoms. These attacks—also called relapses or exacerbations—are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

SPMS follows an initial relapsing-remitting course. Some people who are diagnosed with RRMS will eventually transition to a secondary progressive course in which there is a progressive worsening of neurologic function (accumulation of disability) over time. SPMS can be further characterized as either active (with relapses and/or evidence of new MRI activity during a specified period of time) or not active, as well as with progression (evidence of disability accumulation over time, with or without relapses or new MRI activity) or without progression.

PPMS is characterized by worsening neurologic function (accumulation of disability) from the onset of symptoms, without early relapses or remissions. PPMS can be further characterized as either active (with an occasional relapse and/or evidence of new MRI activity over a specified period of time) or not active, as well as with progression (evidence of disability accumulation over time, with or without relapse or new MRI activity) or without progression.

In certain embodiments, the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases, ophthalmic diseases and ocular diseases.

In certain embodiments, the IRAK4 mediated disease is selected from the group consisting from rheumatoid arthritis, psoriatic arthritis, osteoarthritis, systemic lupus, neuropsychiatric lupus, erythematosus, lupus nephritis, ankylosing spondylitis, osteoporosis, systemic sclerosis, multiple sclerosis, neuromyelitis optica, psoriasis, type I diabetes, type II diabetes, inflammatory bowel disease, Cronh's disease, ulcerative colitis, hyperimmunoglobulinemia D, periodic fever syndrome, Cryopyrin-associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, deficiency of IL-1 receptor antagonist, Alzheimer's disease, Parkinson's disease, and cancer.

The compounds, or pharmaceutically acceptable salts thereof described herein may be used to decrease the expression or activity of IRAK4, or to otherwise affect the properties and/or behavior of IRAK4 polypeptides or polynucleotides, e.g., stability, phosphorylation, kinase activity, interactions with other proteins, etc. in a cell.

One embodiment of the present disclosure includes a method of decreasing the expression or activity of IRAK4, or to otherwise affect the properties and/or behavior of IRAK4 polypeptides or polynucleotides in a subject comprising administering to said subject an effective amount of at least one compound described herein, or a pharmaceutically acceptable salt thereof.

One embodiment of the present disclosure includes a method for treating an inflammatory disease in a subject, the method comprising administering to the subject a therapeutically effective amount of a compound described herein, or a pharmaceutically acceptable salt thereof, thereby treating the inflammatory disease in the subject.

In one embodiment, the inflammatory disease is a pulmonary disease or a disease of the airway.

In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout.

One embodiment of the present disclosure includes a method for treating an autoimmune disease, cancer, cardiovascular disease, a disease of the central nervous system, a disease of the skin, an ophthalmic disease and condition, and bone disease in a subject, the method comprising administering to the subject a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the autoimmune disease is selected from rheumatoid arthritis, systemic lupus erythematosus, neuropsychiatric lupus, multiple sclerosis, neuromyelitis optica, diabetes, systemic sclerosis, and Sjogren's syndrome.

In one embodiment, the autoimmune disease is type 1 diabetes.

In one embodiment, the cancer is selected from Waldenstrim's macroglobulinemia, solid tumors, skin cancer, and lymphoma.

In one embodiments, the cancer is selected from lymphoma, leukemia, and myelodysplastic syndrome.

In one embodiment, the leukemia is acute myelogenous leukemia (AML) or chronic lymphocytic leukemia (CLL), and the lymphoma is non-Hodgkin's Lymphoma (NHL), small lymphocytic lymphoma (SLL), macroglobulinemia/lymphoplasmacytic lymphoma (WM/LPL), or DLBC lymphomas.

In one embodiment, the cardiovascular disease is selected from stroke and atherosclerosis.

In one embodiment, the disease of the central nervous system is a neurodegenerative disease.

In one embodiment, the disease of the skin is selected from rash, contact dermatitis, psoriasis, and atopic dermatitis.

In one embodiment, the bone disease is selected from osteoporosis and osteoarthritis.

In one embodiment, the inflammatory bowel disease is selected from Crohn's disease and ulcerative colitis.

One embodiment of the present disclosure includes a method for treating an ischemic fibrotic disease, the method comprising administering to the subject a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the present disclosure includes a method for treating post-organ transplantation fibrosis, the method comprising administering to the subject a therapeutically effective amount of a compound described herein, or a pharmaceutically acceptable salt thereof, thereby treating post-organ transplantation fibrosis in the subject.

One embodiment of the present disclosure includes a method for treating hypertensive or diabetic end organ disease in a subject, the method comprising administering to the subject a therapeutically effective amount of a compound described herein, or a pharmaceutically acceptable salt thereof, thereby treating hypertensive or diabetic end organ disease in the subject.

One embodiment of the present disclosure includes a method for treating hypertensive kidney disease in a subject, the method comprising administering to the subject a therapeutically effective amount of a compound described herein, or a pharmaceutically acceptable salt thereof, thereby treating hypertensive kidney disease in the subject.

One embodiment of the present disclosure includes a method for treating idiopathic pulmonary fibrosis (IPF) in a subject, the method comprising administering to the subject a therapeutically effective amount of a compound described herein, or a pharmaceutically acceptable salt thereof, thereby treating IPF in the subject.

One embodiment of the present disclosure includes a method for treating scleroderma or systemic sclerosis in a subject, the method comprising administering to the subject a therapeutically effective amount of a compound described herein, or a pharmaceutically acceptable salt thereof, thereby treating scleroderma or systemic sclerosis in the subject.

One embodiment of the invention includes a method for treating liver cirrhosis in a subject, the method comprising administering to the subject a therapeutically effective amount of a compound described herein, or a pharmaceutically acceptable salt thereof, thereby treating liver cirrhosis in the subject.

One embodiment of the invention includes a method for treating fibrotic diseases in a subject wherein tissue injury and/or inflammation are present, the method comprising administering to the subject a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys.

It is contemplated that the present disclosure, methods and compositions described herein can be used as an antifibrotic, or used to treat, prevent, and/or reduce the severity and damage from fibrosis.

It is additionally contemplated that the present disclosure, methods and compositions described herein can be used to treat, prevent, and/or reduce the severity and damage from fibrosis.

It is further contemplated that the present disclosure, methods and compositions described herein can used as an anti-inflammatory, used to treat inflammation.

Some non-limiting examples of organs include: kidney, hearts, lungs, stomach, liver, pancreas, hypothalamus, stomach, uterus, bladder, diaphragm, pancreas, intestines, colon, and so forth.

In certain embodiments, the present disclosure relates to the aforementioned methods, wherein said compound is administered parenterally.

In certain embodiments, the present disclosure relates to the aforementioned methods, wherein said compound is administered intramuscularly, intravenously, subcutaneously, orally, pulmonary, rectally, intrathecally, topically or intranasally.

In certain embodiments, the present disclosure relates to the aforementioned methods, wherein said compound is administered systemically.

In certain embodiments, the present disclosure relates to the aforementioned methods, wherein said subject is a mammal.

In certain embodiments, the present disclosure relates to the aforementioned methods, wherein said subject is a primate.

In certain embodiments, the present disclosure relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”.

The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two.

Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog RS system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

Pharmacology and Utility

Compounds of the present disclosure have been found to modulate IRAK4 activity and may be beneficial for the treatment of neurological, neurodegenerative and other additional diseases

Another aspect of the invention provides a method for treating or lessening the severity of a disease, disorder, or condition associated with the modulation of IRAK4 in a subject, which comprises administering to the subject a compound of Formula (I) or a pharmaceutically acceptable salt thereof.

In certain embodiments, the present invention provides a method of treating a condition, disease or disorder implicated by a deficiency of IRAK4 activity, the method comprising administering a composition comprising a compound of Formula (I) to a subject, preferably a mammal (e.g., a human), in need of treatment thereof.

As used herein, an “effective amount” and a “therapeutically effective amount” can used interchangeably. It means an amount effective for treating or lessening the severity of one or more of the diseases, disorders or conditions as recited above.

The compounds and compositions, according to the methods of the present disclosure, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

Combination Therapy

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present disclosure includes the use of a combination of an IRAK inhibitor compound as provided in the compound of formula (I) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of formula (I) or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present disclosure and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present disclosure that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the disclosure may be administered with the following therapeutic agents: Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, prarnipexole, bromocriptine, pergolide, trihexephendyl, and amantadine; agents for treating Multiple Sclerosis (MS) such as Tecfidera® and beta interferon (e.g., Avonex® and Rebif), Copaxone®, and mitoxantrone; treatments for asthma such as albuterol and Singulair©; agents for treating schizophrenia such as zyprexa, risperdal, seroquel, and haloperidol; anti-inflammatory agents such as corticosteroids, T F blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3 A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” or “haloalkyl” refers to an alkyl group having at least one halogen substitution.

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC_{1-_4} alkyl” refers to a C₁₋₄ allkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

“Halogen” or “halo” may be fluorine, chlorine, bromine or iodine (preferred halogens as substituents are fluorine and chlorine).

As used herein, the term “halo-substituted-C₁₋₄alkyl” or “C₁₋₄haloalkyl” refers to a C₁₋₄alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “carbocyclic ring” refers to a nonaromatic hydrocarbon ring that is either partially or fully saturated and may exist as a single ring, bicyclic ring (including fused, spiral or bridged carbocyclic rings) or a spiral ring. Unless specified otherwise, the carbocyclic ring generally contains 4- to 7-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a monocyclic ring which is fully saturated which has 4 to 7 ring atoms and which contains 1 to 2 heteroatoms, independently selected from sulfur, oxygen and/or nitrogen. Exemplary heterocyclyl group includes oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl. In some embodiments, the heterocyclyl group is a 4 to 6 membered heterocyclyl group. In some embodiments, a heterocyclyl group contains at least one oxygen ring atom. In some embodiments, a heterocyclyl group is selected from oxtanyl, tetrahydrofuranyl, 1,4-dioxanyl and tetrahydropyranyl.

As used herein the term “spiral” ring means a two-ring system wherein both rings share one common atom. Examples of spiral rings include 5-oxaspiro[2.3]hexane, oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. bicyclo[1.1.1]pentane, bicyclo [2.2.1] heptane and bicyclo [3.2.1] octane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of formula (I), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one embodiment, the present disclosure provides a compound of the Examples as an isolated stereoisomer wherein the compound has one stereocenter and the stereoisomer is in the R configuration.

In one embodiment, the present disclosure provides a compound of the Examples as an isolated stereoisomer wherein the compound has one stereocenter and the stereoisomer is in the S configuration.

In one embodiment, the present disclosure provided a compound of the Examples as an isolated stereoisomer wherein the compound has two stereocenters and the stereoisomer is in the R R configuration.

In one embodiment, the present disclosure provided a compound of the Examples as an isolated stereoisomer wherein the compound has two stereocenters and the stereoisomer is in the R S configuration.

In one embodiment, the present disclosure provided a compound of the Examples as an isolated stereoisomer wherein the compound has two stereocenters and the stereoisomer is in the S R configuration.

In one embodiment, the present disclosure provided a compound of the Examples as an isolated stereoisomer wherein the compound has two stereocenters and the stereoisomer is in the S S configuration.

In one embodiment, the present disclosure provided a compound of the Examples, wherein the compound has one or two stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the formula (I) as defined herein, in free form. In another embodiment, the present disclosure relates to a compound of the formula (I) as defined herein, in salt form. In another embodiment, the present disclosure relates to a compound of the formula (I) as defined herein, in acid addition salt form. In a further embodiment, the present disclosure relates to a compound of the formula (I) as defined herein, in pharmaceutically acceptable salt form. In yet a further embodiment, the present disclosure relates to a compound of the formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Furthermore, the compounds of the present disclosure, including their salts, may also be obtained in the form of their hydrates, or include other solvents used for their crystallization. The compounds of the present disclosure may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the present disclosure, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

The compounds of the present disclosure, including salts, hydrates and solvates thereof, may inherently or by design form polymorphs.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present disclosure having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization.

Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers.

Enantiomers can also be separated by use of a commercially available chiral HPLC column.

The present disclosure further includes any variant of the present processes, in which the reaction components are used in the form of their salts or optically pure material.

Compounds of the invention and intermediates can also be converted into each other according to methods generally known to those skilled in the art.

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### EXEMPLIFICATION

### Abbreviation

CO=carbon monoxide
PE=petroleum ether
EtOAc=ethyl acétate
ESI=electrospray ionisation
MeOH=methanol
EtOH=ethanol
TEA=Triethylamine
T3P®=Propanephosphonic acid anhydride
DCM=dichloromethane
DEA=diethylamine
DIAD=Diisopropyl azodicarboxylate
DMF=dimethylformamide
DPPA=Diphenylphosphoryl azide
EDCI=1-Ethyl-3-(3-dimethylaminopropyl)carbodiimide
HATU=Hexafluorophosphate Azabenzotriazole Tetramethyl Uronium
HBr=hydrobromic acid
HCl=hydrochloric acid
NBS=N-bromosuccinimide
NIS=N-iodosuccinimide
LCMS=liquid chromatography mass spectrometry
HPLC=high pressure liquid chromatography
RED=Rapid Equilibrium Dialysis
THF=tetrahydrofuran
MeCN=ACN=acetonitrile
DMSO=dimetylsulfoxide
AcOH=acetic acid
CDI=carbonyldiimidazole
DMAP=4-dimethylaminopyridine
TFA=trifluoroacetic acid
DIPEA=diisopropylethyl amine
TLC=Thin Layer Chromatography
SFC=Supercritical Fluid Chromatography
Na=Sodium
Ar=Argon
N₂=Nitrogen
μW=microwave
SFC=Supercritical Fluid Chromatography
MBPR=Manual Back Pressure Regulator
ABPR=Automatic Back Pressure Regulator
RPHPLC=Reverse Phase HPLC
KOH=Potassium Hydroxide
NH₄HCO₃=Ammonium Bicarbonate
CO₂=Carbon Dioxide
NH₄OH=Ammonium Hydroxide
Hunigs Base=N,N-diisopropylethylamine
tBuOH=tert-butanol
H₂O₂=hydrogen peroxide
SOCl₂=thionyl chloride
BuLi=n-butyl lithium
NH₄Cl=ammonium chloride
MeLi=methyl lithium
TMSCHN₂=trimethylsilyldiazomethane
MgSO₄=magnesium sulfate
tBuOK=potassium tert-butoxide
NaH=sodium hydride
Xantphos=4,5-Bis(diphenylphosphino)-9,9-dimethylxanthene
Et₂O=diethylether
LiOH·H₂O=lithium hydroxide hydrate
Na2SO4=sodium sulfate
NaHCO₃=sodium bicarbonate
Pd(OAc)₂=Palladium (II) Acetate
NaOH=Sodium Hydroxide
NaCl=Sodium Chloride
XantPhos-Pd-G3=[(4,5-Bis(diphenylphosphino)-9,9-dimethylxanthene)-2-(2′-amino-1,1′-biphenyl)]palladium(II) methanesulfonate
Pd/C=Palladium on Carbon
Pd(dppf)Cl₂ DCM=[1,1′-Bis(diphenylphosphino)ferrocene]dichloropalladium(II), complex with dichloromethane
Pd(dppf)Cl₂=[1,1′-Bis(diphenylphosphino)ferrocene]dichloropalladium(II)

General Methods:

The compounds of the Examples were analyzed or purified according to one of the Purification Methods referred to below unless otherwise described.

Where preparative TLC or silica gel chromatography have been used, one skilled in the art may choose any combination of solvents to purify the desired compound. Silica gel column chromatography was performed using 20-40 μM (particle size), 250-400 mesh, or 400-632 mesh silica gel using either a Teledyne ISCO Combiflash RF or a Grace Reveleris X2 with ELSD purification systems or using pressurized nitrogen (˜10-15 psi) to drive solvent through the column (“flash chromatography”).

Wherein an SCX column has been used, the eluant conditions are MeOH followed by methanolic ammonia.

Except where otherwise noted, reactions were run under an atmosphere of nitrogen. Where indicated, solutions and reaction mixtures were concentrated by rotary evaporation under vacuum.

Analytical Methods

ESI-MS data (also reported herein as simply MS) were recorded using Waters System (Acquity HPLC and a Micromass ZQ mass spectrometer); all masses reported are the m/z of the protonated parent ions unless recorded otherwise.

LC/MS:

A sample was dissolved in a suitable solvent such as MeCN, dimethyl sulfoxide (DMSO), or MeOH and was injected directly into the column using an automated sample handler. The analysis used one of the following methods: (1) acidic method (1.5, 2, 3.5, 4, or 7 min runs, see Acidic LCMS section for additional details vide infra: conducted on a Shimadzu 2010 Series, Shimadzu 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L of trifluoroacetic acid (TFA) in water (solvent A) and 0.75 mL/4 L of TFA in MeCN (solvent B) or (2) basic method (3, 3.5, 7 min runs, see Basic LCMS section for additional details vide infra: conducted on a Shimadzu 2020 Series or Waters Acquity UPLC BEH (MS ionization: ESI) instrument equipped with XBridge Shield RP18, 5 um column (2.1 mm×30 mm, 3.0 mm i.d.) or 2.1 mm×50 mm, C18, 1.7 μm column, eluting with 2 mL/4 L NH₃·H₂O in water (solvent A) and MeCN (solvent B).

The disclosure further includes any variant of the present processes, in which the reaction components are used in the form of their salts or optically pure material. Compounds of the disclosure and intermediates can also be converted into each other according to methods generally known to those skilled in the art.

SFC Analytical Separation

Instrument: Waters UPC2 analytical SFC (SFC-H). Column: ChiralCel OJ, 150×4.6 mm I.D., 3 μm. Mobile phase: A for CO2 and B for Ethanol (0.05% DEA). Gradient: B 40%. Flow rate: 2.5 mL/min. Back pressure: 100 bar. Column temperature: 35° C. Wavelength: 220 nm.

Detectors: Gilson UV/VIS-156 with UV detection at 220/254 nm, Gilson 281 automatic collection, utilizing acidic, basic and neutral methods. For mass-directed peak collection, an ACQUITY QDa Mass Detector (Waters Corporation) was employed.

Preparative SFC Purification

Instrument: MG III preparative SFC (SFC-1). Column: ChiralCel OJ, 250×30 mm I.D., 5 μm. Mobile phase: A for C₀₂ and B for Ethanol (0.1% NH3H2O). Gradient: B 50%. Flow rate: 40 mL/min. Back pressure: 100 bar. Column temperature: 38° C. Wavelength: 220 nm. Cycle time: ˜8 min.
Column: Chiralpak AD-H; 250 mm×30 mm, 5 μm; 40% (EtOH+0.1% DEA)/CO₂
Column: Chiralpak IA; 250 mm×30 mm, 5 μm; 40% (MeOH+0.1% DEA)/CO₂
Column: Chiralpak IB; 250 mm×30 mm, 5 μm; 40% (EtOH+0.1% DEA)/CO₂
Column: Chiralpak AD-H; 250 mm×30 mm, 5 μm; 40% (EtOH+0.1% NH₄OH)/CO₂
Column: Chiralpak OJ-H; 250 mm×30 mm, 5 μm; 30% (EtOH+0.1% NH₄OH)/CO₂
Column: Chiralpak OD; 250 mm×30 mm, 5 μm; 35% (EtOH+0.1% NH₄OH)/CO₂

¹H-NMR

¹H nuclear magnetic resonance (NMR) spectra were in all cases consistent with the proposed structures. The 1H NMR spectra were recorded on a Bruker Avance III HD 500 MHz, Bruker Avance III 500 MHz, Bruker Avance III 400 MHz, Varian-400 VNMRS, or Varian-400 MR. Characteristic chemical shifts (S) are given in parts-per-million downfield from tetramethylsilane (for ¹H-NMR) using conventional abbreviations for designation of major peaks: e.g. s, singlet; d, doublet; t, triplet; q, quartet; dd, double doublet; dt, double triplet; m, multiplet; br, broad. The following abbreviations have been used for common solvents: CDCl₃, deuterochloroform; DMSO-d₆, hexadeuterodimethyl sulfoxide; and MeOH-d₄, deuteron-methanol. Where appropriate, tautomers may be recorded within the NMR data; and some exchangeable protons may not be visible.

Typically, the compounds of Formula (I) can be prepared according to the schemes provided below. The following examples serve to illustrate the invention without limiting the scope thereof. Methods for preparing such compounds are described hereinafter. GENERAL SCHEMES:

Scheme 1, 2, 3, and 4 provide potential routes for making compounds of Formula (I).

Scheme 1:

According to a first process, compounds of Formula (I), may be prepared from compounds of Formulae (II′) and (III′) as illustrated by Scheme 1.

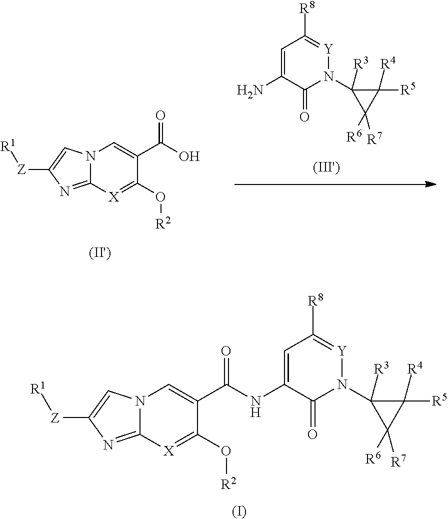

The compound of Formula (I) can be prepared by an amide bond formation of the acid of Formula (II′) and the amine of Formula (III′) in the presence of a suitable coupling agent and organic base in a suitable polar aprotic solvent. Preferred conditions, comprise reaction of the acid of Formula (II′) with the amine of Formula (III′) in the presence of a coupling agent preferably, T3P®, HATU, CDI, HOAt in the presence of EDC, optionally in the presence of N-methyl imidazole, in the presence of a suitable organic base such as TEA, DIPEA or pyridine, optionally in a suitable solvent, such as DMF, DMSO, EtOAc or MeCN at between rt and the reflux temperature of the reaction and optionally in the presence of microwave irradiation.

Scheme 2:

According to a second process, compounds of Formula (II′) can be prepared from compounds of Formulae (IV′), (V′), (VI′), (VII′), and (VIII′) as illustrated by Scheme 2.

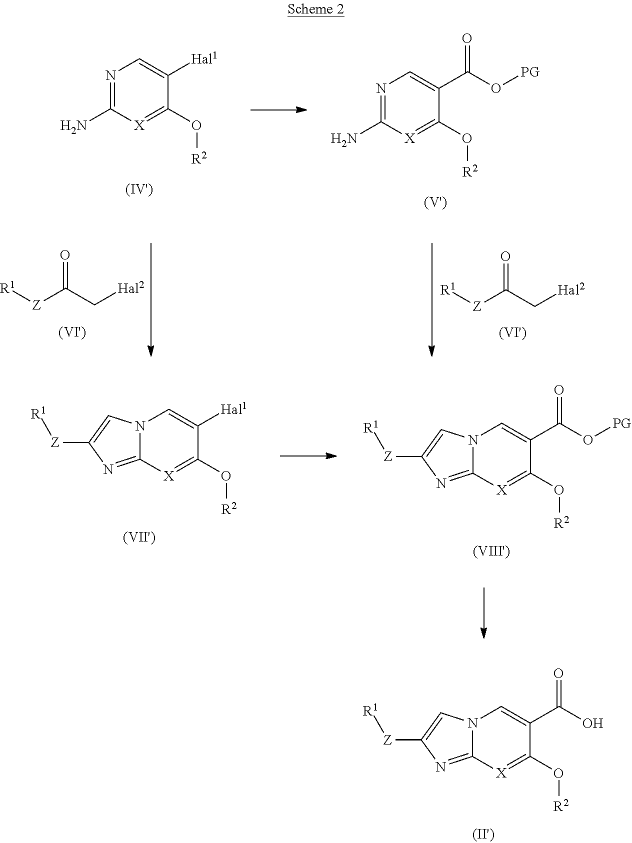

In Scheme 2, Hal¹ is halogen, preferably Br or I; Hal² is halogen, preferably Cl or Br; PG is a carboxylic acid protecting group, typically C₁-C₄ alkyl or phenyl and preferably Me, Et, isopropyl or phenyl; and the remaining variables are as defined above for Formula (I).

Compounds of Formula (V′) may be prepared from the bromide of Formula (IV′) by a palladium catalyzed carbonylation reaction, in the presence of a suitable palladium catalyst, organic base and suitable alcohol at elevated temperature under an atmosphere of CO. When PG is methyl or ethyl, preferred conditions comprise, reaction of the bromide of Formula (IV′) under an atmosphere of CO in the presence of suitable palladium catalyst such as Pd(dppf)Cl₂ or Pd(OAc)₂ with a phosphine-based ligand such PPh₃, an organic base such as TEA in a solvent such as MeOH or EtOH at between 80 and 100° C. Alternatively, when PG is phenyl, compounds of Formula (V′) may be prepared from the bromide of Formula (IV′) by a palladium catalyzed reaction with phenyl formate, in the presence of a suitable palladium catalyst such as such as Xantphos Pd-G3, or a suitable palladium catalyst such as Pd(OAc)₂ with a phosphine-based ligand such as BINAP or XantPhos, an organic base such as TEA, in a solvent such as MeCN at between 80 and 100° C.

Compounds of Formula (VII)′ may be prepared from the amine of Formula (IV′) and the haloketone of Formula (VI′) by a condensation/cyclisation reaction. Preferred conditions comprise reaction of the amine of Formula (IV′) with the haloketone of Formula (VI′) optionally in the presence of a suitable inorganic base such as K₂CO₃ or NaHCO₃ and optionally in the presence of a catalyst such as KI, in a suitable protic solvent such as MeOH, EtOH, n-BuOH, t-BuOH, MeCN or MeCN/toluene at elevated temperature, typically between 60 to 100° C.

Compounds of Formula (VIII′) may be prepared from the amine of Formula (V′) and the haloketone of Formula (VI′) by a condensation/cyclisation reaction as described above.

Alternatively, compounds of Formula (VIII′) may be prepared from the bromide of Formula (VII) by a palladium catalysed carbonylation reaction as described above.

Compounds of Formula (II′) may be prepared by the hydrolysis of the ester of Formula (VIII′) under suitable acidic or basic conditions in a suitable aqueous solvent. Preferred conditions comprise the treatment of the ester of Formula (VIII′) with an alkali metal base such as LiOH, NaOH, K₂CO₃ or Na₂CO₃ in aqueous MeOH and/or THF at between rt and the reflux temperature of the reaction.

Scheme 3:

According to a third process, compounds of Formula (VII′) may be prepared from compounds of Formulae (X′) or (IX′) as illustrated by Scheme 3.

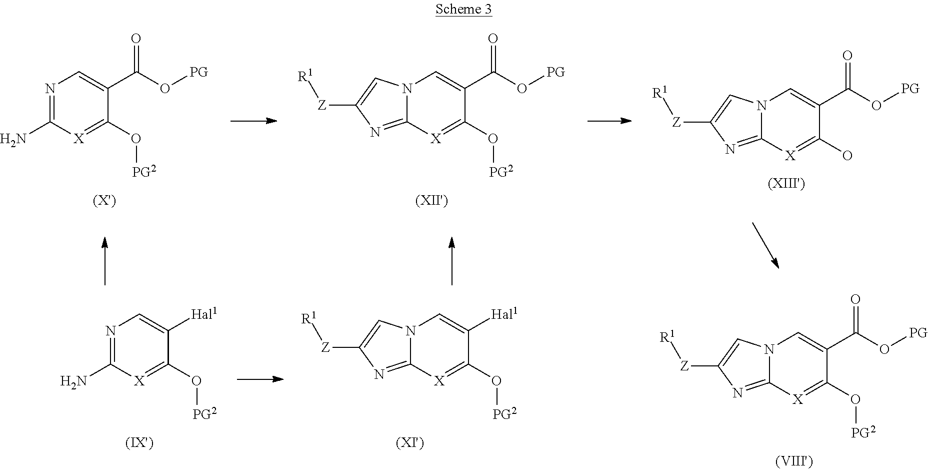

In Scheme 3, Hal¹ is halogen, preferably Br or I; PG is a carboxylic acid protecting group, typically C₁-C₄ alkyl or phenyl and preferably Me, Et, isopropyl or phenyl; PG² is an ether protecting group, typically benzyl, or possibly trialkylsilyl that can be removed orthogonally to PG; and the remaining variables are as defined above for Formula (I).

Compounds of Formula (X′) may be prepared from the bromide of Formula (IX′) by a palladium catalyzed carbonylation reaction, in the presence of a suitable palladium catalyst, organic base and suitable alcohol at elevated temperature under an atmosphere of CO. Preferred conditions comprise, reaction of the bromide of Formula (IX′) under an atmosphere of CO in the presence of suitable palladium catalyst such as Pd(dppf)Cl₂ or Pd(OAc)₂ with a phosphine-based ligand such PPh₃, an organic base such as TEA in a solvent such as MeOH or EtOH at between 80 and 100° C. Alternatively, compounds of Formula (X′) may be prepared from the bromide of Formula (IX′) by a palladium catalyzed reaction with phenyl formate, in the presence of a suitable palladium catalyst such as such as Xantphos Pd-G3, or a suitable palladium catalyst such as Pd(OAc)₂ with a phosphine-based ligand such as BINAP or XantPhos, an organic base such as TEA, in a solvent such as MeCN at between 80 and 100° C.

Compounds of Formula (XI′) or (XII′) may be prepared from the amine of Formula (IX′) or (X) and the haloketone of Formula (VI′) by a condensation/cyclization reaction as described above.

Alternatively, compounds of Formula (XII′) may be prepared from the bromide of Formula (XI′) by a palladium catalysed carbonylation reaction as described above. Compounds of Formula (XIII′) may be prepared by deprotection of PG², typically using hydrogen gas and Pd/C or transfer hydrogenation using Pd/C and ammonium formate in a protic solvent such as MeOH or EtOH.

Compounds of Formula (VIII′) may be prepared from compounds of Formula (XIII′) by Mitsunobu reaction with the appropriate R² alcohol, using a trialkyl phosphine such as triphenylphosphine, DIAD, and an aprotic solvent such as THF or toluene.

Scheme 4:

According to a fourth process, compounds of Formula (I), may be prepared from compounds of Formulae (III′), (V′), (VI′), (XIV′) and (XV′) as illustrated by Scheme 4.

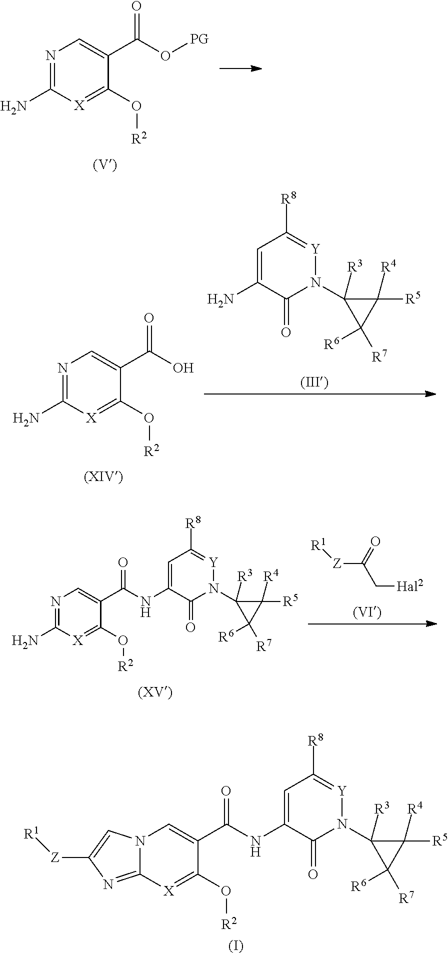

Compounds of Formula (XIV′) may be prepared by the hydrolysis of the ester of Formula (V′) under suitable acidic or basic conditions in a suitable aqueous solvent, as previously described in Scheme 2.

Compounds of Formula (XV′) may be prepared by an amide bond formation of the acid of Formula (XIV′) and the amine of Formula (III′) in the presence of a suitable coupling agent as previously described in Scheme 1.

Compounds of Formula (I) may be prepared from the compound of Formula (XV′) and the haloketone of Formula (VI′) by a condensation/cyclisation reaction as previously described in Scheme 2.

It will be appreciated by those skilled in the art that the experimental conditions set forth in the schemes that follow are illustrative of suitable conditions for effecting the transformations shown, and that it may be necessary or desirable to vary the precise conditions employed for the preparation of the compound of Formula (I). It will be further appreciated that it may be necessary or desirable to carry out the transformations in a different order from that described in the schemes, or to modify one or more of the transformations, to provide the desired compound of the invention.

### Preparation of Intermediates

### Preparation 1: 5-Bromo-4-isopropoxypyridin-2-amine

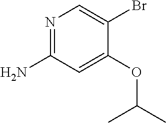

5-Bromo-4-chloro-pyridin-2-amine (50.0 g, 241.0 mmol) was added to a solution of Na (13.85 g, 602.5 mmol) in isopropanol (500 mL) and the reaction heated at 82° C. for 92 h. The reaction mixture was cooled to rt and poured into ice. The resulting precipitate was filtered off, washed with water and dried to afford the title compound as a yellow solid, 43.5 g, 76.5% yield. LCMS m/z=231 [M+H]⁺.

### Preparation 2: Methyl 6-amino-4-isopropoxynicotinate

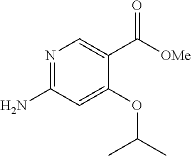

A mixture of 5-bromo-4-isopropoxy-pyridin-2-amine (Preparation 1, 25.0 g, 108.2 mmol), TEA (18.0 mL, 129.8 mmol) and Pd(dppf)Cl₂ (2.37 g, 3.25 mmol) in MeOH (300 mL) was heated at 120° C. under a 40 atm. CO pressure for 48 h. The cooled mixture was concentrated in vacuo and the residue diluted with water (100 mL). The mixture was extracted with EtOAc (2×100 mL), the combined organic extracts dried over Na₂SO₄ and evaporated under reduced pressure to afford methyl 6-amino-4-isopropoxynicotinate (21.0 g, 89.5% yield) as a brown solid. LCMS m/z=211.1 [M+H]^{+ 1}H NMR (500 MHz, CDCl₃) δ: 1.38 (d, 6H), 3.81 (s, 3H), 4.55-4.59 (m, 1H), 4.97 (br s, 1H), 5.93 (s, 1H), 8.54 (s, 1H).

### Preparation 3: Methyl 7-isopropoxy-2-(1-methyl-2-oxabicyclo[2.1.1]hexan-4-yl)imidazo[1,2-a]pyridine-6-carboxylate

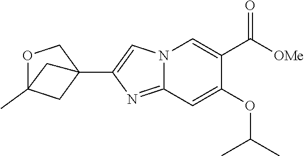 [SMILES: COC(=O)c1cn2cc(C34COC(C)(C3)C4)nc2cc1OC(C)C]

To a solution of methyl 6-amino-4-isopropoxynicotinate [Preparation 2] (1.20 gg, 5.71 mmol) and 2-bromo-1-(1-methyl-2-oxabicyclo[2.1.1]hexan-4-yl)ethan-1-one [preparation 12](1.25 g, 5.71 mmol) in MeCN (7.0 mL) and toluene (7.0 mL) was added NaHCO₃ (1.44 g, 17.1 mmol). The mixture was stirred at 90° C. for 18 h and then cooled to rt. Silica gel and MeOH (10 mL) were added and the mixture was concentrated and dry loaded into a column for purification by silica gel chromatography: eluting through a 40 g silica column with a solvent gradient of 0-50% of solvent A (3:1 EtOAc:EtOH) and solvent B: heptane to provide methyl 7-isopropoxy-2-(1-methyl-2-oxabicyclo[2.1.1]hexan-4-yl)imidazo[1,2-a]pyridine-6-carboxylate, 1.20 g, 3.63 mmol) 63% yield. LCMS m/z=331.2 [M+H]⁺.

### Preparation 4: 7-isopropoxy-2-(1-methyl-2-oxabicyclo[2.1.1]hexan-4-yl)imidazo[1,2-a]pyridine-6-carboxylic acid

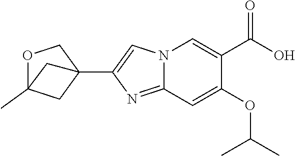 [SMILES: CC(C)Oc1cc2nc(C34COC(C)(C3)C4)cn2cc1C(=O)O]

A solution of methyl 7-isopropoxy-2-(1-methyl-2-oxabicyclo[2.1.1]hexan-4-yl)imidazo[1,2-a]pyridine-6-carboxylate (Preparation 3, 160.5 mg, 0.486 mmol) and NaOH (25.2 mg, 0.632 mmol) in H₂O (2 mL) and MeOH (3 mL) were stirred at rt for 24 h. HCl (10 M, 63.15 μL) was added and the mixture evaporated under reduced pressure to afford 7-isopropoxy-2-(1-methyl-2-oxabicyclo[2.1.1]hexan-4-yl)imidazo[1,2-a]pyridine-6-carboxylic acid, containing NaCl as a white solid. LCMS m/z=317.2 [M+H]⁺.

### Preparation 5: methyl 2-amino-4-isopropoxypyrimidine-5-carboxylate

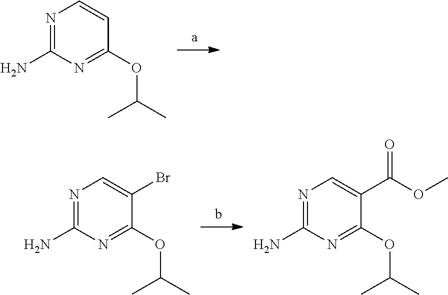

Step a: A mixture of 4-isopropoxypyrimidin-2-amine (5.90 g, 38.5 mmol) and NBS (6.86 g, 38.5 mmol) in CHCl₃ (257 mL) was stirred at rt for 18 h. The mixture was washed with aq. NaHCO₃ soln. and the organic layer evaporated under reduced pressure to afford 5-bromo-4-isopropoxypyrimidin-2-amine. LCMS m/z=232.0 [M+H]^{+ 1}H NMR (500 MHz, MeOH-d₄) S: 1.35 (d, 6H), 5.39 (dq, 1H), 8.00 (s, 1H).

Step b: To a stirred mixture of 5-bromo-4-isopropoxypyrimidin-2-amine (126.3 g, 152 mmol) in MeOH (1200 mL) in a steel bomb were added Pd(dppf)Cl₂ (8.9 g, 10.8 mmol), TEA (60.6 g, 599 mmol, 83.5 mL) at room temperature. Then CO gas was purged into the steel bomb and the stirring was continued at 120° C. for 18 hours. The reaction mixture was allowed to warm up to room temperature and filtered through a pad of celite. The celite pad was washed with excess of methanol and the filtrate was concentrated under vacuum. The residue was washed with water, rinsed with MeOH and dried to afford methyl 2-amino-4-isopropoxypyrimidine-5-carboxylate (59 g, 51.3%). ¹H NMR (METHANOL-d4, 400 MHz) δ 8.58 (s, 1H), 5.49 (td, 1H, J=6.2, 12.4 Hz), 3.81 (s, 3H), 1.37 (d, 6H, J=6.3 Hz).

### Preparation 6: 6-Bromo-7-isopropoxy-2-(1-methyl-2-oxabicyclo[2.1.1]hexan-4-yl)imidazo[1,2-a]pyrimidine

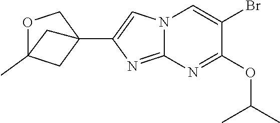 [SMILES: CC(C)Oc1nc2nc(C34COC(C)(C3)C4)cn2cc1Br]

To a solution of 2-bromo-1-(1-methyl-2-oxabicyclo[2.1.1]hexan-4-yl)ethan-1-one [preparation 12] (25.6 g, 117 mmol) in toluene (140 mL) and MeCN (140 mL), 5-bromo-4-isopropoxypyrimidin-2-amine (preparation 5 step A, 27.1 g, 117 mmol) and NaHCO₃ (29.4 g, 350 mmol) were added and the reaction mixture was stirred at 95° C. (overhead stirring) for 18 h. The cooled reaction mixture was filtered through Celite® and the filtrate concentrated in vacuo. The residue was purified by silica gel chromatography (heptane/EtOAc 100/0 to 20/80) to afford 6-bromo-7-isopropoxy-2-(1-methyl-2-oxabicyclo[2.1.1]hexan-4-yl)imidazo[1,2-a]pyrimidine as an orange solid (19.7 g, 48%). ¹H NMR (500 MHz, CDCl₃) δ: 1.44 (d, 6H) 1.53 (s, 3H) 1.93 (dd, 2H) 2.07 (s, 2H) 4.05 (s, 2H), 5.40-5.58 (m, 1H), 7.10 (s, 1H) 8.35 (s, 1H).

### Preparation 7: Phenyl 7-isopropoxy-2-(1-methyl-2-oxabicyclo[2.1.1]hexan-4-yl)imidazo[1,2-a]pyrimidine-6-carboxylate

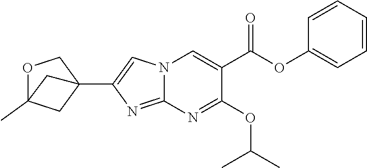 [SMILES: CC(C)Oc1nc2nc(C34COC(C)(C3)C4)cn2cc1C(=O)Oc1ccccc1]

TEA (22.0 mL, 0.16 mol) was added to a mixture of 6-bromo-7-isopropoxy-2-(1-methyl-2-oxabicyclo[2.1.1]hexan-4-yl)imidazo[1,2-a]pyrimidine (Preparation 6, 20.4 g, 57.9 mmol), Pd(OAc)₂ (1.30 g, 5.79 mmol), Xantphos (4.00 g, 6.91 mmol) and phenyl formate (18.0 g, 0.15 mol) in MeCN (120 mL) at rt and the reaction stirred at reflux overnight. The cooled mixture was filtered through Celite® and the filtrate concentrated in vacuo. The crude material was purified by silica gel chromatography (DCM/MeOH 100/0 to 95/5) to afford phenyl 7-isopropoxy-2-(1-methyl-2-oxabicyclo[2.1.1]hexan-4-yl)imidazo[1,2-a]pyrimidine-6-carboxylate as a brown oil (20.0 g, 88% yield). LCMS m/z=394.0 [M+H]^{+ 1}H NMR (500 MHz, CDCl₃) δ: 1.46 (d, 6H), 1.54 (s, 3H), 1.96 (dd, 2H), 2.07-2.17 (m, 2H), 4.08 (s, 2H), 5.63-5.65 (m, 1H), 7.18-7.51 (m, 6H), 9.04 (s, 1H).

### Preparation 8: 7-Isopropoxy-2-(1-methyl-2-oxabicyclo[2.1.1]hexan-4-yl)imidazo[1,2-a]pyrimidine-6-carboxylic acid

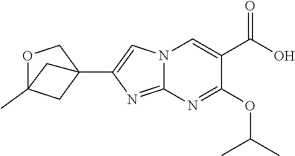 [SMILES: CC(C)Oc1nc2nc(C34COC(C)(C3)C4)cn2cc1C(=O)O]

LiOH·H₂O (2.55 g, 60.8 mmol) was added to a solution of phenyl 7-isopropoxy-2-(1-methyl-2-oxabicyclo[2.1.1]hexan-4-yl)imidazo[1,2-a]pyrimidine-6-carboxylate (Preparation 7, 20.0 g, 50.8 mmol) in THF (80 mL) and water (6 mL) and the reaction was stirred at rt overnight. 4 M HCl in dioxane (2.0 mL, 65.8 mmol) was added, the organic solvents were removed and the aqueous residue was stirred with heptanes: Et₂O 1:1 (100 mL) and then decanted. Et₂O (150 mL) and MeCN (50 mL) were added, the suspension was stirred for 2 h and the phases separated. The resulting precipitate was filtered off and washed with Et₂O to give 7-isopropoxy-2-(1-methyl-2-oxabicyclo[2.1.1]hexan-4-yl)imidazo[1,2-a]pyrimidine-6-carboxylic acid as an off-white solid (10.7 g, 57%). LCMS m/z=318.2 [M+H]⁺.

### Preparation 9: Methyl 6-amino-4-cyclobutoxynicotinate

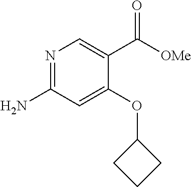 [SMILES: COC(=O)c1cnc(N)cc1OC1CCC1]

Part A: NaH (60% dispersion in mineral oil, 39.15 g, 979 mmol) was added in portions to a solution of cyclobutanol (70.47 g, 979 mmol) in DMF (1 L) at rt and stirred until H₂ evolution ceased. 5-Bromo-4-chloropyridin-2-amine (193.4 g, 932 mmol) was added and the resulting solution stirred at 100° C. for 24 h. The cooled reaction mixture was diluted with water (4 L) and extracted with EtOAc (2×500 mL). The combined organics were washed with H₂O (4×300 mL), dried (Na₂SO₄) and evaporated to dryness in vacuo. The solid residue was crystallized from benzene to give 5-bromo-4-cyclobutoxypyridin-2-amine (142.7 g, 63%).

Part B. 5-Bromo-4-cyclobutoxypyridin-2-amine (142.7 g, 587 mmol), TEA (65.2 g, 646 mmol) and Pd(dppf)Cl2·DCM (14.38 g, 17.6 mmol) were dissolved in dry MeOH (800 mL) and the reaction heated to 140° C. at 40 bar of CO for 12 h. The cooled mixture was concentrated in vacuo, the mixture poured into water (1 L) and extracted with EtOAc (3×200 mL). The combined organics were dried (Na₂SO₄) and evaporated to dryness in vacuo. The residue was recrystallised from isopropanol to afford methyl 6-amino-4-cyclobutoxynicotinate (79.5 g, 61% yield). H NMR (METHANOL-d4, 400 MHz) δ 8.37 (s, 1H), 5.97 (s, 1H), 4.75 (t, 1H, J=7.2 Hz), 3.82 (s, 3H), 2.5-2.6 (m, 2H), 2.2-2.3 (m, 2H), 1.7-2.0 (m, 2H).

### Preparation 10: Isopropyl 2-amino-4-isopropoxypyrimidine-5-carboxylate

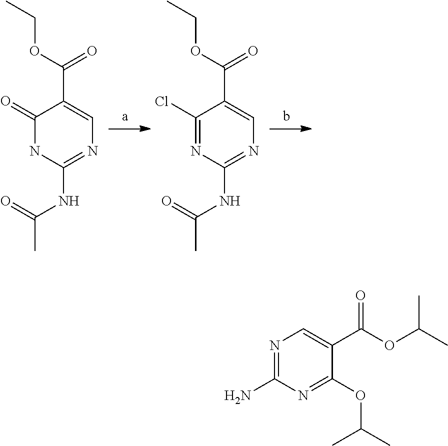 [SMILES: CCOC(=O)c1cnc(NC(C)=O)[nH]c1=O ; CCOC(=O)c1cnc(NC(C)=O)nc1Cl ; CC(C)OC(=O)c1cnc(N)nc1OC(C)C]

Part A: To a suspension of ethyl 2-amino-6-oxo-1,6-dihydropyrimidine-5-carboxylate (71 g, 387 mmol) in AcOH (1.5 L) was added acetic anhydride (73 mL, 773 mmol) and the reaction stirred at reflux for 18 h. The cooled mixture was filtered and the solid washed with hexane and dried at 60° C. for 24 h to afford ethyl 2-acetamido-6-oxo-1,6-dihydropyrimidine-5-carboxylate (80 g, 92% yield).

Part B: ethyl 2-acetamido-6-oxo-1,6-dihydropyrimidine-5-carboxylate (80 g, 356 mmol) was dissolved in POCl₃ (800 mL) and the reaction mixture was heated to 60° C. for 16 h. The excess of POCl₃ was evaporated in vacuum then the residue was poured into ice. The mixture was extracted with dichloromethane and the combined organic layers were dried over anhydrous Na₂SO₄ and evaporated under reduced pressure to obtained 87 g of crude residue containing ethyl 2-acetamido-4-chloropyrimidine-5-carboxylate (85% purity).

Part C: To a solution of Na (9.9 g, 420 mmol) in isopropanol (1500 mL) was added in portion ethyl 2-acetamido-4-chloropyrimidine-5-carboxylate (85% purity) (30 g, 123 mmol) at 20° C. The reaction mixture was stirred for 12 h at r.t. The mixture was evaporated, dissolved in water, extracted with ethyl acetate, and the combined organic layers were dried over anhydrous Na₂SO₄ and evaporated under reduced pressure to obtained 12 g of crude residue contained isopropyl 2-amino-4-isopropoxypyrimidine-5-carboxylate (80% purity). The crude residue was crystallized from mixture ethyl acetate/hexane to afford 4.4 g of isopropyl 2-amino-4-isopropoxypyrimidine-5-carboxylate.

### Preparation 11a: methyl (S)-2-amino-4-(sec-butoxy)pyrimidine-5-carboxylate

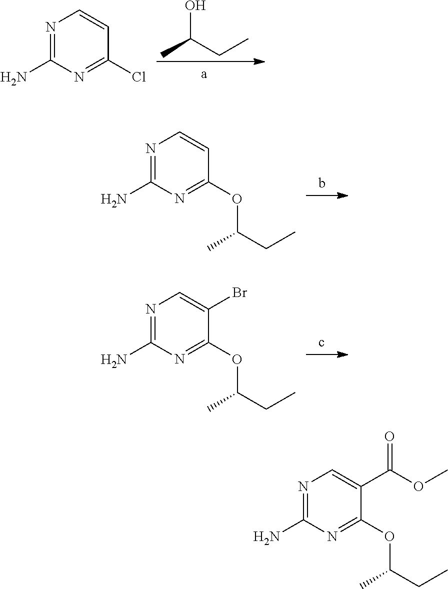

Part A: t-BuOK (3.02 g, 27 mmol) was added portion-wise to a solution of (R)-butan-2-ol (2.13 g, 29 mmol) in DMSO (20 mL) with vigorous stirring and internal cooling with ice. The mixture was stirred at r.t. for 30 min before an addition of 4-chloropyrimidin-2-amine (2.32 g, 17 mmol) and the resultant solution was heated to 90° C. and stirred overnight. The mixture was cooled down to rt, poured into water and extracted with EtOAc. The organic layer was washed with water, brine, dried over Na₂SO₄ and evaporated in vacuo. The crude residue was purified by silica gel chromatography to obtain (S)-4-(sec-butoxy)pyrimidin-2-amine (2.1 g, 70% yield).

Part B: (S)-4-(sec-butoxy)pyrimidin-2-amine (2.13 g, 29 mmol) was dissolved in CH₂Cl₂ (50 mL), followed by an addition of NBS (2.47 g, 14 mmol), portion-wise at 0-10° C. Upon completion, the mixture was allowed to warm to rt and stirred for 2 h and then was diluted with water. The organic layer was washed with water, brine, dried over Na2SO₄ filtered and evaporated in vacuo to afford (S)-5-bromo-4-(sec-butoxy)pyrimidin-2-amine (2.9 g, 94% yield).

Part C: To a mixture of (S)-5-bromo-4-(sec-butoxy)pyrimidin-2-amine (2.9 g, 15.2 mmol) in MeOH (70 mL), stirred in a steel bomb, were added Pd(dppf)Cl₂ (0.89 g, 0.108 mmol) and TEA (1.46 g, 14.4 mmol) at r.t. and after that the steel vessel was tightly closed. Then CO gas was purged into the steel bomb and the stirring was continued at 120° C. for 18 hours. The reaction mixture was allowed to cool down to room temperature and filtered through a pad of celite. The celite pad was washed with excess of methanol and the filtrate was concentrated under vacuum. The residue was diluted with water and extracted with EtOAc. The organic layer was washed with water, brine, dried over Na₂SO₄ and evaporated in vacuo. The crude material was purified by silica gel chromatography to afford the target methyl (S)-2-amino-4-(sec-butoxy)pyrimidine-5-carboxylate (1.9 g, 70% yield). ¹H NMR (400 MHz, DMSO-d₆) δ 8.51 (d, J=1.9 Hz, 1H), 7.29 (s, 2H), 5.20 (qd, J=6.1, 1.9 Hz, 1H), 3.69 (d, J=1.9 Hz, 3H), 1.63 (qd, J=7.4, 6.3, 3.7 Hz, 2H), 1.25 (dd, J=6.2, 1.9 Hz, 3H), 0.91 (t, J=7.4, 1.9 Hz, 3H).

### Preparation 11b: methyl (R)-2-amino-4-(sec-butoxy)pyrimidine-5-carboxylate

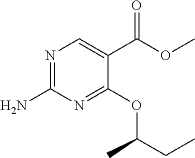 [SMILES: CC[C@@H](C)Oc1nc(N)ncc1C(=O)OC]

Methyl (R)-2-amino-4-(sec-butoxy)pyrimidine-5-carboxylate was obtained following the procedure described in Preparation 11a starting from (S)-butan-2-ol (2.5 g, 56% yield). ¹H NMR (400 MHz, DMSO-d₆) δ 8.50 (s, 1H), 7.28 (s, 2H), 5.19 (q, J=6.2 Hz, 1H), 3.68 (d, J=1.4 Hz, 3H), 1.67-1.56 (m, 2H), 1.25 (d, J=6.2 Hz, 3H), 0.91 (t, J=7.5 Hz, 3H).

### Preparation 12: 2-Bromo-1-(1-methyl-2-oxabicyclo[2.1.1]hexan-4-yl)ethan-1-one

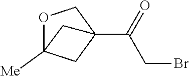 [SMILES: CC12CC(C(=O)CBr)(CO1)C2]

Oxalyl chloride (1.19 mL, 14.1 mmol) was added to 1-methyl-2-oxabicyclo[2.1.1]hexane-4-carboxylic acid (1.00 g, 7.03 mmol) in DCM (12.0 mL) at 0° C., and the reaction stirred at rt for 18 h. The solution was evaporated under reduced pressure to provide 1-methyl-2-oxabicyclo[2.1.1]hexane-4-carbonyl chloride, which was used immediately in the next step.

TMSCHN₂ (2 M, 7.74 mL) was added to a solution of 1-methyl-2-oxabicyclo[2.1.1]hexane-4-carbonyl chloride (2.26 g, 14.1 mmol) in THF (12 mL) at 0° C. and the reaction stirred for 1.5 h at 0° C. HBr (4.78 mL, 48%, 42.2 mmol) was added drop-wise and the reaction stirred for a further 1.5 h. The reaction was diluted with EtOAc and basified with saturated aqueous NaHCO₃ to pH 9, and the layers separated. The aqueous phase was extracted with EtOAc (3×50 mL), the combined organic extracts were washed with brine (50 mL), dried over MgSO₄, filtered and evaporated under reduced pressure to afford 2-bromo-1-(1-methyl-2-oxabicyclo[2.1.1]hexan-4-yl)ethan-1-one. ¹H NMR (500 MHz, CDCl₃) δ: 1.47 (s, 3H), 1.93 (d, 2H), 2.02 (d, 2H), 3.98-4.00 (m, 4H).

### Alternative Synthesis

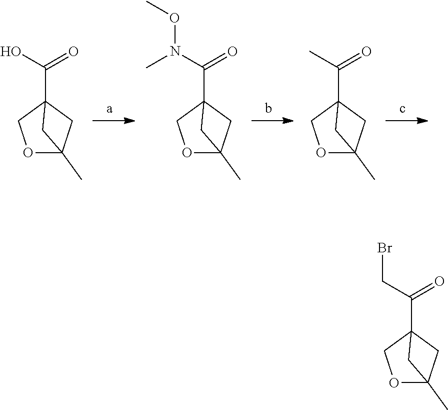

Part A: CDI (20.5 g, 127 mmol) was added portion-wise to a solution of 1-methyl-2-oxabicyclo[2.1.1]hexane-4-carboxylic acid (15.0 g, 106 mmol) in DCM (300 mL) and the mixture was stirred for 5 h at rt. N-methoxymethanamine hydrochloride (10.2 g, 106 mmol) was added and the resulting mixture was stirred at rt overnight. The reaction was poured into a mixture of water and ice and extracted with DCM (2×100 mL). The combined organics were washed with brine, dried (Na₂SO₄) and evaporated to dryness under reduced pressure to give N-methoxy-N,1-dimethyl-2-oxabicyclo[2.1.1]hexane-4-carboxamide as a yellow oil (18.2 g). LCMS m/z=186.2 [M+H]⁺.

Part B: A solution N-methoxy-N,1-dimethyl-2-oxabicyclo[2.1.1]hexane-4-carboxamide (18.20 g, 98.26 mmol) in Et₂O (150 mL) was cooled to −15° C. and 1.6 M MeLi in Et₂O (19.8 mL, 98.3 mmol) added dropwise. The reaction mixture was warmed to 0° C. for 1.5 h and then warmed to rt. The reaction was quenched with saturated aqueous NH₄Cl and extracted with Et₂O (2×50 mL). The combined organics were washed with brine, dried (Na₂SO₄) and evaporated to dryness in vacuo to afford 1-(1-methyl-2-oxabicyclo[2.1.1]hexan-4-yl)ethan-1-one as a yellow oil (13.5 g, 98%) which was used without further purification.

Part C: A solution of 1-(1-methyl-2-oxabicyclo[2.1.1]hexan-4-yl)ethan-1-one (13.5 g, 96.3 mmol) in DCM (90 mL) and MeOH (15 mL) was cooled at 0° C. and a solution of Br2 (15.4 g, 96.30 mmol) in DCM (25 mL) was added dropwise and the reaction was stirred at 0-15° C. for 2 h. The reaction was washed (2×NaHCO₃) and extracted with DCM (2×50 mL). The combined organics were dried (Na₂SO₄) and evaporated at 30° C. to afford 2-bromo-1-(1-methyl-2-oxabicyclo[2.1.1]hexan-4-yl)ethan-1-one (19.5 g, crude) as a yellow oil. ¹H NMR (CDCl₃, 400 MHz) δ 3.99 (s, 4H), 2.0-2.1 (m, 2H), 1.8-2.0 (m, 2H), 1.48 (s, 3H).

### Preparation 13: 2-Bromo-1-(1-methyl-2-oxabicyclo[2.2.2]octan-4-yl)ethan-1-one

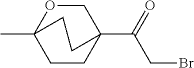

DMF (few drops), followed by oxalyl dichloride (5.43 mL, 63 mmol) were added dropwise to a solution of 1-methyl-2-oxabicyclo[2.2.2]octane-4-carboxylic acid (7.15 g, 42 mmol) in DCM (150 mL) under Ar and the reaction stirred at rt overnight. The mixture was evaporated under reduced pressure to provide 1-methyl-2-oxabicyclo[2.2.2]octane-4-carbonyl chloride. This was dissolved in DCM (50 mL), cooled to 0° C., and ethereal diazomethane (3 eq. in 1 L Et₂O) was added and the reaction stirred for 30 min. A stream of Ar was passed through the solution to remove excess diazomethane and the solution was evaporated under reduced pressure. The residue was purified by silica gel column chromatography eluting with (30% EtOAc in hexane) to provide 3-diazo-1-(1-methyl-2-oxabicyclo[2.2.2]octan-4-yl)propan-1-one (5.0 g, 26 mmol), which was dissolved in DCM (200 mL). The solution cooled to 0° C., excess 40% aqueous HBr was added and the mixture was stirred for 1 h. The layers were separated, the organic layer was washed with saturated aqueous Na₂CO₃ and dried over Na₂SO₄. The filtrate was evaporated under reduced pressure to afford 2-bromo-1-(1-methyl-2-oxabicyclo[2.2.2]octan-4-yl)ethan-1-one (5.3 g, 83%) as a brown crystalline solid. 1H NMR (400 MHz, CHLOROFORM-d) δ ppm 1.06 (s, 3H) 1.56-1.65 (m, 2H) 1.78-1.98 (m, 6H) 3.93 (t, J=1.51 Hz, 2H) 3.98 (s, 2H).

### Preparation 14: 1-(2-oxabicyclo[2.2.1]heptan-4-yl)-2-bromoethan-1-one

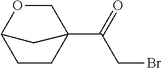 [SMILES: O=C(CBr)C12CCC(C1)OC2]

1-(2-oxabicyclo[2.2.1]heptan-4-yl)-2-bromoethan-1-one was obtained as a yellow oil, 5.2 g, from 2-oxabicyclo[2.2.1]heptane-4-carboxylic acid, following a similar procedure to that described in Preparation 13. ¹H NMR (400 MHz, CHLOROFORM-d) δ ppm 1.77-1.95 (m, 4H) 2.00-2.13 (m, 2H) 3.80 (d, J=7.03 Hz, 1H) 3.95 (dd, J=7.15, 3.64 Hz, 1H) 4.07 (s, 2H) 4.49 (t, J=2.13 Hz, 1H).

### Preparation 15: 2-bromo-1-(1-methyl-2-oxabicyclo[2.2.1]heptan-4-yl)ethan-1-one

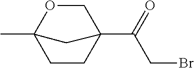

2-bromo-1-(1-methyl-2-oxabicyclo[2.2.1]heptan-4-yl)ethan-1-one was obtained as a yellow oil from 1-methyl-2-oxabicyclo[2.2.1]heptane-4-carboxylic acid, following a similar procedure to that described in Preparation 13. ¹H NMR (400 MHz, CHLOROFORM-d) δ ppm 1.45 (s, 3H) 1.74-1.90 (m, 3H) 1.93-2.04 (m, 2H) 2.08-2.17 (m, 1H) 3.87 (d, J=7.03 Hz, 1H) 4.03-4.08 (m, 3H).

### Preparation 16: 2-bromo-1-(1-ethyl-2-oxabicyclo[2.2.1]heptan-4-yl)ethan-1-one

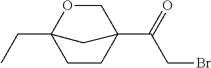

2-bromo-1-(1-ethyl-2-oxabicyclo[2.2.1]heptan-4-yl)ethan-1-one (6.2 g, 85%) was obtained as a yellow oil in a similar manner to that described in Preparation 13 starting from 1-ethyl-2-oxabicyclo[2.2.1]heptane-4-carboxylic acid. ¹H NMR (400 MHz, Chloroform-d) δ 4.0-4.1 (m, 3H), 3.9-3.9 (m, 1H), 2.1-2.2 (m, 1H), 2.0-2.0 (m, 1H), 1.9-2.0 (m, 1H), 1.7-1.8 (m, 5H), 1.01 (t, 3H, J=7.5 Hz).

### Preparation 17: 2-bromo-1-(1-(fluoromethyl)-2-oxabicyclo[2.2.1]heptan-4-yl)ethan-1-one

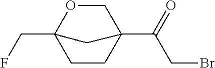 [SMILES: O=C(CBr)C12CCC(CF)(C1)OC2]

2-bromo-1-(1-(fluoromethyl)-2-oxabicyclo[2.2.1]heptan-4-yl)ethan-1-one (7.2 g, 90%) was obtained as a yellow oil in a similar manner to that described in Preparation 13 starting from 1-(fluoromethyl)-2-oxabicyclo[2.2.1]heptane-4-carboxylic acid. ¹H NMR (CHLOROFORM-d, 400 MHz) δ 4.69 (q, 1H, J=10.1 Hz), 4.57 (q, 1H, J=10.1 Hz), 4.10 (dd, 1H, J=3.5, 7.0 Hz), 4.05 (s, 2H), 3.95 (d, 1H, J=7.0 Hz), 2.1-2.2 (m, 2H), 2.0-2.1 (m, 1H), 1.8-2.0 (m, 3H)

### Preparation 18: 2-bromo-1-(1-(methoxymethyl)-2-oxabicyclo[2.2.1]heptan-4-yl)ethan-1-one

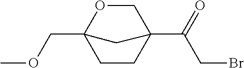

2-bromo-1-(1-(methoxymethyl)-2-oxabicyclo[2.2.1]heptan-4-yl)ethan-1-one was obtained as a yellow solid, (6.0 g, 76%), from 1-(methoxymethyl)-2-oxabicyclo[2.2.1]heptane-4-carboxylic acid, following a similar procedure to that described in Preparation 13. 1H NMR (400 MHz, Chloroform-d) δ 4.11-4.01 (m, 3H), 3.90 (d, J=7.1 Hz, 1H), 3.70-3.57 (m, 2H), 3.44 (d, J=0.7 Hz, 3H), 2.16-2.07 (m, 2H), 2.07-1.95 (m, 1H), 1.92-1.81 (m, 3H).

### Preparation 19: 2-bromo-1-(1-(fluoromethyl)-2-oxabicyclo[2.1.1]hexan-4-yl)ethan-1-one

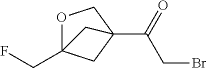 [SMILES: O=C(CBr)C12COC(CF)(C1)C2]

2-bromo-1-(1-(fluoromethyl)-2-oxabicyclo[2.1.1]hexan-4-yl)ethan-1-one was obtained as a yellow oil from 1-(fluoromethyl)-2-oxabicyclo[2.1.1]hexane-4-carboxylic acid, following a similar procedure to that described in Preparation 13. 1H NMR (400 MHz, CHLOROFORM-d) 8 ppm 2.02-2.09 (m, 2H) 2.21-2.29 (m, 2H) 4.01 (s, 2H) 4.08 (s, 2H) 4.58 (s, 1H) 4.70 (s, 1H).

### Preparation 20: 1-(2-oxabicyclo[2.1.1]hexan-4-yl)-2-bromoethan-1-one

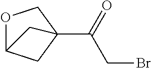

1-(2-oxabicyclo[2.1.1]hexan-4-yl)-2-bromoethan-1-one (2.70 g, 83% overall yield) was obtained as a yellow solid from 2-oxabicyclo[2.1.1]hexane-4-carboxylic acid, following a similar procedure to that described in Preparation 13. ¹H NMR (CHLOROFORM-d, 400 MHz) δ 4.60 (t, 1H, J=1.3 Hz), 4.02 (s, 2H), 3.97 (s, 2H), 2.2-2.3 (m, 2H), 1.9-2.0 (m, 2H).

### Preparation 21: 2-bromo-1-[1-(methoxymethyl)-2-oxabicyclo[2.1.1]hexan-4-yl]ethan-1-one

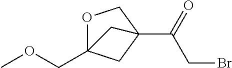

2-bromo-1-[1-(methoxymethyl)-2-oxabicyclo[2.1.1]hexan-4-yl]ethan-1-one ((7.3 g, 85% yield) was obtained as a yellow oil from 1-(methoxymethyl)-2-oxabicyclo[2.1.1]hexane-4-carboxylic acid, following a similar procedure to that described in Preparation 13. ¹H NMR (400 MHz, Chloroform-d) δ 3.99 (dd, 1H, J=3.6, 7.2 Hz), 3.96 (s, 2H), 3.82 (d, 1H, J=7.0 Hz), 3.5-3.6 (m, 2H), 3.35 (s, 3H), 2.0-2.1 (m, 2H), 1.8-1.8 (m, 2H).

### Preparation 22: 2-bromo-1-(1-ethyl-2-oxabicyclo[2.1.1]hexan-4-yl)ethan-1-one

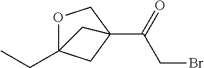

2-bromo-1-(1-ethyl-2-oxabicyclo[2.1.1]hexan-4-yl)ethan-1-one was obtained as a yellow oil from 1-ethyl-2-oxabicyclo[2.1.1]hexane-4-carboxylic acid, following a similar procedure to that described in Preparation 13. ¹H NMR (400 MHz, CHLOROFORM-d) δ ppm 0.99 (t, J=7.53 Hz, 3H) 1.80 (q, J=7.53 Hz, 2H) 1.87-1.92 (m, 2H) 2.01-2.07 (m, 2H) 3.99 (s, 2H) 4.01 (s, 2H).

### Preparation 23: (1S,4S)-1-methyl-2-oxabicyclo[2.2.1]heptane-4-carboxylic acid and (1R,4R)-1-methyl-2-oxabicyclo[2.2.1]heptane-4-carboxylic acid

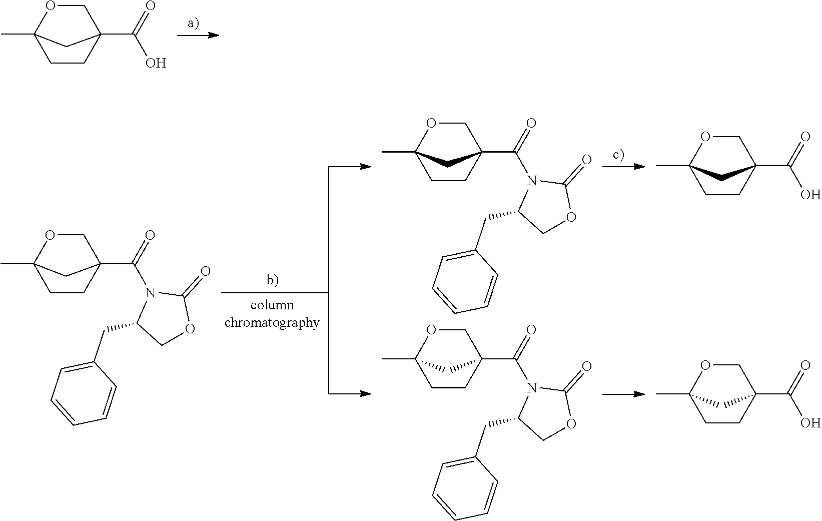

Step a: To a solution of 1-methyl-2-oxabicyclo[2.2.1]heptane-4-carboxylic acid (39.0 g, 249 mmol) in dichloromethane was added a few drops of DMF. The resulting mixture was cooled with an ice bath and SOCl₂ (19 mL, 262 mmol) was added dropwise. The reaction mixture was heated at reflux for 1.5 h, cooled and evaporated to provide 1-methyl-2-oxabicyclo[2.2.1]heptane-4-carbonyl chloride as a residue that was used immediately (below). A solution of (S)-4-benzyloxazolidin-2-one (29.2 g, 249 mmol) in THF was cooled to −60° C., and a THF solution of 2.5 M BuLi (99.6 mL, 249 mmol) was added. The mixture was stirred at −60° C. for 1 h. After that, the reaction mixture was cooled to −78° C. and a solution of above acid chloride in THF was added dropwise. The resulting mixture was allowed to warm to room temperature and stirred overnight. Next, to the reaction mixture, cooled with water bath, was added NH₄Cl (20.2 g, 374 mmol) and THF was removed under reduced pressure. The water phase was extracted with dichloromethane three times, dried over anhydrous Na₂SO₄ and evaporated under reduced pressure to obtain a mixture of diastereomers (4S)-4-benzyl-3-(1-methyl-2-oxabicyclo[2.2.1]heptane-4-carbonyl)oxazolidin-2-one (70 g, 100%).

Step b: The isomers were separated via column chromatography to obtain 16.9 g of (S)-4-benzyl-3-((1S,4S)-1-methyl-2-oxabicyclo[2.2.1]heptane-4-carbonyl)oxazolidin-2-one (48.2% yield) and 17.5 g of (S)-4-benzyl-3-((1R,4R)-1-methyl-2-oxabicyclo[2.2.1]heptane-4-carbonyl)oxazolidin-2-one (50% yield).

### (1S,4S)-1-methyl-2-oxabicyclo[2.2.1]heptane-4-carboxylic acid

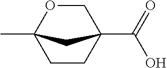

Step c: To a solution of (S)-4-benzyl-3-((1S,4S)-1-methyl-2-oxabicyclo[2.2.1]heptane-4-carbonyl)oxazolidin-2-one (16.9 g, 53.6 mmol) in a mixture of THF:H₂O (4:1), cooled with an ice bath, was added dropwise 35% H₂O₂ (20.8 mL, 214 mmol) followed by portion-wise addition of LiOH·H₂O (4.5 g, 107 mmol). The reaction mixture was stirred for 1 h at 0° C. at which time Na₂SO₃ (33.7 g, 268 mmol) was added in portions. The resulting mixture was allowed to warm to rt and stirred for 20 min. The THF was removed under reduced pressure and the aqueous phase was washed with DCM (3×25 mL). The remaining aqueous phase was acidified with 3 N HCl to pH-3, extracted with DCM (3×50 mL). The combined organic layers from the acidified extraction were dried (Na₂SO₄), filtered and evaporated under reduced pressure to obtain 5.96 g of (1S,4S)-1-methyl-2-oxabicyclo[2.2.1]heptane-4-carboxylic acid (71.2% yield)¹H NMR (METHANOL-d4, 400 MHz) δ 7.91 (s, 1H), 3.9-4.1 (m, 1H), 3.78 (d, 1H, J=7.0 Hz), 2.1-2.2 (m, 1H), 1.6-2.0 (m, 5H), 1.40 (s, 3H).

### (1R,4R)-1-methyl-2-oxabicyclo[2.2.1]heptane-4-carboxylic acid

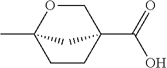

Enantiomer (1R,4R)-1-methyl-2-oxabicyclo[2.2.1]heptane-4-carboxylic acid was obtained in a manner similar to that described for (1S,4S)-1-methyl-2-oxabicyclo[2.2.1]heptane-4-carboxylic acid (Step c) starting from diastereomer (S)-4-benzyl-3-((1R,4R)-1-methyl-2-oxabicyclo[2.2.1]heptane-4-carbonyl)oxazolidin-2-one. ¹H NMR (METHANOL-d4, 400 MHz) δ 7.91 (s, 1H), 3.99 (dd, 1H, J=3.3, 6.8 Hz), 3.77 (d, 1H, J=6.8 Hz), 2.1-2.3 (m, 1H), 1.6-2.0 (m, 5H), 1.40 (s, 3H).

### Preparation 24a and 24b: 2-bromo-1-((1S,4S)-1-methyl-2-oxabicyclo[2.2.1]heptan-4-yl)ethan-1-one and 2-bromo-1-((1R,4R)-1-methyl-2-oxabicyclo[2.2.1]heptan-4-yl)ethan-1-one

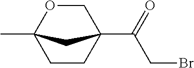

2-bromo-1-((1S,4S)-1-methyl-2-oxabicyclo[2.2.1]heptan-4-yl)ethan-1-one was obtained as a yellow oil from (1S,4S)-1-methyl-2-oxabicyclo[2.2.1]heptane-4-carboxylic acid, following a similar procedure to that described in Preparation 13. ¹H NMR (CHLOROFORM-d, 400 MHz) δ 4.0-4.1 (m, 3H), 3.87 (d, 1H, J=7.0 Hz), 2.1-2.2 (m, 1H), 1.9-2.0 (m, 2H), 1.7-1.9 (m, 3H), 1.45 (s, 3H).

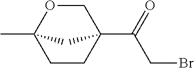

2-bromo-1-((1R,4R)-1-methyl-2-oxabicyclo[2.2.1]heptan-4-yl)ethan-1-one was obtained as a yellow oil from (1R,4R)-1-methyl-2-oxabicyclo[2.2.1]heptane-4-carboxylic acid, following a similar procedure to that described in Preparation 13. ¹H NMR (CHLOROFORM-d, 400 MHz) δ 4.0-4.1 (m, 3H), 3.87 (d, 1H, J=7.0 Hz), 2.0-2.2 (m, 1H), 1.7-2.0 (m, 5H), 1.45 (s, 3H).

### Preparation 25: 7-(sec-butoxy)-2-((1S,4R)-1-methyl-2-oxabicyclo[2.2.1]heptan-4-yl)imidazo[1,2-a]pyridine-6-carboxylic acid

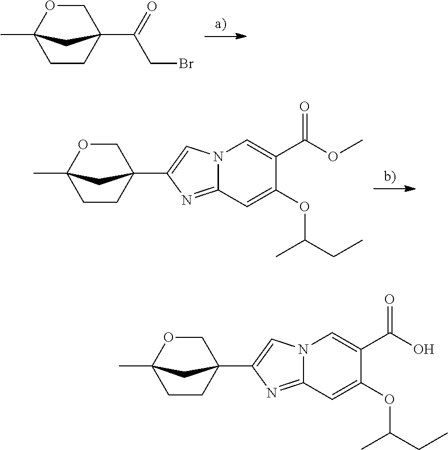

Step a: To a mixture of methyl 6-amino-4-(sec-butoxy)nicotinate [preparation 57] (109 mg, 486.05 μmol), 2-bromo-1-[(1S,4S)-1-methyl-2-oxabicyclo[2.2.1]heptan-4-yl]ethanone (128 mg, 549 μmol) and sodium bicarbonate (123 mg, 1.46 mmol) was added MeCN (2 mL) and toluene (1.5 mL). The mixture was heated to 90° C. overnight. The mixture was partitioned between EtOAc and water. The layers were separated and the aqueous layer was extracted with EtOAc (3×15 mL). The combined organic phases were concentrated, purified by silica gel chromatography (EtOAc in heptane 50-100%) to get 7-(sec-butoxy)-2-((1S,4R)-1-methyl-2-oxabicyclo[2.2.1]heptan-4-yl)imidazo[1,2-a]pyridine-6-carboxylic acid as a yellow oil (159 mg, 91% yield). LCMS (ESI) m/z 359.0 (M+H)⁺. ¹H NMR (METHANOL-d4, 400 MHz) δ 8.8-8.9 (m, 1H), 7.58 (d, 1H, J=0.8 Hz), 6.84 (s, 1H), 4.5-4.6 (m, 1H), 4.05 (dd, 1H, J=3.5, 6.5 Hz), 3.9-3.9 (m, 4H), 2.1-2.2 (m, 2H), 1.9-2.0 (m, 2H), 1.7-1.9 (m, 4H), 1.47 (s, 3H), 1.39 (d, 3H, J=6.0 Hz), 1.05 (t, 3H, J=7.4 Hz).

Step b: 1M NaOH (881.6 uL, 881.6 μmol) was added to a mixture of methyl 7-(sec-butoxy)-2-((1S,4R)-1-methyl-2-oxabicyclo[2.2.1]heptan-4-yl)imidazo[1,2-a]pyridine-6-carboxylate (158 mg, 440.8 μmol) in THF (1 mL) and MeOH (1 mL). The mixture was stirred at rt overnight. Added 1 N HCl (882 μL). The mixture was concentrated to get 7-(sec-butoxy)-2-((1S,4R)-1-methyl-2-oxabicyclo[2.2.1]heptan-4-yl)imidazo[1,2-a]pyridine-6-carboxylic acid as an off-white solid (203 mg, 100% yield), which was used for the next step (as 2·NaCl salt) without further purification LCMS (ESI) m/z 344.9 (M+H)⁺.

### Preparation 26: 2-(2-oxabicyclo[2.1.1]hexan-4-yl)-7-isopropoxyimidazo[1,2-a]pyrimidine-6-carboxylic acid

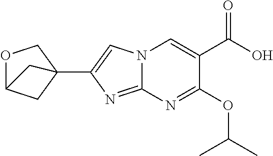 [SMILES: CC(C)Oc1nc2nc(C34COC(C3)C4)cn2cc1C(=O)O]

2-(2-oxabicyclo[2.1.1]hexan-4-yl)-7-isopropoxyimidazo[1,2-a]pyrimidine-6-carboxylic acid (as 1.5·NaCl salt) was obtained as pale brown solid (160 mg, yield 41% over two steps), from 1-(2-oxabicyclo[2.1.1]hexan-4-yl)-2-bromoethan-1-one [preparation 20] and methyl 2-amino-4-isopropoxy-pyrimidine-5-carboxylate [preparation 5], following a similar procedure to that described in Preparation 25. LCMS m/z=303.9 [M+H]+ 1H NMR (METHANOL-d4, 400 MHz) δ 9.0-9.2 (m, 1H), 7.59 (s, 1H), 5.4-5.7 (m, 1H), 4.67 (t, 1H, J=1.0 Hz), 3.96 (s, 2H), 2.27 (td, 2H, J=1.2, 5.0 Hz), 1.8-2.0 (m, 2H), 1.47 (d, 6H, J=6.3 Hz).

### Preparation 27: (R)-7-(sec-butoxy)-2-(1-methyl-2-oxabicyclo[2.1.1]hexan-4-yl)imidazo[1,2-a]pyrimidine-6-carboxylic acid

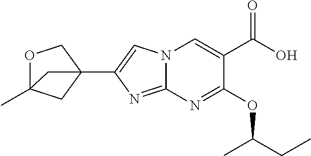 [SMILES: CC[C@@H](C)Oc1nc2nc(C34COC(C)(C3)C4)cn2cc1C(=O)O]

(R)-7-(sec-butoxy)-2-(1-methyl-2-oxabicyclo[2.1.1]hexan-4-yl)imidazo[1,2-a]pyrimidine-6-carboxylic acid (as 1.5·NaCl salt) was obtained as an off-white powder (261 mg, yield 31% over two steps), from 2-bromo-1-(1-methyl-2-oxabicyclo[2.1.1]hexan-4-yl)ethan-1-one [preparation 12] and methyl (R)-2-amino-4-(sec-butoxy)pyrimidine-5-carboxylate [preparation 11b] following a similar procedure to that described in Preparation 25. LCMS m/z=332.1 [M+H]⁺. H NMR (METHANOL-d4, 400 MHz) δ 9.11 (s, 1H), 7.57 (s, 1H), 5.3-5.4 (m, 1H), 4.01 (s, 2H), 2.1-2.2 (m, 2H), 1.7-2.0 (m, 4H), 1.51 (s, 3H), 1.44 (d, 3H, J=6.3 Hz), 1.03 (t, 3H, J=7.4 Hz).

### Preparation 28: (S)-7-(sec-butoxy)-2-(1-methyl-2-oxabicyclo[2.1.1]hexan-4-yl)imidazo[1,2-a]pyrimidine-6-carboxylic acid

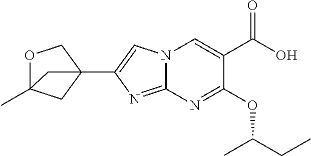 [SMILES: CC[C@H](C)Oc1nc2nc(C34COC(C)(C3)C4)cn2cc1C(=O)O]

(S)-7-(sec-butoxy)-2-(1-methyl-2-oxabicyclo[2.1.1]hexan-4-yl)imidazo[1,2-a]pyrimidine-6-carboxylic acid (as 1.5·NaCl salt) was obtained as an off-white powder (348 mg, yield 42% over two steps), from 2-Bromo-1-(1-methyl-2-oxabicyclo[2.1.1]hexan-4-yl)ethan-1-one [preparation 12] and methyl (S)-2-amino-4-(sec-butoxy)pyrimidine-5-carboxylate [preparation 11a] following a similar procedure to that described in Preparation 25. LCMS m/z=332.1 [M+H]⁺. 1H NMR (methanol-d4, 400 MHz) δ 9.08 (s, 1H), 7.56 (s, 1H), 5.3-5.4 (m, 1H), 3.99 (s, 2H), 2.1-2.2 (m, 2H), 1.8-1.9 (m, 3H), 1.74 (ddd, 1H, J=5.6, 7.6, 13.6 Hz), 1.49 (s, 3H), 1.42 (d, 3H, J=6.3 Hz), 1.01 (t, 3H, J=7.5 Hz).

### Preparation 29: 7-isopropoxy-2-(1-(methoxymethyl)-2-oxabicyclo[2.1.1]hexan-4-yl)imidazo[1,2-a]pyrimidine-6-carboxylic acid

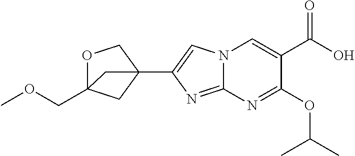 [SMILES: COCC12CC(c3cn4cc(C(=O)O)c(OC(C)C)nc4n3)(CO1)C2]

7-isopropoxy-2-(1-(methoxymethyl)-2-oxabicyclo[2.1.1]hexan-4-yl)imidazo[1,2-a]pyrimidine-6-carboxylic acid (as 1.5·NaCl salt) was obtained as an off-white powder (160 mg, yield 37% over two steps) from 2-bromo-1-(1-(methoxymethyl)-2-oxabicyclo[2.1.1]hexan-4-yl)ethan-1-one and methyl 2-amino-4-isopropoxy-pyrimidine-5-carboxylate [preparation 5] following a similar procedure to that described in Preparation 25. LCMS m/z=347.9 [M+H]⁺. 1H NMR (METHANOL-d4, 400 MHz) δ 9.0-9.2 (m, 1H), 7.59 (s, 1H), 5.54 (quin, 1H, J=6.2 Hz), 4.04 (s, 2H), 3.72 (s, 2H), 3.44 (s, 3H), 2.1-2.3 (m, 2H), 1.94 (dd, 2H, J=1.8, 4.5 Hz), 1.4-1.5 (m, 6H).

### Preparation 30: 2-(2-oxabicyclo[2.1.1]hexan-4-yl)-7-cyclobutoxyimidazo[1,2-a]pyrimidine-6-carboxylic acid

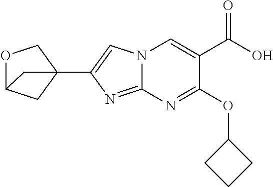 [SMILES: O=C(O)c1cn2cc(C34COC(C3)C4)nc2nc1OC1CCC1]

2-(2-oxabicyclo[2.1.1]hexan-4-yl)-7-cyclobutoxyimidazo[1,2-a]pyrimidine-6-carboxylic acid (as 2·NaCl salt) was obtained as an off-white powder (181 mg, yield 41% over two steps), from 2-bromo-1-{2-oxabicyclo[2.1.1]hexan-4-yl}ethan-1-one [Preparation 20] and methyl 2-amino-4-cyclobutoxypyrimidine-5-carboxylate [Preparation 52] following a similar procedure to that described in Preparation 25. LCMS m/z=315.9 [M+H]⁺. ¹H NMR (METHANOL-d4, 400 MHz) δ 9.0-9.2 (m, 1H), 7.58 (s, 1H), 5.38 (dd, 1H, J=7.0, 7.8 Hz), 4.66 (t, 1H, J=1.0 Hz), 3.96 (s, 2H), 2.5-2.6 (m, 2H), 2.2-2.4 (m, 4H), 1.6-2.0 (m, 4H).

### Preparation 31: 2-(2-oxabicyclo[2.1.1]hexan-4-yl)-7-cyclobutoxyimidazo[1,2-a]pyridine-6-carboxylic acid

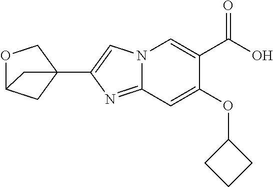 [SMILES: O=C(O)c1cn2cc(C34COC(C3)C4)nc2cc1OC1CCC1]

2-(2-oxabicyclo[2.1.1]hexan-4-yl)-7-cyclobutoxyimidazo[1,2-a]pyridine-6-carboxylic acid (as 2·NaCl salt) was obtained as an off-white powder (304 mg, yield 70% over two steps) starting with methyl 6-amino-4-cyclobutoxynicotinate [Preparation 9] and 1-(2-oxabicyclo[2.1.1]hexan-4-yl)-2-bromoethan-1-one [Preparation 20] following a similar procedure to that described in Preparation 25. LCMS m/z=314.9 [M+H]⁺. ¹H NMR (METHANOL-d4, 400 MHz) δ 8.71 (s, 1H), 7.76 (s, 1H), 6.77 (s, 1H), 4.9-5.0 (m, 1H), 4.68 (t, 1H, J=1.0 Hz), 3.97 (s, 2H), 2.5-2.7 (m, 2H), 2.2-2.4 (m, 4H), 1.7-2.1 (m, 4H).

### Preparation 32: 7-Cyclobutoxy-2-(1-methyl-2-oxabicyclo[2.2.1]heptan-4-yl)imidazo[1,2-a]pyridine-6-carboxylic acid

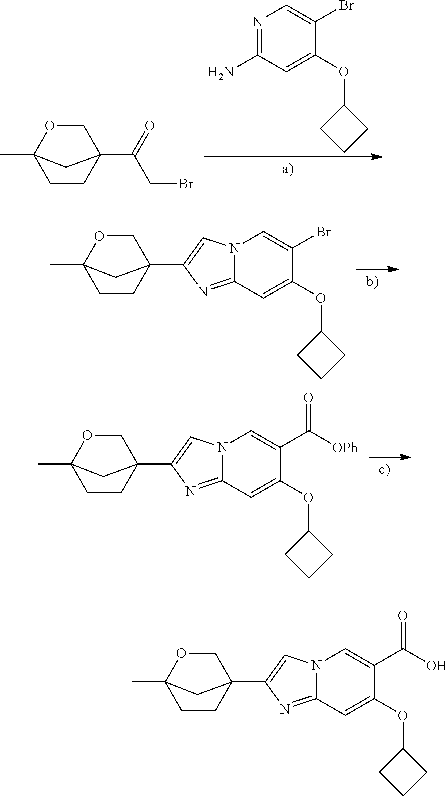

Step a: A mixture of 2-bromo-1-(1-methyl-2-oxabicyclo[2.2.1]heptan-4-yl)ethan-1-one (Preparation 15, 575 mg, 2.47 mmol), 5-bromo-4-cyclobutoxypyridin-2-amine (Preparation 9, Part A, 500 mg, 2.06 mmol) and NaHCO₃ (518 mg, 6.17 mmol) in MeCN (6 mL) and toluene (4 mL) was heated at 90° C. overnight. The reaction mixture was partitioned between EtOAc and brine and the aqueous layer was extracted with EtOAc (2×10 mL). The combined organics were dried (Na₂SO₄) and evaporated to dryness in vacuo. The residue was purified by column chromatography on silica gel eluting with EtOAc to afford 6-bromo-7-cyclobutoxy-2-(1-methyl-2-oxabicyclo[2.2.1]heptan-4-yl)imidazo[1,2-a]pyridine as a pale brown oil (636 mg, 81%). ¹H NMR (400 MHz, MeOH-d₄) δ:1.47 (s, 3H), 1.70-2.00 (m, 6H), 2.10-2.30 (m, 4H), 2.50-2.70 (m, 2H), 3.91 (d, 1H), 4.03 (dd, 1H), 4.84 (d, 1H), 6.71 (s, 1H), 7.50 (s, 1H), 8.60 (s, 1H).

Step b: TEA (344 mg, 3.40 mmol) was added to a mixture of 6-bromo-7-cyclobutoxy-2-(1-methyl-2-oxabicyclo[2.2.1]heptan-4-yl)imidazo[1,2-a]pyridine (512 mg, 1.36 mmol), Pd(OAc)₂ (21.4 mg, 0.095 mmol), Xantphos (63.0 mg, 0.109 mmol) and phenyl formate (415 mg, 3.40 mmol) in MeCN (6 mL) and the mixture heated at 80° C. for 4.5 h. The cooled reaction was partitioned between EtOAc and brine, the aqueous layer extracted with EtOAc and the combined organics were evaporated to dryness in vacuo. The residue was purified by column chromatography on silica gel eluting with EtOAc to afford phenyl 7-cyclobutoxy-2-(1-methyl-2-oxabicyclo[2.2.1]heptan-4-yl)imidazo[1,2-a]pyridine-6-carboxylate as a pale-yellow oil (499 mg, 87.0%). LCMS m/z=419.3 [M+H]⁺. ¹H NMR (METHANOL-d4, 400 MHz) δ 9.15 (s, 1H), 7.65 (s, 1H), 7.4-7.5 (m, 2H), 7.3-7.4 (m, 1H), 7.27 (d, 2H, J=8.3 Hz), 6.75 (s, 1H), 4.9-4.9 (m, 1H), 4.06 (dd, 1H, J=3.5, 6.5 Hz), 3.94 (d, 1H, J=6.3 Hz), 2.5-2.7 (m, 2H), 2.1-2.4 (m, 4H), 1.8-2.0 (m, 6H), 1.48 (s, 3H).

Step c: NaOH (1 M, 2.36 mL) was added to a solution of phenyl 7-cyclobutoxy-2-(1-methyl-2-oxabicyclo[2.2.1]heptan-4-yl)imidazo[1,2-a]pyridine-6-carboxylate (493 mg, 1.18 mmol) in MeOH (2 mL) and THF (2 mL) and the mixture stirred at 40° C. for 4.5 h. The reaction mixture was neutralised by the addition of 1 N HCl (2.36 mL) and the resulting clear solution concentrated and lyophilized over 72 h to afford 7-cyclobutoxy-2-(1-methyl-2-oxabicyclo[2.2.1]heptan-4-yl)imidazo[1,2-a]pyridine-6-carboxylic acid as an off-white solid (519 mg, yield 100%, 78% pure). LCMS m/z=343.1 [M+H]⁺.

### Preparation 33: 7-cyclobutoxy-2-(1-methyl-2-oxabicyclo[2.1.1]hexan-4-yl)imidazo[1,2-a]pyridine-6-carboxylic acid

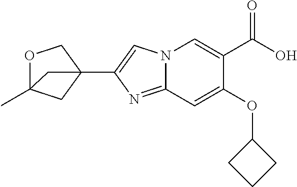 [SMILES: CC12CC(c3cn4cc(C(=O)O)c(OC5CCC5)cc4n3)(CO1)C2]

7-cyclobutoxy-2-(1-methyl-2-oxabicyclo[2.1.1]hexan-4-yl)imidazo[1,2-a]pyridine-6-carboxylic acid was obtained as an off-white solid (401 mg, yield 50% over three steps, 72% pure), from 2-bromo-1-(1-methyl-2-oxabicyclo[2.1.1]hexan-4-yl)ethan-1-one [Preparation 12] and 5-bromo-4-cyclobutoxypyridin-2-amine [Preparation 9, step A] following a similar procedure to that described in Preparation 32. LCMS m/z=329.1 [M+H]⁺.

### Preparation 34: methyl 7-hydroxy-2-(1-methyl-2-oxabicyclo[2.1.1]hexan-4-yl)imidazo[1,2-a]pyridine-6-carboxylate

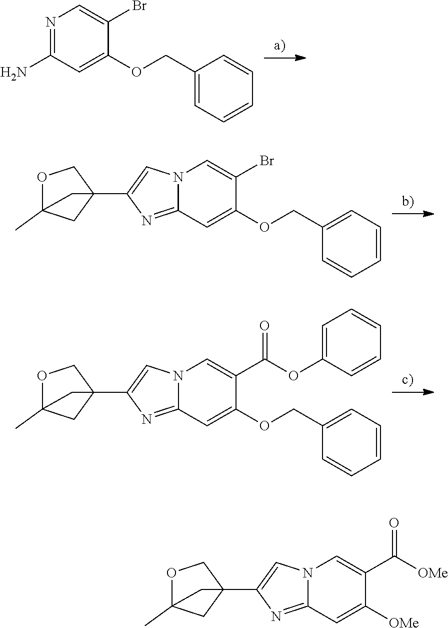

Step a: 4-benzyloxy-5-bromo-pyridin-2-amine (2.6 g, 9.4 mmol) was dissolved in ACN (94 mL). Potassium Carbonate (2.6 g, 18.9 mmol) was added followed by 2-bromo-1-(1-methyl-2-oxabicyclo[2.1.1]hexan-4-yl)ethan-1-one [preparation 12] (2.49 g, 11.35 mmol). The mixture was stirred at 90° C. for 18 h. The mixture was concentrated and purified by silica gel chromatography eluting with a gradient of 0-100% EtOAc-heptane to provide 7-(benzyloxy)-6-bromo-2-(1-methyl-2-oxabicyclo[2.1.1]hexan-4-yl)imidazo[1,2-a]pyridine (50% yield). LCMS m/z=400.9 (M+2).

Step b: 7-benzyloxy-6-bromo-2-(1-methyl-2-oxabicyclo[2.1.1]hexan-4-yl)imidazo[1,2-a]pyridine (431 mg g, 1.08 mmol) was dissolved in ACN (3.6 mL), phenyl formate (395 mg, 3.24 mmol) was added followed by XantPhos-Pd-G3 (28.78 mg, 32.4 μmol) and triethylamine (0.45 mL, 3.24 mmol). The atmosphere was switched to nitrogen and then the mixture stirred at 90° C. overnight. The reaction was then cooled to room temperature and diluted with water, the reaction was extracted with EtOAc and dried over sodium sulfate. The crude was then purified by silica gel chromatography using a gradient of 0-100% EtOAc and heptane to afford phenyl 7-(benzyloxy)-2-(1-methyl-2-oxabicyclo[2.1.1]hexan-4-yl)imidazo[1,2-a]pyridine-6-carboxylate (81% yield). LCMS m/z=441.0 (M+1).

Step c: phenyl 7-benzyloxy-2-(1-methyl-2-oxabicyclo[2.1.1]hexan-4-yl)imidazo[1,2-a]pyridine-6-carboxylate (5.00 g, 11.4 mmol) was dissolved in MeOH (113.5 mL), Pd/C (604 mg, 567.5 umol, 10% purity) was added and Ammonium formate (7.2 g, 114 mmol). The reaction was warmed to 60 for 1 h, cooled to room temperature and filtered on celite then concentrated. The material was then purified by silica gel chromatography using a gradient of 0-100% EtOAc-Heptane, and then lyophilized to afford methyl 7-hydroxy-2-(1-methyl-2-oxabicyclo[2.1.1]hexan-4-yl)imidazo[1,2-a]pyridine-6-carboxylate (70% yield). LCMS ES+289.1 (M+H).

### Preparation 35: methyl 7-hydroxy-2-(1-methyl-2-oxabicyclo[2.2.1]heptan-4-yl)imidazo[1,2-a]pyridine-6-carboxylate

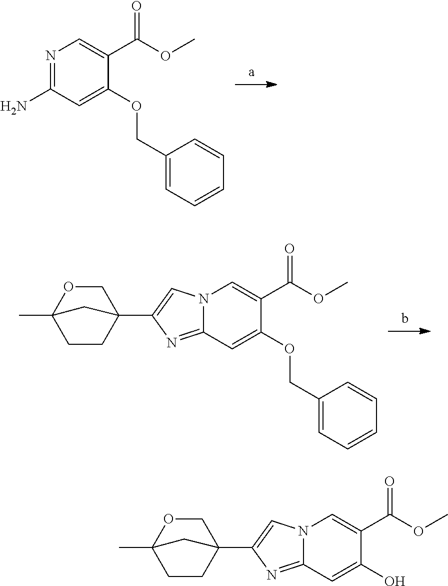

Step a: A mixture of methyl 6-amino-4-(benzyloxy)nicotinate (6.91 g, 29.7 mmol), 2-bromo-1-(1-methyl-2-oxabicyclo[2.2.1]heptan-4-yl)ethan-1-one (preparation 15, 5.1 g, 19.8 mmol) and NaHCO₃ (2.49 g, 29.7 mmol) in toluene/acetonitrile (100 mL, 1:1) was refluxed for 20 h and all volatiles were evaporated in vacuo. The residue was recrystallized from hexane to give methyl 7-(benzyloxy)-2-(1-methyl-2-oxabicyclo[2.2.1]heptan-4-yl)imidazo[1,2-a]pyridine-6-carboxylate (3.1 g, 40% yield).

Step b: Pd/C (10%, 0.5 g) was added to a solution of methyl 7-(benzyloxy)-2-(1-methyl-2-oxabicyclo[2.2.1]heptan-4-yl)imidazo[1,2-a]pyridine-6-carboxylate (3.08 g, 7.85 mmol) in MeOH (50 mL) and the resulting mixture was hydrogenated in autoclave (120 bar, r.t.) for 3 h. The catalyst was filtered off and the filtrates were evaporated to give methyl 7-hydroxy-2-(1-methyl-2-oxabicyclo[2.2.1]heptan-4-yl)imidazo[1,2-a]pyridine-6-carboxylate (2.3 g, 97% yield). LCMS ES+303.0 (M+H)⁺.

### Preparation 36: (S)-7-((1-fluoropropan-2-yl)oxy)-2-(1-methyl-2-oxabicyclo[2.1.1]hexan-4-yl)imidazo[1,2-a]pyridine-6-carboxylic acid

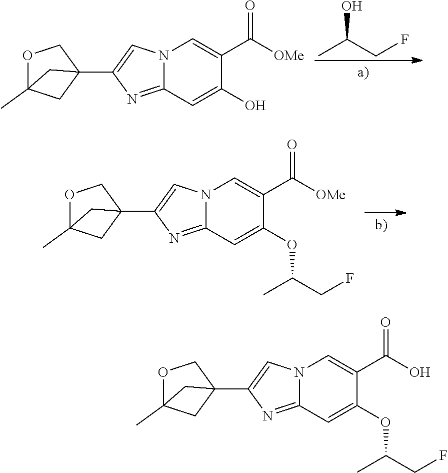

Step a: Triphenylphosphine (157 mg, 600 μmol) was premixed with DIAD (118 μL, 600 mol) in THF (2 mL) for 5 minutes. Then (2R)-1-fluoropropan-2-ol (52 μL, 600 μmol) was added and stirred 5 minutes followed by the addition of methyl 7-hydroxy-2-(1-methyl-2-oxabicyclo[2.1.1]hexan-4-yl)imidazo[1,2-a]pyridine-6-carboxylate [Preparation 34] (115 mg, 0.4 mmol). The resulting reaction was stirred at rt for 30 min, the mixture was loaded to a normal phase column eluted with 100% EtOAc to collect methyl (S)-7-((1-fluoropropan-2-yl)oxy)-2-(1-methyl-2-oxabicyclo[2.1.1]hexan-4-yl)imidazo[1,2-a]pyridine-6-carboxylate as an oil (86 mg, 62% yield). LCMS (ESI) m/z 348.9 (M+H)⁺.

Step b: 1M NaOH (494 uL, 493 umol) was added to a mixture of methyl (S)-7-((1-fluoropropan-2-yl)oxy)-2-(1-methyl-2-oxabicyclo[2.1.1]hexan-4-yl)imidazo[1,2-a]pyridine-6-carboxylate (86 mg, 247 umol) in THF (1 mL) and MeOH (1 mL). The mixture was stirred at rt for 2 h. It was then treated with 1N HCl (0.5 mL) and concentrated in vacuo to provide (S)-7-((1-fluoropropan-2-yl)oxy)-2-(1-methyl-2-oxabicyclo[2.1.1]hexan-4-yl)imidazo[1,2-a]pyridine-6-carboxylic acid (as 2·NaCl salt) as a solid (108 mg, 97% yield). LCMS (ESI) m/z 334.9 (M+H)⁺. 1H NMR (METHANOL-d4, 500 MHz) δ 8.7-8.8 (m, 1H), 7.76 (s, 1H), 7.08 (s, 1H), 4.97 (br d, 1H, J=5.5 Hz), 4.5-4.7 (m, 2H), 4.03 (s, 2H), 2.16 (dd, 2H, J=1.5, 4.6 Hz), 1.94 (dd, 2H, J=1.8, 4.9 Hz), 1.52 (s, 3H), 1.43 (dd, 3H, J=1.5, 6.4 Hz).

### Preparation 37: (R)-7-((1-fluoropropan-2-yl)oxy)-2-(1-methyl-2-oxabicyclo[2.1.1]hexan-4-yl)imidazo[1,2-a]pyridine-6-carboxylic acid

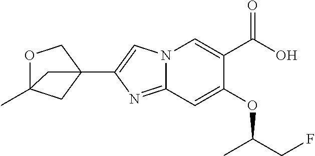 [SMILES: C[C@H](CF)Oc1cc2nc(C34COC(C)(C3)C4)cn2cc1C(=O)O]

(R)-7-((1-fluoropropan-2-yl)oxy)-2-(1-methyl-2-oxabicyclo[2.1.1]hexan-4-yl)imidazo[1,2-a]pyridine-6-carboxylic acid (as 2·NaCl salt) was obtained as an off-white solid (95 mg, 52% yield) from (S)-1-fluoropropan-2-ol following a similar procedure to that described in Preparation 36. LCMS (ESI) m/z 334.9 (M+H)⁺. ¹H NMR (METHANOL-d4, 500 MHz) δ 8.72 (s, 1H), 7.75 (s, 1H), 7.07 (s, 1H), 4.97 (br dd, 1H, J=5.8, 10.7 Hz), 4.5-4.7 (m, 2H), 4.03 (s, 2H), 2.1-2.3 (m, 2H), 1.94 (dd, 2H, J=1.5, 4.6 Hz), 1.52 (s, 3H), 1.43 (dd, 3H, J=1.5, 6.4 Hz).

### Preparation 38: 7-(((S)-1-fluoropropan-2-yl)oxy)-2-(1-methyl-2-oxabicyclo[2.2.1]heptan-4-yl)imidazo[1,2-a]pyridine-6-carboxylic acid

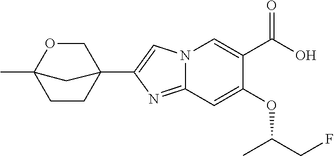 [SMILES: C[C@@H](CF)Oc1cc2nc(C34CCC(C)(C3)OC4)cn2cc1C(=O)O]

7-(((S)-1-fluoropropan-2-yl)oxy)-2-(1-methyl-2-oxabicyclo[2.2.1]heptan-4-yl)imidazo[1,2-a]pyridine-6-carboxylic acid (as 2·NaCl salt) was obtained as an off-white solid (38 mg, yield 43%) from methyl 7-hydroxy-2-(1-methyl-2-oxabicyclo[2.2.1]heptan-4-yl)imidazo[1,2-a]pyridine-6-carboxylate [Preparation 35], following a similar procedure to that described in Preparation 36. LCMS (ESI) m/z 348.9 (M+H)⁺. ¹H NMR (METHANOL-d4, 400 MHz) δ 8.70 (s, 1H), 7.75 (s, 1H), 7.08 (s, 1H), 5.0-5.0 (m, 1H), 4.7-4.7 (m, 1H), 4.5-4.6 (m, 1H), 4.0-4.1 (m, 1H), 3.9-4.0 (m, 1H), 2.1-2.3 (m, 3H), 2.0-2.1 (m, 2H), 1.8-2.0 (m, 2H), 1.49 (s, 3H), 1.43 (dd, 3H, J=1.5, 6.3 Hz)

### Preparation 39: 2-amino-N-(1-cyclopropyl-2-oxo-1,2-dihydropyridin-3-yl)-4-isopropoxypyrimidine-5-carboxamide

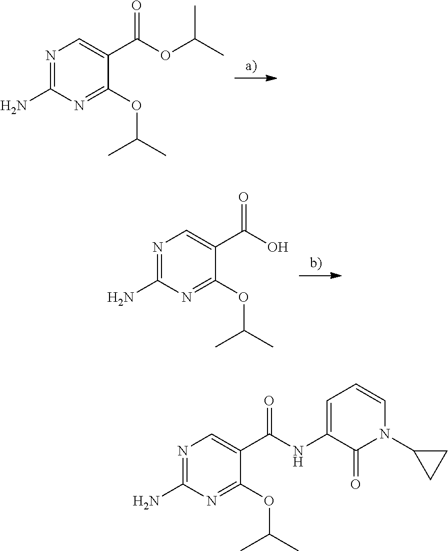 [SMILES: CC(C)OC(=O)c1cnc(N)nc1OC(C)C ; CC(C)Oc1nc(N)ncc1C(=O)O ; CC(C)Oc1nc(N)ncc1C(=O)Nc1cccn(C2CC2)c1=O]

Step a: To a solution of isopropyl 2-amino-4-isopropoxy-pyrimidine-5-carboxylate (preparation 10, 1.2 g, 5 mmol) in THF (4 mL) and MeOH (4 mL) was added NaOH (1 M, 10 mL). The mixture was stirred at rt for 3 days. The reaction mixture was neutralized (acidified) by adding eq. of 1N HCl (pH-3-4), concentrated and lyophilized to yield 2-amino-4-isopropoxypyrimidine-5-carboxylic acid (2 NaCl) as a pale brown powder (1.57 g, 100% yield), which was used in the next step without further purification

Step b: To the mixture of 2-amino-4-isopropoxy-pyrimidine-5-carboxylic acid (157 mg, 500 umol, 2·NaCl), 3-amino-1-cyclopropyl-pyridin-2-one HCl salt (97 mg, 524 umol), HATU (200 mg, 524 umol) in DMF (0.8 mL) was added Hunig's base (348 uL 2 mmol). The mixture was stirred at rt for 2 days. The reaction mixture was partitioned between EtOAc and water. The solid was filtered and washed with water, followed by MeCN to provide 2-amino-N-(1-cyclopropyl-2-oxo-1,2-dihydropyridin-3-yl)-4-isopropoxypyrimidine-5-carboxamide as a pale yellow solid (83 mg, 50% yield). LCMS (ESI) m/z 330.1 (M+H)⁺; 1H NMR (METHANOL-d4, 400 MHz) δ 8.79 (s, 1H), 8.52 (dd, 1H, J=1.8, 7.3 Hz), 7.31 (dd, 1H, J=1.8, 7.0 Hz), 6.36 (t, 1H, J=7.3 Hz), 5.72 (quin, 1H, J=6.3 Hz), 3.4-3.5 (m, 1H), 1.58 (d, 6H, J=6.3 Hz), 1.1-1.3 (m, 2H), 0.9-1.0 (m, 2H).

### Preparation 40: 2-amino-N-(1-(cis-2-fluorocyclopropyl)-2-oxo-1,2-dihydropyridin-3-yl)-4-isopropoxypyrimidine-5-carboxamide

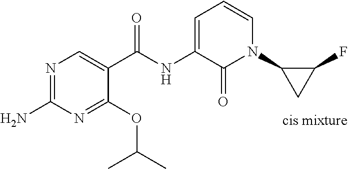 [SMILES: CC(C)Oc1nc(N)ncc1C(=O)Nc1cccn([C@@H]2C[C@@H]2F)c1=O]

To a mixture of 2-amino-4-isopropoxy-pyrimidine-5-carboxylic acid (197.2 mg, 1.00 mmol), 3-amino-1-[cis-2-fluorocyclopropyl]pyridin-2-one [Preparation 63] (202 mg, 1.20 mmol), HATU (400 mg, 1.05 mmol) in DMF (5 mL) was added Hunigs base (610 μL, 3.50 mmol). The mixture was stirred at rt for 6 days. The mixture was concentrated, the residue was partitioned between EtOAc and brine/water. The aqueous layer was extracted with EtOAc. The combined organic phases were dried over MgSO4, filtered and concentrated. The residue was triturated with MeCN/MeOH, the solid was washed with EtOAc, to get a pale brown solid (220 mg, 63% yield). LCMS (ESI) m/z 348.1 (M+H)⁺; 1H NMR (METHANOL-d4, 400 MHz) δ 10.44 (s, 1H), 8.79 (s, 1H), 8.55 (dd, 1H, J=1.8, 7.5 Hz), 7.36 (dd, 1H, J=0.9, 6.9 Hz), 6.39 (t, 1H, J=7.3 Hz), 5.72 (quin, 1H, J=6.3 Hz), 4.9-5.1 (m, 1H), 3.4-3.5 (m, 1H), 1.5-1.6 (m, 8H).

### Preparation 41: (R)-2-amino-4-(sec-butoxy)-N-(1-cyclopropyl-2-oxo-1,2-dihydropyridin-3-yl)pyrimidine-5-carboxamide

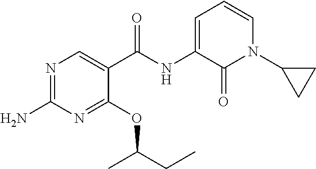 [SMILES: CC[C@@H](C)Oc1nc(N)ncc1C(=O)Nc1cccn(C2CC2)c1=O]

(R)-2-amino-4-(sec-butoxy)-N-(1-cyclopropyl-2-oxo-1,2-dihydropyridin-3-yl)pyrimidine-5-carboxamide was obtained as an off-white solid (490 mg, yield 71% over two steps) from methyl (R)-2-amino-4-(sec-butoxy)pyrimidine-5-carboxylate [Preparation 11b], following a similar procedure to that described in Preparation 39. LCMS (ESI) m/z 343.9 (M+H)⁺; 1H NMR (METHANOL-d4, 400 MHz) δ 8.79 (s, 1H), 8.52 (dd, 1H, J=1.8, 7.5 Hz), 7.31 (dd, 1H, J=1.6, 6.9 Hz), 6.36 (t, 1H, J=7.2 Hz), 5.58 (dd, 1H, J=6.0, 13.3 Hz), 3.4-3.5 (m, 1H), 2.12 (td, 1H, J=7.4, 13.9 Hz), 1.86 (ddd, 1H, J=5.8, 7.7, 13.7 Hz), 1.53 (d, 3H, J=6.3 Hz), 1.1-1.2 (m, 2H), 1.03 (t, 3H, J=7.5 Hz), 0.9-1.0 (m, 2H).

### Preparation 42: (S)-2-amino-4-(sec-butoxy)-N-(1-cyclopropyl-2-oxo-1,2-dihydropyridin-3-yl)pyrimidine-5-carboxamide

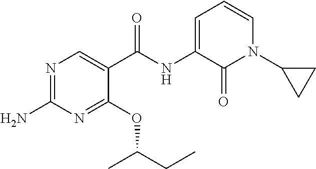 [SMILES: CC[C@H](C)Oc1nc(N)ncc1C(=O)Nc1cccn(C2CC2)c1=O]

(S)-2-amino-4-(sec-butoxy)-N-(1-cyclopropyl-2-oxo-1,2-dihydropyridin-3-yl)pyrimidine-5-carboxamide was obtained as an off-white solid (526 mg, 77% yield over two steps) from methyl (S)-2-amino-4-(sec-butoxy)pyrimidine-5-carboxylate [Preparation 11a], following a similar procedure to that described in Preparation 39. LCMS (ESI) m/z 343.9 (M+H)⁺; 1H NMR (METHANOL-d4, 400 MHz) δ 8.79 (s, 1H), 8.52 (dd, 1H, J=1.8, 7.5 Hz), 7.31 (dd, 1H, J=1.8, 7.0 Hz), 6.36 (t, 1H, J=7.2 Hz), 5.5-5.6 (m, 1H), 3.4-3.5 (m, 1H), 2.1-2.2 (m, 1H), 1.8-2.0 (m, 1H), 1.53 (d, 3H, J=6.3 Hz), 1.1-1.2 (m, 2H), 0.9-1.1 (m, 5H).

### Preparation 43: Methyl (S)-7-(sec-butoxy)-2-(1-methyl-2-oxabicyclo[2.1.1]hexan-4-yl)imidazo[1,2-a]pyridine-6-carboxylate

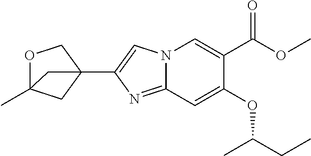

DIAD (561 mg, 2.77 mmol, 0.5 mL) and Triphenylphosphine (727 mg, 2.77 mmol) were mixed in THF (17 mL) for 10 minutes. (2R)-butan-2-ol (160 mg, 2.17 mmol, 0.2 mL) was added and stirred further for 10 minutes. Methyl 7-hydroxy-2-(1-methyl-2-oxabicyclo[2.1.1]hexan-4-yl)imidazo[1,2-a]pyridine-6-carboxylate [Preparation 34] (500 mg, 1.73 mmol) was then added to the mixture and stirred overnight. The resulting was purified by silica gel chromatography using a gradient of 0-100% EtOAc-Heptane to afford methyl (S)-7-(sec-butoxy)-2-(1-methyl-2-oxabicyclo[2.1.1]hexan-4-yl)imidazo[1,2-a]pyridine-6-carboxylate, the material was contaminated with triphenylphosphine oxide, but was carried to the next step without further purification. LCMS ES+345.4 (M+1).

### Preparation 44: (S)-7-(sec-butoxy)-2-(1-methyl-2-oxabicyclo[2.1.1]hexan-4-yl)imidazo[1,2-a]pyridine-6-carboxylic acid

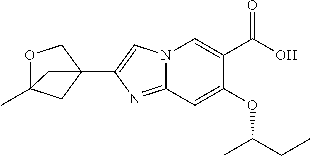 [SMILES: CC[C@H](C)Oc1cc2nc(C34COC(C)(C3)C4)cn2cc1C(=O)O]

methyl 2-(1-methyl-2-oxabicyclo[2.1.1]hexan-4-yl)-7-[(1S)-1-methylpropoxy]imidazo[1,2-a]pyridine-6-carboxylate (500 mg, 1.45 mmol) was dissolved in THF (9 mL), MeOH (3 mL), water (3 mL). Lithium hydroxide (173 mg, 7.26 mmol) was added and the reaction stirred at room temperature overnight. The resulting was concentrated, acidified to pH 2 with HCl 1 N and taken in ACN to be purified by reverse phase flash chromatography using a gradient of 5-80% ACN-water and no modifier. The collected fractions were lyophilized to afford (S)-7-(sec-butoxy)-2-(1-methyl-2-oxabicyclo[2.1.1]hexan-4-yl)imidazo[1,2-a]pyridine-6-carboxylic acid. LCMS ES+331.4 (M+1).

### Preparation 45: 7-isopropoxy-2-(1-methyl-2-oxabicyclo[2.2.2]octan-4-yl)imidazo[1,2-a]pyrimidine-6-carboxylic acid

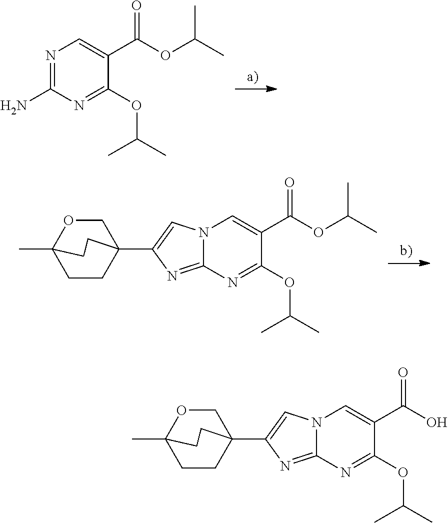

Step a: A mixture of isopropyl 2-amino-4-isopropoxy-pyrimidine-5-carboxylate (preparation 10, 650 mg, 2.72 mmol), 2-bromo-1-(1-methyl-2-oxabicyclo[2.2.2]octan-4-yl)ethanone (preparation 13, 839 mg, 3.40 mmol), NaHCO₃ (684 mg, 8.15 mmol) in MeCN (3.8 mL) and Toluene (3.8 mL) was heated at 90° C. for 16 h. Silica and MeOH were added and the mixture was concentrated and purified by silica gel column chromatography (dry load, 24 g, 0-50% gradient of 3:1 EtOAC/EtOH in heptanes) to obtain isopropyl 7-isopropoxy-2-(1-methyl-2-oxabicyclo[2.2.2]octan-4-yl)imidazo[1,2-a]pyrimidine-6-carboxylate (700 mg, 1.81 mmol, 66% yield). (LCMS (ESI) m/z 388.5 (M+H)⁺.

Step b: A mixture of isopropyl 7-isopropoxy-2-(1-methyl-2-oxabicyclo[2.2.2]octan-4-yl)imidazo[1,2-a]pyrimidine-6-carboxylate (700 mg, 1.81 mmol) and lithium; hydroxide; hydrate (151 mg, 3.61 mmol) in MeOH (283 μL), THF (2.0 mL), water (510 μL) was stirred overnight at room temperature before being neutralized with a 4 M hydrochloric acid solution in dioxane. The mixture was concentrated to afford 7-isopropoxy-2-(1-methyl-2-oxabicyclo[2.2.2]octan-4-yl)imidazo[1,2-a]pyrimidine-6-carboxylic acid, which was used without further purification in next reaction. Assumed 100% yield. LCMS (ESI) m/z 346.1 (M+H)⁺.

### Preparation 46: 3-amino-1-(2,2-dimethylcyclopropyl)pyridin-2(1H)-one

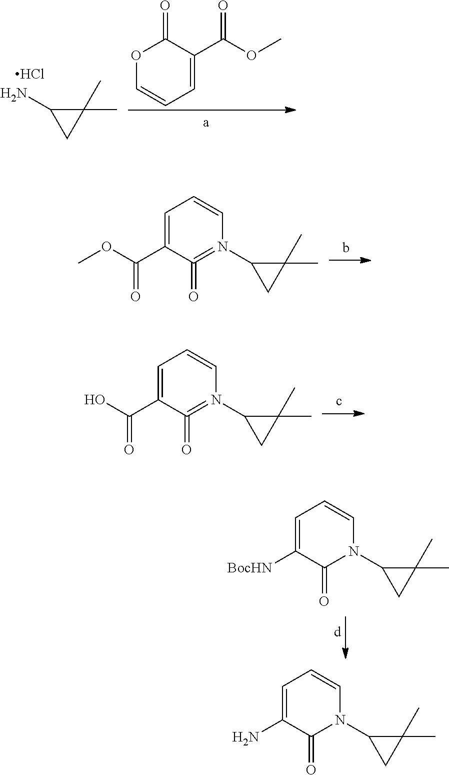

Step a: To a solution of compound methyl 2-oxo-2H-pyran-3-carboxylate (500 mg, 3.24 mmol) and compound 2,2-dimethylcyclopropan-1-amine hydrochloride (395 mg, 3.24 mmol) in DMF (5 mL) was added TEA (657 mg, 6.49 mmol (0.9 mL) at 0° C. After 30 min, DMAP (79.2 mg, 649 μmol) was added, followed by and EDCI (808 mg, 4.22 mmol). The resulting mixture was stirred at rt for 12 h. The mixture was diluted with water (30 mL) and the mixture was extracted with EtOAc (3×50 mL). The combined organic layers were washed with brine (50 mL), dried (Na₂SO₄) and filtered. The filtrate was concentrated and the residue was purified by silica gel chromatography, eluting with (PE/EtOAc=3/1 to 0/1) to give methyl 1-(2,2-dimethylcyclopropyl)-2-oxo-1,2-dihydropyridine-3-carboxylate (220 mg, 30% yield) as yellow oil. LCMS (ESI) m/z 222.0 (M+H)⁺. ¹HNMR (400 MHz, CHLOROFORM-d) δ ppm=8.17 (d, J=7.0 Hz, 1H), 7.51 (d, J=7.0 Hz, 1H), 6.19 (t, J=6.5 Hz, 1H), 3.91 (s, 3H), 3.15-3.10 (m, 1H), 1.31 (s, 3H), 1.00-0.95 (m, 1H), 0.86 (s, 3H), 0.80-0.75 (m, 1H).

Step b: To a solution of compound methyl 1-(2,2-dimethylcyclopropyl)-2-oxo-1,2-dihydropyridine-3-carboxylate (220 mg, 994 μmol) in MeOH (2 mL) and water (1 mL) was added LiOH (71 mg, 3.0 mmol). The mixture was stirred at 20° C. for 1 h. The reaction mixture diluted with aqueous HCl (1 M) to pH=5, and water was added (20 mL). The mixture was extracted with EtOAc (30 mL×3) and the combined organic layers were dried (Na₂SO₄) and filtered. The filtrate was concentrated in vacuo to give compound 1-(2,2-dimethylcyclopropyl)-2-oxo-1,2-dihydropyridine-3-carboxylic acid (200 mg, 97% yield) as a yellow solid, which was used without further purification. LCMS (ESI) m/z 207.9 (M+H)⁺.

Step c: To a solution of compound 1-(2,2-dimethylcyclopropyl)-2-oxo-1,2-dihydropyridine-3-carboxylic acid (150 mg, 723 μmol) in t-BuOH (10 mL) was added DPPA (298 mg, 1.09 mmol, 0.2 mL) and TEA (219 mg, 2.17 mmol, 0.3 mL). The mixture was stirred at 90° C. for 12 h. The reaction mixture was concentrated in vacuo and the residue was purified by silica gel chromatography, eluting with (PE/EtOAc=1/0 to 3/1) to give compound tert-butyl (1-(2,2-dimethylcyclopropyl)-2-oxo-1,2-dihydropyridin-3-yl)carbamate (70 mg, 35% yield) as yellow oil.

Step d: To a solution compound tert-butyl (1-(2,2-dimethylcyclopropyl)-2-oxo-1,2-dihydropyridin-3-yl)carbamate (80 mg, 287 μmol) in EtOAc (1 mL) was added an EtOAc solution of HCl (4 M, 4.00 mL). The mixture was stirred at 20° C. for 1 h. The solution was concentrated in vacuo to give compound 3-amino-1-(2,2-dimethylcyclopropyl)pyridin-2(1H)-one (60 mg, 97% yield, HCl) as a yellow solid, which was of sufficient purity for use in the next reaction. LCMS (ESI) m/z 178.7 (M+H)⁺. ¹HNMR (500 MHz, DMSO-d₆) δ ppm=7.20-7.10 (m, 1H), 6.95-6.85 (m, 1H), 6.11 (t, J=7.0 Hz, 1H), 3.10-3.00 (m, 1H), 1.19 (s, 3H), 1.00-0.95 (m, 1H), 0.85-0.80 (m, 1H), 0.71 (s, 3H).

### Preparation 47: 3-amino-1-(1-methylcyclopropyl)pyridin-2(1H)-one hydrochloride

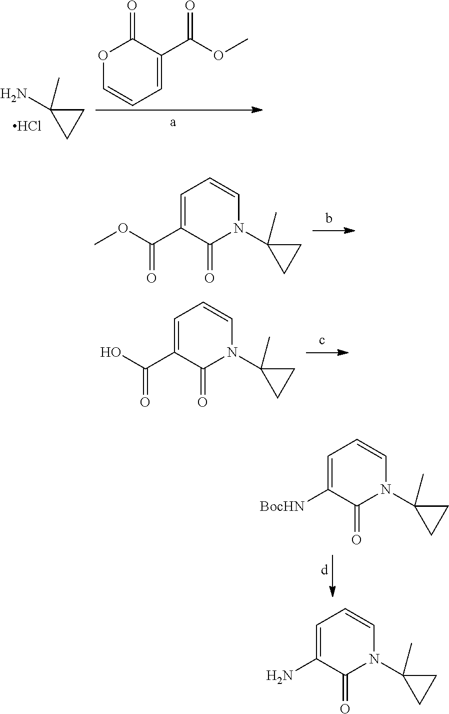

Step a: To a solution of methyl 2-oxo-2H-pyran-3-carboxylate (1.00 g, 6.49 mmol) and 1-methylcyclopropan-1-amine hydrochloride (768 mg, 7.14 mmol) in DMF (50 mL) was added TEA (1.31 g, 13.0 mmol) at 0° C. The mixture was stirred at 0° C. for 30 min and EDCI (1.62 g, 8.43 mmol) and DMAP (159 mg, 1.30 mmol) were added. The resulting mixture was stirred at 25° C. for 12 h. The mixture was diluted with water (100 mL) and the mixture was extracted with EtOAc (3×100 mL). The combined organic layers were washed with brine (100 mL), dried (Na₂SO₄) and filtered. The filtrate was concentrated and the residue was purified by silica gel chromatography (PE/EtOAc=3/1 to 0/1) to give methyl 1-(1-methylcyclopropyl)-2-oxo-1,2-dihydropyridine-3-carboxylate (220 mg, 16% yield) as a yellow oil. LCMS (ESI) m/z 207.9 (M+H)⁺. ¹HNMR (500 MHz, CHLOROFORM-d) δ ppm=8.14 (dd, J=7.0, 2.0 Hz, 1H), 7.66 (d, J=6.5 Hz, 1H), 6.21 (t, J=7.0 Hz, 1H), 3.91 (s, 3H), 1.54 (s, 3H), 1.05-0.95 (m, 4H).

Step b: To a solution of methyl 1-(1-methylcyclopropyl)-2-oxo-1,2-dihydropyridine-3-carboxylate (250 mg, 1.21 mmol) in MeOH (2 mL) and water (1 mL) was added LiOH (86.7 mg, 3.62 mmol). The mixture was stirred at 20° C. for 16 h. The reaction mixture was acidified with 1 M aqueous HCl to pH=5, and it was further diluted with water (20 mL). The mixture was extracted with EtOAc (3×20 mL). The combined organic layers were dried (Na₂SO₄) filtered and concentrated in vacuo to give 1-(1-methylcyclopropyl)-2-oxo-1,2-dihydropyridine-3-carboxylic acid (210 mg, 90% yield) as a yellow solid. ¹HNMR (400 MHz, DMSO-d₆) δ ppm=14.70 (brs, 1H), 8.33 (dd, J=7.2, 2.0 Hz, 1H), 8.23 (dd, J=6.6, 2.0 Hz, 1H), 6.66 (t, J=7.2 Hz, 1H), 1.46 (s, 3H), 1.10-1.00 (m, 2H), 0.95-0.85 (m, 2H).

Step c: To a solution of 1-(1-methylcyclopropyl)-2-oxo-1,2-dihydropyridine-3-carboxylic acid (210 mg, 1.09 mmol) in t-BuOH (10 mL) was added DPPA (449 mg, 1.63 mmol) and TEA (220 mg, 2.17 mmol). The mixture was stirred at 90° C. for 12 h. The reaction mixture was concentrated in vacuo and the residue was purified by silica gel chromatography (5%-20% PE in EtOAc) to give tert-butyl (1-(1-methylcyclopropyl)-2-oxo-1,2-dihydropyridin-3-yl)carbamate (140 mg, 48.7% yield) as a yellow oil. LCMS (ESI) m/z 265.0 (M+H)⁺.

Step d: To a solution of tert-butyl (1-(1-methylcyclopropyl)-2-oxo-1,2-dihydropyridin-3-yl)carbamate (50 mg, 190 μmol) in EtOAc (1 mL) was added an EtOAc solution of HCl (4 M, 2.5 mL). The mixture was stirred at 20° C. for 1 h. The mixture was concentrated in vacuo to give 3-amino-1-(1-methylcyclopropyl)pyridin-2(1H)-one hydrochloride (35 mg, 2.2% yield) as a yellow solid. ¹H NMR (500 MHz, DMSO-d₆) δ ppm=7.50-7.40 (m, 1H), 7.20-7.10 (m, 1H), 6.20 (t, J=7.0 Hz, 1H), 1.42 (s, 3H), 1.00-0.90 (m, 4H).

### Preparation 48: 5-bromo-4-cyclopropoxypyridin-2-amine

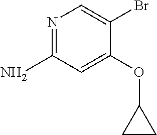 [SMILES: Nc1cc(OC2CC2)c(Br)cn1]

5-bromo-4-cyclopropoxypyridin-2-amine was obtained in a similar to that described in preparation 1, using cyclopropanol. 900 mg, 88.2% yield. LCMS m/z=229.0 [M+H]⁺.

### Preparation 49: 6-bromo-7-cyclopropoxy-2-(1-methyl-2-oxabicyclo[2.1.1]hexan-4-yl)imidazo[1,2-a]pyridine

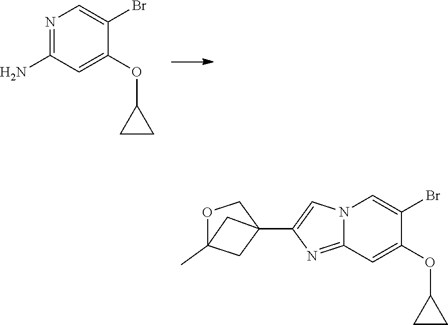 [SMILES: Nc1cc(OC2CC2)c(Br)cn1 ; CC12CC(c3cn4cc(Br)c(OC5CC5)cc4n3)(CO1)C2]

6-bromo-7-cyclopropoxy-2-(1-methyl-2-oxabicyclo[2.1.1]hexan-4-yl)imidazo[1,2-a]pyridine was obtained in a similar fashion to that described in preparation 6, using 5-bromo-4-cyclopropoxypyridin-2-amine. LCMS ES+349.0 (M+1)⁺.

### Preparation 50: phenyl 7-cyclopropoxy-2-(1-methyl-2-oxabicyclo[2.1.1]hexan-4-yl)imidazo[1,2-a]pyridine-6-carboxylate

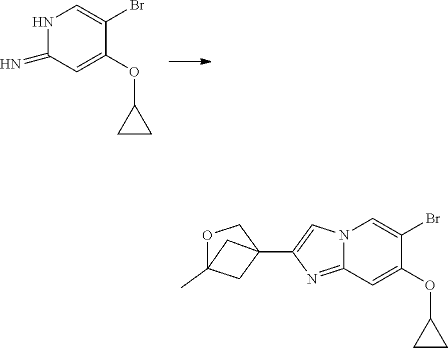 [SMILES: N=c1cc(OC2CC2)c(Br)c[nH]1 ; CC12CC(c3cn4cc(Br)c(OC5CC5)cc4n3)(CO1)C2]

Phenyl 7-cyclopropoxy-2-(1-methyl-2-oxabicyclo[2.1.1]hexan-4-yl)imidazo[1,2-a]pyridine-6-carboxylate was obtained in a similar fashion to that described in preparation 7, using 6-bromo-7-cyclopropoxy-2-(1-methyl-2-oxabicyclo[2.1.1]hexan-4-yl)imidazo[1,2-a]pyridine.

LCMS ES+391.2 (M+1)⁺.

### Preparation 51: 7-cyclopropoxy-2-(1-methyl-2-oxabicyclo[2.1.1]hexan-4-yl)imidazo[1,2-a]pyridine-6-carboxylic acid

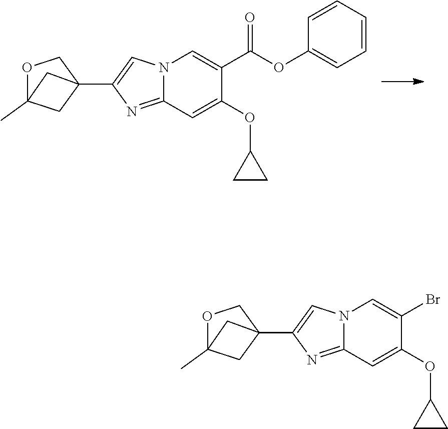 [SMILES: CC12CC(c3cn4cc(C(=O)Oc5ccccc5)c(OC5CC5)cc4n3)(CO1)C2 ; CC12CC(c3cn4cc(Br)c(OC5CC5)cc4n3)(CO1)C2]

7-cyclopropoxy-2-(1-methyl-2-oxabicyclo[2.1.1]hexan-4-yl)imidazo[1,2-a]pyridine-6-carboxylic acid was obtained in a similar fashion to that described in Preparation 8, using phenyl 7-cyclopropoxy-2-(1-methyl-2-oxabicyclo[2.1.1]hexan-4-yl)imidazo[1,2-a]pyridine-6-carboxylate. LCMS ES+315.2 (M+1)⁺.

### Preparation 52: methyl 2-amino-4-cyclobutoxypyrimidine-5-carboxylate

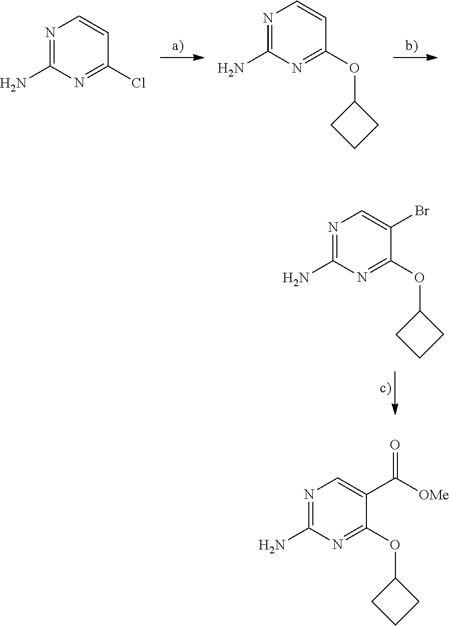 [SMILES: Nc1nccc(Cl)n1 ; Nc1nccc(OC2CCC2)n1 ; Nc1ncc(Br)c(OC2CCC2)n1 ; COC(=O)c1cnc(N)nc1OC1CCC1]

Step a: Cyclobutyl alcohol (15.7 g, 217 mmol) was dissolved in dioxane (200 mL) followed by addition of t-BuOK (25.5 g, 277 mmol) under vigorous stirring. The reaction mixture was stirred at rt for 30 min before a solution of 4-chloropyrimidin-2-amine (28.11 g, 217 mmol) in DMSO (50 mL) was added. The mixture was slowly heated to 60° C. and stirred overnight at the same temperature. Upon completion, the reaction mixture was cooled to rt and concentrated. The residue was diluted with water and extracted with ethyl acetate (100 mL) and the organic layer was washed with water, brine, dried over Na₂SO₄, and evaporated in vacuo to afford 28 g of 4-cyclobutoxypyrimidin-2-amine (93% yield), which was used without further purification. LCMS m/z=166.0 [M+H]⁺.

Step b: To a solution of 4-cyclobutoxypyrimidin-2-amine (28 g, 170 mmol) in CHCl₃ (300 mL) was added NBS (30.3 g, 170 mmol) in portions at 10° C. The resulting mixture was stirred at rt for 2 h and diluted with water. The layers were separated and the organic layer was washed with water, brine, dried over Na2SO4 and evaporated in vacuo to afford 5-bromo-4-cyclobutoxypyrimidin-2-amine (37.2 g, 90% yield), which was used in the next step without further purification.

Step c: To a stirred mixture of 5-bromo-4-cyclobutoxypyrimidin-2-amine (37.2 g, 152 mmol) in MeOH (600 mL) in a steel bomb were added Pd(dppf)Cl₂ (2.49 g, 0.3 mmol), triethylamine (18.5 g, 183 mmol) at rt and then the steel vessel was sealed. Then CO gas was purged into the steel bomb and the stirring was continued at 120° C. for 18 hours. The reaction mixture was allowed to cool to rt, filtered through a pad of celite. The celite pad was washed with excess of MeOH and the filtrate was concentrated. The residue was washed with water, rinsed with MeOH and dried to afford methyl 2-amino-4-cyclobutoxypyrimidine-5-carboxylate (27.1 g, 80% yield). ¹H NMR (METHANOL-d4, 400 MHz) δ 8.59 (s, 1H), 5.2-5.4 (m, 1H), 3.82 (s, 3H), 2.4-2.6 (m, 2H), 2.1-2.3 (m, 2H), 1.8-1.9 (m, 1H), 1.6-1.8 (m, 1H).

### Preparation 53: 4-Cyclobutoxy-5-iodopyrimidin-2-amine

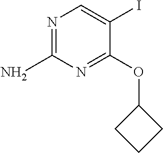 [SMILES: Nc1ncc(I)c(OC2CCC2)n1]

To a solution of 4-cyclobutoxypyrimidin-2-amine [preparation 52] 4.20 g, 25.43 mmol) in DCM (100 mL) was added NIS (5.72 g, 25.43 mmol) at 0° C. under N₂ and the reaction stirred at 25° C. for 16 hr. The mixture was quenched with saturated Na₂SO₃ aq. (200 mL) and the layers separated. The organic layer was washed with brine (200 mL) and dried over Na₂SO4, filtered and concentrated in vacuo. The residue was purified by Combiflash® eluting with PE/EtOAc (0 to 1/1) to afford the title compound (5.50 g, 66.8% yield) as a yellow solid. LCMS m/z=292.5 [M+H]⁺.

### Preparation 54: 7-Cyclobutoxy-6-iodo-2-(1-methyl-2-oxabicyclo[2.1.1]hexan-4-yl)imidazo[1,2-a]pyrimidine

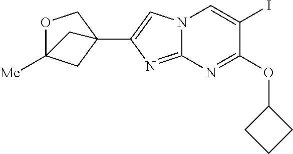 [SMILES: CC12CC(c3cn4cc(I)c(OC5CCC5)nc4n3)(CO1)C2]

To a solution of 4-cyclobutoxy-5-iodopyrimidin-2-amine [preparation 53] (600 mg, 2.06 mmol) and 2-bromo-1-(1-methyl-2-oxabicyclo[2.1.1]hexan-4-yl)ethan-1-one [preparation 12](600 mg, 2.74 mmol) in t-BuOH (10 mL) was added NaHCO₃ (346.1 mg, 4.12 mmol) and the reaction stirred at 100° C. for 16 hr. The mixture was concentrated in vacuo and the residue was purified by CombiFlash® eluting with PE/EtOAc (0 to 1/1) to afford 7-Cyclobutoxy-6-iodo-2-(1-methyl-2-oxabicyclo[2.1.1]hexan-4-yl)imidazo[1,2-a]pyrimidine (431 mg, 51% yield) as a yellow solid. LCMS m/z=412.1 [M+H]⁺.

### Preparation 55: Methyl 7-cyclobutoxy-2-(1-methyl-2-oxabicyclo[2.1.1]hexan-4-yl)imidazo[1,2-a]pyrimidine-6-carboxylate

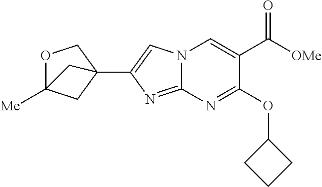 [SMILES: COC(=O)c1cn2cc(C34COC(C)(C3)C4)nc2nc1OC1CCC1]

To a solution of 7-cyclobutoxy-6-iodo-2-(1-methyl-2-oxabicyclo[2.1.1]hexan-4-yl)imidazo[1,2-a]pyrimidine [preparation 54] (431 mg, 1.05 mmol) in MeOH (20 mL) was added Pd(dppf)Cl₂ (76.8 mg, 105.00 μmol) and TEA (1.06 g, 10.50 mmol) and the mixture was degassed with CO, then stirred at 80° C. under CO (50 psi) for 16 hr. The cooled mixture was concentrated in vacuo and the residue purified by CombiFlash® (PE/EtOAc=0 to 1/1) to afford the title compound (283 mg, 76.1% yield) as a brown solid. LCMS m/z=344.2 [M+H]⁺.

### Preparation 56: 7-cyclobutoxy-2-(1-methyl-2-oxabicyclo[2.1.1]hexan-4-yl)imidazo[1,2-a]pyrimidine-6-carboxylic acid

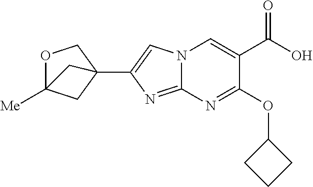 [SMILES: CC12CC(c3cn4cc(C(=O)O)c(OC5CCC5)nc4n3)(CO1)C2]

LiOH·H₂O (59.2 mg, 2.47 mmol) was added to a solution of methyl 7-cyclobutoxy-2-(1-methyl-2-oxabicyclo[2.1.1]hexan-4-yl)imidazo[1,2-a]pyrimidine-6-carboxylate [preparation 55] (283 mg, 824.16 μmol) in MeOH (3 mL) and water (3 mL) and the reaction stirred at 25° C. for 16 hr. The mixture was diluted with saturated HCl aq. to pH=7, then concentrated in vacuo. The residue was co-evaporated with toluene to afford 7-cyclobutoxy-2-(1-methyl-2-oxabicyclo[2.1.1]hexan-4-yl)imidazo[1,2-a]pyrimidine-6-carboxylic acid (200 mg, 67.0% yield) as black oil. LCMS m/z=330.2 [M+H]⁺.

### Preparation 57: methyl 6-amino-4-(sec-butoxy)nicotinate

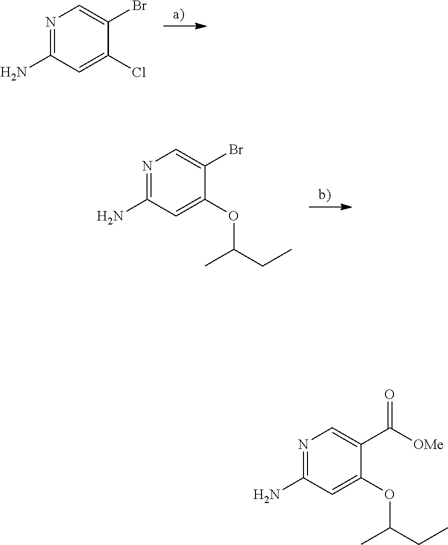 [SMILES: Nc1cc(Cl)c(Br)cn1 ; CCC(C)Oc1cc(N)ncc1Br ; CCC(C)Oc1cc(N)ncc1C(=O)OC]

Step a: NaH (60% dispersion in mineral oil, 16.19 g, 405 mmol) was added in portions to a stirred solution of butan-2-ol (34.24 g, 463 mmol) in DMF (600 mL) at rt. After evolution of hydrogen ceased, 5-bromo-4-chloropyridin-2-amine (80 g, 386 mmol) was added and the resulting solution was heated at 100° C. for 48 h. After cooling to rt, the mixture was diluted with water (3000 mL) and extracted with EtOAc (2×500 mL). The combined extracts were washed with water (4×300 mL), dried (Na₂SO₄) and concentrated in vacuo. The solid residue was recrystallized from hexane/CH₂Cl₂ mixture to give 5-bromo-4-(sec-butoxy)pyridin-2-amine (56.6 g, 60% yield).

Step b: 5-bromo-4-(sec-butoxy)pyridin-2-amine (56.6 g, 231 mmol), triethylamine (24.5 g, 243 mmol) and Pd(dppf)₂Cl₂·CH₂Cl₂ (3.77 g, 4.6 mmol) were dissolved in dry MeOH (800 mL). The reaction mixture was heated at 140° C. in a high pressure vessel at 40 bar CO pressure for 12 h. The solvent was evaporated and the mixture was poured into water (1000 mL). The mixture was extracted with EtOAc (3×200 mL) and the organics were dried over Na₂SO₄ and evaporated to dryness. Crystallization from i-PrOH provided methyl 6-amino-4-(sec-butoxy)nicotinate (17.0 g, 33% yield). LCMS m/z=225.1 [M+H]⁺; 1H NMR (METHANOL-d4, 400 MHz): δ (ppm) 8.36 (s, 1H), 6.12 (s, 1H), 4.46 (sxt, J=6.1 Hz, 1H), 3.81 (s, 3H), 1.64-1.88 (m, 2H), 1.35 (d, J=6.0 Hz, 3H), 1.02 (t, J=7.4 Hz, 3H)

### Preparation 58: 7-isopropoxy-2-(1-methyl-2-oxabicyclo[2.2.1]heptan-4-yl)imidazo[1,2-a]pyridine-6-carboxylic acid

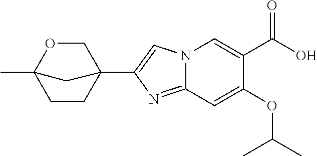 [SMILES: CC(C)Oc1cc2nc(C34CCC(C)(C3)OC4)cn2cc1C(=O)O]

7-isopropoxy-2-(1-methyl-2-oxabicyclo[2.2.1]heptan-4-yl)imidazo[1,2-a]pyridine-6-carboxylic acid (as 2·NaCl salt) was obtained as pale brown solid (680 mg, yield 76% over two steps), from 2-bromo-1-(1-methyl-2-oxabicyclo[2.2.1]heptan-4-yl)ethan-1-one [preparation 15] and methyl 6-amino-4-isopropoxynicotinate [preparation 2], following a similar procedure to that described in Preparation 25. LCMS m/z=331.1 [M+H]⁺.

### Preparation 59A and B: 7-isopropoxy-2-((1S,4R)-1-methyl-2-oxabicyclo[2.2.1]heptan-4-yl)imidazo[1,2-a]pyridine-6-carboxylic acid and 7-isopropoxy-2-((1R,4S)-1-methyl-2-oxabicyclo[2.2.1]heptan-4-yl)imidazo[1,2-a]pyridine-6-carboxylic acid

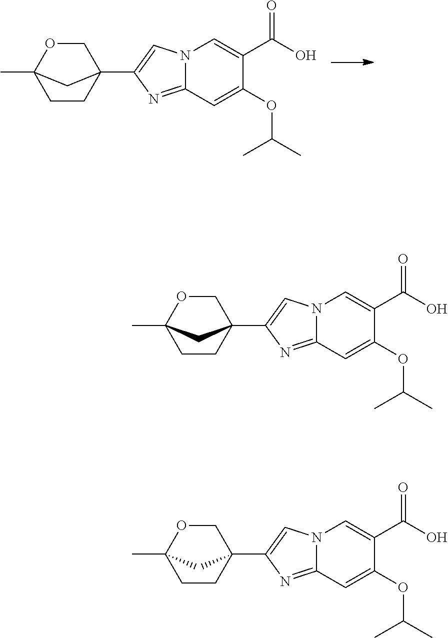

Chiral separation of racemic 7-isopropoxy-2-(1-methyl-2-oxabicyclo[2.2.1]heptan-4-yl)imidazo[1,2-a]pyridine-6-carboxylic acid (as 2·NaCl salt) [preparation 58] (100 mg, 224 ummol) using method: LUX Cellulose-2 LC 30×250 mm, 5 um; 40% MeOH w/ No Modifier in CO₂ (flow rate: 100 mL/min, ABPR 120 bar, MBPR 40 psi, column temp 40 deg C.) provided as Peak 1: 7-isopropoxy-2-((1S,4R)-1-methyl-2-oxabicyclo[2.2.1]heptan-4-yl)imidazo[1,2-a]pyridine-6-carboxylic acid (33 mg, yield 45%). ¹H NMR (400 MHz, METHANOL-d4) δ 8.63 (s, 1H), 7.74 (s, 1H), 6.96 (s, 1H), 4.78-4.85 (m, 1H), 4.03 (dd, J=3.26, 6.53 Hz, 1H), 3.90-3.98 (m, 1H), 1.76-2.28 (m, 6H), 1.46 (d, J=6.02 Hz, 6H). Absolute stereochemistry was confirmed by X-ray crystallography.

Peak 2: 7-isopropoxy-2-((1R,4S)-1-methyl-2-oxabicyclo[2.2.1]heptan-4-yl)imidazo[1,2-a]pyridine-6-carboxylic acid (32 mg, yield 43%). 1H NMR (400 MHz, METHANOL-d4) δ 8.63 (s, 1H), 7.74 (s, 1H), 6.96 (s, 1H), 4.83 (td, J=6.12, 12.11 Hz, 1H), 4.03 (dd, J=3.26, 6.78 Hz, 1H), 3.95 (d, J=6.78 Hz, 1H), 1.79-2.26 (m, 6H), 1.46 (d, J=6.02 Hz, 6H).

### Preparation 60: 3-amino-1-(2,2-difluorocyclopropyl)pyridin-2(1H)-one

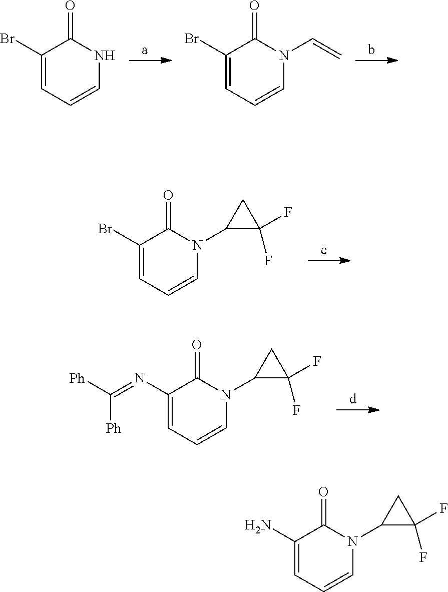

Step a: To a solution of 3-bromo-1H-pyridin-2-one (200 mg, 1.15 mmol) and vinylboronic acid (165 mg, 2.30 mmol) in Dioxane (10 mL) was added diacetoxycopper (229 mg, 1.26 mmol) 2-(2-pyridyl)pyridine (197 mg, 1.26 mmol) and Na₂CO₃ (365 mg, 3.45 mmol). The mixture was stirred at 50° C. for 48 hrs. The mixture was filtered and the filtrate was concentrated in vacuo to give a crude residue, which was purified by silica gel column chromatography (heptane/EtOAc=10/1 to 3/1) to give 3-bromo-1-vinyl-pyridin-2-one (90.4 mg, 452 μmol, 39.3% yield) as a white solid. LCMS m/z=201.8 [M+H]⁺

Step b: To a solid mixture of (2-chloro-2,2-difluoro-acetyl)oxysodium (689 mg, 4.52 mmol) and 3-bromo-1-vinyl-pyridin-2-one (90.4 mg, 452 μmol) was added Diglyme (4 mL). The vial was sealed with a teflon-lined cap; the mixture was purged with N₂ for 10 mins. The N₂ line was removed and the vial was heated at 170° C. for 12 h. Conversion was around 20%. Another batch of (2-chloro-2,2-difluoro-acetyl)oxysodium (689 mg, 4.52 mmol) was added to the vial, purged for another 10 mins and heated at 170° C. for 12 h. The process was repeated another two times to drive the reaction to completion. The mixture was filtered and concentrated. The residual was purified by silica gel column chromatography (heptane/EtOAc=10/1) to give 3-bromo-1-(2,2-difluorocyclopropyl)pyridin-2-one (53.0 mg, 212 μmol, 46.9% yield) as a white solid. LCMS m/z=251.7 [M+H]⁺

Step c: Charged the vial with 3-bromo-1-(2,2-difluorocyclopropyl)pyridin-2-one (53.0 mg, 212 μmol), Sodium tert-butoxide (40.7 mg, 424 μmol), Pd₂(dba)₃ (5.82 mg, 6.36 μmol), [1-(2-diphenylphosphanyl-1-naphthyl)-2-naphthyl]-diphenyl-phosphane (10.6 mg, 16.9 μmol).

The vial was capped with a teflon-lined cap and flushed with N₂. To the vial was added diphenylmethanimine (46.1 mg, 254 μmol, 42.6 μL) and Toluene (2.00 mL). The resulting mixture was heated at 100° C. for 16 hrs. The mixture was filtered through a pad of celite, eluted with DCM. The filtrate was concentrated and purified by silica gel column chromatography (0-100% heptane:EtOAc) to give 3-(benzhydrylideneamino)-1-(2,2-difluorocyclopropyl)pyridin-2-one (23.1 mg, 65.9 μmol, 31.1% yield) as a yellow solid.

Step d: To a solution of 3-(benzhydrylideneamino)-1-(2,2-difluorocyclopropyl)pyridin-2-one (23.1 mg, 65.9 μmol) in DCM (0.5 mL) and MeOH (0.5 mL) was added HCl (4M, 82 μL) in dioxane. The mixture was stirred at 22° C. for 3 hrs. The reaction was concentrated in vacuo and used the crude as is. LCMS m/z=186.9 [M+H]⁺

### Preparation 61: rac-(Trans)-3-amino-1-(2-fluorocyclopropyl)pyridin-2(1H)-one

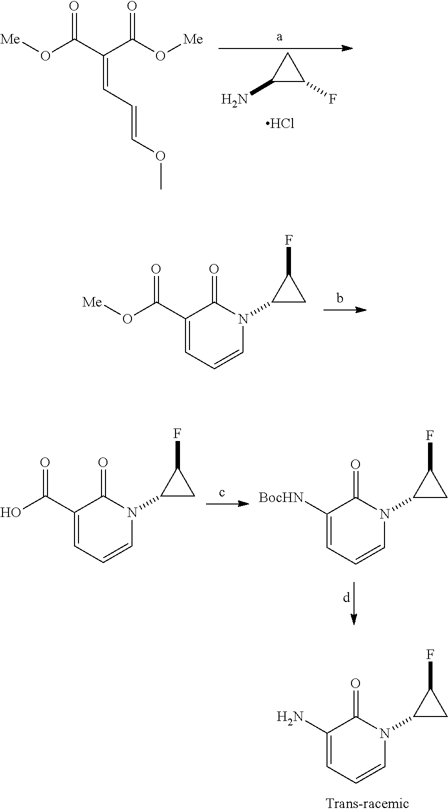

Step a: In a 30 mL vial, a mixture of racemic (trans)-2-fluorocyclopropanamine hydrochloride (279 mg, 2.50 mmol), dimethyl 2-[(E)-3-methoxyprop-2-enylidene]propanedioate (500 mg, 2.50 mmol) and triethylamine (278 mg, 2.75 mmol, 383 μL) in MeOH (3 mL) was stirred at rt for 15 h. Volatiles were evaporated under reduced pressure and the resulting residue was partitioned between dichloromethane and water. The organic layer was separated, dried over Na₂SO₄, filtered and concentrated in vacuo to obtain dimethyl racemic-(E)-2-(Trans)-(3-((2-fluorocyclopropyl)amino)allylidene)malonate. The crude material was dissolved in ethanol (3 mL) followed by the addition of solid KOH (263 mg, 4.69 mmol). The reaction mixture was stirred at rt for 1 h and then refluxed for 2 h. After that, the resulting mixture was evaporated in vacuo and the residue was dissolved in water and neutralized with conc. HCl. The aqueous solution was extracted with EtOAc (10 mL×3) and the combined organic layers were dried, filtered and concentrated to give a residual oil. It was purified by mass-directed HPLC to give methyl 1-Trans-(2-fluorocyclopropyl)-2-oxo-1,2-dihydropyridine-3-carboxylate (257 mg, 1.22 mmol, 48.6% yield) as a colorless film. LCMS m/z=211.9 [M+H]⁺; ¹H NMR (400 MHz, MeOH-d₄) δ: 1.51 (dddd, J=11.07, 8.82, 7.22, 6.27 Hz, 1H) 1.69-1.82 (m, 1H) 3.69-3.80 (m, 1H) 3.85 (s, 3H) 4.74-4.92 (m, 1H) 6.42 (t, J=7.03 Hz, 1H) 7.79 (dd, J=6.78, 2.01 Hz, 1H) 8.21 (dd, J=7.28, 2.26 Hz, 1H).

Step b: NaOH (97.2 mg, 2.43 mmol) was added to a mixture of methyl 1-Trans-(2-fluorocyclopropyl)-2-oxo-1,2-dihydropyridine-3-carboxylate (257 mg, 1.22 mmol) in THF (2 mL) and MeOH (2 mL) at rt and stirred for 5 h. The reaction mixture was dried under vacuum to give racemic 1-Trans-(2-fluorocyclopropyl)-2-oxo-1,2-dihydropyridine-3-carboxylic acid as a sodium salt. The Material was used without further purification in the next step.

Step c: To a solution of 1-Trans-(2-fluorocyclopropyl)-2-oxo-1,2-dihydropyridine-3-carboxylic acid (50.0 mg, 253 μmol) in t-BuOH (3 mL) was added DPPA (105 mg, 380 mol, 82.0 μL) and triethylamine (51.3 mg, 507 μmol, 70.7 μL). The mixture was stirred at 90° C. for 12 h. The reaction mixture was concentrated in vacuo to give the residue, which was purified by silica gel chromatography (PE/EtOAc=20/1 to 5/1) to give racemic tert-butyl (1-Trans-(2-fluorocyclopropyl)-2-oxo-1,2-dihydropyridin-3-yl)carbamate (57.8 mg, 215 mol, 84.9% yield) as a yellow oil. LCMS m/z=269.1 [M+H]⁺

Step d: To a solution of racemic tert-butyl (1-Trans-(2-fluorocyclopropyl)-2-oxo-1,2-dihydropyridin-3-yl)carbamate (142 mg, 527 μmol) in dioxane (2 mL) was added HCl (4 M in dioxane, 659 μL). The mixture was stirred at 22° C. for 14 h. Solvent was removed to provide rac-(Trans)-3-amino-1-(2-fluorocyclopropyl)pyridin-2(1H)-one, which was used without further purification.

### Preparation 62A and B: (S)-7-((1,1-difluoropropan-2-yl)oxy)-2-(1-methyl-2-oxabicyclo[2.1.1]hexan-4-yl)imidazo[1,2-a]pyridine-6-carboxylic acid and (R)-7-((1,1-difluoropropan-2-yl)oxy)-2-(1-methyl-2-oxabicyclo[2.1.1]hexan-4-yl)imidazo[1,2-a]pyridine-6-carboxylic acid

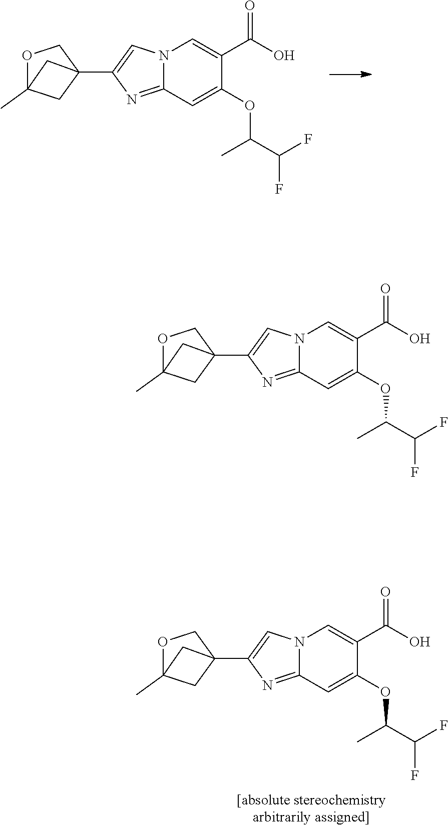 [SMILES: CC(Oc1cc2nc(C34COC(C)(C3)C4)cn2cc1C(=O)O)C(F)F ; C[C@H](Oc1cc2nc(C34COC(C)(C3)C4)cn2cc1C(=O)O)C(F)F ; C[C@@H](Oc1cc2nc(C34COC(C)(C3)C4)cn2cc1C(=O)O)C(F)F]

Racemic 7-((1,1-difluoropropan-2-yl)oxy)-2-(1-methyl-2-oxabicyclo[2.1.1]hexan-4-yl)imidazo[1,2-a]pyridine-6-carboxylic acid was obtained from racemic 1,1-difluoropropan-2-ol, following a similar procedure to that described in Preparation 36 and purified by SFC: CHIRALPAK AD-H 30×250 mm, 5 um Method: 20% IPA w/0.1% DEA in CO₂ (flow rate: 100 mL/min, ABPR 120 bar, MBPR 60 psi, column temp 40 deg C.) to obtain Peak 1: (S)-7-((1,1-difluoropropan-2-yl)oxy)-2-(1-methyl-2-oxabicyclo[2.1.1]hexan-4-yl)imidazo[1,2-a]pyridine-6-carboxylic acid (>99% ee). LCMS m/z=353.4 [M+H]⁺. Stereochemistry was arbitrarily assigned.

Peak 2: (R)-7-((1,1-difluoropropan-2-yl)oxy)-2-(1-methyl-2-oxabicyclo[2.1.1]hexan-4-yl)imidazo[1,2-a]pyridine-6-carboxylic acid (96% ee), LCMS m/z=353.4 [M+H]⁺. Stereochemistry was arbitrarily assigned.

### Preparation 63: rac-(Cis)-3-amino-1-(2-fluorocyclopropyl)pyridin-2(1H)-one

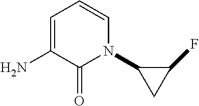

Cis-racemic 3-amino-1-(2-fluorocyclopropyl)pyridin-2(1H)-one was prepared from (cis)-2-fluorocyclopropan-1-amine in a similar fashion to that described in Preparation 61. LCMS (ESI) m/z 165.2 (M+H)⁺.

### Preparation 64: 7-isopropoxy-2-(1-methyl-2-oxabicyclo[2.2.1]heptan-4-yl)imidazo[1,2-a]pyrimidine-6-carboxylic acid

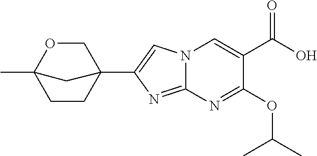 [SMILES: CC(C)Oc1nc2nc(C34CCC(C)(C3)OC4)cn2cc1C(=O)O]

Part A: A mixture of 2-bromo-1-(1-methyl-2-oxabicyclo[2.2.1]heptan-4-yl)ethan-1-one [preparation 15] (560 mg, 2.40 mmol), isopropyl 2-amino-4-isopropoxypyrimidine-5-carboxylate [preparation 10] (479 mg, 2.0 mmol) and NaHCO₃ (504 mg, 6.0 mmol) in MeCN (6.0 mL) and toluene (4.0 mL) was heated at 90° C. overnight. The cooled mixture was partitioned between EtOAc and brine, the layers separated and the aqueous layer was extracted with EtOAc. The combined organic phases were dried, filtered and concentrated in vacuo. The crude was purified by silica gel column chromatography eluting with EtOAc/heptanes (50/50 to 100/0) to give isopropyl 7-isopropoxy-2-(1-methyl-2-oxabicyclo[2.2.1]heptan-4-yl)imidazo[1,2-a]pyrimidine-6-carboxylate, as pale yellow oil. ¹H NMR (400 MHz, MeOH-d₄) δ: 1.39 (d, 6H), 1.45 (d, 6H), 1.47 (s, 3H), 1.76-2.21 (m, 6H), 3.91 (d, 1H), 4.04 (dd, 1H), 5.23 (td, 1H), 5.52 (quin, 1H), 7.48 (s, 1H), 9.15 (s, 1H).

Part B: A solution of isopropyl 7-isopropoxy-2-(1-methyl-2-oxabicyclo[2.2.1]heptan-4-yl)imidazo[1,2-a]pyrimidine-6-carboxylate (275 mg, 0.736 mmol) in 1M NaOH (736 μL), THF (2.0 mL) and MeOH (2.0 mL) was stirred at rt for 2 h. The mixture was acidified to pH 3 using 1N HCl, the solution evaporated under reduced pressure and the solid lyophilised to provide 7-isopropoxy-2-(1-methyl-2-oxabicyclo[2.2.1]heptan-4-yl)imidazo[1,2-a]pyrimidine-6-carboxylic acid, as a white powder. LCMS m/z=332.2 [M+H]⁺.

### Preparation 65: 7-cyclobutoxy-2-(1-methyl-2-oxabicyclo[2.2.2]octan-4-yl)imidazo[1,2-a]pyridine-6-carboxylic acid

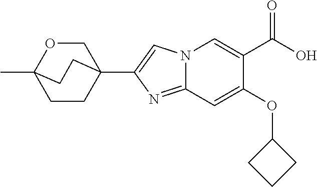 [SMILES: CC12CCC(c3cn4cc(C(=O)O)c(OC5CCC5)cc4n3)(CC1)CO2]

The title compound was prepared in a similar fashion to that described in preparation 64, starting from 2-bromo-1-(1-methyl-2-oxabicyclo[2.2.2]octan-4-yl)ethan-1-one [preparation 13] and methyl 6-amino-4-cyclobutoxynicotinate [preparation 9] yielding an off-white solid. LCMS m/z=346.9 [M+H]⁺.

### Preparation 66: 2-amino-4-cyclobutoxypyrimidine-5-carboxylic acid

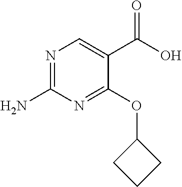 [SMILES: Nc1ncc(C(=O)O)c(OC2CCC2)n1]

To a suspension of methyl 2-amino-4-cyclobutoxypyrimidine-5-carboxylate [preparation 52](10.7 g, 47.8 mmol) in MeOH was added aq. solution of NaOH (2.87 g, 71.7 mmol in 50 mL of water). The mixture was heated to 50° C. and stirred for 5 h. Upon completion of the reaction, the mixture was concentrated. The residue was diluted with water and acidified with citric acid. The precipitated solid was collected, washed with water, rinsed with MeOH and dried to afford 2-amino-4-cyclobutoxypyrimidine-5-carboxylic acid (6.3 g, 63%), which was used without further purification.

### Preparation 67: Methyl 2-(2-oxabicyclo[2.1.1]hexan-4-yl)-7-isopropoxyimidazo[1,2-a]pyridine-6-carboxylate

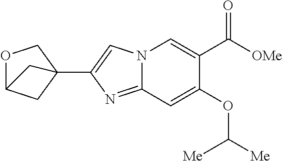 [SMILES: COC(=O)c1cn2cc(C34COC(C3)C4)nc2cc1OC(C)C]

A mixture of methyl 6-amino-4-isopropoxynicotinate [preparation 2] (1.30 g, 6.18 mmol), 1-(2-oxabicyclo[2.1.1]hexan-4-yl)-2-bromoethan-1-one [preparation 20] (1.52 g, 7.42 mmol) and NaHCO₃ (623 mg, 7.42 mmol) in MeCN (25 mL) and toluene (25 mL) was heated in a sealed tube at 90° C. for 14 h. The mixture was cooled to rt, diluted with H₂O (100 mL) and extracted with DCM (3×50 mL). The combined organics were washed with brine, dried (Na₂SO₄) and evaporated to dryness in vacuo to afford methyl 2-(2-oxabicyclo[2.1.1]hexan-4-yl)-7-isopropoxyimidazo[1,2-a]pyridine-6-carboxylate as a yellow oil (1.65 g). LCMS m/z=317.2 [M+H]⁺.

### Preparation 68: 2-(2-Oxabicyclo[2.1.1]hexan-4-yl)-7-isopropoxyimidazo[1,2-a]pyridine-6-carboxylic acid

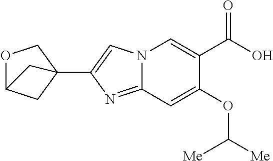 [SMILES: CC(C)Oc1cc2nc(C34COC(C3)C4)cn2cc1C(=O)O]

A mixture of methyl 2-(2-oxabicyclo[2.1.1]hexan-4-yl)-7-isopropoxyimidazo[1,2-a]pyridine-6-carboxylate [preparation 67] (1.65 g, 5.22 mmol), LiOH·H₂O (218.8 mg, 5.22 mmol) in THF (50 mL) and water (5 mL) was stirred at rt for 16 h. The THF was removed in vacuo, H₂O (50 mL) and activated carbon (1 g) were added and the mixture filtered. The filtrate was acidified with conc. HCl to pH 3-4 and the precipitate was filtered, washed with water and air-dried to afford 2-(2-oxabicyclo[2.1.1]hexan-4-yl)-7-isopropoxyimidazo[1,2-a]pyridine-6-carboxylic acid (1.30 g, 76.4% yield) as a yellow solid. LCMS m/z=303.0 [M+H]⁺.

### Preparation 69: 2-(2-oxabicyclo[2.2.1]heptan-4-yl)-7-isopropoxyimidazo[1,2-a]pyridine-6-carboxylic acid

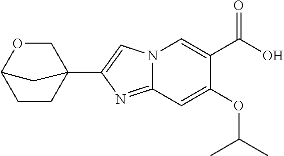 [SMILES: CC(C)Oc1cc2nc(C34CCC(C3)OC4)cn2cc1C(=O)O]

The title compound was prepared in a similar fashion to that described for 7-isopropoxy-2-(1-methyl-2-oxabicyclo[2.2.1]heptan-4-yl)imidazo[1,2-a]pyrimidine-6-carboxylic acid [preparation 64], except starting with 1-(2-oxabicyclo[2.2.1]heptan-4-yl)-2-bromoethan-1-one [preparation 14] instead of 2-bromo-1-(1-methyl-2-oxabicyclo[2.2.1]heptan-4-yl)ethan-1-one. LCMS m/z=317.1 [M+H]⁺.

### Preparation 70: 7-ethoxy-2-(1-methyl-2-oxabicyclo[2.1.1]hexan-4-yl)imidazo[1,2-a]pyrimidine-6-carboxylic acid

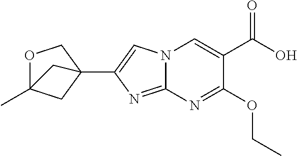 [SMILES: CCOc1nc2nc(C34COC(C)(C3)C4)cn2cc1C(=O)O]

The title compound was prepared in a similar fashion to that described for 7-isopropoxy-2-(1-methyl-2-oxabicyclo[2.2.1]heptan-4-yl)imidazo[1,2-a]pyrimidine-6-carboxylic acid [preparation 64], except starting with 2-bromo-1-(1-methyl-2-oxabicyclo[2.1.1]hexan-4-yl)ethan-1-one [preparation 12] instead of 2-bromo-1-(1-methyl-2-oxabicyclo[2.2.1]heptan-4-yl)ethan-1-one. LCMS m/z=304.1 [M+H]⁺.

### Preparation 71: Methyl 6-amino-5-fluoro-4-isopropoxynicotinate

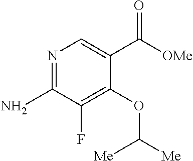 [SMILES: COC(=O)c1cnc(N)c(F)c1OC(C)C]

1-Chloromethyl-4-fluoro-1,4-diazoniabicyclo[2.2.2]octane bis(tetrafluoroborate) (2.53 g, 7.14 mmol) was added to a solution of methyl 6-amino-4-isopropoxynicotinate [preparation 2] (500 mg, 2.38 mmol) in CHCl₃ (12 mL) and water (12 mL) and the reaction stirred for 18 h. The layers were separated, the organic phase dried, concentrated in vacuo, and purified by column chromatography to afford methyl 6-amino-5-fluoro-4-isopropoxynicotinate (118 mg, 21.7% yield). LCMS m/z=229.0 [M+H]^{+ 1}H NMR (500 MHz, CDCl₃) δ:1.37 (dd, 6H) 3.85 (s, 3H) 4.71 (td, 1H) 5.26 (br s, 2H) 8.39 (s, 1H).

### Preparation 72: Methyl 8-fluoro-7-isopropoxy-2-(1-methyl-2-oxabicyclo[2.1.1]hexan-4-yl)imidazo[1,2-a]pyridine-6-carboxylate

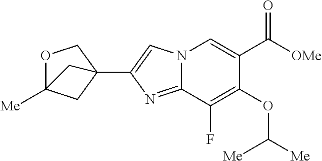 [SMILES: COC(=O)c1cn2cc(C34COC(C)(C3)C4)nc2c(F)c1OC(C)C]

A mixture of methyl 6-amino-5-fluoro-4-isopropoxynicotinate [preparation 71] (140 mg, 0.613 mmol), 2-bromo-1-(1-methyl-2-oxabicyclo[2.1.1]hexan-4-yl)ethan-1-one [preparation 12] (134 mg, 0.613 mmol) and NaHCO₃ (155 mg, 1.84 mmol) in EtOH (1.5 mL) was heated at 80° C. for 18 h. The cooled mixture was dry loaded onto silica gel and purified by column chromatography on silica gel to afford methyl 8-fluoro-7-isopropoxy-2-(1-methyl-2-oxabicyclo[2.1.1]hexan-4-yl)imidazo[1,2-a]pyridine-6-carboxylate (100 mg, 46.8% yield). LCMS m/z=349.0 [M+H]⁺.

### Preparation 73: 8-Fluoro-7-isopropoxy-2-(1-methyl-2-oxabicyclo[2.1.1]hexan-4-yl)imidazo[1,2-a]pyridine-6-carboxylic acid

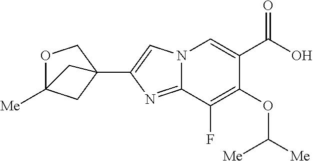 [SMILES: CC(C)Oc1c(C(=O)O)cn2cc(C34COC(C)(C3)C4)nc2c1F]

A mixture of methyl 8-fluoro-7-isopropoxy-2-(1-methyl-2-oxabicyclo[2.1.1]hexan-4-yl)imidazo[1,2-a]pyridine-6-carboxylate [preparation 72] (110 mg, 0.280 mmol) in MeOH (932 μL), H₂O (932 μL) and THF (932 μL) was treated with LiOH (20.1 mg, 0.839 mmol) and the reaction stirred for 2 h. The solution was acidified and evaporated under reduced pressure to afford 8-fluoro-7-isopropoxy-2-(1-methyl-2-oxabicyclo[2.1.1]hexan-4-yl)imidazo[1,2-a]pyridine-6-carboxylic acid. LCMS m/z=335.0 [M+H]⁺.

### Preparation 74: 7-cyclobutoxy-2-(1-(fluoromethyl)-2-oxabicyclo[2.1.1]hexan-4-yl)imidazo[1,2-a]pyridine-6-carboxylic acid

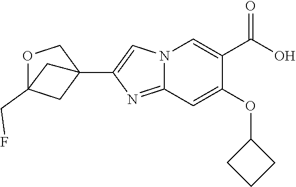 [SMILES: O=C(O)c1cn2cc(C34COC(CF)(C3)C4)nc2cc1OC1CCC1]

The title compound was prepared in a similar fashion to that described in preparation 64, starting from 2-bromo-1-(1-(fluoromethyl)-2-oxabicyclo[2.1.1]hexan-4-yl)ethan-1-one [preparation 19] and methyl 6-amino-4-cyclobutoxynicotinate [preparation 9] yielding an off-white solid. LCMS m/z=357.2 [M+H]⁺.

### Preparation 75: methyl 2-amino-4-(sec-butoxy)pyrimidine-5-carboxylate

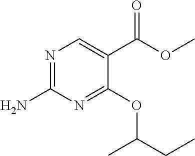 [SMILES: CCC(C)Oc1nc(N)ncc1C(=O)OC]

The title compound, methyl 2-amino-4-(sec-butoxy)pyrimidine-5-carboxylate, was synthesized in a similar fashion to that described in Preparation 11a, but starting with racemic 2-butanol, instead of (R)-butan-2-ol.

### Preparation 76: Trans-racemic 3-amino-1-(2-methylcyclopropyl)pyridin-2(1H)-one hydrochloride

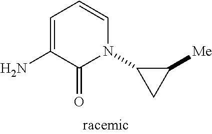

Trans-racemic 3-amino-1-(2-methylcyclopropyl)pyridin-2(1H)-one hydrochloride was prepared from Trans-2-methylcyclopropan-1-amine hydrochloride in a similar fashion to that described in Preparation 61. LCMS (ESI) m/z 169.0 (M+H)⁺.

### Preparation 77: Trans-3-amino-1-(2-ethylcyclopropyl)pyridin-2(1H)-one hydrochloride

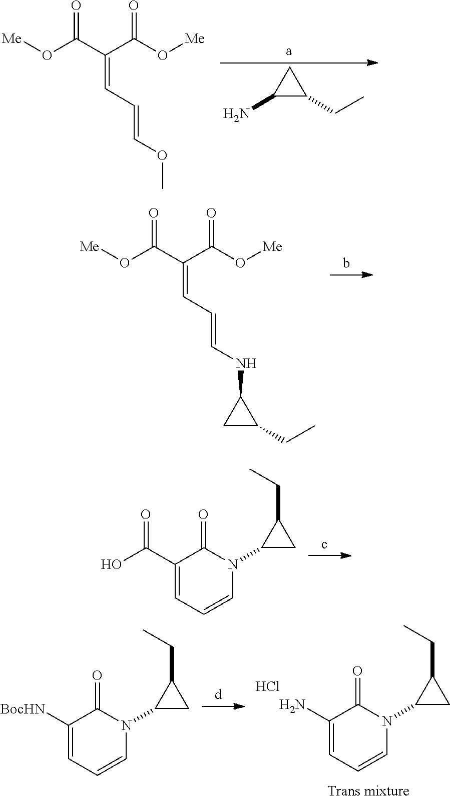

Step a: To a solution of dimethyl (E)-2-(3-methoxyallylidene)malonate (500 mg, 2.50 mmol) and trans-2-ethylcyclopropan-1-amine (212.67 mg, 1.75 mmol) in MeOH (5 mL) was added TEA (278.01 mg, 2.75 mmol) and the reaction stirred at 50° C. for 16 h. The mixture was quenched with water (30 mL) and extracted with EtOAc (50 mL). The combined organic layer was washed with brine (20 mL), dried over Na₂SO₄, filtered and concentrated in vacuo to give trans-dimethyl 2-((E)-3-(2-ethylcyclopropyl)amino)allylidene)malonate (600 mg, 95% yield) as yellow oil. LCMS m/z=253.9 [M+H]⁺

Step b: To a solution of trans-dimethyl 2-((E)-3-(2-ethylcyclopropyl)amino)allylidene)malonate (600 mg, 2.37 mmol) in EtOH (10 mL) was added KOH (212.25 mg, 3.79 mmol) and the reaction was stirred at 20° C. for 16 h. The aqueous phase was adjusted to pH=3 using aq. HCl and extracted with EtOAc (20 mL×3). The combined organic layer was washed with brine (20 mL), dried over Na₂SO₄, filtered and concentrated in vacuo to give trans-1-(2-ethylcyclopropyl)-2-oxo-1,2-dihydropyridine-3-carboxylic acid (500 mg, 97% yield) as yellow solid. LCMS m/z=207.4 [M+H]⁺

Step c: To a solution of trans-1-(2-ethylcyclopropyl)-2-oxo-1,2-dihydropyridine-3-carboxylic acid (250 mg, 1.21 mmol) in t-BuOH (15 mL) was added DPPA (498.01 mg, 1.81 mmol) and TEA (366.23 mg, 3.62 mmol) and the reaction stirred at 90° C. for 12 h. The mixture was concentrated in vacuo to give the residue, which was purified by Combiflash® (PE/EtOAc=3/1) to give trans-tert-butyl (1-(2-ethylcyclopropyl)-2-oxo-1,2-dihydropyridin-3-yl)carbamate (180 mg, 51% yield) as yellow oil. LCMS m/z=279.0 [M+H]⁺

Step d: To a solution of trans-tert-butyl (1-(2-ethylcyclopropyl)-2-oxo-1,2-dihydropyridin-3-yl)carbamate (180 mg, 0.641 mmol) was added HCl/dioxane (10 mL) and the reaction stirred at 20° C. for 1 h. The mixture was concentrated in vacuo to give trans-3-amino-1-(2-ethylcyclopropyl)pyridin-2(1H)-one hydrochloride (115 mg, 97% yield) as a yellow solid. LCMS m/z=179.0 [M+H]⁺

### Preparation 78: Trans-3-amino-1-(2-methoxycyclopropyl)pyridin-2(1H)-one hydrochloride

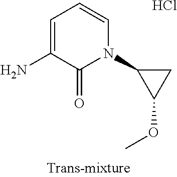

Trans-3-amino-1-(2-methoxycyclopropyl)pyridin-2(1H)-one hydrochloride was prepared as a yellow solid, 160 mg, from dimethyl (E)-2-(3-methoxyallylidene)malonate and trans-2-methoxycyclopropan-1-amine, following a similar procedure to that described in Preparation 77. LCMS m/z=180.9 [M+H]⁺

### Preparation 79: 3-amino-1-(spiro[2.2]pentan-1-yl)pyridin-2(1H)-one

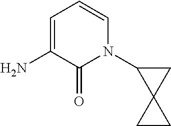 [SMILES: Nc1cccn(C2CC23CC3)c1=O]

3-Amino-1-(spiro[2.2]pentan-1-yl)pyridin-2(1H)-one was obtained from spiro[2.2]pentan-1-amine hydrochloride and dimethyl (E)-2-(3-methoxyallylidene)malonate, following a similar procedure to that described in Preparation 77.

### Preparations 80 and 81: Cis-3-amino-1-(2-(difluoromethyl)-2-methylcyclopropyl)pyridin-2(1H)-one hydrochloride and trans-3-amino-1-(2-(difluoromethyl)-2-methylcyclopropyl)pyridin-2(1H)-one hydrochloride

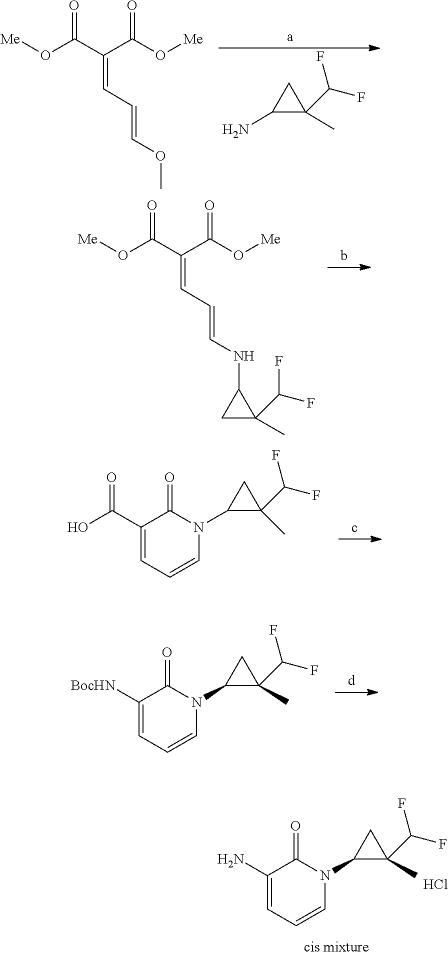

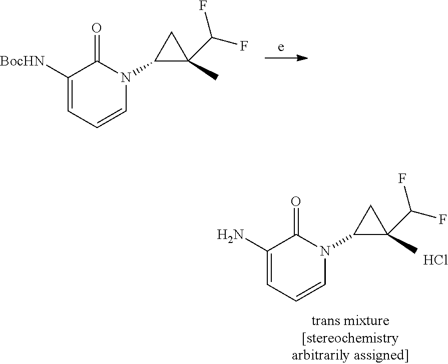 [SMILES: CC(C)(C)OC(=O)Nc1cccn([C@@H]2C[C@]2(C)C(F)F)c1=O ; C[C@]1(C(F)F)C[C@H]1n1cccc(N)c1=O ; Cl]

Step a: To a solution of dimethyl (E)-2-(3-methoxyallylidene)malonate (500 mg, 2.50 mmol) in MeOH (50 mL) was added 2-(difluoromethyl)-2-methylcyclopropan-1-amine hydrochloride (393.6 mg, 2.50 mmol) and TEA (505.47 mg, 5.0 mmol) and the reaction stirred at 50° C. for 2 h. The mixture was concentrated in vacuo to give dimethyl 2-((E)-3-((2-(difluoromethyl)-2-methylcyclopropyl)amino)allylidene)malonate (640 mg, 89% yield) which was used directly in the next reaction. LCMS m/z=289.8 [M+H]⁺

Step b: To a solution of dimethyl 2-((E)-3-((2-(difluoromethyl)-2-methylcyclopropyl)amino)allylidene)malonate (640 mg, 2.21 mmol) in EtOH (30 mL) was added KOH (198.61 mg, 3.54 mmol) and the mixture stirred at 25° C. for 16 h. The reaction was concentrated in vacuo to give a residue which was diluted with water (30 mL) and the pH adjusted to 4 with aq. HCl. The mixture was extracted with EtOAc (50 mL), the combined organic layers washed with brine (30 mL), dried over Na₂SO₄, filtered and concentrated in vacuo to give 1-(2-(difluoromethyl)-2-methylcyclopropyl)-2-oxo-1,2-dihydropyridine-3-carboxylic acid (450 mg, 83.6% yield) as yellow solid. 1H NMR (500 MHz, CDCl₃) δ ppm: 14.07 (s, 1H), 8.55 (dd, J=2.0, 7.0 Hz, 1H), 7.62 (dd, J=2.0, 6.5 Hz, 1H), 6.60 (t, J=7.0 Hz, 1H), 6.11-5.87 (m, 1H), 1.73 (t, J=7.7 Hz, 1H), 1.35-1.32 (m, 1H), 1.29-1.21 (m, 1H), 1.01 (s, 3H).

Step c: To a solution of 1-(2-(difluoromethyl)-2-methylcyclopropyl)-2-oxo-1,2-dihydropyridine-3-carboxylic acid (450 mg, 1.85 mmol) in t-BuOH (20 mL) was added TEA (561.69 mg, 5.55 mmol) and DPPA (763.80 mg, 2.78 mmol) and the reaction was stirred at 90° C. for 16 h. The reaction was diluted with water (30 mL) and extracted with EtOAc (50 mL×3). The combined organic layers were washed with NH₄Cl (30 mL), dried over Na₂SO₄, filtered and concentrated in vacuo. The residue was purified by silica gel column chromatography (PE:EtOAc=1:0 to 1:1) to give:

Peak 1, cis-tert-butyl (1-(2-(difluoromethyl)-2-methylcyclopropyl)-2-oxo-1,2-dihydropyridin-3-yl)carbamate [stereochemistry arbitrarily assigned] (190 mg, 32.7% yield). 1HNMR (500 MHz, CDCl₃) δ ppm: 7.99 (d, J=7.0 Hz, 1H), 7.67 (s, 1H), 6.87 (dd, J=2.0, 7.0 Hz, 1H), 6.23 (t, J=7.0 Hz, 1H), 6.03-5.80 (m, 1H), 3.50 (dd, J=5.5, 8.0 Hz, 1H), 1.58 (dd, J=7.0, 8.0 Hz, 1H), 1.51 (s, 9H), 1.08-1.04 (m, 1H), 0.96 (s, 3H) and

Peak 2: trans-tert-butyl (1-(2-(difluoromethyl)-2-methylcyclopropyl)-2-oxo-1,2-dihydropyridin-3-yl)carbamate [stereochemistry arbitrarily assigned], (35 mg, 6.02% yield) as yellow oil. 1HNMR (500 MHz, CDCl₃) δ ppm: 9.92 (d, J=6.5 Hz, 1H), 9.61 (s, 1H), 8.87 (dd, J=1.5, 7.0 Hz, 1H), 8.16 (t, J=7.0 Hz, 1H), 7.57-7.34 (m, 1H), 5.20-5.17 (m, 1H), 3.53-3.50 (m, 1H), 3.47 (s, 9H), 3.38 (s, 3H), 3.18 (t, J=7.5 Hz, 1H).

Step d: To cis-tert-butyl (1-(2-(difluoromethyl)-2-methylcyclopropyl)-2-oxo-1,2-dihydropyridin-3-yl)carbamate (190 mg, 604.5 μmol) was added HCl/dioxane (20 mL) and the reaction stirred at 25° C. for 4 h. The mixture was concentrated in vacuo, the residue was diluted with EtOAc (10 mL) and filtered. The filter cake was evaporated in vacuo to give cis-3-amino-1-(2-(difluoromethyl)-2-methylcyclopropyl)pyridin-2(1H)-one hydrochloride (100 mg, 66.0% yield) as white solid. LCMS m/z=214.6 [M+H]⁺

Step e: To trans-tert-butyl (1-(2-(difluoromethyl)-2-methylcyclopropyl)-2-oxo-1,2-dihydropyridin-3-yl)carbamate (35.0 mg, 111.35 μmol) was added HCl/Dioxane (10 mL) and the reaction mixture stirred at 25° C. for 16 h. The mixture was concentrated in vacuo to give trans-3-amino-1-(2-(difluoromethyl)-2-methylcyclopropyl)pyridin-2(1H)-one hydrochloride (27.91 mg, crude). LCMS m/z=214.7 [M+H]⁺

### Preparation 82: 3-amino-1-((1R,2S)-2-methylcyclopropyl)pyridin-2(1H)-one hydrochloride

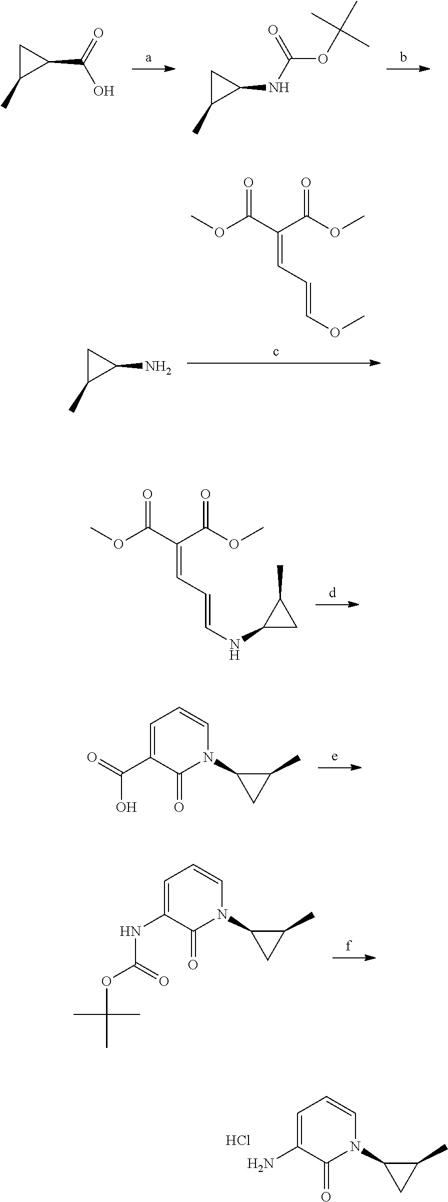

Step a: To a solution of (1R,2S)-2-methylcyclopropane-1-carboxylic acid (2.16 g, 21.58 mmol) in t-BuOH (20 mL) was added DPPA (6.53 g, 23.73 mmol) and TEA (7.20 g, 71.20 mmol) and the reaction was stirred at 90° C. for 72 h under N₂ atmosphere. Sat. aq. NaHCO₃ solution (30 mL) was added and the mixture extracted with EtOAc (30 mL×3). The combined organic layers were washed with brine (100 mL), dried over Na₂SO₄, filtered and concentrated. The crude material was purified by silica gel column chromatography (PE/EtOAc=15/1 to 5/1) to give tert-butyl ((1R,2S)-2-methylcyclopropyl)carbamate (2.7 g, 73.1% yield) as yellow solid. 1H NMR: (400 MHz, CDCl₃) δ ppm 4.56 (br s, 1H), 2.54 (br s, 1H), 1.45 (s, 9H), 1.06 (d, J=6.0 Hz, 3H), 0.97-0.82 (m, 2H), 0.10-0.02 (m, 1H).

Step b: To a solution of tert-butyl ((1R,2S)-2-methylcyclopropyl)carbamate (2.7 g, 15.77 mmol) in dioxane (10 mL) was added HCl/dioxane (4 M, 10 mL) and the reaction was stirred at 20° C. for 12 h under N₂ atmosphere. The mixture was concentrated under reduced pressure to give (1R,2S)-2-methylcyclopropan-1-amine hydrochoride (1.1 g, 64.9% yield) as yellow solid. 1H NMR: (400 MHz, DMSO-d₆) δ ppm 8.45 (br s, 2H), 2.54-2.49 (m, 1H), 1.21 (d, J=6.4 Hz, 3H), 1.10-0.99 (m, 1H), 0.93-0.85 (m, 1H), 0.57-0.50 (m, 1H).

Step c: To a solution of (1R,2S)-2-methylcyclopropan-1-amine hydrochoride (1.1 g, 10.22 mmol) in MeOH (20 mL) was added dimethyl (E)-2-(3-methoxyallylidene)malonate (3.07 g, 15.34 mmol) and TEA (3.10 g, 30.67 mmol) and the reaction was stirred at 20° C. for 2 h under N₂. The residue was purified by silica gel column chromatography (PE/EtOAc=5/1 to 1/1) to give dimethyl 2-((E)-3-(((1R,2S)-2-methylcyclopropyl)amino)allylidene)malonate (750 mg, 30.7% yield) as yellow oil. LCMS m/z=240.0 [M+H]⁺

Step d: A mixture of dimethyl 2-((E)-3-(((1R,2S)-2-methylcyclopropyl)amino)allylidene)malonate (750 mg, 3.13 mmol) in EtOH (5 mL) and KOH (299.0 mg, 5.33 mmol) was stirred at 25° C. for 1 h and 90° C. for a further 2 h. The resulting mixture was evaporated under reduced pressure and the residue was dissolved in water (10 mL) and the pH adjusted to 4-5 with 1M HCl. The mixture was extracted with EtOAc (10 mL×3), the organic layers were washed with brine (30 mL), dried over Na₂SO₄, filtered and concentrated to give 1-((1R,2S)-2-methylcyclopropyl)-2-oxo-1,2-dihydropyridine-3-carboxylic acid (580 mg, 95.8% yield) as a yellow solid which was used in the next step without further purification. 1H NMR (400 MHz, CDCl₃) δ ppm 14.33 (s, 1H), 8.52 (dd, J=7.2, 2.0 Hz, 1H), 7.67 (dd, J=6.8, 2.0 Hz, 1H), 6.54 (t, J=7.0 Hz, 1H), 3.52-3.46 (m, 1H), 1.56-1.50 (m, 1H), 1.38-1.31 (m, 1H), 0.88 (d, J=6.4 Hz, 3H), 0.78-0.73 (m, 1H).

Step e: To a mixture of 1-((1R,2S)-2-methylcyclopropyl)-2-oxo-1,2-dihydropyridine-3-carboxylic acid (580 mg, 3.0 mmol) in t-BuOH (3 mL) and TEA (455.67 mg, 4.50 mmol) was added DPPA (991.41 mg, 3.60 mmol) and the reaction mixture was stirred at 90° C. for 2 h. Water (20 mL) was added and the mixture was extracted with EtOAc (20 mL×3). The combined organic layers were washed with brine (50 mL), dried over Na₂SO4, filtered and concentrated. The crude material was purified by silica gel column chromatography (PE/EtOAc=5/1 to 1/1) to give tert-butyl (1-((1R,2S)-2-methylcyclopropyl)-2-oxo-1,2-dihydropyridin-3-yl)carbamate (460 mg, 58.0% yield) as a yellow solid. LCMS m/z=265.0 [M+H]⁺

Step f: A mixture of tert-butyl (1-((1R,2S)-2-methylcyclopropyl)-2-oxo-1,2-dihydropyridin-3-yl)carbamate (600 mg, 2.27 mmol) in dioxane (5 mL) and HCl/dioxane (4 M, 10 mL) was stirred at 40° C. for 12 h. The mixture was concentrated under reduced pressure, the residue was diluted with water (9 mL) and MeCN (3 mL) then lyophilised to give 3-amino-1-((1R,2S)-2-methylcyclopropyl)pyridin-2(1H)-one hydrochloride (416 mg, 91.2% yield) as a yellow solid. LCMS m/z=165.1 [M+H]⁺

### Preparation 83: 3-amino-1-((1S,2R)-2-methylcyclopropyl)pyridin-2(1H)-one hydrochloride

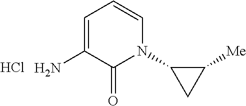 [SMILES: Cl ; C[C@@H]1C[C@@H]1n1cccc(N)c1=O]

3-Amino-1-((1S,2R)-2-methylcyclopropyl)pyridin-2(1H)-one hydrochloride was obtained as a yellow solid, from (1S,2R)-2-methylcyclopropane-1-carboxylic acid, following the steps described in Preparation 82. LCMS m/z=165.2 [M+H]⁺. (400 MHz, CD₃OD) δ ppm 7.66-7.71 (m, 2H), 6.37-6.44 (m, 1H), 3.42-3.46 (m, 1H), 1.41-1.53 (m, 1H), 1.26-1.33 (m, 1H), 0.85-0.91 (m, 1H), 0.83 (d, J=6.4 Hz, 3H).

### Preparations 84A and 85A. 3-amino-1-((1R,2R)-2-fluorocyclopropyl)pyridin-2(1H)-one hydrochloride and 3-amino-1-((1S,2S)-2-fluorocyclopropyl)pyridin-2(1H)-one hydrochloride

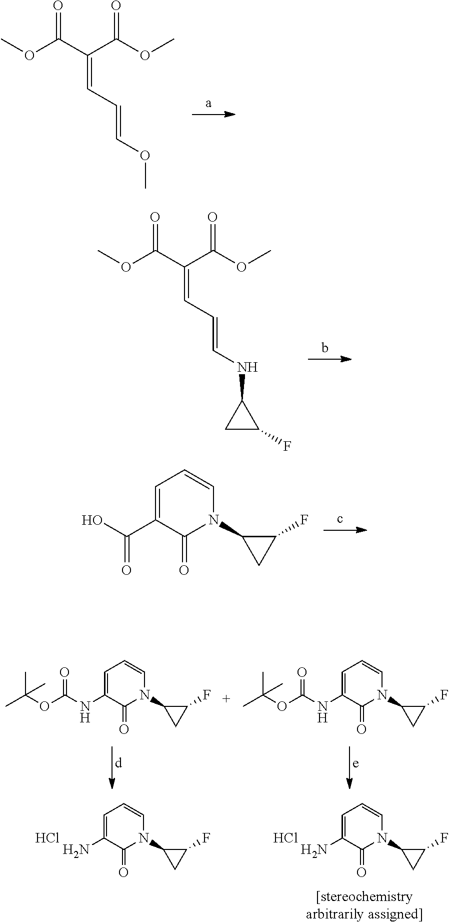

Step a. To a solution of dimethyl (E)-2-(3-methoxyallylidene)malonate (4.99 g, 24.92 mmol) in MeOH (50 mL) was added trans-2-fluorocyclopropanamine (2.78 g, 24.92 mmol), TEA (5.04 g, 49.85 mmol) and the reaction stirred at 25° C. for 16 h. The mixture was concentrated in vacuo, the residue was diluted with water (50 mL) and extracted with EtOAc (50 mL×3). The combined organic layer was washed with brine (50 mL), dried over Na₂SO₄ and filtered. The filtrate was concentrated in vacuo to give trans dimethyl 2-((E)-3-((2-fluorocyclopropyl)amino)allylidene)malonate (6.8 g, crude) as yellow oil and it was used directly in the next step.

Step b. To a solution of trans dimethyl 2-((E)-3-((2-fluorocyclopropyl)amino)allylidene)malonate (6.7 g, 27.55 mmol) in EtOH (100 mL) was added KOH (2.47 g, 44.07 mmol) and the mixture was stirred at 25° C. for 3 h. The reaction mixture was acidifed to pH 5 using 1M HCl, diluted with water (300 mL) and extracted with EtOAc (200 mL×3). The combined organic layer was washed with brine (100 mL), dried over Na₂SO₄ and filtered. The filtrate was concentrated in vacuo to give trans-1-(2-fluorocyclopropyl)-2-oxo-1,2-dihydropyridine-3-carboxylic acid (4 g, crude) as brown solid. LCMS m/z=197.6 [M+H]⁺

Step c. To a solution of trans-1-(2-fluorocyclopropyl)-2-oxo-1,2-dihydropyridine-3-carboxylic acid (4 g, 20.29 mmol) in t-BuOH (100 mL) was added DPPA (8.37 g, 30.43 mmol) and TEA (6.16 g, 60.86 mmol) and the reaction stirred at 90° C. for 16 h. The mixture was concentrated and then water (300 mL) was added. The mixture was extracted with EtOAc (300 mL×3), the combined organic layers were washed with brine (200 mL), dried over Na₂SO₄, filtered and concentrated in vacuo. The residue was purified by CombiFlash® (PE/EtOAc=1/1) and the product was further purified by SFC (Column: ChiralPak AD-3 150×4.6 mm I.D., 3 um, Mobile phase: A: CO₂ B: Ethanol (0.05% DEA), Gradient: from 5% to 40% of B in 4.5 min, Flow rate: 2.5 mL/min Column temp.:40° C.) to give tert-butyl (1-((1R,2R)-2-fluorocyclopropyl)-2-oxo-1,2-dihydropyridin-3-yl)carbamate (560 mg, 9.8% yield). RT=2.555 min. LCMS m/z=268.1 [M+H]+ and tert-butyl (1-((1S,2S)-2-fluorocyclopropyl)-2-oxo-1,2-dihydropyridin-3-yl)carbamate (560 mg, 9.8% yield) as brown solid. RT=2.842 min. LCMS m/z=268.1 [M+H]⁺

Step d. tert-Butyl (1-((1R,2R)-2-fluorocyclopropyl)-2-oxo-1,2-dihydropyridin-3-yl)carbamate (560 mg, 2.09 mmol) was dissolved in HCl/dioxane (30 mL) and the mixture was stirred at 25° C. for 16 h. The mixture was concentrated in vacuo to give 3-amino-1-((1R,2R)-2-fluorocyclopropyl)pyridin-2(1H)-one hydrochloride (Stereochemistry arbitrarily assigned), (400 mg, 93.7% yield) as white solid. LCMS m/z=168.9 [M+H]⁺

Step e. tert-Butyl (1-((1R,2R)-2-fluorocyclopropyl)-2-oxo-1,2-dihydropyridin-3-yl)carbamate (560 mg, 2.09 mmol) was dissolved in HCl/dioxane (30 mL) and the mixture was stirred at 25° C. for 16 h. The mixture was concentrated in vacuo to give 3-amino-1-((1S,2S)-2-fluorocyclopropyl)pyridin-2(1H)-one hydrochloride (400 mg, 93.7% yield) as white solid. LCMS m/z=168.9 [M+H]⁺

### Preparation 84B: 3-amino-1-((1R,2R)-2-fluorocyclopropyl)pyridin-2(1H)-one hydrochloride

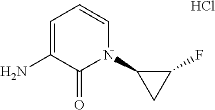 [SMILES: Nc1cccn([C@@H]2C[C@H]2F)c1=O ; Cl]

3-Amino-1-((1R,2R)-2-fluorocyclopropyl)pyridin-2(1H)-one hydrochloride was obtained from (1R,2R)-2-fluorocyclopropane-1-carboxylic acid, following the steps described in Preparation 82.

### Preparation 85B: 3-amino-1-((1S,2S)-2-fluorocyclopropyl)pyridin-2(1H)-one hydrochloride

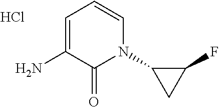 [SMILES: Cl ; Nc1cccn([C@H]2C[C@@H]2F)c1=O]

3-Amino-1-((1S,2S)-2-fluorocyclopropyl)pyridin-2(1H)-one hydrochloride was obtained from (1S,2S)-2-fluorocyclopropane-1-carboxylic acid, following the steps described in Preparation 82

### Preparations 86A and 87A. 3-amino-1-((1R,2S)-2-fluorocyclopropyl)pyridin-2(1H)-one and 3-amino-1-((1S,2R)-2-fluorocyclopropyl)pyridin-2(1H)-one

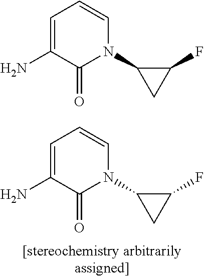

Cis 3-amino-1-(2-fluorocyclopropyl)pyridin-2(1H)-one (Preparation 63) was further purified by SFC using CHIRALPAK IG 30×250 mm, 5 um, Method: 30% EtOH w/ 0.1% DEA in CO₂, (flow rate: 100 mL/min, ABPR 120 bar, MBPR 40 psi, column temp 40° C.) to provide:

Peak 1: 3-amino-1-((1R,2S)-2-fluorocyclopropyl)pyridin-2(1H)-one (stereochemistry arbitrarily assigned). LCMS m/z=169.1 [M+H]⁺

Peak 2: 3-amino-1-((1S,2R)-2-fluorocyclopropyl)pyridin-2(1H)-one (stereochemistry arbitrarily assigned). LCMS m/z=169.1 [M+H]⁺

### Preparations 87B: 3-amino-1-((1S,2R)-2-fluorocyclopropyl)pyridin-2(1H)-one hydrochloride

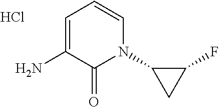 [SMILES: Cl ; Nc1cccn([C@H]2C[C@H]2F)c1=O]

3-Amino-1-((1S,2R)-2-fluorocyclopropyl)pyridin-2(1H)-one hydrochloride was obtained from (1S,2R)-2-fluorocyclopropane-1-carboxylic acid, following the steps described in Preparation 82. 1H NMR (400 MHz, DMSO) δ ppm 7.57-7.60 (m, 1H), 7.47-7.51 (m, 1H), 6.28 (t, J=7.2 Hz, 1H), 4.88-5.13 (m, 1H), 3.33-3.44 (m, 1H), 1.52-1.66 (m, 1H), 1.37-1.51 (m, 1H).

### Preparation 88: 2-bromo-1-(4-methyl-2-oxabicyclo[2.1.1]hexan-1-yl)ethan-1-one

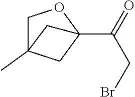

2-Bromo-1-(4-methyl-2-oxabicyclo[2.1.1]hexan-1-yl)ethan-1-one was obtained from 4-methyl-2-oxabicyclo[2.1.1]hexane-1-carboxylic acid, following a similar procedure to that described in Preparation 13.

### Preparation 89: 2-bromo-1-(1-(methoxymethyl)-2-oxabicyclo[2.1.1]hexan-4-yl)ethan-1-one

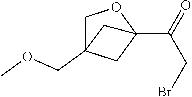

2-Bromo-1-(1-(methoxymethyl)-2-oxabicyclo[2.1.1]hexan-4-yl)ethan-1-one was obtained from 1-(methoxymethyl)-2-oxabicyclo[2.1.1]hexane-4-carboxylic acid following a similar procedure to that described in Preparation 13.

### Preparation 90: 7-cyclobutoxy-2-(1-(methoxymethyl)-2-oxabicyclo[2.1.1]hexan-4-yl)imidazo[1,2-a]pyrimidine-6-carboxylic acid

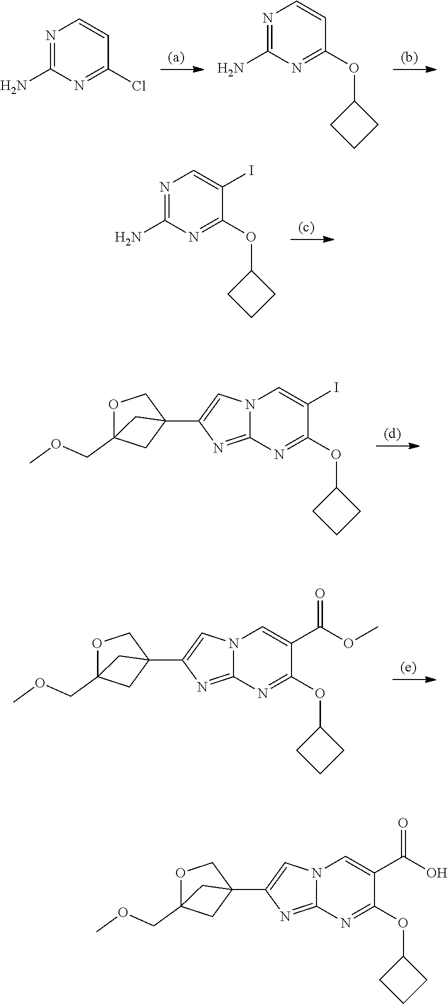

Step a: To a solution of cyclobutanol (2.23 g, 30.88 mmol) in THF (3 mL) was added NaH (1.24 g, 30.88 mmol, 60% purity) at 0° C. and the mixture stirred for 0.5 h. 4-Chloropyrimidin-2-amine (1 g, 7.72 mmol) was added and the reaction mixture was stirred at 25° C. for 16 h. The reaction was quenched with water (10 mL), extracted with EtOAc (10 mL×3), the combined organic layer was washed with brine (10 mL), dried over Na₂SO₄ and filtered. The filtrate was concentrated in vacuo and the residue purified by Combiflash® (PE/EtOAc=20/1 to 10/1) to give 4-cyclobutoxypyrimidin-2-amine (800 mg, 56.5% yield) as a colorless oil. 1H NMR: (500 MHz, DMSO-d₆) δ ppm: 7.94 (d, J=6.0 Hz, 1H), 6.46 (s, 2H), 5.94 (d, J=6.0 Hz, 1H), 5.10-5.04 (m, 1H), 2.50-2.35 (m, 2H), 2.04-2.00 (m, 2H), 1.60-1.58 (m, 1H), 1.57-1.54 (m, 1H).

Step b: To a solution of 4-cyclobutoxypyrimidin-2-amine (800 mg, 4.84 mmol) in DCM (100 mL) was added NIS (980.61 mg, 4.36 mmol) and the reaction stirred at 25° C. for 16 h. The reaction was diluted with saturated Na₂SO₃ aq. (10 mL) and extracted with EtOAc (30 mL×3). The combined organic layer was washed with brine (20 mL), dried over Na₂SO₄ and filtered. The filtrate was concentrated in vacuo and the residue was purified by Combiflash® (PE/EtOAc=5/1 to 1/1) to give 4-cyclobutoxy-5-iodopyrimidin-2-amine (600 mg, 42.6% yield) as a yellow solid. LCMS m/z=291.8 [M+H]⁺

Step (c): To a solution of 4-cyclobutoxy-5-iodopyrimidin-2-amine (616.15 mg, 2.47 mmol) and 2-bromo-1-(1-(methoxymethyl)-2-oxabicyclo[2.1.1]hexan-4-yl)ethan-1-one (600 mg, 2.06 mmol) in t-BuOH (10 mL) was added NaHCO₃ (346.31 mg, 4.12 mmol) and the reaction stirred at 100° C. for 16 h. The reaction mixture was concentrated in vacuo and the residue was purified by Combiflash® (PE/EtOAc=1:1) to give 7-cyclobutoxy-6-iodo-2-(1-(methoxymethyl)-2-oxabicyclo[2.1.1]hexan-4-yl)imidazo[1,2-a]pyrimidine (400 mg, 44.0% yield) as yellow oil. LCMS m/z=442.0 [M+H]⁺

Step d: To a solution of 7-cyclobutoxy-6-iodo-2-(1-(methoxymethyl)-2-oxabicyclo[2.1.1]hexan-4-yl)imidazo[1,2-a]pyrimidine (400 mg, 0.907 mmol) in MeOH (60 mL) was added TEA (917.28 mg, 9.06 mmol) and Pd(dppf)Cl₂ (66.33 mg, 0.091 mmol), the mixture was degassed with CO and stirred at 80° C. under CO (50 Psi) for 48 h. The mixture was concentrated in vacuo and the residue was purified by Combiflash® (PE/EtOAc=1/1) to give methyl 7-cyclobutoxy-2-(1-(methoxymethyl)-2-oxabicyclo[2.1.1]hexan-4-yl)imidazo[1,2-a]pyrimidine-6-carboxylate (350 mg, 93.1% yield) as a white solid. LCMS m/z=374.2 [M+H]⁺

Step e: To a solution of methyl 7-cyclobutoxy-2-(1-(methoxymethyl)-2-oxabicyclo[2.1.1]hexan-4-yl)imidazo[1,2-a]pyrimidine-6-carboxylate (350 mg, 0.744 mmol) in water (3 mL) and MeOH (3 mL) was added LiOH (80.55 mg, 3.21 mmol) and the reaction stirred at 15° C. for 16 h. The reaction mixture was concentrated in vacuo, the residue was diluted with water (10 mL) and the pH adjusted using HCl a.q (1 M) to pH=3. The mixture was lyophlized to give 7-cyclobutoxy-2-(1-(methoxymethyl)-2-oxabicyclo[2.1.1]hexan-4-yl)imidazo[1,2-a]pyrimidine-6-carboxylic acid (crude) as a yellow solid. LCMS m/z=360.1 [M+H]⁺

### Preparations 91 and 92: 7-isopropoxy-2-((1S,4R)-1-methyl-2-oxabicyclo[2.2.1]heptan-4-yl)imidazo[1,2-a]pyrimidine-6-carboxylic acid and 7-isopropoxy-2-((1R,4S)-1-methyl-2-oxabicyclo[2.2.1]heptan-4-yl)imidazo[1,2-a]pyrimidine-6-carboxylic acid

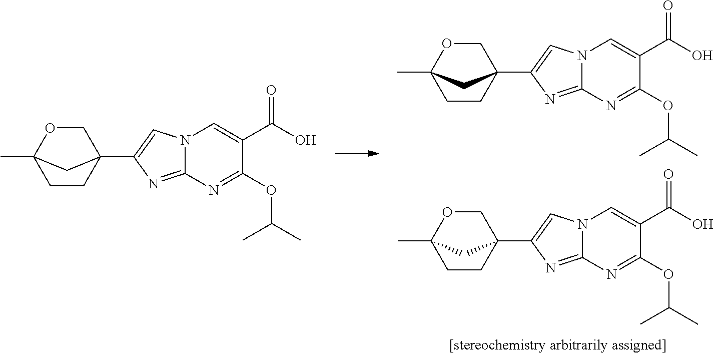

7-Isopropoxy-2-(1-methyl-2-oxabicyclo[2.2.1]heptan-4-yl)imidazo[1,2-a]pyrimidine-6-carboxylic acid (Preparation 64, 800 mg, 2.41 mmol) was purified by SFC (Column: Chiralpak IC-3 100×4.6 mm, 3 μm; Mobile phase: A: CO₂ B:40% EtOH (0.05% DEA); Flow rate: 2.8 mL/min; Column temp.: 35° C.; ABPR: 1500 psi) to give 7-isopropoxy-2-((1S,4R)-1-methyl-2-oxabicyclo[2.2.1]heptan-4-yl)imidazo[1,2-a]pyrimidine-6-carboxylic acid (345 mg, 43% yield) and 7-isopropoxy-2-((1R,4S)-1-methyl-2-oxabicyclo[2.2.1]heptan-4-yl)imidazo[1,2-a]pyrimidine-6-carboxylic acid (380 mg, 47% yield) both as brown solids.

### Preparations 93 and 94: 8-fluoro-7-isopropoxy-2-((1S,4R)-1-methyl-2-oxabicyclo[2.2.1]heptan-4-yl)imidazo[1,2-a]pyridine-6-carboxylic acid and 8-fluoro-7-isopropoxy-2-((1R,4S)-1-methyl-2-oxabicyclo[2.2.1]heptan-4-yl)imidazo[1,2-a]pyridine-6-carboxylic acid

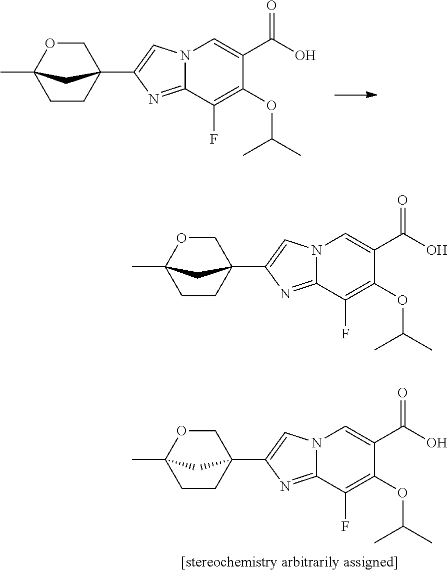 [SMILES: CC(C)Oc1c(C(=O)O)cn2cc([C@@]34CC[C@@](C)(C3)OC4)nc2c1F ; CC(C)Oc1c(C(=O)O)cn2cc([C@@]34CC[C@@](C)(C3)OC4)nc2c1F ; CC(C)Oc1c(C(=O)O)cn2cc([C@]34CC[C@](C)(C3)OC4)nc2c1F]

8-Fluoro-7-isopropoxy-2-(1-methyl-2-oxabicyclo[2.2.1]heptan-4-yl)imidazo[1,2-a]pyridine-6-carboxylic acid (Example 67 and 68, step c, 500 mg, 1.44 mmol) was purified by prep-SFC: DAICEL CHIRALPAK AY-H (250 mm*30 mm, 5 um), 40° C.; mobile phase: 40% of 0.05% DEA/IPA to give:

Peak 1: 8-fluoro-7-isopropoxy-2-((1S,4R)-1-methyl-2-oxabicyclo[2.2.1]heptan-4-yl)imidazo[1,2-a]pyridine-6-carboxylic acid [stereochemistry arbitrarily assigned] (200 mg, retention time=3.719 min) as a white solid. LCMS m/z=349.1 [M+H]+ and

Peak 2: 8-fluoro-7-isopropoxy-2-((1R,4S)-1-methyl-2-oxabicyclo[2.2.1]heptan-4-yl)imidazo[1,2-a]pyridine-6-carboxylic acid [stereochemistry arbitrarily assigned] (200 mg, retention time=3.967 min) as white solid. LCMS m/z=349.3 [M+H]⁺

### Preparations 95 and 96: 7-isopropoxy-2-((1S,4R)-1-(methoxymethyl)-2-oxabicyclo[2.2.1]heptan-4-yl)imidazo[1,2-a]pyrimidine-6-carboxylic acid and 7-isopropoxy-2-((1R,4S)-1-(methoxymethyl)-2-oxabicyclo[2.2.1]heptan-4-yl)imidazo[1,2-a]pyrimidine-6-carboxylic acid

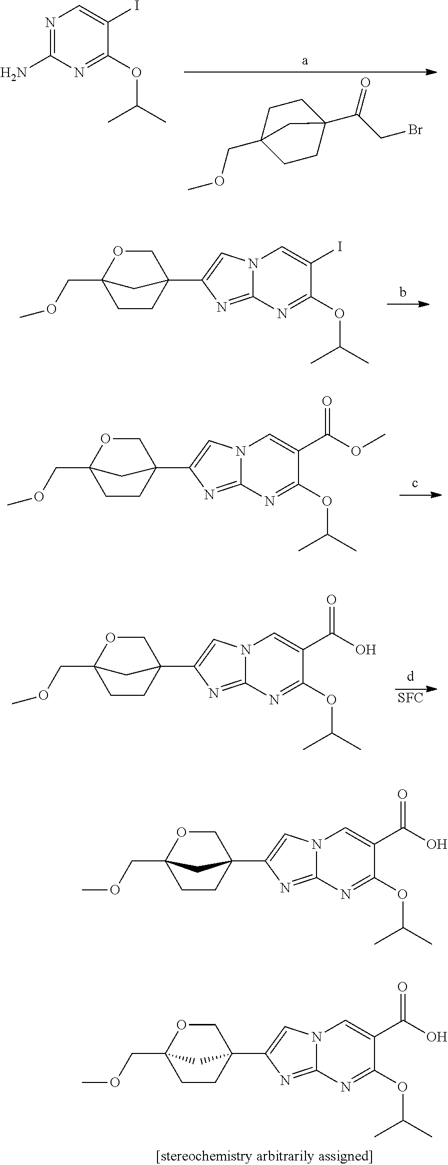

Step a: To a solution of 2-bromo-1-(1-(methoxymethyl)-2-oxabicyclo[2.2.1]heptan-4-yl)ethan-1-one (Preparation 18, 530.31 mg, 1.90 mmol) and 5-iodo-4-isopropoxypyrimidin-2-amine (500 mg, 1.90 mmol) in t-BuOH (20 mL) was added NaHCO₃ (319.27 mg, 3.80 mmol) and the reaction stirred at 100° C. for 16 h. The reaction mixture was concentrated and the residue was purified by Combiflash® (PE/EtOAc=1/1) to give 6-iodo-7-isopropoxy-2-(1-(methoxymethyl)-2-oxabicyclo[2.2.1]heptan-4-yl)imidazo[1,2-a]pyrimidine (700 mg, 83.1% yield) as yellow oil. LCMS m/z=444.0 [M+H]⁺

Step b: To a solution of 6-iodo-7-isopropoxy-2-(1-(methoxymethyl)-2-oxabicyclo[2.2.1]heptan-4-yl)imidazo[1,2-a]pyrimidine (700 mg, 1.58 mmol) in MeOH (60 mL) was added TEA (2.2 mL, 15.79 mmol) and Pd(dppf)Cl₂ (115.55 mg, 0.158 mmol), the mixture was degassed with CO and then stirred at 80° C. under CO (50 Psi) for 48 h. The mixture was concentrated in vacuo and the residue was purified by CombiFlash® (PE/EtOAc=1/1) to give methyl 7-isopropoxy-2-(1-(methoxymethyl)-2-oxabicyclo[2.2.1]heptan-4-yl)imidazo[1,2-a]pyrimidine-6-carboxylate (500 mg, 84.3% yield) as a white solid. LCMS m/z=376.2 [M+H]⁺

Step c: To a solution of methyl 7-isopropoxy-2-(1-(methoxymethyl)-2-oxabicyclo[2.2.1]heptan-4-yl)imidazo[1,2-a]pyrimidine-6-carboxylate (500 mg, 1.33 mmol) in water (10 mL) and MeOH (10 mL) was added NaOH (159.8 mg, 4.0 mmol) and the reaction stirred at 15° C. for 16 h. The reaction mixture was concentrated to give the residue which was diluted with water (10 mL) and the pH adjusted to 3 using 1M HCl. The mixture was lyophilized to give 7-isopropoxy-2-(1-(methoxymethyl)-2-oxabicyclo[2.2.1]heptan-4-yl)imidazo[1,2-a]pyrimidine-6-carboxylic acid (500 mg, 98.7% yield) as a yellow solid. LCMS m/z=362.1 [M+H]⁺

Step d: 7-Isopropoxy-2-(1-(methoxymethyl)-2-oxabicyclo[2.2.1]heptan-4-yl)imidazo[1,2-a]pyrimidine-6-carboxylic acid (500 mg, 1.31 mmol) was purified by prep-SFC (Column: DAICEL CHIRALPAK AY-H (250 mm×30 mm, 5 um), 40° C.; Mobile Phase: 40% of 0.05% DEA/IPA to give:

Peak 1: 7-isopropoxy-2-((1S,4R)-1-(methoxymethyl)-2-oxabicyclo[2.2.1]heptan-4-yl)imidazo[1,2-a]pyrimidine-6-carboxylic acid [stereochemistry arbitrarily assigned] (150 mg, 30.0% yield, retention time=1.96 min). LCMS m/z=362.2 [M+H]⁺

Peak 2: 7-isopropoxy-2-((1R,4S)-1-(methoxymethyl)-2-oxabicyclo[2.2.1]heptan-4-yl)imidazo[1,2-a]pyrimidine-6-carboxylic acid [stereochemistry arbitrarily assigned] (150 mg, 30.0% yield, retention time=2.10 min) as white solid. LCMS m/z=362.1 [M+H]⁺

### EXAMPLES

### Example 1: 7-cyclobutoxy-N-(1-cyclopropyl-2-oxo-1,2-dihydropyridin-3-yl)-2-(1-methyl-2-oxabicyclo[2.2.2]octan-4-yl)imidazo[1,2-a]pyridine-6-carboxamide

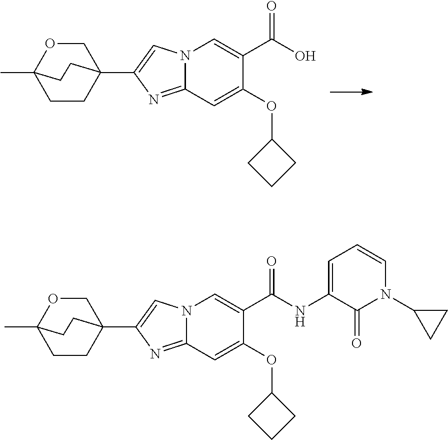 [SMILES: CC12CCC(c3cn4cc(C(=O)O)c(OC5CCC5)cc4n3)(CC1)CO2 ; CC12CCC(c3cn4cc(C(=O)Nc5cccn(C6CC6)c5=O)c(OC5CCC5)cc4n3)(CC1)CO2]

An EtOAc solution of T3P® (0.42 mmol, 250 uL, 50% w/w) was added to 7-cyclobutoxy-2-(1-methyl-2-oxabicyclo[2.2.2]octan-4-yl)imidazo[1,2-a]pyridine-6-carboxylic acid [preparation 65] (30 mg, 0.084 mmol) and 3-amino-1-cyclopropyl-pyridin-2-one (15 mg, 0.10 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H2O, Modifier: 0.1% NH₄OH) to provide 7-cyclobutoxy-N-(1-cyclopropyl-2-oxo-1,2-dihydropyridin-3-yl)-2-(1-methyl-2-oxabicyclo[2.2.2]octan-4-yl)imidazo[1,2-a]pyridine-6-carboxamide (16.2 mg, 39% yield). LCMS (ESI) m/z 488.9 (M+H)⁺. 1H NMR (500 MHz, DMSO-d6) δ ppm 0.89-0.96 (m, 2H) 1.03-1.08 (m, 2H) 1.10 (s, 3H) 1.74-1.89 (m, 5H) 1.91-2.01 (m, 3H) 2.03-2.10 (m, 2H) 2.55-2.65 (m, 5H) 3.92 (s, 2H) 5.21 (br t, J=7.02 Hz, 1H) 6.16-6.41 (m, 1H) 7.04 (s, 1H) 7.38 (dd, J=6.71, 1.83 Hz, 1H) 8.04 (s, 1H) 8.46 (dd, J=7.32, 1.83 Hz, 1H) 9.41 (s, 1H) 10.68 (s, 1H).

### Example 2: N-(1-cyclopropyl-2-oxo-1,2-dihydropyridin-3-yl)-2-(1-ethyl-2-oxabicyclo[2.1.1]hexan-4-yl)-7-isopropoxyimidazo[1,2-a]pyridine-6-carboxamide

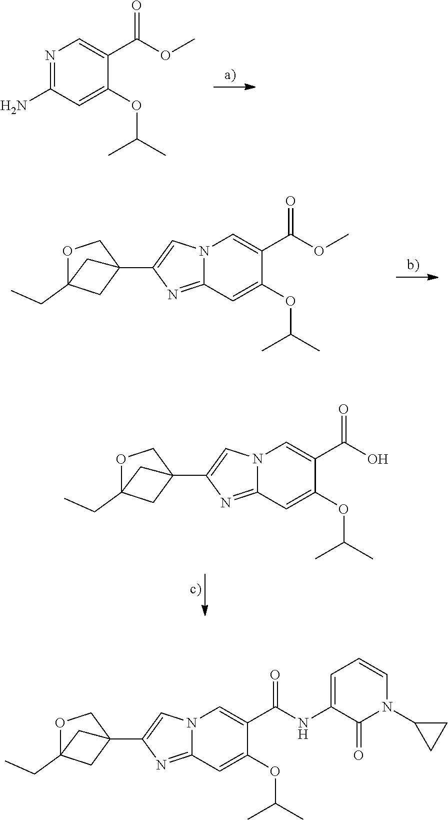

Step a: A mixture of methyl 6-amino-4-isopropoxynicotinate [preparation 2] (300 mg, 1.43 mmol), 2-bromo-1-(1-ethyl-2-oxabicyclo[2.1.1]hexan-4-yl)ethanone (333 mg, 1.43 mmol) and NaHCO₃ (359 mg, 4.28 mmol) in MeCN (2.3 mL) and toluene (3.4 mL) was heated at 90° C. for 16 h. Silica and MeOH were added and the mixture was concentrated and purified by silica gel column chromatography (dry load, 24 g, 0-70% gradient of 3:1 EtOAC/EtOH in heptanes) to obtain methyl 2-(1-ethyl-2-oxabicyclo[2.1.1]hexan-4-yl)-7-isopropoxy-imidazo[1,2-a]pyridine-6-carboxylate (400 mg, 1.16 mmol, 81% yield). (LCMS (ESI) m/z 344.9 (M+H)⁺.

Step b: A mixture of methyl 2-(1-ethyl-2-oxabicyclo[2.1.1]hexan-4-yl)-7-isopropoxy-imidazo[1,2-a]pyridine-6-carboxylate (200 mg, 580 μmol) and lithium hydroxide hydrate (48 g, 1.16 mmol) in MeOH (0.2 mL), THF (1.5 mL) and water (0.4 mL) was stirred overnight at room temperature before being neutralized with a 4.0 M hydrochloric acid solution in dioxane. The mixture was concentrated to obtain 2-(1-ethyl-2-oxabicyclo[2.1.1]hexan-4-yl)-7-isopropoxy-imidazo[1,2-a]pyridine-6-carboxylic acid, which was used without further purification in next reaction. Assumed 100% yield. LCMS (ESI) m/z 330.9 (M+H)⁺.

Step c: T3P (0.72 mmol, 430 μL, 50% purity in EtOAc) was added to 2-(1-ethyl-2-oxabicyclo[2.1.1]hexan-4-yl)-7-isopropoxy-imidazo[1,2-a]pyridine-6-carboxylic acid (48 mg, 0.14 mmol) and 3-amino-1-cyclopropyl-pyridin-2-one (24 mg, 0.16 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide N-(1-cyclopropyl-2-oxo-1,2-dihydropyridin-3-yl)-2-(1-ethyl-2-oxabicyclo[2.1.1]hexan-4-yl)-7-isopropoxyimidazo[1,2-a]pyridine-6-carboxamide (33.9 mg, 50% yield). LCMS (ESI) m/z 463.0 (M+H)⁺. 1H NMR (500 MHz, DMSO-d6) δ ppm 0.90-0.93 (m, 2H) 0.94-0.97 (m, 3H) 1.03-1.08 (m, 2H) 1.54 (d, J=5.49 Hz, 6H) 1.74-1.78 (m, 4H) 2.01 (br d, J=3.05 Hz, 2H) 3.50 (tt, J=7.71, 3.89 Hz, 1H) 3.90 (s, 2H) 5.07 (dt, J=12.06, 5.88 Hz, 1H) 6.31 (t, J=7.02 Hz, 1H) 7.24 (s, 1H) 7.34 (dd, J=7.02, 1.53 Hz, 1H) 7.90 (s, 1H) 8.45 (dd, J=7.32, 1.83 Hz, 1H) 9.24 (s, 1H) 10.74 (s, 1H).

### Example 3: N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyridine-6-carboxamide

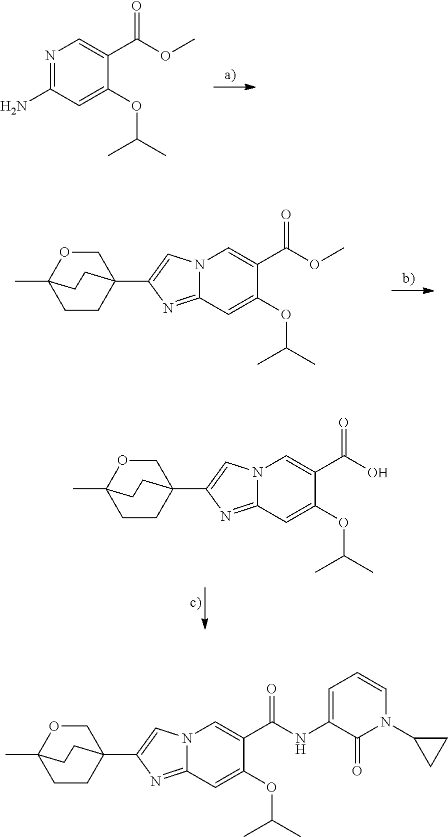

Step a: A mixture of sodium; hydrogen carbonate (718 mg, 8.55 mmol), methyl 6-amino-4-isopropoxynicotinate [preparation 2] (600 mg, 2.85 mmol) and 2-bromo-1-(1-methyl-2-oxabicyclo[2.2.2]octan-4-yl)ethanone (preparation 13, 704 mg, 2.85 mmol) in MeCN (4.1 mL) and Toluene (4.1 mL) was heated at 90° C. for 16 h. Silica and MeOH were added and the mixture was concentrated and purified by silica gel column chromatography (dry load, 24 g, 0-50% gradient of 3:1 EtOAC/EtOH in heptanes) to obtain methyl 7-isopropoxy-2-(1-methyl-2-oxabicyclo[2.2.2]octan-4-yl)imidazo[1,2-a]pyridine-6-carboxylate (900 mg, 2.51 mmol, 88% yield). (LCMS (ESI) m/z 359.2 (M+H)⁺.

Step b: A mixture of methyl 7-isopropoxy-2-(1-methyl-2-oxabicyclo[2.2.2]octan-4-yl)imidazo[1,2-a]pyridine-6-carboxylate (400 mg, 1.12 mmol) and lithium; hydroxide; hydrate (93 mg, 2.23 mmol) in MeOH (560 μL), THF (4.0 mL), water (1.0 mL) was stirred overnight at room temperature before being neutralized with a 4.0 M hydrochloric acid solution in dioxane. The mixture was concentrated to obtain 7-isopropoxy-2-(1-methyl-2-oxabicyclo[2.2.2]octan-4-yl)imidazo[1,2-a]pyridine-6-carboxylic acid, which was used without further purification in next reaction. Assumed 100% yield. LCMS (ESI) m/z 345.2 (M+H)⁺.

Step c: T3P (0.55 mmol, 330 μL, 50% purity in EtOAc) was added to 7-isopropoxy-2-(1-methyl-2-oxabicyclo[2.2.2]octan-4-yl)imidazo[1,2-a]pyridine-6-carboxylic acid (38.0 mg, 110 μmol) and 3-amino-1-cyclopropyl-pyridin-2-one (20 mg, 0.13 mmol, hydrochloride) in Pyridine (1.9 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H2O, Modifier: 0.1% NH4OH) to provide N-(1-cyclopropyl-2-oxo-3-pyridyl)-7-isopropoxy-2-(1-methyl-2-oxabicyclo[2.2.2]octan-4-yl)imidazo[1,2-a]pyridine-6-carboxamide (27 mg, 51% yield). LCMS (ESI) m/z 476.9 (M+H)⁺. 1H NMR (500 MHz, DMSO-d6) δ ppm 0.89-0.94 (m, 2H) 1.02-1.07 (m, 2H) 1.08 (s, 3H) 1.54 (d, J=6.10 Hz, 6H) 1.70-1.78 (m, 2H) 1.79-1.86 (m, 2H) 1.87-1.94 (m, 2H) 2.02-2.10 (m, 2H) 3.50 (dt, J=7.78, 3.74 Hz, 1H) 3.91 (s, 2H) 5.04-5.14 (m, 1H) 6.27-6.35 (m, 1H) 7.24 (s, 1H) 7.34 (dd, J=7.32, 1.83 Hz, 1H) 7.84 (br s, 1H) 8.44 (dd, J=7.32, 1.83 Hz, 1H) 9.25 (br s, 1H) 10.72 (s, 1H).

### Examples 4 & 5: N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide and N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide

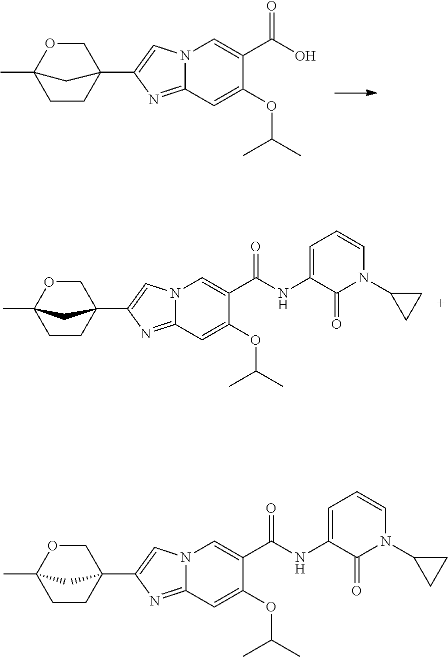

T3P (1.21 mmol, 720 uL, 50% purity in EtOAc) was added to 7-isopropoxy-2-(1-methyl-2-oxabicyclo[2.2.1]heptan-4-yl)imidazo[1,2-a]pyridine-6-carboxylic acid [preparation 58] and 3-amino-1-cyclopropyl-pyridin-2-one (43 mg, 0.29 mmol, Hydrochloride) in Pyridine (1.6 mL) at rt. After stirring overnight, the mixture was diluted with water and extracted with DCM and then EtOAc, dried over MgSO₄, filtered, concentrated, and purified via chiral SFC (CHIRALPAK IB 30×250 mm, 5 um Method: 40% MeOH w/ 0.1% DEA in CO2; flow rate: 100 mL/min, ABPR 120 bar, MBPR 40 psi, column temp 40 deg C.) to obtain Peak 1, Example 4: N-(1-cyclopropyl-2-oxo-3-pyridyl)-7-isopropoxy-2-[(1S,4R)-1-methyl-2-oxabicyclo[2.2.1]heptan-4-yl]imidazo[1,2-a]pyridine-6-carboxamide (24.1 mg, 46.89 μmol, 19% yield, stereochemistry arbitrarily assigned); LCMS (ESI) m/z 463.0 (M+H)⁺; 1H NMR (400 MHz, CHLOROFORM-d) δ ppm 0.90-0.95 (m, 2H) 1.15-1.21 (m, 2H) 1.49 (s, 3H) 1.65 (d, J=6.27 Hz, 6H) 1.79-1.90 (m, 2H) 1.91-1.95 (m, 1H) 1.98-2.04 (m, 1H) 2.05-2.21 (m, 2H) 3.41-3.52 (m, 1H) 3.98 (d, J=6.53 Hz, 1H) 4.11 (dd, J=6.53, 3.26 Hz, 1H) 4.83 (spt, J=6.11 Hz, 1H) 6.23 (t, J=7.15 Hz, 1H) 6.98 (s, 1H) 7.05 (dd, J=7.03, 1.76 Hz, 1H) 7.32 (s, 1H) 8.55 (dd, J=7.53, 1.76 Hz, 1H) 8.97 (s, 1H) 10.78 (s, 1H) and Peak 2, Example 5: N-(1-cyclopropyl-2-oxo-3-pyridyl)-7-isopropoxy-2-[(1R,4S)-1-methyl-2-oxabicyclo[2.2.1]heptan-4-yl]imidazo[1,2-a]pyridine-6-carboxamide (22.3 mg, 47.7 μmol, 19% yield, stereochemistry arbitrarily assigned); LCMS (ESI) m/z 463.0 (M+H)⁺; 1H NMR (400 MHz, CHLOROFORM-d) δ ppm 0.90-0.98 (m, 2H) 1.15-1.22 (m, 2H) 1.50 (s, 3H) 1.65 (d, J=6.02 Hz, 6H) 1.80-1.90 (m, 2H) 1.92-1.96 (m, 1H) 1.99-2.04 (m, 1H) 2.06-2.22 (m, 2H) 3.43-3.53 (m, 1H) 3.98 (d, J=6.27 Hz, 1H) 4.12 (dd, J=6.40, 3.39 Hz, 1H) 4.83 (dt, J=12.11, 6.12 Hz, 1H) 6.23 (t, J=7.28 Hz, 1H) 6.99 (s, 1H) 7.05 (dd, J=6.90, 1.38 Hz, 1H) 7.32 (s, 1H) 8.55 (dd, J=7.40, 1.38 Hz, 1H) 8.97 (s, 1H) 10.78 (s, 1H).

### Example 6: N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

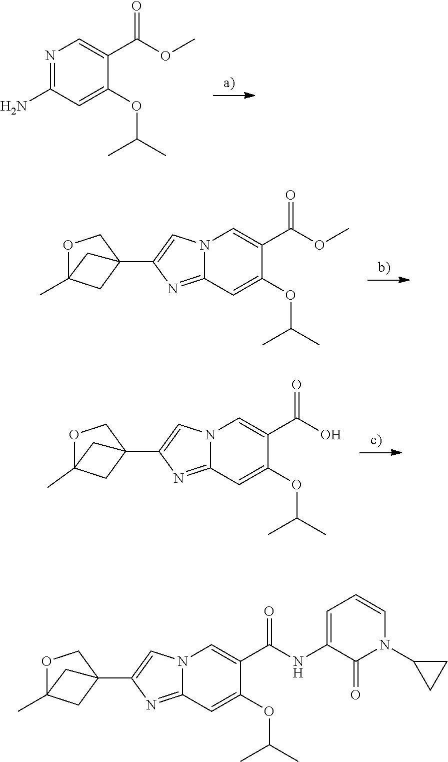

Step a: A mixture of methyl 6-amino-4-isopropoxynicotinate [preparation 2] (1.60 g, 7.61 mmol), 2-bromo-1-(1-ethyl-2-oxabicyclo[2.1.1]hexan-4-yl)ethanone (1.67 g, 7.6 mmol) and NaHCO₃ (1.92 g, 22.8 mmol) in MeCN (9.5 mL) and toluene (9.5 mL) was heated at 90° C. for 16 h. Silica and MeOH were added and the mixture was concentrated and purified by silica gel column chromatography (dry load, 40 g, 0-50% gradient of 3:1 EtOAC/EtOH in heptanes) to obtain methyl 7-isopropoxy-2-(1-methyl-2-oxabicyclo[2.1.1]hexan-4-yl)imidazo[1,2-a]pyridine-6-carboxylate (1.80 g, 5.45 mmol, 71% yield). (LCMS (ESI) m/z 331.1 (M+H)⁺.

Step b: A mixture of methyl 7-isopropoxy-2-(1-methyl-2-oxabicyclo[2.1.1]hexan-4-yl)imidazo[1,2-a]pyridine-6-carboxylate (1.35 g, 4.09 mmol) and lithium hydroxide hydrate (343 mg, 8.18 mmol) in MeOH (1.0 mL), THF (7.4 mL) and water (1.8 mL) was stirred overnight at room temperature before being neutralized with a 4.0 M hydrochloric acid solution in dioxane. The mixture was concentrated to obtain 7-isopropoxy-2-(1-methyl-2-oxabicyclo[2.1.1]hexan-4-yl)imidazo[1,2-a]pyridine-6-carboxylic acid, which was used without further purification in next reaction. Assumed 100% yield. LCMS (ESI) m/z 317.1 (M+H)⁺.

Step c: T3P (0.47 mmol, 282 μL, 50% purity in EtOAc) was added to -isopropoxy-2-(1-methyl-2-oxabicyclo[2.1.1]hexan-4-yl)imidazo[1,2-a]pyridine-6-carboxylic acid (30.0 mg, 94.8 μmol) and 3-amino-1-cyclopropyl-pyridin-2-one (18.5 mg, 0.12 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 mm; Mobile phase A: MeCN; Mobile phase B: H2O; Modifier: 0.1% TFA) to provide N-(1-cyclopropyl-2-oxo-3-pyridyl)-7-isopropoxy-2-(1-methyl-2-oxabicyclo[2.1.1]hexan-4-yl)imidazo[1,2-a]pyridine-6-carboxamide (15.1 mg, 26.8 μmol, 28% yield, Trifluoroacetic acid). LCMS (ESI) m/z 448.9 (M+H)⁺. 1H NMR (500 MHz, DMSO-d6) δ ppm 0.88-0.99 (m, 2H) 1.01-1.11 (m, 2H) 1.46 (s, 3H) 1.58 (d, J=6.10 Hz, 6H) 1.87 (dd, J=4.27, 1.83 Hz, 2H) 2.14 (dd, J=4.27, 1.22 Hz, 2H) 3.49-3.53 (m, 1H) 3.94 (s, 2H) 5.16-5.29 (m, 1H) 6.33 (t, J=7.32 Hz, 1H) 7.32-7.42 (m, 2H) 8.13 (s, 1H) 8.44 (dd, J=7.32, 1.22 Hz, 1H) 9.41 (s, 1H) 10.66 (s, 1H).

### Examples 7 & 8: N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyridine-6-carboxamide and N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyridine-6-carboxamide

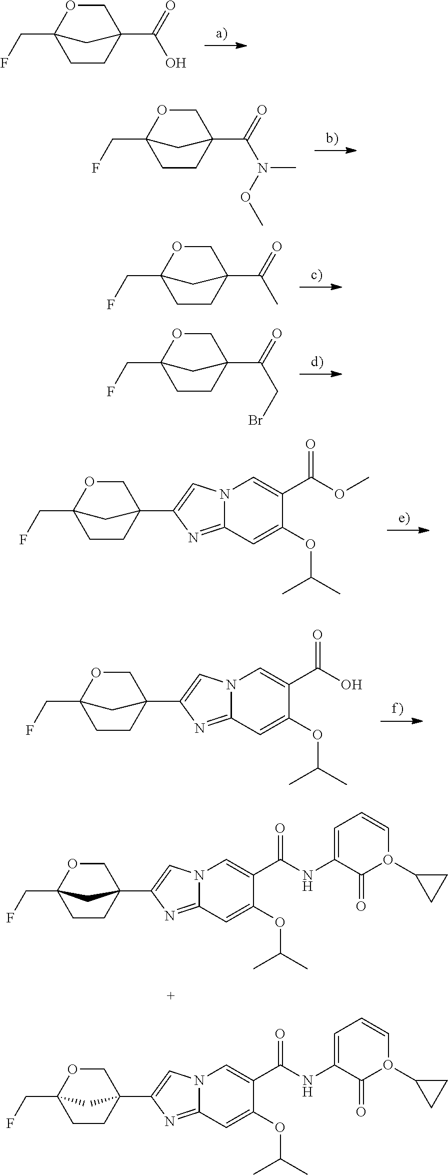

Step a: di(imidazol-1-yl)methanone (1.12 g, 6.89 mmol) was added to 1-(fluoromethyl)-2-oxabicyclo[2.2.1]heptane-4-carboxylic acid (1.0 g, 5.74 mmol) in DCM (8.20 mL) at rt. After stirring for 2 h, N-methoxymethanamine; hydrochloride (560 mg, 5.74 mmol) was added and stirred at rt overnight. The mixture was poured into water, extracted three times with DCM, dried MgSO₄, filtered, concentrated, purified by silica gel column (12 g, 0-100% 3; 1 EtOAc:EtOH in heptanes) to obtain 1-(fluoromethyl)-N-methoxy-N-methyl-2-oxabicyclo[2.2.1]heptane-4-carboxamide (600 mg, 2.76 mmol, 48.0% yield). (LCMS (ESI) m/z 218.0 (M+H)⁺.

Step b: Methyllithium (1.6 M, 2.16 mL) was added to 1-(fluoromethyl)-N-methoxy-N-methyl-2-oxabicyclo[2.2.1]heptane-4-carboxamide (600 mg, 2.76 mmol) in THF (5.5 mL) at −78° C. under nitrogen atmosphere. After 30 min, the mixture was warmed to 0° C. and then allowed to warm to rt over a period of 2 h. The reaction was quenched with sat. aq. NH₄Cl, extracted three times with EtOAc, washed with brine, dried over MgSO₄, filtered, and concentrated, to obtain 1-[1-(fluoromethyl)-2-oxabicyclo[2.2.1]heptan-4-yl]ethanone (450 mg, 2.61 mmol, 94% yield) which was used directly in next reaction without further purification.

Step c: dibromocopper (817 mg, 3.66 mmol) was added to 1-[1-(fluoromethyl)-2-oxabicyclo[2.2.1]heptan-4-yl]ethanone (450 mg, 2.61 mmol) in EtOH (6.5 mL) at rt. The mixture was heated at 70° C. for 25 min, then cooled to rt, diluted with water and EtOAc, extracted three times with EtOAc, washed with sat. aq. NaHCO₃, then brine, dried over MgSO₄, filtered, concentrated, to obtain 2-bromo-1-[1-(fluoromethyl)-2-oxabicyclo[2.2.1]heptan-4-yl]ethanone (500 mg, 1.99 mmol, 76% yield) which was used directly in next reaction without further purification.

Step d: A mixture of methyl 6-amino-4-isopropoxynicotinate [preparation 2] (400 mg, 1.90 mmol), 2-bromo-1-[1-(fluoromethyl)-2-oxabicyclo[2.2.1]heptan-4-yl]ethanone (477 mg, 1.90 mmol), NaHCO₃ (479 mg, 5.71 mmol) in Acetonitrile (2.2 mL) and Toluene (3.3 mL) was heated at 90° C. for 16 h. Silica and MeOH were added and the mixture was concentrated and purified by silica gel column chromatography (dry load, 24 g, 0-70% gradient of 3:1 EtOAC/EtOH in heptanes) to obtain methyl 2-[(−1-(fluoromethyl)-2-oxabicyclo[2.2.1]heptan-4-yl]-7-isopropoxy-imidazo[1,2-a]pyridine-6-carboxylate (0.50 g, 72% yield). (LCMS (ESI) m/z 363.0 (M+H)⁺.

Step e: A mixture of methyl 2-[(−1-(fluoromethyl)-2-oxabicyclo[2.2.1]heptan-4-yl]-7-isopropoxy-imidazo[1,2-a]pyridine-6-carboxylate (320 mg, 0.88 mmol) and lithium; hydroxide; hydrate (74.0 mg, 1.77 mmol) in Methanol (275 μL), THF (1.99 mL), water (496 μL) was stirred overnight at room temperature before being neutralized with a 4.0 M hydrochloric acid solution in dioxane. The mixture was concentrated to obtain 2-(1-(fluoromethyl)-2-oxabicyclo[2.2.1]heptan-4-yl)-7-isopropoxyimidazo[1,2-a]pyridine-6-carboxylic acid, which was used without further purification in next reaction. Assumed 100% yield. LCMS (ESI) m/z 348.9 (M+H)⁺.

Step f: T3P (0.47 mmol, 282 uL, 50% purity in EtOAc) was added to 2-(1-(fluoromethyl)-2-oxabicyclo[2.2.1]heptan-4-yl)-7-isopropoxyimidazo[1,2-a]pyridine-6-carboxylic acid and (77 mg, 0.22 mmol) and 3-amino-1-cyclopropyl-pyridin-2-one (36.5 mg, 0.24 mmol) in Pyridine (1.5 mL) at rt. After stirring overnight, the mixture was diluted with water and extracted with DCM and then EtOAc, dried over MgSO4, filtered and concentrated. The crude material was purified by SFC: CHIRALPAK IB 30×250 mm, 5 um Method: 45% MeOH w/ 0.1% DEA in CO₂ (flow rate: 100 mL/min, ABPR 120 bar, MBPR 40 psi, column temp 40 deg C.) to obtain Peak 1, Example 7: N-(1-cyclopropyl-2-oxo-3-pyridyl)-2-[(1S,4R)-1-(fluoromethyl)-2-oxabicyclo[2.2.1]heptan-4-yl]-7-isopropoxy-imidazo[1,2-a]pyridine-6-carboxamide (17.2 mg, 16% yield, stereochemistry arbitrarily assigned); LCMS (ESI) m/z 480.9 (M+H)⁺.; ¹⁹F NMR (470 MHz, CHLOROFORM-d) δ ppm −75.83 (s, 1 F); ¹H NMR (500 MHz, CHLOROFORM-d) δ ppm 0.88-0.97 (m, 2H) 1.15-1.22 (m, 2H) 1.66 (d, J=6.10 Hz, 6H) 1.92-2.03 (m, 3H) 2.15-2.29 (m, 3H) 3.42-3.51 (m, 1H) 4.06 (d, J=6.71 Hz, 1H) 4.16 (dd, J=6.41, 3.36 Hz, 1H) 4.61-4.69 (m, 1H) 4.71-4.79 (m, 1H) 4.85 (dt, J=12.21, 6.10 Hz, 1H) 6.24 (t, J=7.33 Hz, 1H) 7.01 (s, 1H) 7.06 (dd, J=6.71, 1.83 Hz, 1H) 7.35 (s, 1H) 8.56 (dd, J=7.33, 1.83 Hz, 1H) 8.99 (s, 1H) 10.78 (s, 1H) and Peak 2, Example 8: N-(1-cyclopropyl-2-oxo-3-pyridyl)-2-[(1R,4S)-1-(fluoromethyl)-2-oxabicyclo[2.2.1]heptan-4-yl]-7-isopropoxy-imidazo[1,2-a]pyridine-6-carboxamide (20.2 mg, 18.0% yield, stereochemistry arbitrarily assigned) LCMS (ESI) m/z 480.9 (M+H)⁺; ¹⁹F NMR (470 MHz, CHLOROFORM-d) δ ppm −75.79 (s, 1 F); 1H NMR (500 MHz, CHLOROFORM-d) δ ppm 0.89-0.95 (m, 2H) 1.15-1.23 (m, 2H) 1.66 (d, J=6.10 Hz, 6H) 1.91-2.03 (m, 3H) 2.12-2.28 (m, 3H) 3.43-3.49 (m, 1H) 4.05 (d, J=6.71 Hz, 1H) 4.16 (dd, J=6.41, 3.36 Hz, 1H) 4.61-4.70 (m, 1H) 4.72-4.78 (m, 1H) 4.85 (dt, J=12.21, 6.10 Hz, 1H) 6.24 (t, J=7.02 Hz, 1H) 7.01 (s, 1H) 7.06 (dd, J=6.71, 1.83 Hz, 1H) 7.35 (s, 1H) 8.56 (dd, J=7.94, 1.83 Hz, 1H) 8.99 (s, 1H) 10.78 (s, 1H).

### Example 9: N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyridine-6-carboxamide

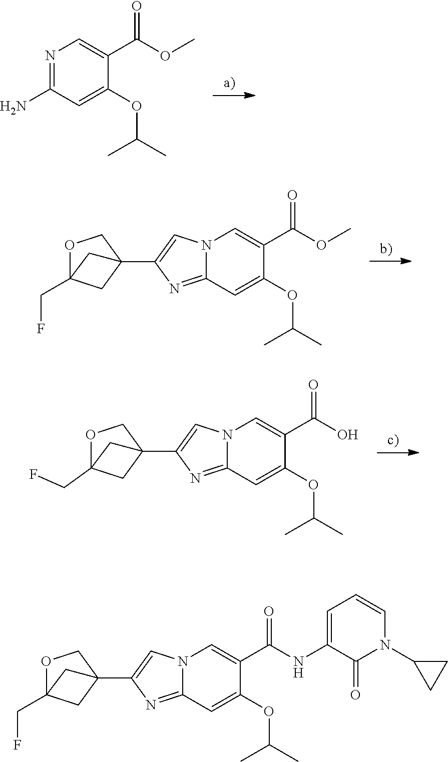

Step a: A mixture of methyl 6-amino-4-isopropoxynicotinate [preparation 2] (500 mg, 2.38 mmol), 2-bromo-1-[1-(fluoromethyl)-2-oxabicyclo[2.1.1]hexan-4-yl]ethanone (620 mg, 2.62 mmol), NaHCO₃ (599 mg, 7.14 mmol) in Acetonitrile (2.7 mL) and Toluene (4.1 mL) was heated at 90° C. for 16 h. Silica and MeOH were added and the mixture was concentrated and purified by silica gel column chromatography (dry load, 40 g, 0-50% gradient of 3:1 EtOAC/EtOH in heptanes) to obtain methyl 2-[1-(fluoromethyl)-2-oxabicyclo[2.1.1]hexan-4-yl]-7-isopropoxy-imidazo[1,2-a]pyridine-6-carboxylate (650 mg, 1.87 mmol, 78% yield). (LCMS (ESI) m/z 348.9 (M+H)⁺.

Step b: A mixture of methyl 2-[1-(fluoromethyl)-2-oxabicyclo[2.1.1]hexan-4-yl]-7-isopropoxy-imidazo[1,2-a]pyridine-6-carboxylate (200 mg, 574.11 μmol) and lithium; hydroxide; hydrate (48.0 mg, 1.15 mmol) in Methanol (574 μL), THF (4.1 mL), water (1.0 mL) was stirred overnight at room temperature before being neutralized with a 4.0 M hydrochloric acid solution in dioxane. The mixture was concentrated to obtain 2-[1-(fluoromethyl)-2-oxabicyclo[2.1.1]hexan-4-yl]-7-isopropoxy-imidazo[1,2-a]pyridine-6-carboxylic acid, which was used without further purification in next reaction. Assumed 100% yield. LCMS (ESI) m/z 334.9 (M+H)⁺.

Step c: T3P (0.57 mmol, 338 μL, 50% purity in EtOAc) was added to 2-[1-(fluoromethyl)-2-oxabicyclo[2.1.1]hexan-4-yl]-7-isopropoxy-imidazo[1,2-a]pyridine-6-carboxylic acid (38 mg, 0.11 mmol) and 3-amino-1-cyclopropyl-pyridin-2-one (20 mg, 0.13 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyridine-6-carboxamide (36.7 mg, 66.0% yield). LCMS (ESI) m/z 467.0 (M+H)⁺. ¹⁹F NMR (470 MHz, DMSO-d₆) δ ppm −74.81 (m, 1 F); 1H NMR (500 MHz, DMSO-d6) δ ppm 0.88-0.95 (m, 2H) 1.01-1.11 (m, 2H) 1.53 (d, J=6.10 Hz, 6H) 1.86 (dd, J=4.27, 1.22 Hz, 2H) 2.14 (dd, J=4.27, 1.22 Hz, 2H) 3.47-3.53 (m, 1H) 3.50 (td, J=7.63, 3.66 Hz, 1H) 3.97 (s, 2H) 4.64-4.78 (m, 2H) 4.98-5.10 (m, 1H) 6.30 (t, J=7.32 Hz, 1H) 7.22 (s, 1H) 7.33 (dd, J=6.71, 1.83 Hz, 1H) 7.89 (s, 1H) 8.45 (dd, J=7.63, 1.53 Hz, 1H) 9.21 (s, 1H) 10.76 (s, 1H).

### Example 10: N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide

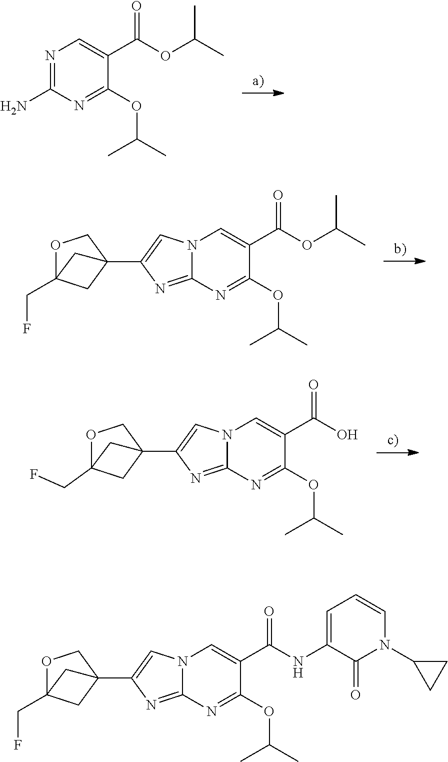

Step a: A mixture of isopropyl 2-amino-4-isopropoxy-pyrimidine-5-carboxylate (500 mg, 2.09 mmol), 2-bromo-1-[1-(fluoromethyl)-2-oxabicyclo[2.1.1]hexan-4-yl]ethanone (544.93 mg, 2.30 mmol), sodium; hydrogen carbonate (526.64 mg, 6.27 mmol) in Acetonitrile (2.7 mL) and Toluene (4.1 mL) was heated at 90° C. for 16 h. Silica and MeOH were added and the mixture was concentrated and purified by silica gel column chromatography (dry load, 40 g, 0-100% gradient of 3:1 EtOAC/EtOH in heptanes) to obtain isopropyl 2-[1-(fluoromethyl)-2-oxabicyclo[2.1.1]hexan-4-yl]-7-isopropoxy-imidazo[1,2-a]pyrimidine-6-carboxylate (500 mg, 1.32 mmol, 63% yield) (LCMS (ESI) m/z 378.0 (M+H)⁺.

Step b: A mixture of isopropyl 2-[1-(fluoromethyl)-2-oxabicyclo[2.1.1]hexan-4-yl]-7-isopropoxy-imidazo[1,2-a]pyrimidine-6-carboxylate (160 mg, 423.94 μmol) and lithium; hydroxide; hydrate (35.0 mg, 847 μmol) in Methanol (423 μL), THF (3.05 mL), water (763 μL) was stirred overnight at room temperature before being neutralized with a 4.0 M hydrochloric acid solution in dioxane. The mixture was concentrated to obtain 2-[1-(fluoromethyl)-2-oxabicyclo[2.1.1]hexan-4-yl]-7-isopropoxy-imidazo[1,2-a]pyrimidine-6-carboxylic acid, which was used without further purification in next reaction. Assumed 100% yield. LCMS (ESI) m/z 335.9 (M+H)⁺.

Step c: T3P (0.57 mmol, 338 μL, 50% purity in EtOAc) was added to 2-[1-(fluoromethyl)-2-oxabicyclo[2.1.1]hexan-4-yl]-7-isopropoxy-imidazo[1,2-a]pyrimidine-6-carboxylic acid (38 mg, 0.11 mmol) and 3-amino-1-cyclopropyl-pyridin-2-one (20 mg, 0.13 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H2O, Modifier: 0.1% NH₄OH) to provide N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide (27.6 mg, 35.0% yield). LCMS (ESI) m/z 467.9 (M+H)⁺. ₁₉F NMR (470 MHz, DMSO-d₆) δ ppm −74.12 (s, 1 F); 1H NMR (500 MHz, DMSO-d6) δ ppm 0.89-0.95 (m, 2H) 1.03-1.08 (m, 2H) 1.58 (d, J=6.10 Hz, 6H) 1.85-1.94 (m, 2H) 2.19 (br d, J=3.05 Hz, 2H) 3.51 (td, J=7.63, 3.66 Hz, 1H) 3.98 (s, 2H) 4.65-4.79 (m, 2H) 5.63 (quin, J=6.10 Hz, 1H) 6.29-6.37 (m, 1H) 7.36 (dd, J=7.02, 1.53 Hz, 1H) 7.84 (s, 1H) 8.43 (dd, J=7.32, 1.83 Hz, 1H) 9.55 (s, 1H) 10.68 (s, 1H).

### Example 11: N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyridine-6-carboxamide

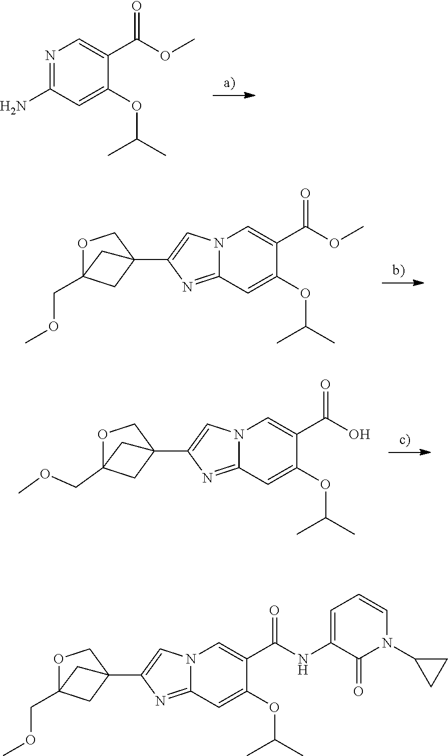

Step a: A mixture of methyl 6-amino-4-isopropoxynicotinate [preparation 2] (200 mg, 951 mol), 2-bromo-1-[1-(methoxymethyl)-2-oxabicyclo[2.1.1]hexan-4-yl]ethanone (237 mg, 951 μmol), NaHCO₃ (239 mg, 2.85 mmol) in Acetonitrile (1.5 mL) and Toluene (2.3 mL) was heated at 90° C. for 16 h. Silica and MeOH were added and the mixture was concentrated and purified by silica gel column chromatography (dry load, 40 g, 0-50% gradient of 3:1 EtOAC/EtOH in heptanes) to obtain methyl 7-isopropoxy-2-[1-(methoxymethyl)-2-oxabicyclo[2.1.1]hexan-4-yl]imidazo[1,2-a]pyridine-6-carboxylate (300 mg, 832 μmol, 87.50% yield). (LCMS (ESI) m/z 361.0 (M+H)⁺.

Step b: A mixture of methyl 7-isopropoxy-2-[1-(methoxymethyl)-2-oxabicyclo[2.1.1]hexan-4-yl]imidazo[1,2-a]pyridine-6-carboxylate (150 mg, 416 μmol) and LiOH·H₂O (34.9 mg, 832 μmol) in Methanol (416 μL), THF (3.0 mL), water (749 μL) was stirred overnight at room temperature before being neutralized with a 4.0 M hydrochloric acid solution in dioxane. The mixture was concentrated to obtain 7-isopropoxy-2-[1-(methoxymethyl)-2-oxabicyclo[2.1.1]hexan-4-yl]imidazo[1,2-a]pyridine-6-carboxylic acid, which was used without further purification in next reaction. Assumed 100% yield. LCMS (ESI) m/z 346.9 (M+H)⁺.

Step c: T3P (0.57 mmol, 338 μL, 50% purity in EtOAc) was added to 7-isopropoxy-2-[1-(methoxymethyl)-2-oxabicyclo[2.1.1]hexan-4-yl]imidazo[1,2-a]pyridine-6-carboxylic acid (48 mg, 0.14 mmol) and 3-amino-1-cyclopropyl-pyridin-2-one (27 mg, 0.18 mmol, hydrochloride) in Pyridine (1.0 mL) at rt. After stirring overnight, the mixture was diluted with water and extracted with DCM and then EtOAc, dried over MgSO4, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyridine-6-carboxamide (25.8 mg, 39.0% yield). LCMS (ESI) m/z 478.9 (M+H)⁺. 1H NMR (500 MHz, DMSO-d6) δ ppm 0.90-0.93 (m, 2H) 1.05 (dd, J=7.94, 1.83 Hz, 2H) 1.53 (d, J=6.10 Hz, 6H) 1.80 (dd, J=4.27, 1.83 Hz, 2H) 2.07 (dd, J=4.58, 1.53 Hz, 2H) 3.33 (s, 2H) 3.50 (tt, J=7.71, 3.89 Hz, 1H) 3.63 (s, 3H) 3.92 (s, 2H) 5.04 (dt, J=12.06, 5.88 Hz, 1H) 6.30 (t, J=7.32 Hz, 1H) 7.21 (s, 1H) 7.33 (dd, J=6.71, 1.83 Hz, 1H) 7.88 (s, 1H) 8.45 (dd, J=7.32, 1.83 Hz, 1H) 9.20 (s, 1H) 10.75 (s, 1H).

### Examples 12 and 13: N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide and N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide

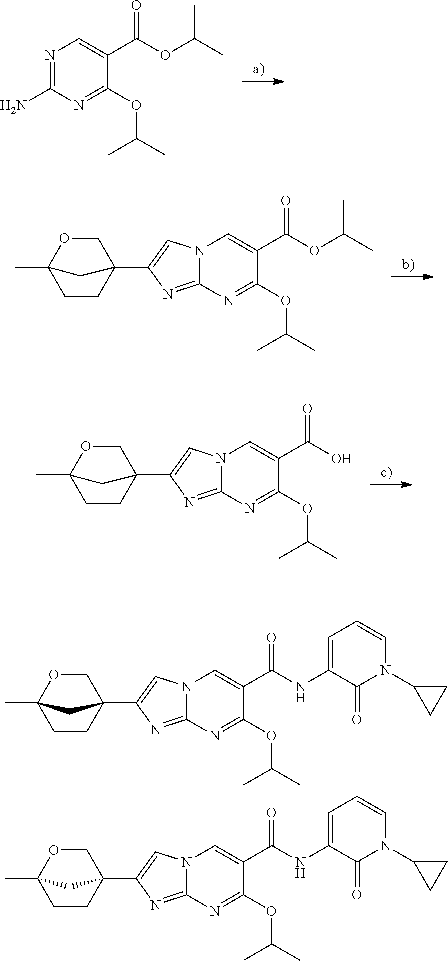

Step a: A mixture of isopropyl 2-amino-4-isopropoxy-pyrimidine-5-carboxylate (300 mg, 1.25 mmol), 2-bromo-1-[1-methyl-2-oxabicyclo[2.2.1]heptan-4-yl]ethanone (350 mg, 1.50 mmol), sodium; hydrogen carbonate (316 mg, 3.76 mmol) in Acetonitrile (2.0 mL) and Toluene (3.0 mL) was heated at 90° C. for 16 h. Silica and MeOH were added and the mixture was concentrated and purified by silica gel column chromatography (dry load, 12 g, 0-70% gradient of 3:1 EtOAC/EtOH in heptanes) to obtain isopropyl 7-isopropoxy-2-[1-methyl-2-oxabicyclo[2.2.1]heptan-4-yl]imidazo[1,2-a]pyrimidine-6-carboxylate (350 mg, 937 μmol, 74% yield). (LCMS (ESI) m/z 374.0 (M+H)⁺.

Step b: A mixture of isopropyl 7-isopropoxy-2-[1-methyl-2-oxabicyclo[2.2.1]heptan-4-yl]imidazo[1,2-a]pyrimidine-6-carboxylate (70.0 mg, 187 μmol) and LiOH·H₂O (15.7 mg, 374 μmol) in Methanol (231 μL), THF (1.7 mL), water (420 μL) was stirred overnight at rt before being neutralized with a 4.0 M hydrochloric acid solution in dioxane. The mixture was concentrated to obtain 7-isopropoxy-2-[1-methyl-2-oxabicyclo[2.2.1]heptan-4-yl]imidazo[1,2-a]pyrimidine-6-carboxylic acid, which was used without further purification in next reaction assuming 100% yield. LCMS (ESI) m/z 331.9 (M+H)⁺.

Step c: T3P (0.57 mmol, 338 μL, 50% purity in EtOAc) was added to 7-isopropoxy-2-[1-methyl-2-oxabicyclo[2.2.1]heptan-4-yl]imidazo[1,2-a]pyrimidine-6-carboxylic acid (63.0 mg, 190 μmol) and 3-amino-1-cyclopropyl-pyridin-2-one (34.2 mg, 228 μmol) in Pyridine (1.5 mL) at rt. After stirring overnight, the mixture was diluted with water and extracted with DCM and then EtOAc, dried over MgSO4, filtered and concentrated. The crude material was purified by SFC: CHIRALPAK IB 30×250 mm, 5 um Method: 40% EtOH w/ 0.1% DEA in CO₂ (flow rate: 100 mL/min, ABPR 120 bar, MBPR 40 psi, column temp 40 deg C.) to provide, Peak 1, N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide, Example 12, stereochemistry arbitrarily assigned (11.6 mg, 13% yield); LCMS (ESI) m/z 464.0 (M+H)⁺. 1H NMR (400 MHz, CHLOROFORM-d) δ ppm 0.94 (br d, J=3.76 Hz, 2H) 1.19 (br d, J=7.53 Hz, 2H) 1.50 (s, 3H) 1.65 (d, J=6.27 Hz, 6H) 1.85 (br dd, J=12.05, 4.77 Hz, 2 H) 1.94 (br d, J=9.54 Hz, 1H) 2.03 (br d, J=9.04 Hz, 1H) 2.09 (br s, 1H) 2.21 (br d, J=4.52 Hz, 1H) 3.42-3.55 (m, 1H) 3.97 (d, J=6.78 Hz, 1H) 4.14 (dd, J=6.40, 3.39 Hz, 1H) 5.84-5.94 (m, 1H) 6.24 (t, J=7.15 Hz, 1H) 7.07 (br d, J=6.78 Hz, 1H) 7.23 (s, 1H) 8.53 (br d, J=7.03 Hz, 1H) 9.11-9.18 (m, 1H) 10.80 (s, 1H) and Peak 2, N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide, Example 13, stereochemistry arbitrarily assigned (12.3 mg, 14% yield); LCMS (ESI) m/z 464.0 (M+H)⁺; 1H NMR (400 MHz, CHLOROFORM-d) δ ppm 0.94 (br s, 2H) 1.20 (br s, 2H) 1.50 (s, 3H) 1.64-1.68 (m, 6H) 1.83-1.98 (m, 3H) 2.03 (br d, J=9.04 Hz, 1H) 2.09 (br s, 1H) 2.20 (br s, 1H) 3.48 (br s, 1H) 3.97 (br d, J=6.78 Hz, 1H) 4.15 (br s, 1H) 5.89 (br d, J=6.27 Hz, 1H) 6.25 (br d, J=7.53 Hz, 1H) 7.08 (br s, 1H) 7.21-7.27 (m, 1H) 8.54 (br s, 1H) 9.15 (s, 1H) 10.80 (br s, 1H).

### Examples 14 and 15: (R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide and (S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide

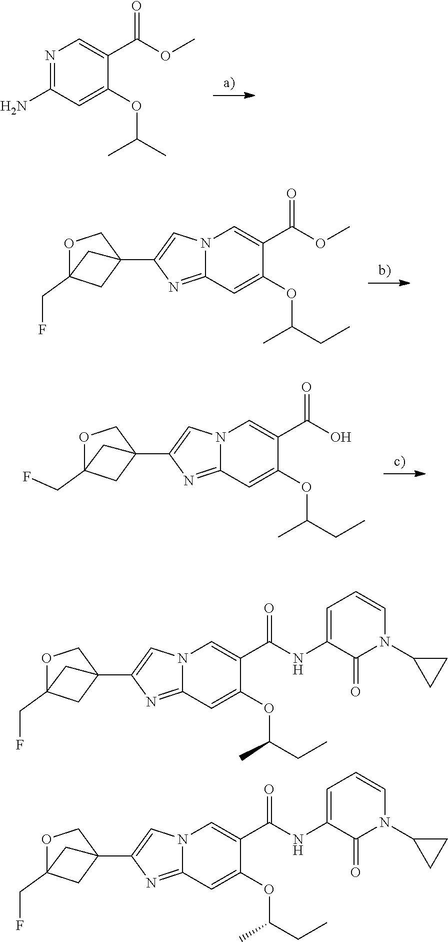

Step a: A mixture of methyl 6-amino-4-[1-methylpropoxy]pyridine-3-carboxylate (300 mg, 1.34 mmol), 2-bromo-1-[1-(fluoromethyl)-2-oxabicyclo[2.1.1]hexan-4-yl]ethanone (348 mg, 1.47 mmol), NaHCO₃ (337 mg, 4.01 mmol) in Acetonitrile (2.4 mL) and Toluene (3.6 mL) was heated at 90° C. for 16 h. Silica and MeOH were added and the mixture was concentrated and purified by silica gel column chromatography (dry load, 24 g, 0-50% gradient of 3:1 EtOAC/EtOH in heptanes) to obtain methyl 2-[1-(fluoromethyl)-2-oxabicyclo[2.1.1]hexan-4-yl]-7-[1-methylpropoxy]imidazo[1,2-a]pyridine-6-carboxylate (400 mg, 1.10 mmol, 82% yield). (LCMS (ESI) m/z 363.2 (M+H)⁺.

Step b: A mixture of methyl 2-[1-(fluoromethyl)-2-oxabicyclo[2.1.1]hexan-4-yl]-7-[1-methylpropoxy]imidazo[1,2-a]pyridine-6-carboxylate (400 mg, 1.10 mmol) and lithium; hydroxide; hydrate (92 mg, 2.21 mmol) in Methanol (735 μL), THF (5.3 mL), water (1.3 mL) was stirred overnight at room temperature before being neutralized with a 4.0 M hydrochloric acid solution in dioxane. The mixture was concentrated to obtain 2-[1-(fluoromethyl)-2-oxabicyclo[2.1.1]hexan-4-yl]-7-[1-methylpropoxy]imidazo[1,2-a]pyridine-6-carboxylic acid, which was used without further purification in next reaction. Assumed 100% yield. LCMS (ESI) m/z 349.2 (M+H)⁺.

Step c: T3P (2.01 mmol, 1.20 mL, 50% purity in EtOAc) was added to 2-[1-(fluoromethyl)-2-oxabicyclo[2.1.1]hexan-4-yl]-7-[1-methylpropoxy]imidazo[1,2-a]pyridine-6-carboxylic acid (140 mg, 402 μmol) and 3-amino-1-cyclopropyl-pyridin-2-one (97.5 mg, 522 μmol, Hydrochloride) in Pyridine (2.0 mL) at rt. After stirring overnight, the mixture was diluted with water and extracted with DCM and then EtOAc, dried over MgSO₄, filtered and concentrated. The crude material was purified by chiral SFC: CHIRALPAK IB 30×250 mm, 5 um Method: 40% MeOH w/ 0.1% DEA in CO₂ (flow rate: 100 mL/min, ABPR 120 bar, MBPR 40 psi, column temp 40 deg C.) to provide Peak 1: (R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide, Example 14, stereochemistry arbitrarily assigned (28.1 mg, 14% yield). LCMS (ESI) m/z 481.3 (M+H)⁺; 19F NMR (376 MHz, CHLOROFORM-d) δ ppm −75.67 (s, 1 F); 1H NMR (400 MHz, CHLOROFORM-d) δ ppm 0.88-0.96 (m, 2H) 1.04 (t, J=7.40 Hz, 3H) 1.15-1.24 (m, 2H) 1.62 (d, J=6.02 Hz, 3H) 1.84-1.95 (m, 1H) 2.09 (dd, J=4.52, 1.51 Hz, 2H) 2.26 (dd, J=4.64, 1.38 Hz, 3H) 3.46 (dt, J=7.72, 3.55 Hz, 1H) 4.16 (s, 2H) 4.56-4.63 (m, 1H) 4.66 (s, 1H) 4.78 (s, 1H) 6.23 (t, J=7.28 Hz, 1H) 7.03 (s, 1H) 7.06 (dd, J=6.78, 1.76 Hz, 1H) 7.39 (s, 1H) 8.55 (dd, J=7.28, 1.76 Hz, 1H) 9.01 (s, 1H) 10.79 (s, 1H) and Peak 2: (S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide Example 15, stereochemistry arbitrarily assigned (28.1 mg, 14% yield). LCMS (ESI) m/z 481.3 (M+H)⁺; 19F NMR (376 MHz, CHLOROFORM-d) δ ppm −75.69 (s, 1 F); 1H NMR (400 MHz, CHLOROFORM-d) δ ppm 0.90-0.96 (m, 2H) 1.04 (t, J=7.40 Hz, 3H) 1.15-1.22 (m, 2H) 1.62 (d, J=6.02 Hz, 3H) 1.84-1.94 (m, 1H) 2.06-2.11 (m, 2H) 2.17-2.29 (m, 3H) 3.46 (tt, J=7.59, 3.83 Hz, 1H) 4.15 (s, 2H) 4.55-4.64 (m, 1H) 4.66 (s, 1H) 4.78 (s, 1H) 6.23 (t, J=7.15 Hz, 1H) 7.02 (s, 1H) 7.06 (dd, J=7.03, 1.76 Hz, 1H) 7.39 (s, 1H) 8.55 (dd, J=7.53, 1.76 Hz, 1H) 9.00 (s, 1H) 10.79 (s, 1H).

### Example 16 and 17: 7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide and 7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide

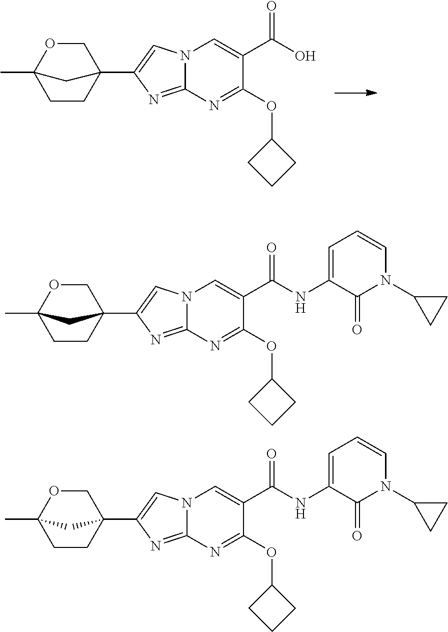

T3P (582 μmol, 346 μL, 50% purity in EtOAc) was added to 7-(cyclobutoxy)-2-[1-methyl-2-oxabicyclo[2.2.1]heptan-4-yl]imidazo[1,2-a]pyrimidine-6-carboxylic acid (40 mg, 116 μmol) and 3-amino-1-cyclopropyl-pyridin-2-one (22.7 mg, 151 μmol) in Pyridine (1.2 mL) at rt. After stirring overnight, the mixture was diluted with water and extracted with DCM and then EtOAc, dried over MgSO₄, filtered and concentrated. The crude material was purified by chrial SFC: CHIRALPAK IB 30×250 mm, 5 μm Method: 45% MeOH w/ No Modifier in CO₂ (flow rate: 100 mL/min, ABPR 120 bar, MBPR 40 psi, column temp 40 deg C.) to provide Peak 1: 7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide, Example 16, stereochemistry arbitrarily assigned (3.3 mg, 6% yield). LCMS (ESI) m/z 475.9 (M+H)⁺. 1H NMR (400 MHz, CHLOROFORM-d) δ ppm 0.92-0.99 (m, 2H) 1.16-1.23 (m, 2H) 1.49 (s, 3H) 1.73-1.89 (m, 3H) 1.93 (d, J=9.54 Hz, 1H) 1.99-2.13 (m, 3H) 2.18 (ddd, J=9.04, 5.77, 2.76 Hz, 1H) 2.57-2.65 (m, 2H) 2.66-2.74 (m, 2H) 3.45-3.54 (m, 1H) 3.97 (d, J=6.53 Hz, 1H) 4.14 (dd, J=6.53, 3.51 Hz, 1H) 5.58-5.69 (m, 1H) 6.25 (t, J=7.28 Hz, 1H) 7.08 (dd, J=6.90, 1.88 Hz, 1H) 7.23 (s, 1H) 8.54 (dd, J=7.53, 1.76 Hz, 1H) 9.15 (s, 1H) 10.83 (s, 1H) and Peak 2: 7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide, Example 17, stereochemistry arbitrarily assigned (3.3 mg. 6% yield). LCMS (ESI) m/z 475.9 (M+H)⁺; 1H NMR (400 MHz, CHLOROFORM-d) δ ppm 0.92-0.98 (m, 2H) 1.16-1.24 (m, 2H) 1.49 (s, 3H) 1.76-1.89 (m, 3H) 1.93 (d, J=9.54 Hz, 1H) 1.99-2.13 (m, 1H) 1.99-2.13 (m, 2H) 2.15-2.27 (m, 1H) 2.57-2.76 (m, 4H) 3.49 (tt, J=7.59, 3.83 Hz, 1H) 3.97 (d, J=6.53 Hz, 1H) 4.14 (dd, J=6.40, 3.39 Hz, 1H) 5.63 (quin, J=7.22 Hz, 1H) 6.25 (t, J=7.28 Hz, 1H) 7.08 (dd, J=7.03, 1.76 Hz, 1H) 7.23 (s, 1H) 8.54 (dd, J=7.53, 1.76 Hz, 1H) 9.16 (s, 1H) 10.83 (s, 1H).

### Example 18: N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

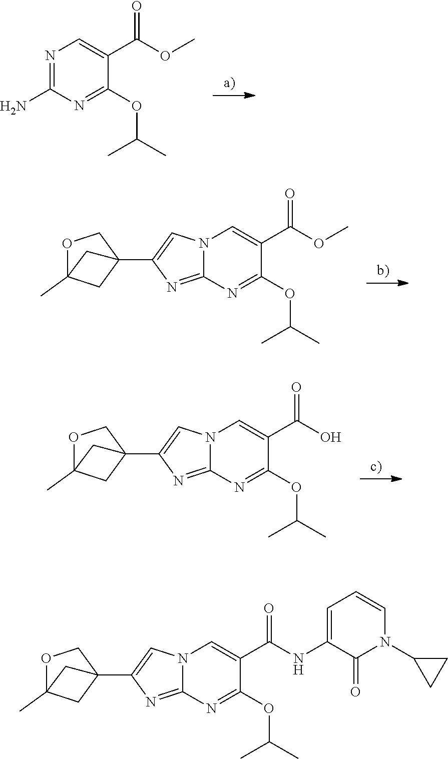

Step a: A mixture of methyl 2-amino-4-isopropoxy-pyrimidine-5-carboxylate [preparation 5](8.0 g, 37.88 mmol), 2-bromo-1-(1-methyl-2-oxabicyclo[2.1.1]hexan-4-yl)ethan-1-one [preparation 12] (11.62 g, 53.03 mmol) and NaHCO₃ (9.55 g, 113.63 mmol) in MeCN (43.3 mL) and toluene (64.9 mL) was heated at 90° C. for 16 h. The solvents were removed under reduced pressure. Then silica and MeOH were added and the mixture was concentrated and purified by silica gel column chromatography (dry load, 220 g, 5-50% gradient of 3:1 EtOAC/EtOH in heptanes) to obtain methyl 7-isopropoxy-2-(1-methyl-2-oxabicyclo[2.1.1]hexan-4-yl)imidazo[1,2-a]pyrimidine-6-carboxylate (6.28 g, 18.95 mmol, 50% yield). LCMS (ESI) m/z 331.9 (M+H)⁺. 1H NMR (400 MHz, CHLOROFORM-d) δ ppm 1.45 (d, J=6.27 Hz, 6H) 1.54 (s, 3H) 1.91-1.98 (m, 2H) 2.10 (dd, J=4.64, 1.63 Hz, 2H) 3.93 (s, 3H) 4.07 (s, 2H) 5.62 (spt, J=6.19 Hz, 1H) 7.17 (s, 1H) 8.85 (s, 1H).

Step b: A mixture of methyl 7-isopropoxy-2-(1-methyl-2-oxabicyclo[2.1.1]hexan-4-yl)imidazo[1,2-a]pyrimidine-6-carboxylate (6.28 g, 18.9 mmol) and lithium hydroxide hydrate (1.59 g, 37.9 mmol) in MeOH (6.3 mL), THF (45.5 mL) and water (11.4 mL) was stirred 4 h at room temperature before being neutralized with a 4.0 M hydrochloric acid solution in dioxane. After removal of solvents, the mixture was washed with toluene, EtOAc, and heptanes to obtain 7-isopropoxy-2-(1-methyl-2-oxabicyclo[2.1.1]hexan-4-yl)imidazo[1,2-a]pyrimidine-6-carboxylic acid, which was used without further purification in the next reaction. Assumed 100% yield. LCMS (ESI) m/z 317.9 (M+H)⁺. 1H NMR (400 MHz, DMSO-d6) δ ppm 1.35 (d, J=6.02 Hz, 6H) 1.42 (s, 3H) 1.74 (dd, J=4.39, 1.63 Hz, 2H) 1.98 (dd, J=4.39, 1.63 Hz, 2H) 3.86 (s, 2H) 5.36 (quin, J=6.21 Hz, 1H) 7.58 (s, 1H) 9.27 (s, 1H).

Step c: To a mixture of 7-isopropoxy-2-(1-methyl-2-oxabicyclo[2.1.1]hexan-4-yl)imidazo[1,2-a]pyrimidine-6-carboxylic acid [preparation 8 or step b above] (3.0 g, 9.45 mmol) and 3-amino-1-cyclopropyl-pyridin-2-one (1.85 g, 9.93 mmol, Hydrochloride) in DMF (31.3 mL) was added HATU (3.78 g, 9.93 mmol), followed by DIPEA (28.36 mmol, 4.9 mL) at room temperature. After stirring for 16 h, the reaction mixture was concentrated, then diluted with brine and DCM. The aqueous phase was extracted two times with DCM and then two more times with EtOAc. The organic layer was dried over MgSO₄, filtered, concentrated and purified by silica gel column chromatography (dry load, 220 g, 5-50% gradient of 3:1 EtOAC/EtOH in heptanes) to obtain N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (2.3 g). The material was recrystallized three times in 12 Volumes of a 3:1 mixture of EtOAc:EtOH to obtain N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (0.9 g, 21% yield). LCMS (ESI) m/z 450.0 (M+H)⁺. 1H NMR (400 MHz, CHLOROFORM-d) δ ppm 0.94 (br d, J=4.02 Hz, 2H) 1.16-1.25 (m, 2H) 1.55 (s, 3H) 1.66 (d, J=6.27 Hz, 6H) 1.96 (dd, J=4.64, 1.38 Hz, 2H) 2.11 (d, J=4.52 Hz, 2H) 3.48 (dt, J=7.47, 3.67 Hz, 1H) 4.09 (s, 2H) 5.86 (dt, J=12.55, 6.27 Hz, 1H) 6.24 (t, J=7.15 Hz, 1H) 7.07 (dd, J=7.03, 1.51 Hz, 1H) 7.27 (s, 1H) 8.53 (dd, J=7.28, 1.76 Hz, 1H) 9.16 (s, 1H) 10.80 (s, 1H).

### Example 19: (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide

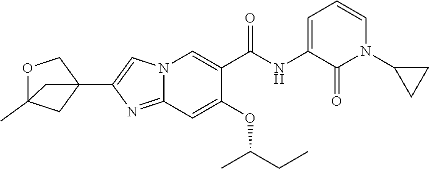 [SMILES: CC[C@H](C)Oc1cc2nc(C34COC(C)(C3)C4)cn2cc1C(=O)Nc1cccn(C2CC2)c1=O]

2-(1-methyl-2-oxabicyclo[2.1.1]hexan-4-yl)-7-[(1S)-1-methylpropoxy]imidazo[1,2-a]pyridine-6-carboxylic acid [preparation 44] (40 mg, 121 μmol) was dissolved in DMF (3 mL). DIPEA (47 mg, 363 μmol, 63 μL) was added followed by HATU (46 mg, 121 μmol). 3-amino-1-cyclopropyl-pyridin-2-one (22 mg, 121 μmol, HCl salt) was then added and stirred at room temperature overnight. The resulting was concentrated under high vacuum and purified by reverse phase HPLC conditions; Waters XSelect CSH Prep C18 5 um OBD 19×100 mm gradient 5 to 40% ACN-water with Ammonium formate as a modifier to afford (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide. LCMS ES+463.5 (M+1). ¹H NMR (500 MHz, DMSO-d6) δ 10.68 (s, 1H), 9.38 (br s, 1H), 8.44 (dd, J=1.53, 7.32 Hz, 1H), 8.08 (br s, 1H), 7.27-7.43 (m, 2H), 6.32 (t, J=7.17 Hz, 1H), 4.91-5.09 (m, 1H), 3.93 (s, 2H), 3.49-3.54 (m, 1H), 2.04-2.16 (m, 3H), 1.79-1.89 (m, 3H), 1.53 (d, J=5.95 Hz, 3H), 1.45 (s, 3H), 1.02-1.09 (m, 2H), 0.98 (t, J=7.40 Hz, 3H), 0.87-0.95 (m, 2H).

### Example 20: 7-(sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide

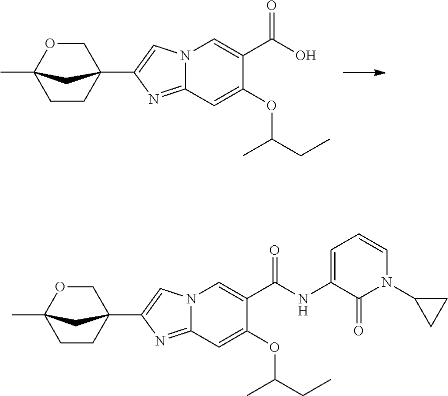

To the mixture of 2-[(1S,4R)-1-methyl-2-oxabicyclo[2.2.1]heptan-4-yl]-7-sec-butoxy-imidazo[1,2-a]pyridine-6-carboxylic acid [Preparation 25] (27.3 mg, 79.1 μmol, 2NaCl), 3-amino-1-cyclopropyl-pyridin-2-one HCl salt (13.6 mg, 90.6 μmol), HATU (33.2 mg, 87.0 mol) in DMF (0.8 mL) was added Hunig's base (31.0 mg, 240 μmol, 40 μL). The mixture was stirred at rt over weekend. The reaction mixture was partitioned between EtOAc and water. The aqueous layer was extracted with EtOAc. The combined organic phases were concentrated and purified by normal phase silica gel column (12 g, EtOAc 100%-EtOAc/EtOH 7/1) to get an off-white powder after lyophilization (28.5 mg, 75% yield). LCMS (ESI) m/z 476.9 (M+H)⁺. 1H NMR (METHANOL-d4, 500 MHz) δ 9.13 (s, 1H), 8.58 (dd, 1H, J=1.8, 7.3 Hz), 7.68 (s, 1H), 7.33 (dd, 1H, J=1.5, 7.0 Hz), 6.98 (s, 1H), 6.36 (t, 1H, J=7.3 Hz), 4.77 (br d, 1H, J=6.1 Hz), 4.05 (dd, 1H, J=3.4, 6.4 Hz), 3.92 (d, 1H, J=6.1 Hz), 3.4-3.5 (m, 1H), 1.7-2.3 (m, 8H), 1.59 (d, 3H, J=6.1 Hz), 1.46 (s, 3H), 1.1-1.2 (m, 2H), 1.06 (t, 3H, J=7.6 Hz), 0.9-1.0 (m, 2H).

### Examples 21 and 22: 7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide and 7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide

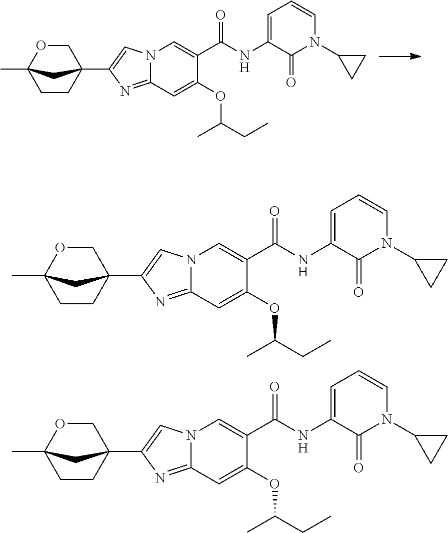

7-(sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide (25.0 mg, 52.5 mol) was purified by SFC CHIRALPAK IB 30×250 mm, 5 um Method: 30% MeOH w/ No Modifier in CO₂ (flow rate: 100 mL/min, ABPR 120 bar, MBPR 40 psi, column temp 40° C.) to provide Peak 1: 7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide, Example 21, stereochemistry arbitrarily assigned (7.6 mg, 30% yield); LCMS (ESI) m/z 476.9 (M+H)⁺. 1H NMR (METHANOL-d4, 400 MHz) δ 9.14 (s, 1H), 8.59 (dd, 1H, J=1.8, 7.5 Hz), 7.69 (s, 1H), 7.35 (dd, 1H, J=1.8, 7.0 Hz), 7.00 (s, 1H), 6.38 (t, 1H, J=7.2 Hz), 4.7-4.8 (m, 1H), 4.07 (dd, 1H, J=3.3, 6.5 Hz), 3.94 (d, 1H, J=6.5 Hz), 3.4-3.5 (m, 1H), 1.8-2.3 (m, 8H), 1.61 (d, 3H, J=6.0 Hz), 1.48 (s, 3H), 1.2-1.2 (m, 2H), 1.07 (t, 3H, J=7.4 Hz), 0.9-1.0 (m, 2H) and Peak 2: 7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide, Example 22, stereochemistry arbitrarily assigned (7.4 mg. 30% yield). LCMS (ESI) m/z 476.9 (M+H)⁺; 1H NMR (METHANOL-d4, 400 MHz) δ 9.15 (s, 1H), 8.59 (dd, 1H, J=1.8, 7.5 Hz), 7.69 (s, 1H), 7.35 (dd, 1H, J=1.8, 7.0 Hz), 7.00 (s, 1H), 6.38 (t, 1H, J=7.2 Hz), 4.7-4.8 (m, 1H), 4.07 (dd, 1H, J=3.5, 6.5 Hz), 3.94 (d, 1H, J=6.5 Hz), 3.4-3.5 (m, 1H), 1.7-2.4 (m, 8H), 1.61 (d, 3H, J=6.0 Hz), 1.48 (s, 3H), 1.1-1.3 (m, 2H), 1.07 (t, 3H, J=7.5 Hz), 0.9-1.0 (m, 2H)

### Example 23: 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide

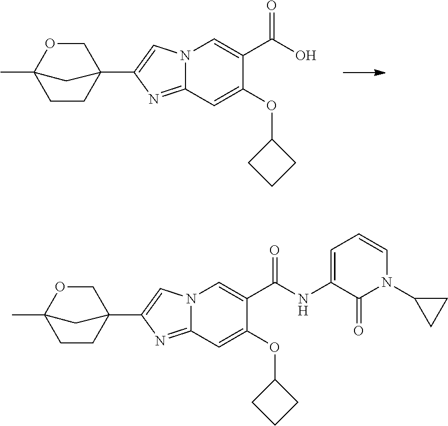 [SMILES: CC12CCC(c3cn4cc(C(=O)O)c(OC5CCC5)cc4n3)(CO1)C2 ; CC12CCC(c3cn4cc(C(=O)Nc5cccn(C6CC6)c5=O)c(OC5CCC5)cc4n3)(CO1)C2]

To a the mixture of 7-(cyclobutoxy)-2-(1-methyl-2-oxabicyclo[2.2.1]heptan-4-yl)imidazo[1,2-a]pyridine-6-carboxylic acid [Preparation 32] (46.0 mg, 105 μmol, 78% pure), 3-amino-1-cyclopropyl-pyridin-2-one (21.0 mg, 139 μmol), HATU (48.0 mg, 126 mol) in DMF (0.8 mL) was added Hunig's base (55 μL, 314 μmol). The mixture was stirred at rt for 3 days (over weekend). The reaction mixture partitioned between EtOAc and water. The aqueous layer was extracted with EtOAc. The combined organic phases were concentrated and purified by normal phase silica gel column (12 g, EtOAc 100%-EtOAc/EtOH 7/1) to get an off-white powder after lyophilization (36 mg, 72% yield). LCMS (ESI) m/z 474.9 (M+H)⁺. 1H NMR (METHANOL-d4, 500 MHz) δ 9.12 (s, 1H), 8.58 (dd, 1H, J=1.8, 7.3 Hz), 7.68 (s, 1H), 7.34 (dd, 1H, J=1.8, 6.7 Hz), 6.80 (s, 1H), 6.37 (t, 1H, J=7.0 Hz), 5.04 (t, 1H, J=7.0 Hz), 4.04 (dd, 1H, J=3.1, 6.7 Hz), 3.92 (d, 1H, J=6.7 Hz), 3.4-3.5 (m, 1H), 2.6-2.7 (m, 4H), 1.8-2.2 (m, 8H), 1.4-1.5 (m, 3H), 1.1-1.2 (m, 2H), 0.97 (br dd, 2H, J=2.1, 3.4 Hz).

### Example 24: 7-isopropoxy-2-(1-methyl-2-oxabicyclo[2.1.1]hexan-4-yl)-N-(1-(1-methylcyclopropyl)-2-oxo-1,2-dihydropyridin-3-yl)imidazo[1,2-a]pyrimidine-6-carboxamide

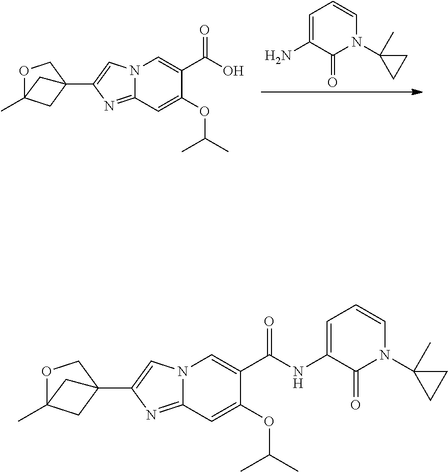 [SMILES: CC(C)Oc1cc2nc(C34COC(C)(C3)C4)cn2cc1C(=O)O ; CC1(n2cccc(N)c2=O)CC1 ; CC(C)Oc1cc2nc(C34COC(C)(C3)C4)cn2cc1C(=O)Nc1cccn(C2(C)CC2)c1=O]

To a solution of 3-amino-1-(1-methylcyclopropyl)pyridin-2(1H)-one hydrochloride [preparation 47] (35 mg, 174 μmol) and 7-isopropoxy-2-(1-methyl-2-oxabicyclo[2.1.1]hexan-4-yl)imidazo[1,2-a]pyrimidine-6-carboxylic acid [preparation 8] (55.3 mg, 174 μmol) in pyridine (1 mL) was added T3P® (1 mL, 50% w/w in EtOAc). The mixture was stirred at 20° C. for 1 h. The reaction mixture was diluted with saturated aqueous NaHCO₃ (30 mL) and extracted with EtOAc (3×20 mL). The combined organic layers were washed with brine (30 mL), dried (Na₂SO₄) and filtered. The filtrate was concentrated and the residue was purified by prep-HPLC (Column: Welch Xtimate C18 150×25 mm×5 μm; Condition: water (10 mm NH₄HCO₃)-ACN; Begin B: 42; End B: 72; Gradient Time (min): 10; 100% B Hold Time (min): 2; Flow Rate (mL/min): 25) to give 7-isopropoxy-2-(1-methyl-2-oxabicyclo[2.1.1]hexan-4-yl)-N-(1-(1-methylcyclopropyl)-2-oxo-1,2-dihydropyridin-3-yl)imidazo[1,2-a]pyrimidine-6-carboxamide (46 mg, 57% yield) as a white solid. LCMS (ESI) m/z 464.1 (M+H)⁺. ¹HNMR (500 MHz, CHLOROFORM-d) δ ppm=10.75 (s, 1H), 9.14 (s, 1H), 8.44 (d, J=7.0 Hz, 1H), 7.24 (s, 1H), 7.17 (d, J=7.0 Hz, 1H), 6.21 (t, J=7.0 Hz, 1H), 5.90-5.80 (m, 1H), 4.08 (s, 2H), 2.20-2.10 (m, 2H), 2.00-1.90 (m, 2H), 1.64 (d, J=6.5 Hz, 6H), 1.55 (s, 3H), 1.53 (s, 3H), 1.10-1.00 (m, 2H), 0.90-0.80 (m, 2H).

### Examples 25 and 26: 7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide and 7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide

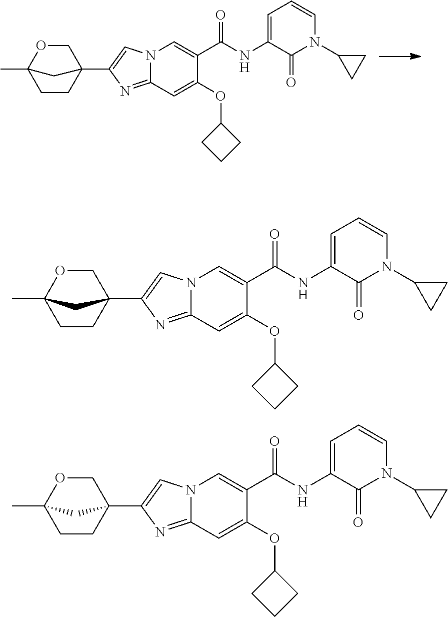

7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide [Example 23] (31 mg, 65 μmol) was purified by SFC CHIRALPAK IB 30×250 mm, 5 um Method: 45% MeOH w/ No Modifier in CO₂ (flow rate: 100 mL/min, ABPR 120 bar, MBPR 40 psi, column temp 40 deg C.) to provide Peak 1: 7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide, Example 25, stereochemistry arbitrarily assigned (10.2 mg, 33% yield); LCMS (ESI) m/z 474.9 (M+H)⁺; 1H NMR (METHANOL-d4, 400 MHz) δ 9.14 (s, 1H), 8.60 (td, 1H, J=1.6, 7.2 Hz), 7.69 (s, 1H), 7.36 (dd, 1H, J=1.3, 7.0 Hz), 6.82 (s, 1H), 6.3-6.4 (m, 1H), 5.0-5.1 (m, 1H), 4.06 (dd, 1H, J=3.5, 6.5 Hz), 3.93 (d, 1H, J=6.5 Hz), 3.4-3.6 (m, 1H), 2.6-2.8 (m, 4H), 1.8-2.3 (m, 8H), 1.48 (s, 3H), 1.1-1.2 (m, 2H), 0.9-1.0 (m, 2H) and Peak 2: 7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide, Example 26, stereochemistry arbitrarily assigned (10.5 mg. 34% yield). LCMS (ESI) m/z 474.9 (M+H)⁺; 1H NMR (METHANOL-d4, 400 MHz) δ 9.14 (s, 1H), 8.60 (dd, 1H, J=1.8, 7.5 Hz), 7.69 (s, 1H), 7.36 (dd, 1H, J=1.8, 7.0 Hz), 6.82 (s, 1H), 6.39 (t, 1H, J=7.2 Hz), 5.06 (quin, 1H, J=7.0 Hz), 4.06 (dd, 1H, J=3.4, 6.4 Hz), 3.94 (d, 1H, J=6.5 Hz), 3.4-3.5 (m, 1H), 2.6-2.7 (m, 4H), 1.8-2.2 (m, 8H), 1.48 (s, 3H), 1.1-1.3 (m, 2H), 0.9-1.0 (m, 2H).

### Example 27: 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide

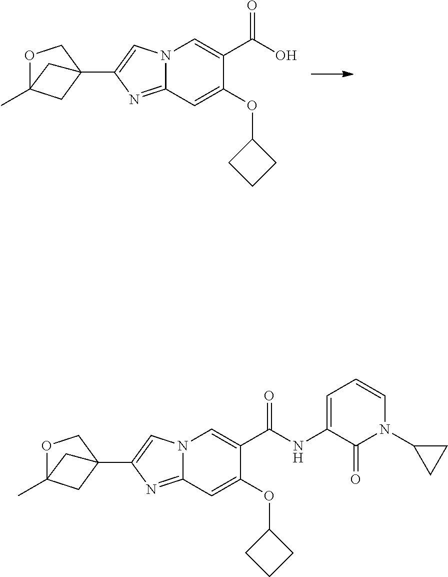 [SMILES: CC12CC(c3cn4cc(C(=O)O)c(OC5CCC5)cc4n3)(CO1)C2 ; CC12CC(c3cn4cc(C(=O)Nc5cccn(C6CC6)c5=O)c(OC5CCC5)cc4n3)(CO1)C2]

To the mixture of 7-(cyclobutoxy)-2-(1-methyl-2-oxabicyclo[2.1.1]hexan-4-yl)imidazo[1,2-a]pyridine-6-carboxylic acid [preparation 33] (31.0 mg, 67.8 μmol), 3-amino-1-cyclopropyl-pyridin-2-one HCl salt (15.2 mg, 81.3 μmol), HATU (28.4 mg, 74.6 μmol) in DMF (0.8 mL) was added Hunig's base (47 μL, 271 μmol). The mixture was stirred at rt overnight. The reaction mixture partitioned between EtOAc and water. The aqueous layer was extracted with EtOAc. The combined organic phases were concentrated and purified by normal phase silica gel column (12 g, eluting with EtOAc 100%-EtOAc/EtOH 7/1) to get an off-white powder after lyophilization (25 mg, 80% yield). LCMS (ESI) m/z 461.0 (M+H)⁺. 1H NMR (METHANOL-d4, 500 MHz) δ 9.16 (s, 1H), 8.61 (dd, 1H, J=1.5, 7.6 Hz), 7.73 (s, 1H), 7.36 (dd, 1H, J=1.8, 6.7 Hz), 6.84 (s, 1H), 6.40 (t, 1H, J=7.0 Hz), 5.07 (quin, 1H, J=7.0 Hz), 4.61 (s, 1H), 4.03 (s, 2H), 3.4-3.5 (m, 1H), 2.6-2.8 (m, 4H), 2.13 (dd, 2H, J=1.5, 4.6 Hz), 2.05 (br d, 1H, J=2.4 Hz), 1.8-1.9 (m, 3H), 1.52 (s, 3H), 1.1-1.2 (m, 2H), 0.9-1.0 (m, 2H).

### Example 28: (S)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide

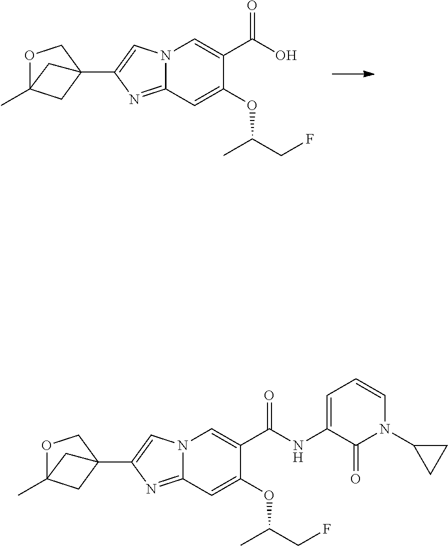 [SMILES: C[C@@H](CF)Oc1cc2nc(C34COC(C)(C3)C4)cn2cc1C(=O)O ; C[C@@H](CF)Oc1cc2nc(C34COC(C)(C3)C4)cn2cc1C(=O)Nc1cccn(C2CC2)c1=O]

To a mixture of 7-[(1S)-2-fluoro-1-methyl-ethoxy]-2-(1-methyl-2-oxabicyclo[2.1.1]hexan-4-yl)imidazo[1,2-a]pyridine-6-carboxylic acid [preparation 36] (21.0 mg, 46.5 μmol, 2NaCl), 3-amino-1-cyclopropyl-pyridin-2-one (10.4 mg, 55.8 μmol, HCl), HATU (19.5 mg, 51.2 μmol) in DMF (0.8 mL) was added Hunig's base (32 μL, 186 μmol). The mixture was stirred at rt overnight. It was partitioned between EtOAc and water. The aqueous layer was extracted with EtOAc. The combined organic phases were concentrated and purified by normal phase silica gel column (12 g, EtOAc 100% to EtOAc/EtOH 7/1) to get a white powder after lyophilization (15 mg, 69%). LCMS (ESI) m/z 467.0 (M+H)⁺. 1H NMR (METHANOL-d4, 500 MHz) δ 9.19 (s, 1H), 8.59 (dd, 1H, J=1.5, 7.6 Hz), 7.77 (s, 1H), 7.36 (dd, 1H, J=1.8, 6.7 Hz), 7.14 (s, 1H), 6.39 (t, 1H, J=7.3 Hz), 5.2-5.3 (m, 1H), 5.0-5.1 (m, 1H), 4.7-4.8 (m, 1H), 4.04 (s, 2H), 3.4-3.6 (m, 1H), 2.14 (dd, 2H, J=1.5, 4.6 Hz), 1.91 (dd, 2H, J=1.5, 4.6 Hz), 1.58 (dd, 3H, J=1.5, 6.4 Hz), 1.52 (s, 3H), 1.1-1.2 (m, 2H), 0.9-1.0 (m, 2H).

### Example 29: (R)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide

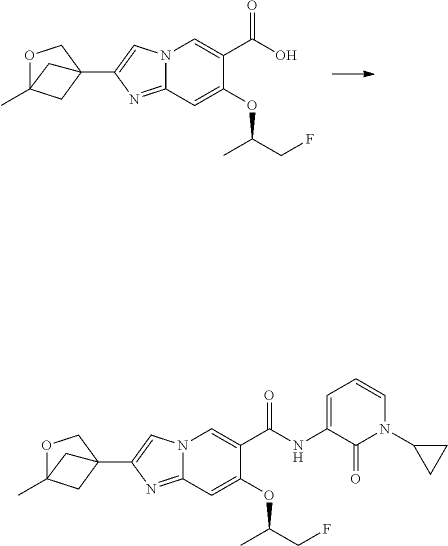 [SMILES: C[C@H](CF)Oc1cc2nc(C34COC(C)(C3)C4)cn2cc1C(=O)O ; C[C@H](CF)Oc1cc2nc(C34COC(C)(C3)C4)cn2cc1C(=O)Nc1cccn(C2CC2)c1=O]

To a mixture of 7-[(1R)-2-fluoro-1-methyl-ethoxy]-2-(1-methyl-2-oxabicyclo[2.1.1]hexan-4-yl)imidazo[1,2-a]pyridine-6-carboxylic acid [preparation 37] (19.0 mg, 56.8 μmol, 2NaCl), 3-amino-1-cyclopropyl-pyridin-2-one (12.7 mg, 68.2 μmol, HCl), HATU (23.8 mg, 62.5 μmol) in DMF (0.8 mL) was added Hunig's base (39 μL, 227 μmol). The mixture was stirred at rt overnight. The reaction mixture was partitioned between EtOAc and water. The aqueous layer was extracted with EtOAc. The combined organic phases were concentrated and purified by normal phase silica gel column (12 g, EtOAc100% to EtOAc/EtOH 7/1) to get an off-white powder after lyophilization (16 mg, 82% yield) LCMS (ESI) m/z 467.0 (M+H)⁺. 1H NMR (METHANOL-d4, 500 MHz) δ 9.19 (s, 1H), 8.59 (dd, 1H, J=1.8, 7.3 Hz), 7.75 (s, 1H), 7.36 (dd, 1H, J=1.8, 7.3 Hz), 7.12 (s, 1H), 6.3-6.5 (m, 1H), 5.2-5.3 (m, 1H), 5.0-5.1 (m, 1H), 4.7-4.8 (m, 1H), 4.03 (s, 2H), 3.4-3.5 (m, 1H), 2.13 (dd, 2H, J=1.5, 4.6 Hz), 1.91 (dd, 2H, J=1.8, 4.9 Hz), 1.58 (dd, 3H, J=1.5, 6.4 Hz), 1.52 (s, 3H), 1.1-1.3 (m, 2H), 0.9-1.0 (m, 2H).

### Example 30: N-(1-cyclopropyl-2-oxo-1,2-dihydropyridin-3-yl)-7-(((S)-1-fluoropropan-2-yl)oxy)-2-(1-methyl-2-oxabicyclo[2.2.1]heptan-4-yl)imidazo[1,2-a]pyridine-6-carboxamide

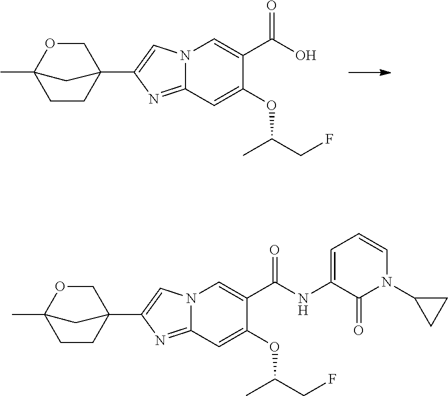 [SMILES: C[C@@H](CF)Oc1cc2nc(C34CCC(C)(C3)OC4)cn2cc1C(=O)O ; C[C@@H](CF)Oc1cc2nc(C34CCC(C)(C3)OC4)cn2cc1C(=O)Nc1cccn(C2CC2)c1=O]

To the mixture of 7-[(1S)-2-fluoro-1-methyl-ethoxy]-2-[(1S,4R)-1-methyl-2-oxabicyclo[2.2.1]heptan-4-yl]imidazo[1,2-a]pyridine-6-carboxylic acid [preparation 38](7.11 mg, 20.4 μmol, 2NaCl), 3-amino-1-cyclopropyl-pyridin-2-one (5.0 mg, 33 mol), HATU (11.0 mg, 28.8 μmol) in DMF (0.8 mL) was added Hunig's base (11 μL, 61.3 mol). The mixture was stirred at rt overnight. The reaction mixture partitioned between EtOAc and water. The aqueous layer was extracted with EtOAc. The combined organic phases were concentrated and purified by normal phase silica gel column (12 g, EtOAc/EtOH 7/1) to get N-(1-cyclopropyl-2-oxo-1,2-dihydropyridin-3-yl)-7-(((S)-1-fluoropropan-2-yl)oxy)-2-(1-methyl-2-oxabicyclo[2.2.1]heptan-4-yl)imidazo[1,2-a]pyridine-6-carboxamide as a mixture of diastereomers and a yellow powder after lyophilization (6 mg, 61% yield). LCMS (ESI) m/z 480.9 (M+H)⁺. 1H NMR (METHANOL-d4, 400 MHz) δ 1H NMR (METHANOL-d4, 500 MHz) δ 9.1-9.3 (m, 1H), 8.47 (dd, 1H, J=1.8, 7.3 Hz), 7.79 (s, 1H), 7.26 (dd, 1H, J=1.5, 7.0 Hz), 7.20 (s, 1H), 6.3-6.3 (m, 1H), 5.20 (dtd, 1H, J=2.4, 6.9, 14.2 Hz), 4.9-5.0 (m, 1H), 4.6-4.7 (m, 1H), 3.93 (dd, 1H, J=3.1, 6.7 Hz), 3.85 (d, 1H, J=6.7 Hz), 3.3-3.4 (m, 1H), 2.0-2.1 (m, 2H), 1.92 (s, 2H), 1.7-1.9 (m, 2H), 1.49 (dd, 3H, J=1.2, 6.7 Hz), 1.38 (s, 3H), 1.0-1.1 (m, 2H), 0.8-0.9 (m, 2H).

### Example 31: 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide

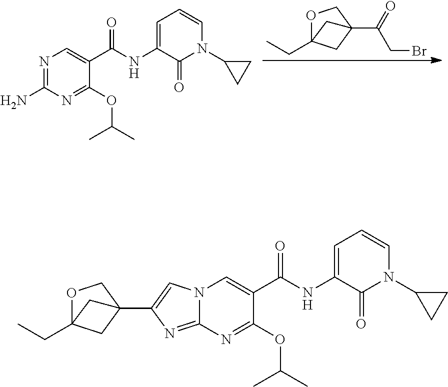

A mixture of 2-bromo-1-(1-ethyl-2-oxabicyclo[2.1.1]hexan-4-yl)ethanone (12.0 mg, 51.5 mol), 2-amino-N-(1-cyclopropyl-2-oxo-3-pyridyl)-4-isopropoxy-pyrimidine-5-carboxamide [preparation 39] (11.3 mg, 34.3 μmol) and sodium bicarbonate (5.7 μL, 146 mol) in MeCN (0.4 mL) and toluene (0.6 mL) was heated to 90° C. in a microwave tube and stirred at 90° C. overnight. The mixture was partitioned between EtOAc and water. The aqueous layer was extracted with EtOAc. The combined organic phases were concentrated and purified by normal phase silica gel column (12 g, EtOAc 100%) to provide 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide as an off-white power (4.1 mg, 26% yield). LCMS (ESI) m/z 464.3 (M+H)⁺. 1H NMR (METHANOL-d4, 400 MHz) δ 9.3-9.4 (m, 1H), 8.56 (dd, 1H, J=1.8, 7.3 Hz), 7.62 (s, 1H), 7.34 (dd, 1H, J=1.8, 7.0 Hz), 6.36 (t, 1H, J=7.2 Hz), 5.78 (quin, 1H, J=6.2 Hz), 4.00 (s, 2H), 3.4-3.5 (m, 1H), 2.1-2.1 (m, 2H), 1.8-1.9 (m, 4H), 1.64 (d, 6H, J=6.3 Hz), 1.1-1.2 (m, 2H), 1.02 (t, 3H, J=7.5 Hz), 0.9-1.0 (m, 2H).

### Example 32: 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide

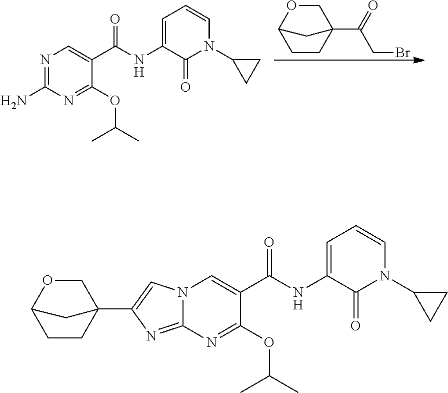

To a mixture of 1-(2-oxabicyclo[2.2.1]heptan-4-yl)-2-bromoethan-1-one [preparation 14](13.1 mg, 59.7 μmol), 2-amino-N-(1-cyclopropyl-2-oxo-3-pyridyl)-4-isopropoxy-pyrimidine-5-carboxamide [preparation 39] (12.9 mg, 39.2 μmol) and sodium bicarbonate (9.87 mg, 117 μmol, 4.6 μL) was added MeCN (0.4 mL) and Toluene (0.6 mL). The mixture was heated to 90° C. in a sealed μW tube and stirred at 90° C. overnight. The mixture was partitioned between EtOAc and water. The aqueous layer was extracted with EtOAc (×2). The combined organic phases were concentrated in vacuo. The residue was purified by normal phase silica gel column (12 g, EtOAc 100% to EtOAc/EtOH 7/1) to get racemic 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide as an off-white solid (8.0 mg, 45% yield). LCMS (ESI) m/z 450.2 (M+H)⁺. 1H NMR (METHANOL-d4, 400 MHz) δ 9.39 (s, 1H), 8.5-8.7 (m, 1H), 7.61 (s, 1H), 7.35 (dd, 1H, J=1.8, 7.0 Hz), 6.37 (t, 1H, J=7.2 Hz), 5.7-5.9 (m, 1H), 4.49 (s, 1H), 3.94 (dd, 1H, J=3.4, 6.4 Hz), 3.85 (d, 1H, J=6.3 Hz), 3.4-3.5 (m, 1H), 1.8-2.2 (m, 6H), 1.64 (d, 6H, J=6.3 Hz), 1.1-1.2 (m, 2H), 0.9-1.0 (m, 2H).

### Example 33: 7-cyclobutoxy-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide

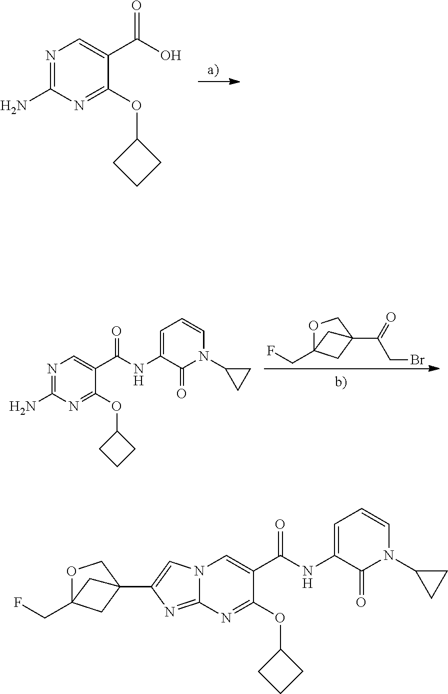

Step a: To the mixture of 2-amino-4-cyclobutoxypyrimidine-5-carboxylic acid [preparation 66] (104.6 mg, 0.5 mmol), 3-amino-1-cyclopropyl-pyridin-2-one (97.4 mg, 522 μmol, HCl), HATU (200.1 mg, 525.0 μmol) in DMF (2 mL) was added Henig's base (348 μL, 2.00 mmol). The mixture was stirred at rt for overnight (2 days). The reaction mixture partitioned between EtOAc and water. The solid was filtered, washed with water, followed by MeCN to get 2-amino-4-cyclobutoxy-N-(1-cyclopropyl-2-oxo-1,2-dihydropyridin-3-yl)pyrimidine-5-carboxamide as an off-white solid (88 mg, 52% yield)

Step b: To the mixture of 2-bromo-1-[1-(fluoromethyl)-2-oxabicyclo[2.1.1]hexan-4-yl]ethanone (12.8 mg, 54.1 μmol), 2-amino-4-(cyclobutoxy)-N-(1-cyclopropyl-2-oxo-3-pyridyl)pyrimidine-5-carboxamide (14.2 mg, 41.6 μmol) and sodium bicarbonate (10.5 mg, 125 μmol, 4.8 μL) in a microwave tube was added MeCN (0.4 mL) and toluene (0.6 mL). The tube was capped, and the mixture was heated at 90° C. overnight. It was partitioned between EtOAc and water. The aqueous layer was extracted with EtOAc (×2). The combined organic phases were concentrated and purified by normal phase silica gel column (12 g, EtOAc in heptane 50-100%) to get 7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide as an off-white power (6.0 mg, 30% yield). LCMS (ESI) m/z 480.2 (M+H)⁺. 1H NMR (METHANOL-d4, 400 MHz) δ 9.42 (s, 1H), 8.60 (dd, 1H, J=1.6, 7.4 Hz), 7.68 (s, 1H), 7.38 (dd, 1H, J=1.5, 7.0 Hz), 6.40 (t, 1H, J=7.2 Hz), 5.5-5.7 (m, 1H), 4.75 (s, 1H), 4.63 (s, 1H), 4.08 (s, 2H), 3.50 (dd, 1H, J=2.3, 4.0 Hz), 2.6-2.7 (m, 4H), 2.26 (dd, 2H, J=1.3, 4.8 Hz), 1.8-2.1 (m, 4H), 1.2-1.2 (m, 2H), 1.0-1.0 (m, 2H).

### Example 34: 2-(2-oxabicyclo[2.1.1]hexan-4-yl)-N-(1-cyclopropyl-2-oxo-1,2-dihydropyridin-3-yl)-7-isopropoxyimidazo[1,2-a]pyridine-6-carboxamide

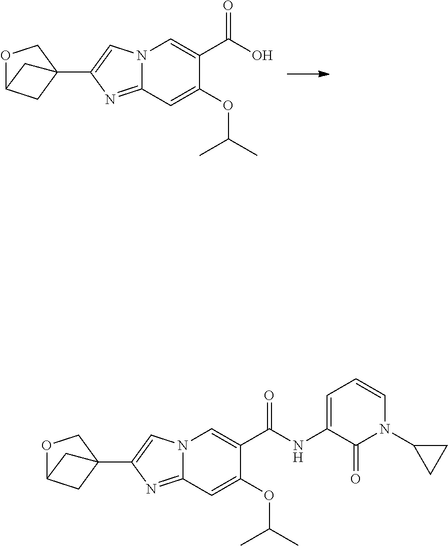 [SMILES: CC(C)Oc1cc2nc(C34COC(C3)C4)cn2cc1C(=O)O ; CC(C)Oc1cc2nc(C34COC(C3)C4)cn2cc1C(=O)Nc1cccn(C2CC2)c1=O]

To the mixture of 2-(2-oxabicyclo[2.1.1]hexan-4-yl)-7-isopropoxyimidazo[1,2-a]pyridine-6-carboxylic acid [preparation 68] (22.6 mg, 74.75 μmol), 3-amino-1-cyclopropyl-pyridin-2-one (16.74 mg, 89.70 μmol, HCl), HATU (31.35 mg, 82.23 μmol) in DMF (0.8 mL) was added Hunigs base (52.08 uL, 299.02 μmol). The mixture was stirred at rt for overnight. It was partitioned between EtOAc/water. The aqueous layer was extracted with EtOAc. The combined organic phases were concentrated and purified by normal phase column (12 g, EtOAc 100% to EtOAc/EtOH 7/1) to get an off-white powder after lyophilization (8 mg, yield 24%). LCMS (ESI) m/z 435.0 (M+H)⁺. 1H NMR (METHANOL-d4, 500 MHz) δ 9.14 (s, 1H), 8.57 (dd, 1H, J=1.5, 7.6 Hz), 7.73 (s, 1H), 7.33 (dd, 1H, J=1.8, 7.3 Hz), 7.01 (s, 1H), 6.37 (t, 1H, J=7.3 Hz), 5.02 (td, 1H, J=6.1, 12.2 Hz), 4.65 (t, 1H, J=1.2 Hz), 3.96 (s, 2H), 3.4-3.5 (m, 1H), 2.2-2.4 (m, 2H), 1.88 (dd, 2H, J=1.8, 4.9 Hz), 1.63 (d, 6H, J=6.1 Hz), 1.1-1.3 (m, 2H), 0.96 (td, 2H, J=1.3, 4.1 Hz).

### Example 35: N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide

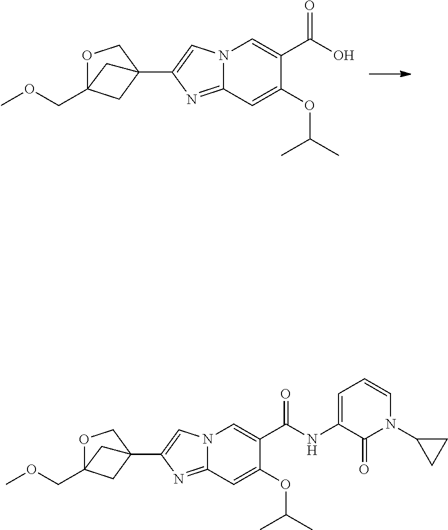

To the mixture of 7-isopropoxy-2-[1-(methoxymethyl)-2-oxabicyclo[2.1.1]hexan-4-yl]imidazo[1,2-a]pyrimidine-6-carboxylic acid [preparation 29] 32 mg, 61.22 umol, 1.5NaCl), 3-amino-1-cyclopropyl-pyridin-2-one (14 mg, 75.01 umol, HCl), HATU (25.67 mg, 67.34 umol) in DMF (1 mL) was added Hunigs base (42.65 uL 244.89 umol). The mixture was stirred at rt for 2 days. It was concentrated and partitioned between EtOAc/water. The aqueous layer was extracted with EtOAc. The combined organic phases were concentrated and purified by normal phase column (12 g, EtOAc 100% to EtOAc/EtOH 7/1) to collect N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide as an off-white powder (24 mg, yield 82%). LCMS m/z=480.2 [M+H]⁺. 1H NMR (METHANOL-d4, 400 MHz) δ 9.37 (s, 1H), 8.55 (dd, 1H, J=1.8, 7.3 Hz), 7.63 (s, 1H), 7.33 (dd, 1H, J=1.8, 7.0 Hz), 6.35 (t, 1H, J=7.3 Hz), 5.77 (td, 1H, J=6.2, 12.5 Hz), 4.03 (s, 2H), 3.71 (s, 2H), 3.4-3.5 (m, 4H), 2.18 (dd, 2H, J=1.6, 4.6 Hz), 1.92 (dd, 2H, J=1.8, 4.5 Hz), 1.64 (d, 6H, J=6.3 Hz), 1.1-1.2 (m, 2H), 0.9-1.0 (m, 2H).

### Example 36: 2-(2-oxabicyclo[2.1.1]hexan-4-yl)-7-cyclobutoxy-N-(1-cyclopropyl-2-oxo-1,2-dihydropyridin-3-yl)imidazo[1,2-a]pyrimidine-6-carboxamide

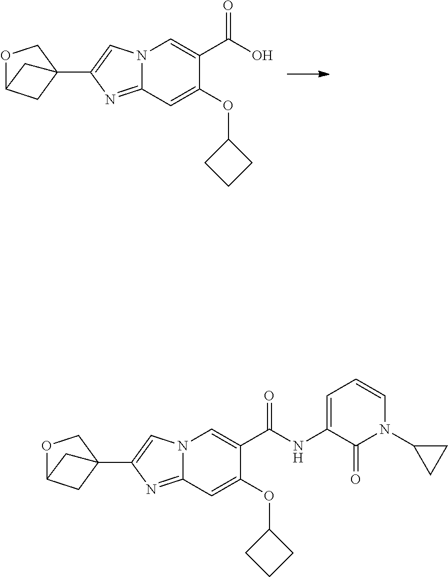 [SMILES: O=C(O)c1cn2cc(C34COC(C3)C4)nc2cc1OC1CCC1 ; O=C(Nc1cccn(C2CC2)c1=O)c1cn2cc(C34COC(C3)C4)nc2cc1OC1CCC1]

2-(2-oxabicyclo[2.1.1]hexan-4-yl)-7-cyclobutoxy-N-(1-cyclopropyl-2-oxo-1,2-dihydropyridin-3-yl)imidazo[1,2-a]pyrimidine-6-carboxamide was obtained from 2-(2-oxabicyclo[2.1.1]hexan-4-yl)-7-cyclobutoxyimidazo[1,2-a]pyrimidine-6-carboxylic acid [preparation 30] as pale yellow solid (13.5 mg, yield 48%), following a similar procedure to that described in Example 20. LCMS m/z=447.9 [M+H]⁺. 1H NMR (METHANOL-d4, 400 MHz) δ 9.42 (s, 1H), 8.60 (dd, 1H, J=1.8, 7.5 Hz), 7.66 (s, 1H), 7.37 (dd, 1H, J=1.8, 7.0 Hz), 6.39 (t, 1H, J=7.3 Hz), 5.5-5.7 (m, 1H), 4.67 (t, 1H, J=1.0 Hz), 3.97 (s, 2H), 3.4-3.6 (m, 1H), 2.6-2.8 (m, 4H), 2.2-2.3 (m, 2H), 2.0-2.1 (m, 2H), 1.8-1.9 (m, 3H), 1.1-1.3 (m, 2H), 0.9-1.0 (m, 2H).

### Example 37: 2-(2-oxabicyclo[2.1.1]hexan-4-yl)-7-cyclobutoxy-N-(1-cyclopropyl-2-oxo-1,2-dihydropyridin-3-yl)imidazo[1,2-a]pyridine-6-carboxamide

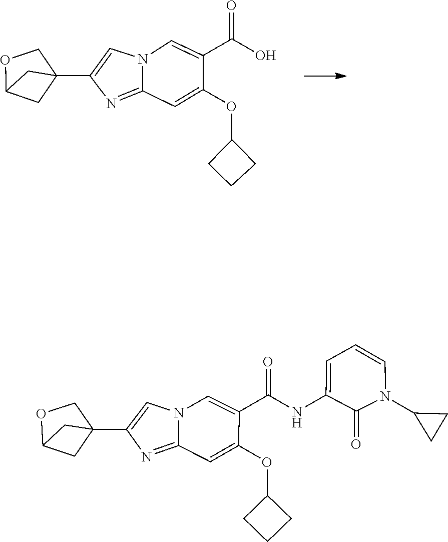 [SMILES: O=C(O)c1cn2cc(C34COC(C3)C4)nc2cc1OC1CCC1 ; O=C(Nc1cccn(C2CC2)c1=O)c1cn2cc(C34COC(C3)C4)nc2cc1OC1CCC1]

2-(2-oxabicyclo[2.1.1]hexan-4-yl)-7-cyclobutoxy-N-(1-cyclopropyl-2-oxo-1,2-dihydropyridin-3-yl)imidazo[1,2-a]pyridine-6-carboxamide was obtained from 2-(2-oxabicyclo[2.1.1]hexan-4-yl)-7-cyclobutoxyimidazo[1,2-a]pyridine-6-carboxylic acid [preparation 31] as pale yellow solid (16 mg, yield 55%), following a similar procedure to that described in Example 20. LCMS m/z=447.2 [M+H]⁺. ¹H NMR (METHANOL-d4, 400 MHz) δ 9.1-9.2 (m, 1H), 8.60 (dd, 1H, J=1.8, 7.5 Hz), 7.76 (s, 1H), 7.36 (dd, 1H, J=1.8, 7.0 Hz), 6.84 (s, 1H), 6.40 (t, 1H, J=7.2 Hz), 5.07 (t, 1H, J=7.5 Hz), 4.67 (t, 1H, J=1.0 Hz), 3.98 (s, 2H), 3.5-3.6 (m, 1H), 2.6-2.8 (m, 4H), 2.27 (d, 2H, J=4.8 Hz), 2.05 (br s, 1H), 1.8-2.0 (m, 3H), 1.1-1.3 (m, 2H), 0.9-1.0 (m, 2H).

### Example 38: 2-(2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide

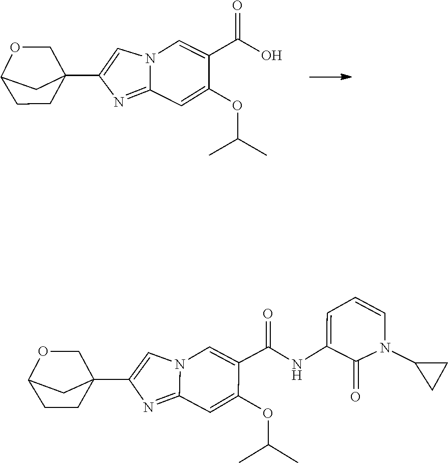 [SMILES: CC(C)Oc1cc2nc(C34CCC(C3)OC4)cn2cc1C(=O)O ; CC(C)Oc1cc2nc(C34CCC(C3)OC4)cn2cc1C(=O)Nc1cccn(C2CC2)c1=O]

2-(2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide was obtained from 2-(2-oxabicyclo[2.2.1]heptan-4-yl)-7-isopropoxyimidazo[1,2-a]pyridine-6-carboxylic acid [preparation 69] as pale yellow solid (39 mg, yield 71%), following a similar procedure to that described in Example 20. LCMS m/z=449.3 [M+H]⁺. 1H NMR (METHANOL-d4, 400 MHz) δ 9.2-9.3 (m, 1H), 8.58 (dd, 1H, J=1.5, 7.5 Hz), 7.81 (s, 1H), 7.35 (dd, 1H, J=1.6, 6.9 Hz), 7.09 (s, 1H), 6.37 (t, 1H, J=7.3 Hz), 5.07 (td, 1H, J=6.0, 12.2 Hz), 4.52 (d, 1H, J=1.3 Hz), 3.8-4.0 (m, 2H), 3.4-3.5 (m, 1H), 1.8-2.2 (m, 6H), 1.65 (d, 6H, J=6.0 Hz), 1.1-1.2 (m, 2H), 0.9-1.0 (m, 2H).

### Examples 39 and 40: 2-((1S,4R)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide and 2-((1R,4S)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide

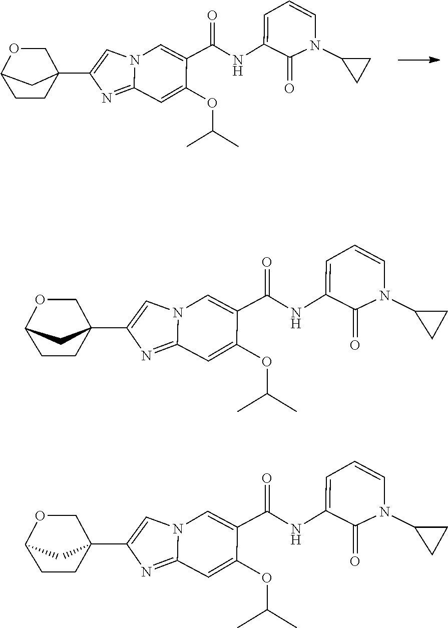 [SMILES: CC(C)Oc1cc2nc(C34CCC(C3)OC4)cn2cc1C(=O)Nc1cccn(C2CC2)c1=O ; CC(C)Oc1cc2nc([C@@]34CC[C@@H](C3)OC4)cn2cc1C(=O)Nc1cccn(C2CC2)c1=O ; CC(C)Oc1cc2nc([C@]34CC[C@H](C3)OC4)cn2cc1C(=O)Nc1cccn(C2CC2)c1=O]

2-(2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide (Example 38, 35 mg, 78.0 umol) was purified by SFC CHIRALPAK IB 30×250 mm, 5 um; Method: 40% MeOH w/ No Modifier in CO₂ (flow rate: 100 mL/min, ABPR 120 bar, MBPR 40 psi, column temp 40 deg C.) to provide Peak 1: 2-((1S,4R)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide, Example 39, stereochemistry arbitrarily assigned (11.6 mg, 33% yield). LCMS (ESI) m/z 449.3 (M+H)⁺; 1H NMR (METHANOL-d4, 400 MHz) δ 9.14 (s, 1H), 8.59 (dd, 1H, J=1.6, 7.4 Hz), 7.72 (s, 1H), 7.35 (dd, 1H, J=1.9, 6.9 Hz), 7.01 (s, 1H), 6.38 (t, 1H, J=7.2 Hz), 5.03 (td, 1H, J=6.1, 12.1 Hz), 4.52 (d, 1H, J=1.5 Hz), 3.96 (dd, 1H, J=3.3, 6.5 Hz), 3.88 (d, 1H, J=6.5 Hz), 3.4-3.5 (m, 1H), 1.8-2.2 (m, 6H), 1.65 (d, 6H, J=6.3 Hz), 1.1-1.2 (m, 2H), 0.9-1.0 (m, 2H) and Peak 2: 2-((1R,4S)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide, Example 40, stereochemistry arbitrarily assigned (11.7 mg. 33% yield). LCMS (ESI) m/z 449.3 (M+H)⁺; 1H NMR (METHANOL-d4, 400 MHz) δ 9.1-9.2 (m, 1H), 8.59 (dd, 1H, J=1.8, 7.5 Hz), 7.72 (s, 1H), 7.35 (dd, 1H, J=1.8, 7.0 Hz), 7.01 (s, 1H), 6.39 (t, 1H, J=7.2 Hz), 5.03 (td, 1H, J=6.1, 12.1 Hz), 4.52 (d, 1H, J=1.5 Hz), 3.96 (dd, 1H, J=3.5, 6.5 Hz), 3.88 (d, 1H, J=6.5 Hz), 3.4-3.5 (m, 1H), 1.9-2.2 (m, 6H), 1.65 (d, 6H, J=6.0 Hz), 1.1-1.2 (m, 2H), 0.9-1.0 (m, 2H).

### Example 41: 7-((R)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

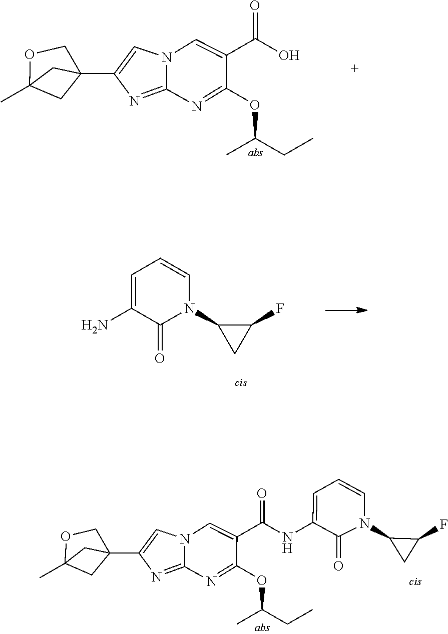

7-((R)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide was obtained as an off-white powder solid (56 mg, yield 71%), from (R)-7-(sec-butoxy)-2-(1-methyl-2-oxabicyclo[2.1.1]hexan-4-yl)imidazo[1,2-a]pyrimidine-6-carboxylic acid [preparation 27], following a similar procedure to that described in Example 20. (mix of diastereomers) LCMS m/z=482.2 [M+H]⁺. ¹H NMR (METHANOL-d4, 400 MHz) d 9.42 (s, 1H), 8.5-8.7 (m, 1H), 7.64 (s, 1H), 7.41 (d, 1H, J=7.0 Hz), 6.3-6.5 (m, 1H), 5.6-5.7 (m, 1H), 4.9-5.1 (m, 1H), 4.02 (s, 2H), 3.44 (br dd, 1H, J=2.9, 5.1 Hz), 2.1-2.3 (m, 3H), 1.8-2.0 (m, 3H), 1.62 (dd, 3H, J=1.8, 6.3 Hz), 1.5-1.6 (m, 2H), 1.52 (s, 3H), 1.0-1.1 (m, 3H).

### Example 42: 7-((S)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

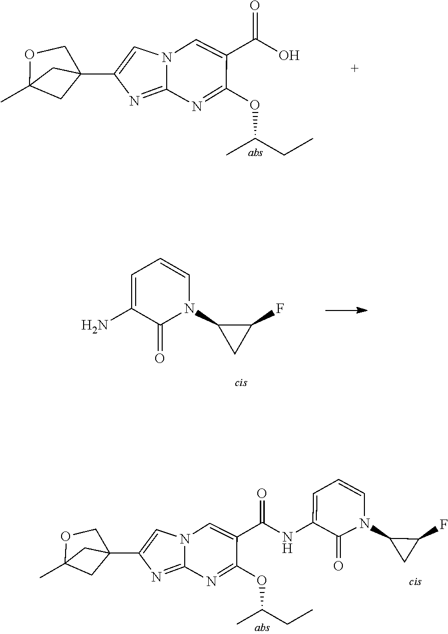

7-((S)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide was obtained as an off-white solid (59 mg, yield 76%), from (S)-7-(sec-butoxy)-2-(1-methyl-2-oxabicyclo[2.1.1]hexan-4-yl)imidazo[1,2-a]pyrimidine-6-carboxylic acid [preparation 28] following a similar procedure to that described in Example 20. (mixture of diastereomers) LCMS m/z=484.4 [M+H]⁺. ¹H NMR (METHANOL-d4, 400 MHz) δ 9.42 (s, 1H), 8.61 (td, 1H, J=2.0, 7.5 Hz), 7.64 (s, 1H), 7.41 (d, 1H, J=6.8 Hz), 6.41 (dt, 1H, J=1.4, 7.2 Hz), 5.64 (q, 1H, J=6.5 Hz), 4.9-5.1 (m, 1H), 4.02 (s, 2H), 3.4-3.5 (m, 1H), 2.1-2.3 (m, 3H), 1.8-2.0 (m, 3H), 1.62 (dd, 3H, J=1.8, 6.3 Hz), 1.5-1.6 (m, 2H), 1.52 (s, 3H), 1.06 (dt, 3H, J=2.1, 7.5 Hz).

### Example 43: N-(1-cyclopropyl-2-oxo-1,2-dihydropyridin-3-yl)-7-ethoxy-2-(1-methyl-2-oxabicyclo[2.1.1]hexan-4-yl)imidazo[1,2-a]pyrimidine-6-carboxamide

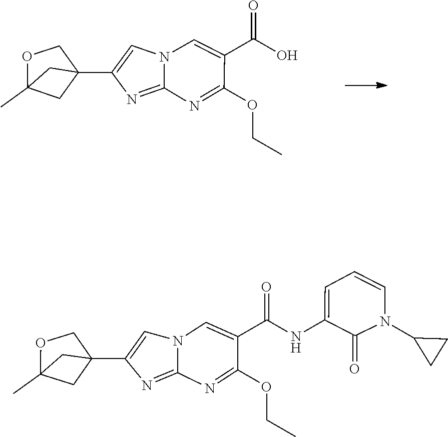 [SMILES: CCOc1nc2nc(C34COC(C)(C3)C4)cn2cc1C(=O)O ; CCOc1nc2nc(C34COC(C)(C3)C4)cn2cc1C(=O)Nc1cccn(C2CC2)c1=O]

To a mixture of 7-ethoxy-2-(1-methyl-2-oxabicyclo[2.1.1]hexan-4-yl)imidazo[1,2-a]pyrimidine-6-carboxylic acid [preparation 70] (19.5 mg, 46.41 umol, 2NaCl), 3-amino-1-cyclopropyl-pyridin-2-one (10 mg, 53.58 umol, HCl), HATU (20.6 mg, 54.03 umol) in DMF (0.8 mL) was added Hunigs base (32.33 uL, 185.63 umol). The mixture was stirred at rt for 3 days. It was partitioned between EtOAc/water. The aqueous layer was extracted with EtOAc. The combined organic phases were concentrated and purified by normal phase silica gel column (12 g, EtOAc 100% to EtOAc/EtOH 10/1) to get an off-white powder after lyophilization (15.5 mg, 77% yield). LCMS m/z=436.2 [M+H]⁺. 1H NMR (METHANOL-d4, 400 MHz) δ 9.37 (s, 1H), 8.54 (dd, 1H, J=1.8, 7.5 Hz), 7.62 (s, 1H), 7.33 (dd, 1H, J=1.8, 7.0 Hz), 6.36 (t, 1H, J=7.3 Hz), 4.79 (q, 2H, J=7.0 Hz), 4.00 (s, 2H), 3.4-3.5 (m, 1H), 2.0-2.2 (m, 2H), 1.8-2.0 (m, 2H), 1.68 (t, 3H, J=7.2 Hz), 1.50 (s, 3H), 1.1-1.2 (m, 2H), 0.9-1.0 (m, 2H).

### Example 44: N-(1-cyclopropyl-2-oxo-1,2-dihydropyridin-3-yl)-2-(1-ethyl-2-oxabicyclo[2.2.1]heptan-4-yl)-7-isopropoxyimidazo[1,2-a]pyrimidine-6-carboxamide

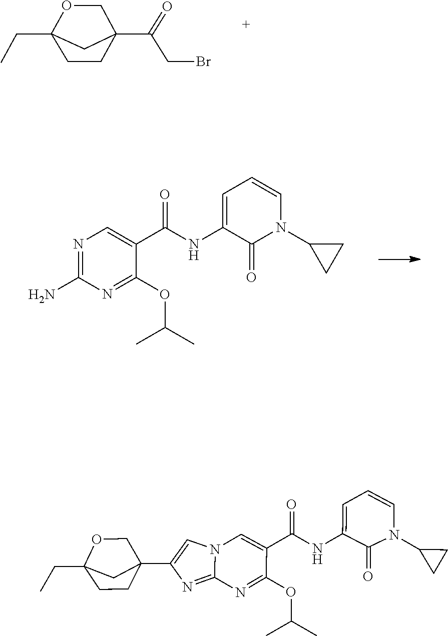 [SMILES: CCC12CCC(C(=O)CBr)(CO1)C2 ; CC(C)Oc1nc(N)ncc1C(=O)Nc1cccn(C2CC2)c1=O ; CCC12CCC(c3cn4cc(C(=O)Nc5cccn(C6CC6)c5=O)c(OC(C)C)nc4n3)(CO1)C2]

N-(1-cyclopropyl-2-oxo-1,2-dihydropyridin-3-yl)-2-(1-ethyl-2-oxabicyclo[2.2.1]heptan-4-yl)-7-isopropoxyimidazo[1,2-a]pyrimidine-6-carboxamide was obtained as an off-white powder solid (56 mg, yield 71%), from 2-bromo-1-(1-ethyl-2-oxabicyclo[2.2.1]heptan-4-yl)ethan-1-one [preparation 16] and 2-amino-N-(1-cyclopropyl-2-oxo-1,2-dihydropyridin-3-yl)-4-isopropoxypyrimidine-5-carboxamide [preparation 39] following a similar procedure to that described in Example 31. (5.6 mg, 29% yield). LCMS m/z=478.2 [M+H]⁺. 1H NMR (METHANOL-d4, 400 MHz) δ 9.2-9.3 (m, 1H), 8.45 (dd, 1H, J=1.8, 7.3 Hz), 7.48 (s, 1H), 7.23 (dd, 1H, J=1.8, 7.0 Hz), 6.25 (t, 1H, J=7.3 Hz), 5.67 (quin, 1H, J=6.3 Hz), 3.92 (dd, 1H, J=3.3, 6.3 Hz), 3.82 (d, 1H, J=6.5 Hz), 3.3-3.4 (m, 1H), 1.6-2.1 (m, 8H), 1.54 (d, 6H, J=6.3 Hz), 1.0-1.1 (m, 2H), 0.93 (t, 3H, J=7.5 Hz), 0.8-0.9 (m, 2H).

### Example 45: N-(1-cyclopropyl-2-oxo-1,2-dihydropyridin-3-yl)-2-(1-(fluoromethyl)-2-oxabicyclo[2.2.1]heptan-4-yl)-7-isopropoxyimidazo[1,2-a]pyrimidine-6-carboxamide

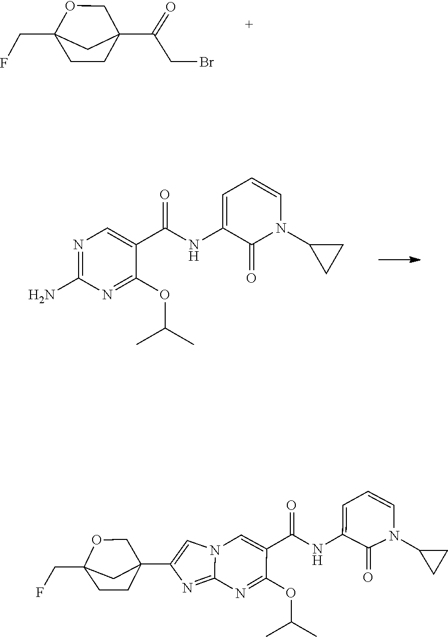 [SMILES: O=C(CBr)C12CCC(CF)(C1)OC2 ; CC(C)Oc1nc(N)ncc1C(=O)Nc1cccn(C2CC2)c1=O ; CC(C)Oc1nc2nc(C34CCC(CF)(C3)OC4)cn2cc1C(=O)Nc1cccn(C2CC2)c1=O]

N-(1-cyclopropyl-2-oxo-1,2-dihydropyridin-3-yl)-2-(1-(fluoromethyl)-2-oxabicyclo[2.2.1]heptan-4-yl)-7-isopropoxyimidazo[1,2-a]pyrimidine-6-carboxamide was obtained as an off-white powder solid (31 mg, yield 32%), from 2-bromo-1-(1-(fluoromethyl)-2-oxabicyclo[2.2.1]heptan-4-yl)ethan-1-one [preparation 17] and 2-amino-N-(1-cyclopropyl-2-oxo-1,2-dihydropyridin-3-yl)-4-isopropoxypyrimidine-5-carboxamide [preparation 39] following a similar procedure to that described in Example 31. LCMS m/z=481.9 [M+H]⁺. 1H NMR (METHANOL-d4, 400 MHz) δ 9.3-9.5 (m, 1H), 8.59 (dd, 1H, J=1.8, 7.5 Hz), 7.64 (s, 1H), 7.36 (dd, 1H, J=1.8, 7.0 Hz), 6.39 (t, 1H, J=7.2 Hz), 5.80 (quin, 1H, J=6.2 Hz), 4.7-4.8 (m, 1H), 4.62 (s, 1H), 4.09 (dd, 1H, J=3.5, 6.5 Hz), 3.99 (d, 1H, J=6.5 Hz), 3.4-3.5 (m, 1H), 2.1-2.3 (m, 3H), 1.9-2.0 (m, 3H), 1.66 (d, 6H, J=6.3 Hz), 1.2-1.2 (m, 2H), 0.9-1.0 (m, 2H).

### Examples 46 and 47: N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyrimidine-6-carboxamide and N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyrimidine-6-carboxamide

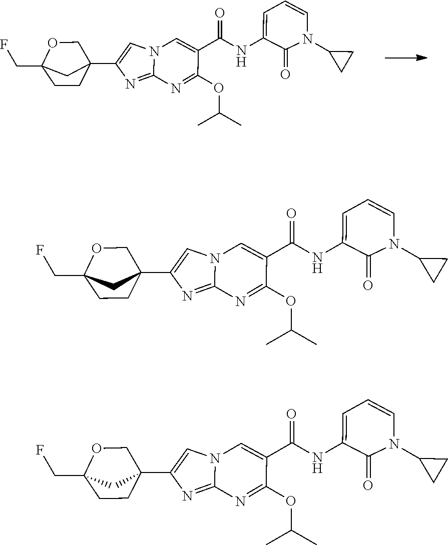 [SMILES: CC(C)Oc1nc2nc(C34CCC(CF)(C3)OC4)cn2cc1C(=O)Nc1cccn(C2CC2)c1=O ; CC(C)Oc1nc2nc([C@@]34CC[C@@](CF)(C3)OC4)cn2cc1C(=O)Nc1cccn(C2CC2)c1=O ; CC(C)Oc1nc2nc([C@]34CC[C@](CF)(C3)OC4)cn2cc1C(=O)Nc1cccn(C2CC2)c1=O]

N-(1-cyclopropyl-2-oxo-1,2-dihydropyridin-3-yl)-2-(1-(fluoromethyl)-2-oxabicyclo[2.2.1]heptan-4-yl)-7-isopropoxyimidazo[1,2-a]pyrimidine-6-carboxamide [Example 45] (26 mg, 54 umol) was separated by SFC CHIRALPAK IB 30×250 mm, 5 um; Method: 40% MeOH w/ No Modifier in CO₂ (flow rate: 100 mL/min, ABPR 120 bar, MBPR 40 psi, column temp 40 deg C.) to provide: Peak 1: N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyrimidine-6-carboxamide, Example 46, stereochemistry arbitrarily assigned (9.4 mg, 36% yield). LCMS (ESI) m/z 481.9 (M+H)⁺; 1H NMR (METHANOL-d4, 400 MHz) δ 9.4 (s, 1H), 8.59 (dd, 1H, J=1.8, 7.5 Hz), 7.63 (s, 1H), 7.36 (dd, 1H, J=1.8, 7.0 Hz), 6.39 (t, 1H, J=7.2 Hz), 5.80 (td, 1H, J=6.3, 12.4 Hz), 4.74 (s, 1H), 4.62 (s, 1H), 4.09 (dd, 1H, J=3.4, 6.7 Hz), 3.99 (d, 1H, J=6.5 Hz), 3.4-3.5 (m, 1H), 1.9-2.3 (m, 6H), 1.66 (d, 6H, J=6.3 Hz), 1.1-1.3 (m, 2H), 0.9-1.0 (m, 2H) and Peak 2: N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyrimidine-6-carboxamide, Example 47, stereochemistry arbitrarily assigned (9.7 mg. 37% yield). LCMS (ESI) m/z 481.9 (M+H)⁺; 1H NMR (METHANOL-d4, 400 MHz) δ 9.4 (s, 1H), 8.59 (dd, 1H, J=1.8, 7.5 Hz), 7.64 (s, 1H), 7.36 (dd, 1H, J=1.8, 6.8 Hz), 6.39 (t, 1H, J=7.3 Hz), 5.80 (td, 1H, J=6.3, 12.5 Hz), 4.74 (s, 1H), 4.62 (s, 1H), 4.09 (dd, 1H, J=3.5, 6.5 Hz), 3.99 (d, 1H, J=6.5 Hz), 3.4-3.5 (m, 1H), 1.8-2.3 (m, 6H), 1.66 (d, 6H, J=6.0 Hz), 1.2-1.2 (m, 2H), 0.9-1.0 (m, 2H).

### Example 48: 7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide

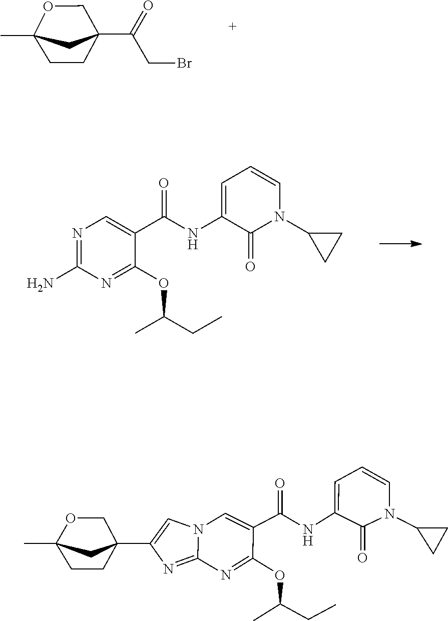 [SMILES: C[C@@]12CC[C@@](C(=O)CBr)(CO1)C2 ; CC[C@@H](C)Oc1nc(N)ncc1C(=O)Nc1cccn(C2CC2)c1=O ; CC[C@@H](C)Oc1nc2nc([C@@]34CC[C@@](C)(C3)OC4)cn2cc1C(=O)Nc1cccn(C2CC2)c1=O]

7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide was obtained as an off white powder (26 mg, yield 27%) from 2-bromo-1-((1S,4S)-1-methyl-2-oxabicyclo[2.2.1]heptan-4-yl)ethan-1-one [preparation 24a] and (R)-2-amino-4-(sec-butoxy)-N-(1-cyclopropyl-2-oxo-1,2-dihydropyridin-3-yl)pyrimidine-5-carboxamide [preparation 41] following a similar procedure to that described in Example 31. LCMS m/z=478.2 [M+H]⁺. 1H NMR (METHANOL-d4, 400 MHz) δ 10.72 (s, 1H), 9.39 (s, 1H), 8.56 (dd, 1H, J=1.8, 7.5 Hz), 7.59 (s, 1H), 7.33 (dd, 1H, J=1.8, 7.0 Hz), 6.36 (t, 1H, J=7.3 Hz), 5.6-5.7 (m, 1H), 4.04 (dd, 1H, J=3.3, 6.5 Hz), 3.91 (d, 1H, J=6.5 Hz), 3.4-3.5 (m, 1H), 2.1-2.3 (m, 2H), 2.0-2.1 (m, 1H), 1.7-2.0 (m, 5H), 1.60 (d, 3H, J=6.3 Hz), 1.46 (s, 3H), 1.1-1.2 (m, 2H), 1.04 (t, 3H, J=7.5 Hz), 0.9-1.0 (m, 2H)

### Example 49: 7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide

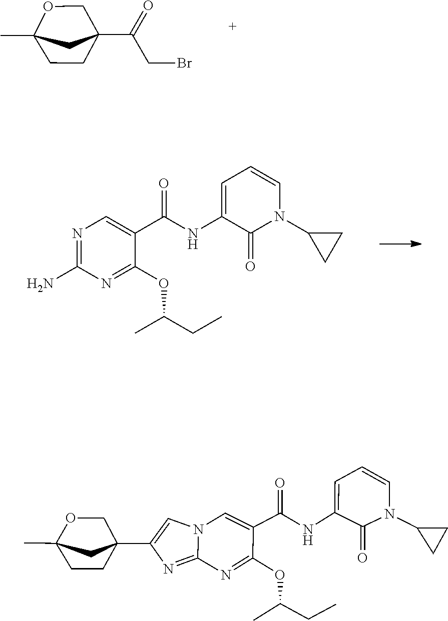 [SMILES: C[C@@]12CC[C@@](C(=O)CBr)(CO1)C2 ; CC[C@H](C)Oc1nc(N)ncc1C(=O)Nc1cccn(C2CC2)c1=O ; CC[C@H](C)Oc1nc2nc([C@@]34CC[C@@](C)(C3)OC4)cn2cc1C(=O)Nc1cccn(C2CC2)c1=O]

7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide was obtained as an off-white powder (31 mg, yield 32%) from 2-bromo-1-((1S,4S)-1-methyl-2-oxabicyclo[2.2.1]heptan-4-yl)ethan-1-one [preparation 24a] and (S)-2-amino-4-(sec-butoxy)-N-(1-cyclopropyl-2-oxo-1,2-dihydropyridin-3-yl)pyrimidine-5-carboxamide [preparation 42] following a similar procedure to that described in Example 31. LCMS m/z=478.2 [M+H]⁺. 1H NMR (METHANOL-d4, 400 MHz) δ 10.62 (s, 1H), 9.56 (s, 1H), 8.58 (dd, 1H, J=1.8, 7.5 Hz), 7.83 (s, 1H), 7.38 (dd, 1H, J=1.8, 7.0 Hz), 6.38 (t, 1H, J=7.3 Hz), 5.6-5.7 (m, 1H), 4.0-4.1 (m, 1H), 3.9-4.0 (m, 1H), 3.4-3.5 (m, 1H), 2.1-2.3 (m, 3H), 1.8-2.1 (m, 5H), 1.65 (d, 3H, J=6.3 Hz), 1.48 (s, 3H), 1.1-1.2 (m, 2H), 1.06 (t, 3H, J=7.4 Hz), 0.9-1.0 (m, 2H).

### Example 50: 7-((S)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide

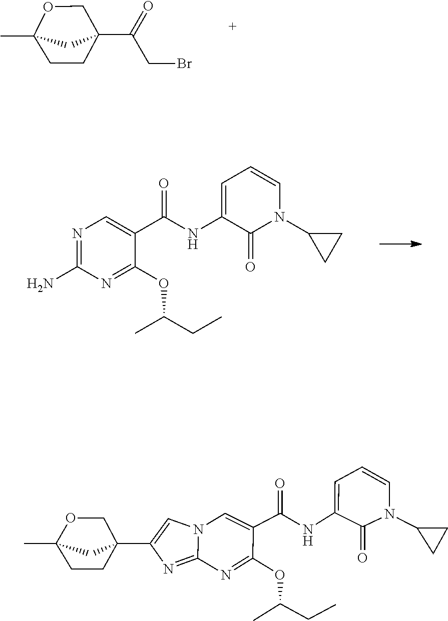 [SMILES: C[C@]12CC[C@](C(=O)CBr)(CO1)C2 ; CC[C@H](C)Oc1nc(N)ncc1C(=O)Nc1cccn(C2CC2)c1=O ; CC[C@H](C)Oc1nc2nc([C@]34CC[C@](C)(C3)OC4)cn2cc1C(=O)Nc1cccn(C2CC2)c1=O]

7-((S)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide was obtained as an off-white powder (45 mg, yield 52%) from 2-bromo-1-((1R,4R)-1-methyl-2-oxabicyclo[2.2.1]heptan-4-yl)ethan-1-one [preparation 24b] and (S)-2-amino-4-(sec-butoxy)-N-(1-cyclopropyl-2-oxo-1,2-dihydropyridin-3-yl)pyrimidine-5-carboxamide [preparation 42] following a similar procedure to that described in Example 31. LCMS m/z=478.2 [M+H]⁺. 1H NMR (METHANOL-d4, 400 MHz) δ 10.70 (s, 1H), 9.35 (s, 1H), 8.53 (dd, 1H, J=1.8, 7.5 Hz), 7.57 (s, 1H), 7.31 (dd, 1H, J=1.5, 7.0 Hz), 6.33 (t, 1H, J=7.3 Hz), 5.5-5.7 (m, 1H), 4.03 (dd, 1H, J=3.3, 6.5 Hz), 3.8-3.9 (m, 1H), 3.4-3.5 (m, 1H), 2.0-2.3 (m, 3H), 1.7-2.0 (m, 5H), 1.59 (d, 3H, J=6.3 Hz), 1.45 (s, 3H), 1.1-1.3 (m, 2H), 1.03 (t, 3H, J=7.4 Hz), 0.9-1.0 (m, 2H).

### Example 51: 7-((R)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide

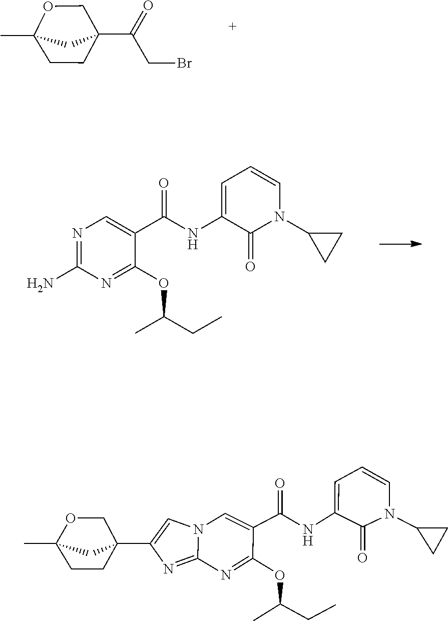 [SMILES: C[C@]12CC[C@](C(=O)CBr)(CO1)C2 ; CC[C@@H](C)Oc1nc(N)ncc1C(=O)Nc1cccn(C2CC2)c1=O ; CC[C@@H](C)Oc1nc2nc([C@]34CC[C@](C)(C3)OC4)cn2cc1C(=O)Nc1cccn(C2CC2)c1=O]

7-((R)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide was obtained as an off-white powder (53 mg, yield 61%) from 2-bromo-1-((1R,4R)-1-methyl-2-oxabicyclo[2.2.1]heptan-4-yl)ethan-1-one [preparation 24b] and (R)-2-amino-4-(sec-butoxy)-N-(1-cyclopropyl-2-oxo-1,2-dihydropyridin-3-yl)pyrimidine-5-carboxamide [preparation 41] following a similar procedure to that described in Example 31. LCMS m/z=478.2 [M+H]⁺. 1H NMR (METHANOL-d4, 400 MHz) δ 9.59 (s, 1H), 8.57 (dd, 1H, J=1.8, 7.3 Hz), 7.88 (s, 1H), 7.38 (dd, 1H, J=1.8, 7.0 Hz), 6.38 (t, 1H, J=7.2 Hz), 5.6-5.8 (m, 1H), 4.0-4.0 (m, 1H), 3.9-4.0 (m, 1H), 3.4-3.5 (m, 1H), 2.1-2.3 (m, 3H), 2.03 (s, 2H), 1.8-2.0 (m, 3H), 1.66 (d, 3H, J=6.3 Hz), 1.48 (s, 3H), 1.1-1.2 (m, 2H), 1.07 (t, 3H, J=7.4 Hz), 0.97 (qd, 2H, J=1.8, 4.1 Hz).

### Example 52: N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide

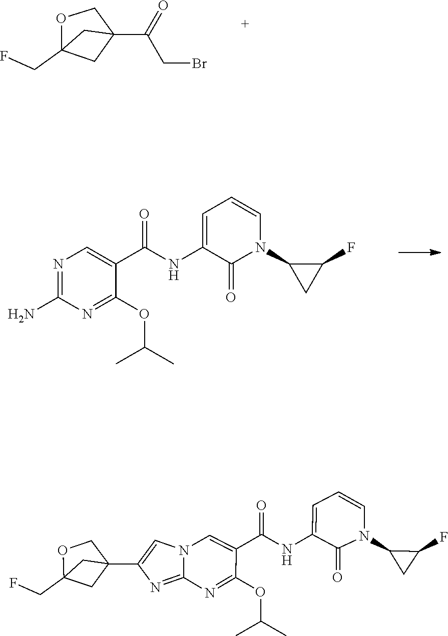 [SMILES: O=C(CBr)C12COC(CF)(C1)C2 ; CC(C)Oc1nc(N)ncc1C(=O)Nc1cccn([C@@H]2C[C@@H]2F)c1=O ; CC(C)Oc1nc2nc(C34COC(CF)(C3)C4)cn2cc1C(=O)Nc1cccn([C@@H]2C[C@@H]2F)c1=O]

N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide was obtained as an off-white powder (16 mg, yield 14%) from 2-bromo-1-(1-(fluoromethyl)-2-oxabicyclo[2.1.1]hexan-4-yl)ethan-1-one [preparation 19] and 2-amino-N-(1-(cis-2-fluorocyclopropyl)-2-oxo-1,2-dihydropyridin-3-yl)-4-isopropoxypyrimidine-5-carboxamide [preparation 40] following a similar procedure to that described in Example 31. LCMS m/z=486.1 [M+H]⁺. 1H NMR (METHANOL-d4, 400 MHz) δ 9.42 (s, 1H), 8.62 (dd, 1H, J=1.8, 7.5 Hz), 7.68 (s, 1H), 7.42 (dd, 1H, J=0.8, 7.0 Hz), 6.42 (t, 1H, J=7.3 Hz), 5.80 (quin, 1H, J=6.1 Hz), 4.9-4.9 (m, 1H), 4.75 (s, 1H), 4.63 (s, 1H), 4.08 (s, 2H), 3.4-3.5 (m, 1H), 2.26 (dd, 2H, J=1.6, 4.6 Hz), 1.99 (dd, 2H, J=1.8, 4.8 Hz), 1.66 (dd, 6H, J=1.4, 6.1 Hz), 1.5-1.6 (m, 2H).

### Example 53: N-(1-cyclopropyl-2-oxo-1,2-dihydropyridin-3-yl)-7-isopropoxy-2-(1-(methoxymethyl)-2-oxabicyclo[2.2.1]heptan-4-yl)imidazo[1,2-a]pyrimidine-6-carboxamide

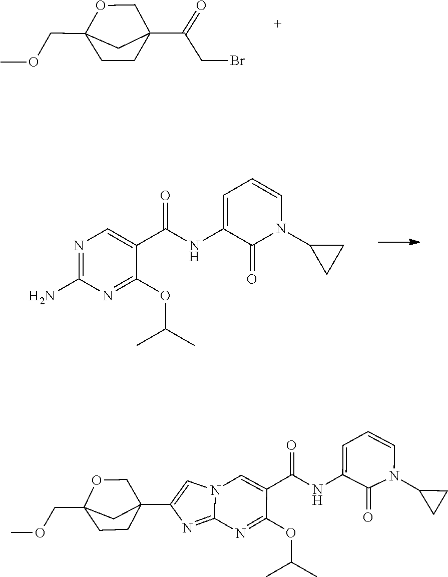 [SMILES: COCC12CCC(C(=O)CBr)(CO1)C2 ; CC(C)Oc1nc(N)ncc1C(=O)Nc1cccn(C2CC2)c1=O ; COCC12CCC(c3cn4cc(C(=O)Nc5cccn(C6CC6)c5=O)c(OC(C)C)nc4n3)(CO1)C2]

N-(1-cyclopropyl-2-oxo-1,2-dihydropyridin-3-yl)-7-isopropoxy-2-(1-(methoxymethyl)-2-oxabicyclo[2.2.1]heptan-4-yl)imidazo[1,2-a]pyrimidine-6-carboxamide was obtained as an off-white powder (58 mg, yield 47%) from 2-bromo-1-(1-(methoxymethyl)-2-oxabicyclo[2.2.1]heptan-4-yl)ethan-1-one [preparation 18] and 2-amino-N-(1-cyclopropyl-2-oxo-1,2-dihydropyridin-3-yl)-4-isopropoxypyrimidine-5-carboxamide [preparation 39] following a similar procedure to that described in Example 31. LCMS m/z=494.3 [M+H]⁺. 1H NMR (METHANOL-d4, 400 MHz) δ 9.40 (s, 1H), 8.59 (dd, 1H, J=1.8, 7.5 Hz), 7.62 (s, 1H), 7.36 (dd, 1H, J=1.8, 7.0 Hz), 6.39 (t, 1H, J=7.2 Hz), 5.80 (quin, 1H, J=6.2 Hz), 4.06 (dd, 1H, J=3.5, 6.5 Hz), 3.96 (d, 1H, J=6.8 Hz), 3.6-3.8 (m, 2H), 3.5-3.5 (m, 1H), 3.4-3.4 (m, 3H), 2.1-2.3 (m, 1H), 2.0-2.1 (m, 2H), 1.8-2.0 (m, 3H), 1.6-1.6 (m, 1H), 1.66 (d, 5H, J=6.3 Hz), 1.2-1.3 (m, 2H), 0.9-1.0 (m, 2H).

### Example 54: 2-(2-oxabicyclo[2.1.1]hexan-4-yl)-N-(1-cyclopropyl-2-oxo-1,2-dihydropyridin-3-yl)-7-isopropoxyimidazo[1,2-a]pyrimidine-6-carboxamide

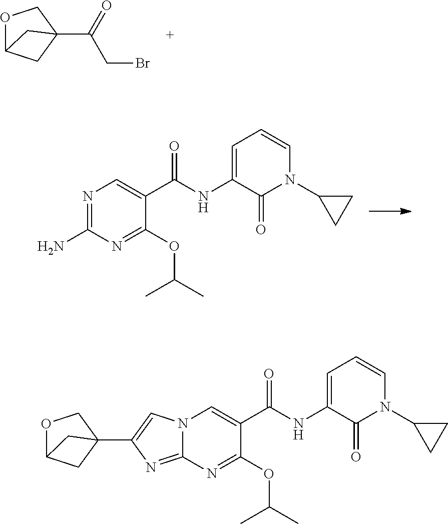 [SMILES: O=C(CBr)C12COC(C1)C2 ; CC(C)Oc1nc(N)ncc1C(=O)Nc1cccn(C2CC2)c1=O ; CC(C)Oc1nc2nc(C34COC(C3)C4)cn2cc1C(=O)Nc1cccn(C2CC2)c1=O]

2-(2-oxabicyclo[2.1.1]hexan-4-yl)-N-(1-cyclopropyl-2-oxo-1,2-dihydropyridin-3-yl)-7-isopropoxyimidazo[1,2-a]pyrimidine-6-carboxamide was obtained as an off-white powder (7 mg, yield 34%) from 1-(2-oxabicyclo[2.1.1]hexan-4-yl)-2-bromoethan-1-one [preparation 20] and 2-amino-N-(1-cyclopropyl-2-oxo-1,2-dihydropyridin-3-yl)-4-isopropoxypyrimidine-5-carboxamide [preparation 39] following a similar procedure to that described in Example 31. LCMS m/z=436.2 [M+H]⁺. 1H NMR (METHANOL-d4, 400 MHz) δ 9.3-9.5 (m, 1H), 8.5-8.6 (m, 1H), 7.64 (s, 1H), 7.35 (d, 1H, J=7.3 Hz), 6.3-6.4 (m, 1H), 5.78 (dd, 1H, J=5.9, 6.7 Hz), 4.65 (d, 1H, J=0.8 Hz), 3.96 (s, 2H), 3.4-3.5 (m, 1H), 2.25 (br d, 2H, J=4.5 Hz), 1.8-1.9 (m, 2H), 1.65 (d, 6H, J=6.3 Hz), 1.18 (br d, 2H, J=6.0 Hz), 0.97 (br d, 2H, J=4.0 Hz).

### Example 55: 7-((R)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide

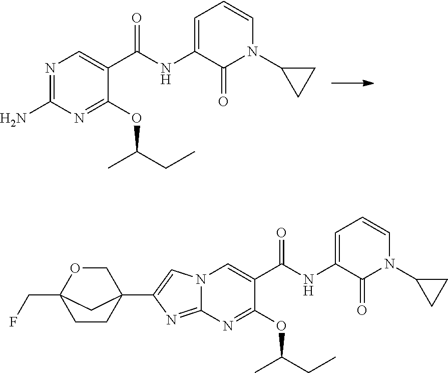 [SMILES: CC[C@@H](C)Oc1nc(N)ncc1C(=O)Nc1cccn(C2CC2)c1=O ; CC[C@@H](C)Oc1nc2nc(C34CCC(CF)(C3)OC4)cn2cc1C(=O)Nc1cccn(C2CC2)c1=O]

7-((R)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide was obtained as an off-white powder (63 mg, yield 51%) from 2-bromo-1-(1-(fluoromethyl)-2-oxabicyclo[2.2.1]heptan-4-yl)ethan-1-one [preparation 17] and (R)-2-amino-4-(sec-butoxy)-N-(1-cyclopropyl-2-oxo-1,2-dihydropyridin-3-yl)pyrimidine-5-carboxamide [preparation 41] following a similar procedure to that described in Example 31. LCMS m/z=496.2 [M+H]⁺. 1H NMR (METHANOL-d4, 400 MHz) δ 10.65 (s, 1H), 9.58 (s, 1H), 8.60 (dd, 1H, J=1.8, 7.3 Hz), 7.87 (s, 1H), 7.39 (dd, 1H, J=1.5, 7.0 Hz), 6.40 (t, 1H, J=7.2 Hz), 5.6-5.8 (m, 1H), 4.6-4.8 (m, 2H), 4.0-4.1 (m, 2H), 3.4-3.6 (m, 1H), 2.1-2.3 (m, 4H), 1.9-2.1 (m, 4H), 1.67 (d, 3H, J=6.3 Hz), 1.1-1.3 (m, 2H), 1.08 (t, 3H, J=7.5 Hz), 0.9-1.0 (m, 2H).

### Example 56: 7-((S)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide

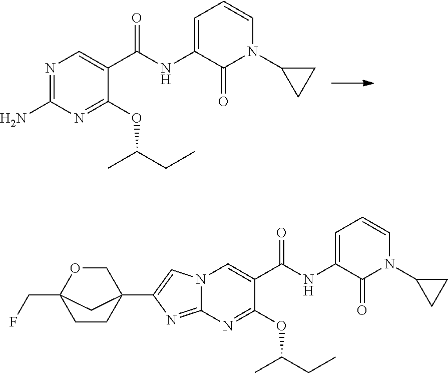 [SMILES: CC[C@H](C)Oc1nc(N)ncc1C(=O)Nc1cccn(C2CC2)c1=O ; CC[C@H](C)Oc1nc2nc(C34CCC(CF)(C3)OC4)cn2cc1C(=O)Nc1cccn(C2CC2)c1=O]

7-((S)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide was obtained as an off-white powder (76 mg, yield 61%) from 2-bromo-1-(1-(fluoromethyl)-2-oxabicyclo[2.2.1]heptan-4-yl)ethan-1-one [preparation 17] and (S)-2-amino-4-(sec-butoxy)-N-(1-cyclopropyl-2-oxo-1,2-dihydropyridin-3-yl)pyrimidine-5-carboxamide [preparation 42] following a similar procedure to that described in Example 31. LCMS m/z=496.3 [M+H]⁺. 1H NMR (METHANOL-d4, 400 MHz) δ 10.66 (s, 1H), 9.5-9.6 (m, 1H), 8.60 (dd, 1H, J=1.8, 7.5 Hz), 7.84 (s, 1H), 7.39 (dd, 1H, J=1.8, 7.0 Hz), 6.40 (t, 1H, J=7.2 Hz), 5.6-5.8 (m, 1H), 4.6-4.8 (m, 2H), 4.0-4.1 (m, 2H), 3.4-3.6 (m, 1H), 2.1-2.3 (m, 4H), 1.9-2.1 (m, 4H), 1.66 (d, 3H, J=6.3 Hz), 1.2-1.3 (m, 2H), 1.08 (t, 3H, J=7.5 Hz), 0.9-1.0 (m, 2H).

### Examples 57 and 58: 7-((R)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide and 7-((R)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

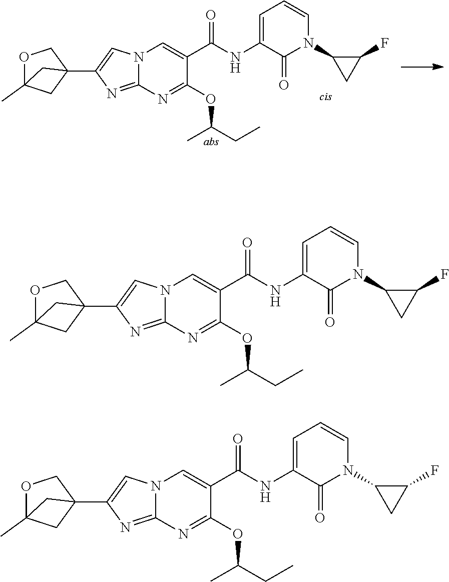 [SMILES: CC[C@@H](C)Oc1nc2nc(C34COC(C)(C3)C4)cn2cc1C(=O)Nc1cccn([C@@H]2C[C@@H]2F)c1=O ; CC[C@@H](C)Oc1nc2nc(C34COC(C)(C3)C4)cn2cc1C(=O)Nc1cccn([C@@H]2C[C@@H]2F)c1=O ; CC[C@@H](C)Oc1nc2nc(C34COC(C)(C3)C4)cn2cc1C(=O)Nc1cccn([C@H]2C[C@H]2F)c1=O]

7-((R)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide [Example 41] (52 mg, 107.99 umol) was separated by SFC (CHIRALPAK IB 30×250 mm, 5 um; Method: 40% MeOH w/ No Modifier in CO₂ (flow rate: 100 mL/min, ABPR 120 bar, MBPR 40 psi, column temp 40 deg C.) to provide:

Peak 1, Example 57: 7-((R)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (12.6 mg, 23% yield). LCMS (ESI) m/z 482.1 (M+H)⁺; 1H NMR (METHANOL-d4, 400 MHz) δ 9.42 (s, 1H), 8.5-8.7 (m, 1H), 7.64 (s, 1H), 7.41 (d, 1H, J=7.0 Hz), 6.3-6.5 (m, 1H), 5.6-5.7 (m, 1H), 4.9-5.1 (m, 1H), 4.02 (s, 2H), 3.44 (br dd, 1H, J=2.9, 5.1 Hz), 2.1-2.3 (m, 3H), 1.8-2.0 (m, 3H), 1.62 (dd, 3H, J=1.8, 6.3 Hz), 1.5-1.6 (m, 2H), 1.52 (s, 3H), 1.0-1.1 (m, 3H). Relative stereochemistry of cyclopropyl substituents is cis, but absolute stereochemistry is arbitrarily assigned.

Peak 2, Example 58: 7-((R)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (18.5 mg. 36% yield). LCMS (ESI) m/z 482.1 (M+H)⁺; 1H NMR (METHANOL-d4, 400 MHz) δ 9.42 (s, 1H), 8.62 (dd, 1H, J=1.8, 7.5 Hz), 7.64 (s, 1H), 7.41 (dd, 1H, J=1.0, 7.0 Hz), 6.3-6.5 (m, 1H), 5.6-5.7 (m, 1H), 4.9-5.1 (m, 1H), 4.03 (s, 2H), 3.4-3.5 (m, 1H), 2.1-2.3 (m, 3H), 1.8-2.0 (m, 3H), 1.5-1.7 (m, 8H), 1.06 (t, 3H, J=7.4 Hz). Relative stereochemistry of cyclopropyl substituents is cis, but absolute stereochemistry is arbitrarily assigned.

### Examples 59 and 60: 7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide and 7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

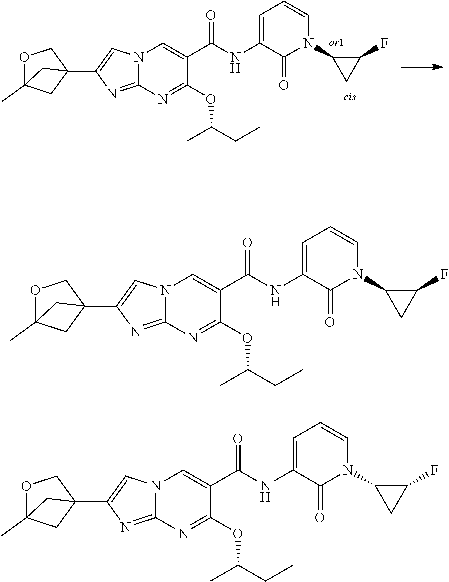 [SMILES: CC[C@H](C)Oc1nc2nc(C34COC(C)(C3)C4)cn2cc1C(=O)Nc1cccn([C@@H]2C[C@@H]2F)c1=O ; CC[C@H](C)Oc1nc2nc(C34COC(C)(C3)C4)cn2cc1C(=O)Nc1cccn([C@@H]2C[C@@H]2F)c1=O ; CC[C@H](C)Oc1nc2nc(C34COC(C)(C3)C4)cn2cc1C(=O)Nc1cccn([C@H]2C[C@H]2F)c1=O]

7-((S)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide [Example 42] (54.00 mg, 112.15 umol) was separated by SFC (CHIRALPAK IB 30×250 mm, 5 um; Method: 40% MeOH w/ No Modifier in CO₂ (flow rate: 100 mL/min, ABPR 120 bar, MBPR 40 psi, column temp 40 deg C.) to provide:

Peak 1, Example 59: 7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (11 mg, 21% yield). LCMS (ESI) m/z 482.1 (M+H)⁺; 1H NMR (METHANOL-d4, 400 MHz) δ 9.42 (s, 1H), 8.62 (dd, 1H, J=1.8, 7.5 Hz), 7.64 (s, 1H), 7.41 (dd, 1H, J=1.0, 7.0 Hz), 6.3-6.5 (m, 1H), 5.6-5.7 (m, 1H), 4.9-5.1 (m, 1H), 4.03 (s, 2H), 3.4-3.5 (m, 1H), 2.1-2.3 (m, 3H), 1.8-2.0 (m, 3H), 1.5-1.7 (m, 8H), 1.06 (t, 3H, J=7.4 Hz). Relative stereochemistry of cyclopropyl substituents is cis, but absolute stereochemistry is arbitrarily assigned.

Peak 2: 7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (6.8 mg. 11% yield). LCMS (ESI) m/z 482.1 (M+H)⁺; 1H NMR (METHANOL-d4,400 MHz) δ 9.42 (s, 1H), 8.61 (dd, 1H, J=1.9, 7.4 Hz), 7.64 (s, 1H), 7.41 (dd, 1H, J=1.0, 7.0 Hz), 6.41 (t, 1H, J=7.3 Hz), 5.5-5.8 (m, 1H), 4.9-5.1 (m, 1H), 4.03 (s, 2H), 3.4-3.5 (m, 1H), 2.1-2.3 (m, 3H), 1.9-2.0 (m, 3H), 1.5-1.7 (m, 8H), 1.0-1.1 (m, 3H). Relative stereochemistry of cyclopropyl substituents is cis, but absolute stereochemistry is arbitrarily assigned.

### Example 61: N-(1-cyclopropyl-2-oxo-1,2-dihydropyridin-3-yl)-8-fluoro-7-isopropoxy-2-(1-methyl-2-oxabicyclo[2.1.1]hexan-4-yl)imidazo[1,2-a]pyridine-6-carboxamide

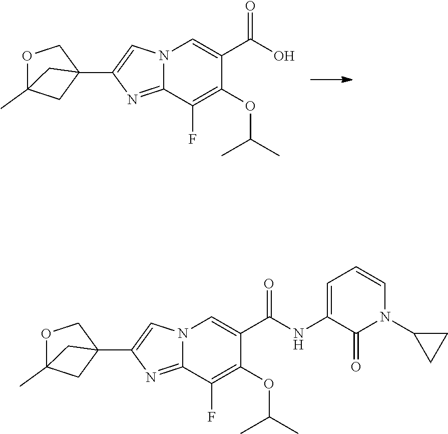

T3P (0.6 mmol, 356 μL, 50% purity in EtOAc) was added to 8-fluoro-7-isopropoxy-2-(1-methyl-2-oxabicyclo[2.1.1]hexan-4-yl)imidazo[1,2-a]pyridine-6-carboxylic acid [preparation 73] (40.0 mg, 0.119 mmol) and 3-amino-1-cyclopropyl-pyridin-2-one (23 mg, 0.16 mmol, hydrochloride) in Pyridine (1.0 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide 7-cyclobutoxy-N-(1-cyclopropyl-2-oxo-1,2-dihydropyridin-3-yl)-2-(1-methyl-2-oxabicyclo[2.2.2]octan-4-yl)imidazo[1,2-a]pyridine-6-carboxamide (30.7 mg, 55.0% yield). LCMS (ESI) m/z 466.9 (M+H)⁺. 1H NMR (500 MHz, DMSO-d6) δ ppm 0.87-0.96 (m, 2H) 1.03-1.09 (m, 2H) 1.43-1.47 (m, 9H) 1.79 (dd, J=4.27, 1.22 Hz, 2H) 2.04 (dd, J=4.27, 1.83 Hz, 2H) 3.50 (ddd, J=11.44, 7.48, 4.27 Hz, 1H) 3.91 (s, 2H) 4.79 (dt, J=12.21, 6.10 Hz, 1H) 6.31 (t, J=7.02 Hz, 1H) 7.35 (dd, J=6.71, 1.83 Hz, 1H) 8.03 (d, J=3.05 Hz, 1H) 8.44 (dd, J=7.32, 1.83 Hz, 1H) 9.09 (d, J=1.22 Hz, 1H) 10.63 (s, 1H). 19F NMR (470 MHz, DMSO-d6) δ ppm −150.32 (s, 1 F).

### Example 62: N-(1-cyclopropyl-2-oxo-1,2-dihydropyridin-3-yl)-7-isopropoxy-2-(3-methoxybicyclo[1.1.1]pentan-1-yl)imidazo[1,2-a]pyridine-6-carboxamide

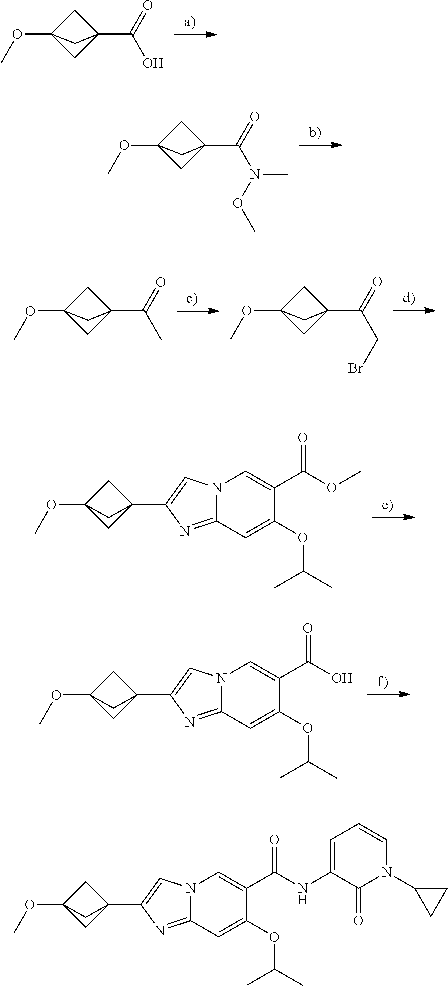

Step a: di(imidazol-1-yl)methanone (684.40 mg, 4.22 mmol) was added to 3-methoxybicyclo[1.1.1]pentane-1-carboxylic acid (500 mg, 3.52 mmol) in DCM (7.03 mL) at rt. After stirring for 2 h, N-methoxymethanamine; hydrochloride (343 mg, 3.52 mmol) was added and stirred at rt overnight. The mixture was poured into water, extracted three times with DCM, dried MgSO4, filtered, concentrated, purified by silica gel column (12 g, 0-100% 3; 1 EtOAc:EtOH in heptanes) to obtain N,3-dimethoxy-N-methyl-bicyclo[1.1.1]pentane-1-carboxamide (330 mg, 1.78 mmol, 51% yield). (LCMS (ESI) m/z 186.0 (M+H)⁺.

Step b: Methyllithium (1.6 M, 1.39 mL) was added to N,3-dimethoxy-N-methyl-bicyclo[1.1.1]pentane-1-carboxamide (330 mg, 1.78 mmol) in THF (3.56 mL) at −78° C. under nitrogen atmosphere. After 30 min, the mixture was warmed to 0° C. and then allowed to warm to rt over a period of 2 h. The reaction was quenched with sat. aq. NH₄Cl, extracted three times with EtOAc, washed with brine, dried over MgSO₄, filtered, and concentrated, to obtain non UV-active 1-(3-methoxy-1-bicyclo[1.1.1]pentanyl)ethanone (200 mg, 1.43 mmol, 80% yield) which was used directly in next reaction without further purification.

Step c: dibromocopper (446 mg, 2.00 mmol) was added to 1-(3-methoxy-1-bicyclo[1.1.1]pentanyl)ethanone (200 mg, 1.43 mmol) in EtOH (3.57 mL) at rt. The mixture was heated at 70° C. for 25 min, then cooled to rt, diluted with water and EtOAc, extracted three times with EtOAc, washed with sat. aq. NaHCO₃, then brine, dried over MgSO4, filtered, concentrated, to obtain 2-bromo-1-(3-methoxy-1-bicyclo[1.1.1]pentanyl)ethanone (250 mg, 1.14 mmol, 79% yield) which was used directly in next reaction without further purification.

Step d: A mixture of methyl 6-amino-4-isopropoxynicotinate [preparation 2] (250 mg, 1.19 mmol), 2-bromo-1-(3-methoxy-1-bicyclo[1.1.1]pentanyl)ethanone (260.52 mg, 1.19 mmol), NaHCO₃ (299.70 mg, 3.57 mmol) in MeCN (1.90 mL) and toluene (2.85 mL) was heated at 90° C. for 16 h. Silica and MeOH were added and the mixture was concentrated and purified by silica gel column chromatography (dry load, 24 g, 0-70% gradient of 3:1 EtOAC/EtOH in heptanes) to obtain ethyl 7-isopropoxy-2-(3-methoxy-1-bicyclo[1.1.1]pentanyl)imidazo[1,2-a]pyridine-6-carboxylate (200 mg, 605 μmol, 51% yield). (LCMS (ESI) m/z 330.9 (M+H)⁺.

Step e: A mixture of methyl 7-isopropoxy-2-(3-methoxy-1-bicyclo[1.1.1]pentanyl)imidazo[1,2-a]pyridine-6-carboxylate (200 mg, 605 μmol) and lithium; hydroxide; hydrate (50.8 mg, 1.21 mmol) in Methanol (605 μL), THF (4.36 mL), water (1.09 mL) was stirred overnight at room temperature before being neutralized with a 4.0 M hydrochloric acid solution in dioxane. The mixture was concentrated to obtain 2-(1-(fluoromethyl)-2-oxabicyclo[2.2.1]heptan-4-yl)-7-isopropoxyimidazo[1,2-a]pyridine-6-carboxylic acid, which was used without further purification in next reaction. Assumed 100% yield. LCMS (ESI) m/z 316.9 (M+H)⁺.

Step f: T3P® (0.75 mmol, 450 uL, 50% purity in EtOAc) was added to 2-(1-(fluoromethyl)-2-oxabicyclo[2.2.1]heptan-4-yl)-7-isopropoxyimidazo[1,2-a]pyridine-6-carboxylic acid (48 mg, 0.15 mmol) and 3-amino-1-cyclopropyl-pyridin-2-one (25 mg, 0.16 mmol) in Pyridine (1 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide N-(1-cyclopropyl-2-oxo-1,2-dihydropyridin-3-yl)-7-isopropoxy-2-(3-methoxybicyclo[1.1.1]pentan-1-yl)imidazo[1,2-a]pyridine-6-carboxamide (24 mg, 35% yield). LCMS (ESI) m/z 449.0 (M+H)⁺. 1H NMR (500 MHz, DMSO-d6) δ ppm 0.91-0.93 (m, 2H) 1.05 (dd, J=7.32, 1.83 Hz, 2H) 1.54 (d, J=6.10 Hz, 6H) 2.18 (s, 6H) 3.27 (s, 3H) 3.48-3.54 (m, 1H) 5.02-5.15 (m, 1H) 6.31 (t, J=7.02 Hz, 1H) 7.21 (s, 1H) 7.34 (dd, J=6.71, 1.83 Hz, 1H) 7.85 (br s, 1H) 8.44 (dd, J=7.32, 1.83 Hz, 1H) 9.25 (s, 1H) 10.72 (s, 1H).

### Examples 63 and 64: (R)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide and (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide

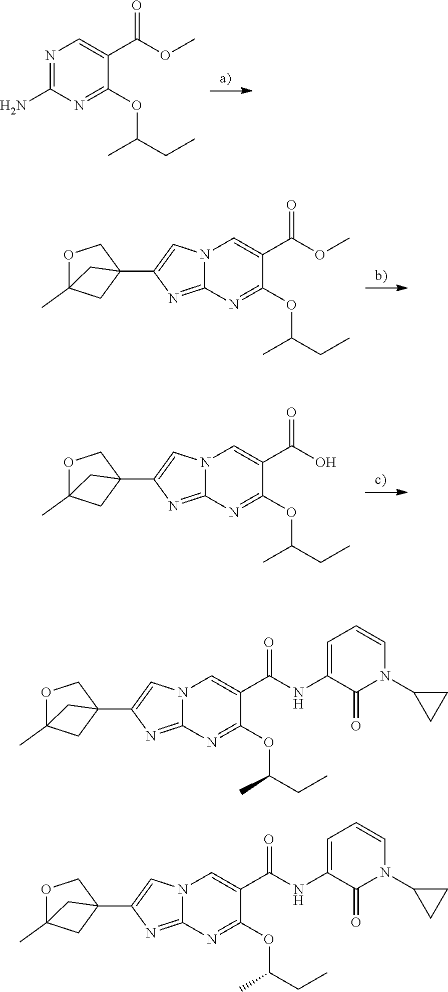

Step a: A mixture of methyl 2-amino-4-(sec-butoxy)pyrimidine-5-carboxylate [preparation 75] (250 mg, 1.11 mmol), 2-bromo-1-(1-methyl-2-oxabicyclo[2.1.1]hexan-4-yl)ethan-1-one [preparation 12](304 mg, 1.39 mmol), NaHCO₃ (280 mg, 3.33 mmol) in MeCN (1.8 mL) and Toluene (2.7 mL) was heated at 90° C. for 16 h. Silica and MeOH were added and the mixture was concentrated and purified by silica gel column chromatography (dry load, 24 g, 0-70% gradient of 3:1 EtOAC/EtOH in heptanes) to obtain methyl 7-(sec-butoxy)-2-(1-methyl-2-oxabicyclo[2.1.1]hexan-4-yl)imidazo[1,2-a]pyrimidine-6-carboxylate (160 mg, 463 μmol, 41% yield). (LCMS (ESI) m/z 346.2 (M+H)⁺.

Step b: A mixture of methyl 7-(sec-butoxy)-2-(1-methyl-2-oxabicyclo[2.1.1]hexan-4-yl)imidazo[1,2-a]pyrimidine-6-carboxylate (160 mg, 463 μmol) and lithium; hydroxide; hydrate (38.9 mg, 926 μmol) in Methanol (308 μL), THF (2.2 mL), water (555 μL) was stirred overnight at room temperature before being neutralized with a 4.0 M hydrochloric acid solution in dioxane. The mixture was concentrated to obtain 7-(sec-butoxy)-2-(1-methyl-2-oxabicyclo[2.1.1]hexan-4-yl)imidazo[1,2-a]pyrimidine-6-carboxylic acid, which was used without further purification in next reaction. Assumed 100% yield. LCMS (ESI) m/z 332.9 (M+H)⁺.

Step c: T3P (2.31 mmol, 1.37 mL, 50% purity) was added to 2-(1-methyl-2-oxabicyclo[2.1.1]hexan-4-yl)-7-[(1R)-1-methylpropoxy]imidazo[1,2-a]pyrimidine-6-carboxylic acid (153 mg, 462 μmol) and 3-amino-1-cyclopropyl-pyridin-2-one (112 mg, 600 μmol, Hydrochloride) in Pyridine (2.3 mL) at rt. After stirring overnight, the mixture was diluted with water and extracted with DCM and then EtOAc, dried over MgSO₄, filtered, concentrated and purified via chiral SFC (CHIRALPAK AD-H 30×250 mm, 5 um Method: 30% EtOH w/ 0.1% DEA in CO₂ (flow rate: 100 mL/min, ABPR 120 bar, MBPR 40 psi, column temp 40 deg C.) to obtain Peak 1: (R)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide, Example 63, stereochemistry arbitrarily assigned (44 mg, 20% yield). LCMS (ESI) m/z 464.3 (M+H)⁺. 1H NMR (400 MHz, CHLOROFORM-d) δ ppm 0.90-0.96 (m, 2H) 1.02 (t, J=7.40 Hz, 3H) 1.16-1.25 (m, 2H) 1.55 (s, 3H) 1.61 (d, J=6.27 Hz, 3H) 1.86-1.95 (m, 1H) 1.96 (dd, J=4.52, 1.76 Hz, 2H) 2.11 (dd, J=4.64, 1.63 Hz, 2H) 2.18 (dd, J=14.05, 7.03 Hz, 1H) 3.44-3.51 (m, 1H) 4.09 (s, 2H) 5.66-5.81 (m, 1H) 6.23 (t, J=7.28 Hz, 1H) 7.07 (dd, J=7.03, 1.76 Hz, 1H) 7.27 (s, 1H) 8.53 (dd, J=7.28, 1.76 Hz, 1H) 9.16 (s, 1H) 10.81 (s, 1H) and Peak 2: (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide, Example 64, stereochemistry arbitrarily assigned (5.5 mg, 2.0% yield). LCMS (ESI) m/z 464.3 (M+H)⁺. 1H NMR (400 MHz, CHLOROFORM-d) δ ppm 0.91-0.97 (m, 2H) 1.03 (t, J=7.53 Hz, 3H) 1.19 (d, J=6.78 Hz, 2H) 1.55 (s, 3H) 1.60-1.61 (m, 3H) 1.84-1.94 (m, 1H) 1.97 (dd, J=4.52, 1.76 Hz, 2H) 2.11 (dd, J=4.52, 1.51 Hz, 2H) 2.19 (dt, J=13.80, 7.15 Hz, 1H) 3.48 (s, 1H) 4.09 (s, 2H) 5.65-5.79 (m, 1H) 6.23 (t, J=7.15 Hz, 1H) 7.07 (dd, J=7.03, 1.76 Hz, 1H) 7.27 (s, 1H) 8.53 (dd, J=7.28, 1.76 Hz, 1H) 9.16 (s, 1H) 10.81 (s, 1H).

### Examples 65 and 66: (R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide and (S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide

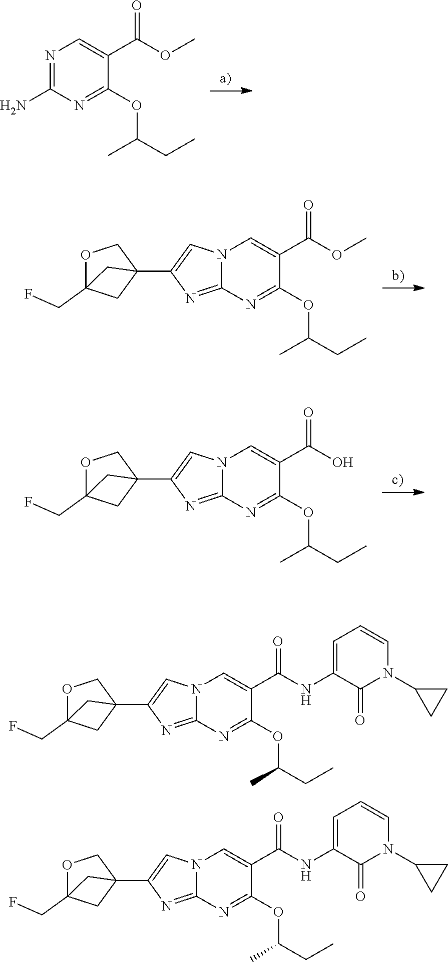

Step a: A mixture of methyl 2-amino-4-(sec-butoxy)pyrimidine-5-carboxylate [preparation 75] (250 mg, 1.11 mmol), 2-bromo-1-[1-(fluoromethyl)-2-oxabicyclo[2.1.1]hexan-4-yl]ethanone (329 mg, 1.39 mmol), NaHCO₃ (280 mg, 3.33 mmol, 129 μL) in Acetonitrile (1.78 mL) and Toluene (2.66 mL) was heated at 90° C. for 16 h. Silica and MeOH were added and the mixture was concentrated and purified by silica gel column chromatography (dry load, 24 g, 0-70% gradient of 3:1 EtOAC/EtOH in heptanes) to obtain methyl 7-(sec-butoxy)-2-(1-(fluoromethyl)-2-oxabicyclo[2.1.1]hexan-4-yl)imidazo[1,2-a]pyrimidine-6-carboxylate (160 mg, 440 μmol, 39% yield). (LCMS (ESI) m/z 364.2 (M+H)⁺.

Step b: A mixture of methyl 7-(sec-butoxy)-2-(1-(fluoromethyl)-2-oxabicyclo[2.1.1]hexan-4-yl)imidazo[1,2-a]pyrimidine-6-carboxylate (160 mg, 440 μmol) and lithium; hydroxide; hydrate (37.0 mg, 881 μmol) in Methanol (293 μL), TH (2.1 mL), water (528 L) was stirred overnight at room temperature before being neutralized with a 4.0 M hydrochloric acid solution in dioxane. The mixture was concentrated to obtain 7-(sec-butoxy)-2-(1-(fluoromethyl)-2-oxabicyclo[2.1.1]hexan-4-yl)imidazo[1,2-a]pyrimidine-6-carboxylic acid, which was used without further purification in next reaction. Assumed 100% yield. LCMS (ESI) m/z 350.2 (M+H)⁺.

Step c: T3P (2.19 mmol, 1.30 mL, 50% purity) was added to 7-(sec-butoxy)-2-(1-(fluoromethyl)-2-oxabicyclo[2.1.1]hexan-4-yl)imidazo[1,2-a]pyrimidine-6-carboxylic acid (153 mg, 438 μmol) and 3-amino-1-cyclopropyl-pyridin-2-one (106.2 mg, 569.3 μmol, Hydrochloride) in Pyridine (2.0 mL) at rt. After stirring for 3 h, the mixture was diluted with water and extracted with DCM and then EtOAc. The combined org. phase was dried over MgSO₄, filtered, concentrated and purified via SFC (CHIRALPAK AD-H 30×250 mm, 5 um Method: 35% MeOH w/ 0.1% DEA in CO₂ (flow rate: 100 mL/min, ABPR 120 bar, MBPR 40 psi, column temp 40 deg C.) to obtain Peak 1: (R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide, Example 65, stereochemistry arbitrarily assigned (18.6 mg, 8% yield). LCMS (ESI) m/z 464.3 (M+H)⁺. 19F NMR (470 MHz, CHLOROFORM-d) δ ppm −75.62 (q, J=6.20 Hz, 1 F); 1H NMR (400 MHz, CHLOROFORM-d) δ ppm 0.91-0.96 (m, 2H) 1.03 (t, J=7.40 Hz, 3H) 1.16-1.23 (m, 2H) 1.61-1.62 (m, 3H) 1.86-1.98 (m, 1H) 2.07 (dd, J=4.52, 1.51 Hz, 2H) 2.19 (dt, J=13.99, 7.18 Hz, 1H) 2.28 (dd, J=4.64, 1.63 Hz, 2H) 3.42-3.53 (m, 1H) 4.16 (s, 2H) 4.64-4.79 (m, 2H) 5.68-5.78 (m, 1H) 6.24 (t, J=7.15 Hz, 1H) 7.08 (dd, J=7.03, 1.76 Hz, 1H) 7.31 (s, 1H) 8.53 (dd, J=7.28, 1.76 Hz, 1H) 9.18 (s, 1H) 10.81 (s, 1H) and Peak 2: (S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide, Example 66, stereochemistry arbitrarily assigned (17.7 mg, 8.0% yield, stereochemistry arbitrarily assigned). LCMS (ESI) m/z 464.3 (M+H)⁺. 19F NMR (470 MHz, CHLOROFORM-d) S ppm −75.62 (q, J=6.20 Hz, 1 F); 1H NMR (400 MHz, CHLOROFORM-d) δ ppm 0.90-0.97 (m, 2H) 1.03 (t, J=7.40 Hz, 3H) 1.16-1.24 (m, 2H) 1.61 (d, J=2.51 Hz, 3H) 1.92 (dt, J=13.93, 6.84 Hz, 1H) 2.07 (dd, J=4.52, 1.51 Hz, 2H) 2.19 (dt, J=14.12, 7.12 Hz, 1H) 2.28 (dd, J=4.64, 1.38 Hz, 2H) 3.48 (dt, J=7.59, 3.61 Hz, 1H) 4.16 (s, 2H) 4.62-4.79 (m, 2H) 5.68-5.78 (m, 1H) 6.24 (t, J=7.28 Hz, 1H) 7.07 (dd, J=7.03, 1.76 Hz, 1H) 7.30 (s, 1H) 8.53 (dd, J=7.40, 1.63 Hz, 1H) 9.18 (s, 1H) 10.81 (s, 1H).

### Examples 67 and 68: N-(1-cyclopropyl-2-oxo-1,2-dihydropyridin-3-yl)-8-fluoro-7-isopropoxy-2-((1S,4R)-1-methyl-2-oxabicyclo[2.2.1]heptan-4-yl)imidazo[1,2-a]pyridine-6-carboxamide and N-(1-cyclopropyl-2-oxo-1,2-dihydropyridin-3-yl)-8-fluoro-7-isopropoxy-2-((1R,4S)-1-methyl-2-oxabicyclo[2.2.1]heptan-4-yl)imidazo[1,2-a]pyridine-6-carboxamide

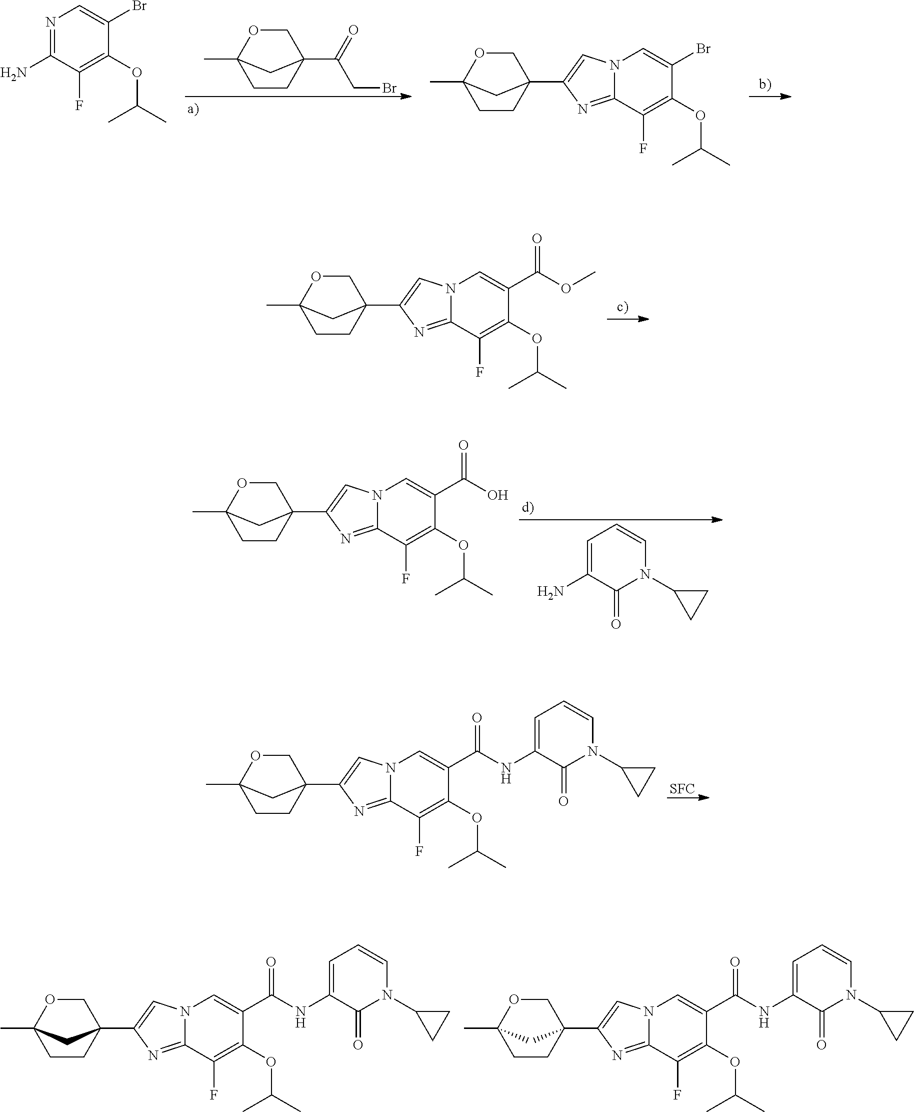

Step a: To a solution of compound 5-bromo-3-fluoro-4-isopropoxypyridin-2-amine (200 mg, 797 μmol) and 2-bromo-1-(1-methyl-2-oxabicyclo[2.2.1]heptan-4-yl)ethan-1-one (Preparation 15, 279 mg, 1.19 mmol) in t-BuOH (10 mL) was added NaHCO₃ (134 mg, 1.59 mmol) at 28° C. The mixture was stirred at 100° C. for 16 h. The mixture was concentrated in vacuo and the residue was purified by silica gel chromatography (PE:EtOAc=1:1) to give compound 6-bromo-8-fluoro-7-isopropoxy-2-(1-methyl-2-oxabicyclo[2.2.1]heptan-4-yl)imidazo[1,2-a]pyridine (256 mg, 668 μmol, 84% yield) as yellow oil. LCMS (ESI) m/z 384.7 (M+H)⁺; ¹H NMR: (500 MHz, CDCl₃) δ: 8.05 (s, 1H), 7.28 (d, J=3.0 Hz, 1H), 4.72-4.65 (m, 1H), 4.09 (dd, J₁=7.0 Hz, J₂=4.0 Hz, 1H), 3.96 (d, J=6.5 Hz, 1H), 2.20-2.13 (m, 1H), 2.09-2.07 (m, 1H), 1.99 (d, J=9.5 Hz, 1H), 1.93 (d, J=9.5 Hz, 1H), 1.86-1.78 (m, 2H), 1.47 (s, 3H), 1.40 (d, J=6.0 Hz, 6H).

Step b: To a solution of 6-bromo-8-fluoro-7-isopropoxy-2-(1-methyl-2-oxabicyclo[2.2.1]heptan-4-yl)imidazo[1,2-a]pyridine (256 mg, 668 μmol) in MeOH (30 mL) was added Pd(dppf)Cl₂ (48.9 mg, 66.8 μmol) and TEA (676 mg, 6.68 mmol) at 28° C. The mixture was degassed 3 times with CO and stirred at 80° C. under CO (50 psi) for 16 h. The mixture was concentrated in vacuo and the residue was purified by silica gel chromatography (PE:EA=1:1) to give methyl 8-fluoro-7-isopropoxy-2-(1-methyl-2-oxabicyclo[2.2.1]heptan-4-yl)imidazo[1,2-a]pyridine-6-carboxylate (230 mg, 96% yield.) as yellow oil. LCMS (ESI) m/z 363.1 (M+H)⁺; ¹H NMR: (400 MHz, CDCl₃) δ: 8.52 (s, 1H), 7.37 (d, J=3.2 Hz, 1H), 4.62-4.56 (m, 1H), 4.11 (d, J=4.4 Hz, 1H), 3.97 (d, J=6.8 Hz, 1H), 3.93 (s, 3H), 2.23-2.15 (m, 1H), 2.12-2.07 (m, 1H), 2.01 (d, J=9.6 Hz, 1H), 1.95 (d, J=9.6 Hz, 1H), 1.88-1.78 (m, 2H), 1.48 (s, 3H), 1.38 (d, J=6.4 Hz, 6H).

Step c: To a solution of methyl 8-fluoro-7-isopropoxy-2-(1-methyl-2-oxabicyclo[2.2.1]heptan-4-yl)imidazo[1,2-a]pyridine-6-carboxylate (230 mg, 634 μmol) in MeOH (9 mL) and H₂O (3 mL) was added LiOH·H₂O (79.9 mg, 1.90 mmol) at 28° C. The reaction was stirred at 28° C. for 3 h. The MeOH was evaporated and the mixture was neutralized with conc. HCl to pH=7 and dried by lyophilization to give 8-fluoro-7-isopropoxy-2-(1-methyl-2-oxabicyclo[2.2.1]heptan-4-yl)imidazo[1,2-a]pyridine-6-carboxylic acid (263 mg, crude, 84% purity) as a white solid. ¹H NMR: (400 MHz, DMSO) S: 8.27 (d, J=18.8 Hz, 1H), 7.70 (s, 1H), 4.58-4.55 (m, 1H), 3.87 (dd, J₁=6.4 Hz, J₂=3.6 Hz, 1H), 3.71 (d, J=6.0 Hz, 1H), 2.06-1.97 (m, 1H), 1.89-1.85 (m, 1H), 1.77-1.73 (m, 2H), 1.61 (d, J=11.6 Hz, 2H), 1.33 (s, 3H), 1.17 (d, J=6.0 Hz, 6H).

Step d: To a solution of compound 8-fluoro-7-isopropoxy-2-(1-methyl-2-oxabicyclo[2.2.1]heptan-4-yl)imidazo[1,2-a]pyridine-6-carboxylic acid (130 mg, 373 μmol) in pyridine (3 mL) was added 3-amino-1-cyclopropylpyridin-2(1H)-one (123 mg, 821 μmol) and T3P® (3 mL, 50% w/w in EtOAc) at 28° C. The mixture was heated at 75° C. for 16 h. The mixture was concentrated in vacuo and the residue was diluted with saturated aqueous NaHCO₃ (20 mL) to pH=7, extracted with EtOAc (3×30 mL). The combined organic layers were washed with brine (50 mL), dried (Na₂SO₄), filtered and concentrated. The residue was purified by prep-HPLC (NH₄HCO₃) to give racemic N-(1-cyclopropyl-2-oxo-1,2-dihydropyridin-3-yl)-8-fluoro-7-isopropoxy-2-(1-methyl-2-oxabicyclo[2.2.1]heptan-4-yl)imidazo[1,2-a]pyridine-6-carboxamide (120 mg, 67% yield) as a white solid. LCMS (ESI) m/z 481.0 (M+H)⁺. Racemic N-(1-cyclopropyl-2-oxo-1,2-dihydropyridin-3-yl)-8-fluoro-7-isopropoxy-2-(1-methyl-2-oxabicyclo[2.2.1]heptan-4-yl)imidazo[1,2-a]pyridine-6-carboxamide was further purified by preparative SFC (Column: DAICEL CHIRALCEL OD-H (250 mm*30 mm, 5 μm); Mobile Phase: from 50% to 50% of 0.1% NH₃H₂O ETOH; Flow Rate (80 ml/min) to give Peak 1: N-(1-cyclopropyl-2-oxo-1,2-dihydropyridin-3-yl)-8-fluoro-7-isopropoxy-2-((1S,4R)-1-methyl-2-oxabicyclo[2.2.1]heptan-4-yl)imidazo[1,2-a]pyridine-6-carboxamide, Example 67, stereochemistry arbitrarily assigned (18.1 mg, 15% yield, >99% ee). LCMS (ESI) m/z 481.2 (M+H)⁺; ¹H NMR: (500 MHz, CDCl₃) δ:10.79 (s, 1H), 8.79 (s, 1H), 8.51 (dd, J₁=7.5 Hz, J₂=2.0 Hz, 1H), 7.40 (d, J=3.0 Hz, 1H), 7.06 (dd, J₁=7.0 Hz, J₂=1.5 Hz, 1H), 6.22 (t, J=7.5 Hz, 1H), 5.00-4.94 (m, 1H), 4.11 (dd, J₁=6.5 Hz, J₂=3.0 Hz, 1H), 3.98 (d, J=6.0 Hz, 1H), 3.46-3.41 (m, 1H), 2.22-2.06 (m, 2H), 2.03-1.94 (m, 2H), 1.90-1.79 (m, 2H), 1.54 (d, J=6.0 Hz, 6H), 1.47 (s, 3H), 1.17 (q, J=7.0 Hz, 2H), 0.91 (q, J=6.5 Hz, 2H).

Peak 2, Example 68: N-(1-cyclopropyl-2-oxo-1,2-dihydropyridin-3-yl)-8-fluoro-7-isopropoxy-2-((1R,4S)-1-methyl-2-oxabicyclo[2.2.1]heptan-4-yl)imidazo[1,2-a]pyridine-6-carboxamide (26.8 mg, 22% yield, >99% ee) as a yellow solid, stereochemistry is arbitrarily assigned. LCMS (ESI) m/z 481.3 (M+H)⁺; ¹H NMR: (500 MHz, CDCl₃) δ:10.79 (s, 1H), 8.79 (s, 1H), 8.52 (d, J=7.5 Hz, 1H), 7.40 (s, 1H), 7.06 (d, J=7.0 Hz, 1H), 6.22 (t, J=7.0 Hz, 1H), 4.98-4.95 (m, 1H), 4.12-3.97 (m, 2H), 3.43 (s, 1H), 2.19-2.09 (m, 2H), 2.03-1.94 (m, 2H), 1.87-1.80 (m, 2H), 1.55 (d, J=6.0 Hz, 6H), 1.48 (s, 3H), 1.17 (d, J=7.0 Hz, 2H), 0.91 (s, 2H).

### Examples 69 and 70: (R)—N-(1-cyclopropyl-2-oxo-1,2-dihydropyridin-3-yl)-2-(1,4-dioxan-2-yl)-7-isopropoxyimidazo[1,2-a]pyridine-6-carboxamide and (S)—N-(1-cyclopropyl-2-oxo-1,2-dihydropyridin-3-yl)-2-(1,4-dioxan-2-yl)-7-isopropoxyimidazo[1,2-a]pyridine-6-carboxamide

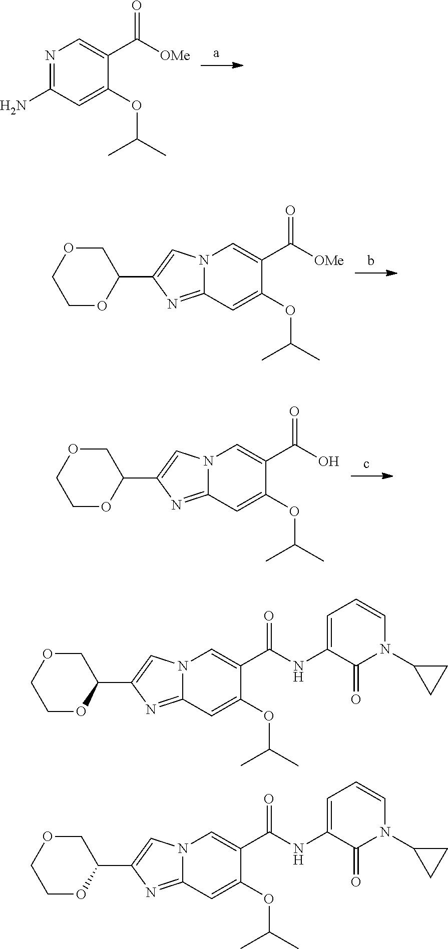

Step a: A mixture of methyl 6-amino-4-isopropoxynicotinate [preparation 2] (300 mg, 1.43 mmol), 2-chloro-1-(1,4-dioxan-2-yl)ethan-1-one (587 mg, 3.57 mmol), NaHCO₃ (359 mg, 4.28 mmol) in ACN (2.3 mL) and toluene (3.4 mL) was heated at 90° C. for 16 h. Silica and MeOH were added and the mixture was concentrated and purified by silica gel column chromatography (dry load, 24 g, 0-30% gradient of 3:1 EtOAC/EtOH in heptanes) to obtain methyl 2-(1,4-dioxan-2-yl)-7-isopropoxyimidazo[1,2-a]pyridine-6-carboxylate (175 mg, 546 μmol, 38% yield). (LCMS (ESI) m/z 321.1 (M+H)⁺.

Step b: A mixture of methyl 2-(1,4-dioxan-2-yl]-7-isopropoxy-imidazo[1,2-a]pyridine-6-carboxylate (75.0 mg, 234 μmol) and LiOH·H₂O (19.6 mg, 468 μmol) in MeOH (234 μL), THF (1.7 mL), water (421 μL) was stirred overnight at rt before being neutralized with a 4.0 M hydrochloric acid solution in dioxane. The mixture was concentrated to obtain 2-(1,4-dioxan-2-yl)-7-isopropoxyimidazo[1,2-a]pyridine-6-carboxylic acid, which was used without further purification in next reaction. Assumed 100% yield. LCMS (ESI) m/z 307.1 (M+H)+.

Step c: T3P (1.18 mmol, 699 μL, 50% purity in EtOAc) added to 2-(1,4-dioxan-2-yl]-7-isopropoxy-imidazo[1,2-a]pyridine-6-carboxylic acid (72.0 mg, 235 μmol) and 3-amino-1-cyclopropyl-pyridin-2-one (57.0 mg, 305 μmol, Hydrochloride) in Pyridine (1.6 mL) at rt. After stirring overnight, the mixture was diluted with water and extracted with DCM and then EtOAc, dried over MgSO₄, filtered, concentrated and purified via chiral SFC (CHIRALPAK IA 30×250 mm, 5 um Method: 50% EtOH w/ 0.1% DEA in CO₂ (flow rate: 100 mL/min, ABPR 120 bar, MBPR 40 psi, column temp 40 deg C.) to obtain Peak 1: (R)—N-(1-cyclopropyl-2-oxo-1,2-dihydropyridin-3-yl)-2-(1,4-dioxan-2-yl)-7-isopropoxyimidazo[1,2-a]pyridine-6-carboxamide, Example 69, stereochemistry arbitrarily assigned (11.4 mg, 11% yield). LCMS (ESI) m/z 439.2 (M+H)⁺. 1H NMR (400 MHz, CHLOROFORM-d) δ ppm 0.89-1.00 (m, 2H) 1.16-1.22 (m, 2H) 1.65 (d, J=2.51 Hz, 3H) 1.67 (d, J=2.26 Hz, 3H) 3.47 (dt, J=7.72, 3.55 Hz, 1H) 3.73-3.85 (m, 3H) 3.94-4.00 (m, 2H) 4.17 (dd, J=11.55, 2.76 Hz, 1H) 4.84-4.94 (m, 2H) 6.24 (t, J=7.15 Hz, 1H) 7.07 (dd, J=6.90, 1.88 Hz, 2H) 7.53 (s, 1H) 8.56 (dd, J=7.53, 1.76 Hz, 1H) 9.01 (s, 1H) 10.77 (s, 1H) and Peak 2: (S)—N-(1-cyclopropyl-2-oxo-1,2-dihydropyridin-3-yl)-2-(1,4-dioxan-2-yl)-7-isopropoxyimidazo[1,2-a]pyridine-6-carboxamide, Example 70, stereochemistry arbitrarily assigned (10.7 mg, 10% yield). LCMS (ESI) m/z 439.2 (M+H)⁺. 1H NMR (400 MHz, CHLOROFORM-d) δ ppm 0.89-0.97 (m, 2H) 1.18 (q, J=6.94 Hz, 2H) 1.65 (d, J=2.26 Hz, 3H) 1.66 (d, J=2.51 Hz, 3H) 3.46 (dt, J=7.59, 3.61 Hz, 1H) 3.73-3.85 (m, 3H) 3.96 (dd, J=6.78, 2.51 Hz, 2H) 4.16 (dd, J=11.55, 2.76 Hz, 1H) 4.82-4.91 (m, 2H) 6.24 (t, J=7.28 Hz, 1H) 7.02 (s, 1H) 7.06 (dd, J=7.03, 1.76 Hz, 1H) 7.53 (s, 1H) 8.56 (dd, J=7.53, 1.76 Hz, 1H) 9.01 (s, 1H) 10.77 (s, 1H).

### Example 71: N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide

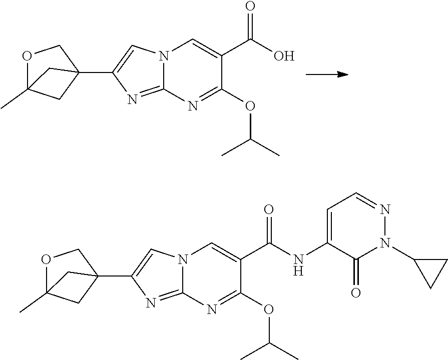

T3P (0.87 mmol, 517 μL, 50% purity in EtOAc) was added to 7-isopropoxy-2-(1-methyl-2-oxabicyclo[2.1.1]hexan-4-yl)imidazo[1,2-a]pyrimidine-6-carboxylic acid [preparation 8](100 mg, 0.217 mmol) and 4-amino-2-cyclopropyl-pyridazin-3-one (53 mg, 0.282 mmol, Hydrochloride) in pyridine (1.6 mL) at rt. After stirring for 4 h, the mixture was diluted with water and extracted with DCM and then EtOAc, dried over MgSO₄, filtered and concentrated. The crude material was purified by silica gel chromatography (12 g, 0-50% gradient of 3:1 EtOAc:EtOH in heptane) to provide N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide (12.6 mg, 13% yield). LCMS (ESI) m/z 450.9 (M+H)⁺. 1H NMR (400 MHz, CHLOROFORM-d) δ ppm 1.05 (dd, J=7.53, 2.26 Hz, 2H) 1.21 (dd, J=4.27, 2.51 Hz, 2H) 1.55 (s, 3H) 1.66 (d, J=6.02 Hz, 6H) 1.96 (dd, J=4.52, 1.51 Hz, 2H) 2.11 (dd, J=4.52, 1.51 Hz, 2H) 4.08 (s, 2H) 4.26 (dt, J=7.78, 3.64 Hz, 1H) 5.88 (quin, J=6.21 Hz, 1H) 7.76 (d, J=4.77 Hz, 1H) 8.21 (d, J=4.77 Hz, 1H) 9.16 (s, 1H) 11.05 (s, 1H).

### Example 72: 7-cyclobutoxy-N-(2-cyclopropyl-3-oxo-2,3-dihydropyridazin-4-yl)-2-(1-methyl-2-oxabicyclo[2.1.1]hexan-4-yl)imidazo[1,2-a]pyrimidine-6-carboxamide

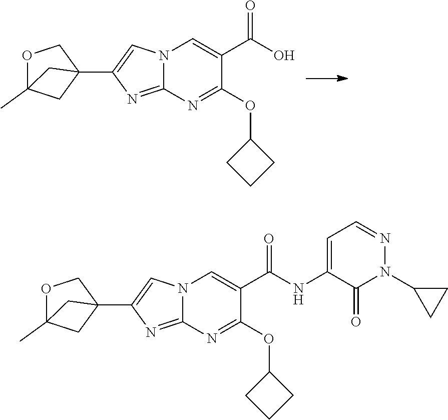 [SMILES: CC12CC(c3cn4cc(C(=O)O)c(OC5CCC5)nc4n3)(CO1)C2 ; CC12CC(c3cn4cc(C(=O)Nc5ccnn(C6CC6)c5=O)c(OC5CCC5)nc4n3)(CO1)C2]

T3P (728 μmol, 433 μL, 50% purity) added to 7-(cyclobutoxy)-2-(1-methyl-2-oxabicyclo[2.1.1]hexan-4-yl)imidazo[1,2-a]pyrimidine-6-carboxylic acid (60 mg, 182 mol) and 4-amino-2-cyclopropyl-pyridazin-3-one (44 mg, 236 μmol, Hydrochloride) in Pyridine (1.2 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 μm; Mobile phase A: MeCN; Mobile phase B: H₂O; Modifier: 0.1% TFA) to provide 7-(cyclobutoxy)-N-(2-cyclopropyl-3-oxo-pyridazin-4-yl)-2-(1-methyl-2-oxabicyclo[2.1.1]hexan-4-yl)imidazo[1,2-a]pyrimidine-6-carboxamide (9.8 mg, 9.0% yield, Trifluoroacetic acid). LCMS (ESI) m/z 463.0 (M+H)⁺. 1H NMR (500 MHz, DMSO-d6) δ ppm 0.99-1.04 (m, 2H) 1.07-1.11 (m, 2H) 1.44 (s, 3H) 1.75-1.81 (m, 3H) 1.93-2.00 (m, 1H) 2.04 (dd, J=4.27, 1.22 Hz, 2H) 2.54-2.61 (m, 4H) 3.90 (s, 2H) 4.14-4.22 (m, 1H) 5.47 (quin, J=7.17 Hz, 1H) 7.80 (s, 1H) 7.96 (d, J=4.27 Hz, 1H) 8.15 (d, J=4.88 Hz, 1H) 9.60 (s, 1H) 10.92 (s, 1H).

### Example 73: N-(2-cyclopropyl-3-oxo-2,3-dihydropyridazin-4-yl)-8-fluoro-7-isopropoxy-2-(1-methyl-2-oxabicyclo[2.1.1]hexan-4-yl)imidazo[1,2-a]pyridine-6-carboxamide

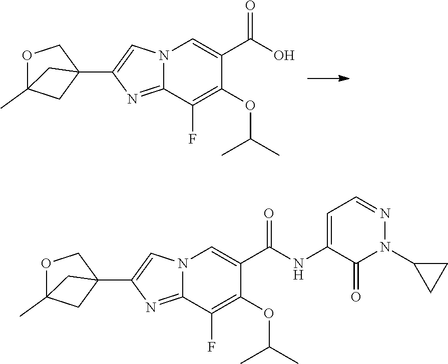

T3P (717 μmol, 427 μL, 50% purity) added to 8-fluoro-7-isopropoxy-2-(1-methyl-2-oxabicyclo[2.1.1]hexan-4-yl)imidazo[1,2-a]pyridine-6-carboxylic acid [preparation 73] (60 mg, 179 μmol) and 4-amino-2-cyclopropyl-pyridazin-3-one (44 mg, 233 μmol, Hydrochloride) in Pyridine (1.2 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 μm; Mobile phase A: MeCN; Mobile phase B: H₂O; Modifier: 0.1% TFA) to provide N-(2-cyclopropyl-3-oxo-pyridazin-4-yl)-8-fluoro-7-isopropoxy-2-(1-methyl-2-oxabicyclo[2.1.1]hexan-4-yl)imidazo[1,2-a]pyridine-6-carboxamide (16.9 mg, 16.0% yield, Trifluoroacetic acid). LCMS (ESI) m/z 467.9 (M+H)⁺. 1H NMR (500 MHz, DMSO-d6) δ ppm 0.99-1.04 (m, 2H) 1.06-1.10 (m, 2H) 1.43-1.46 (m, 3H) 1.47 (d, J=6.10 Hz, 6H) 1.80 (dd, J=4.27, 1.83 Hz, 2H) 2.05 (dd, J=4.58, 1.53 Hz, 2H) 3.92 (s, 2H) 4.14-4.21 (m, 1H) 4.80-4.89 (m, 1H) 7.95 (d, J=4.27 Hz, 1H) 8.05 (d, J=3.05 Hz, 1H) 8.16 (d, J=4.88 Hz, 1H) 9.16 (s, 1H) 10.91 (s, 1H). 19F NMR (470 MHz, DMSO-d6) δ ppm −150.06 (s, 1 F).

### Example 74: N-(2-cyclopropyl-3-oxo-2,3-dihydropyridazin-4-yl)-7-isopropoxy-2-((1S,4R)-1-methyl-2-oxabicyclo[2.2.1]heptan-4-yl)imidazo[1,2-a]pyridine-6-carboxamide

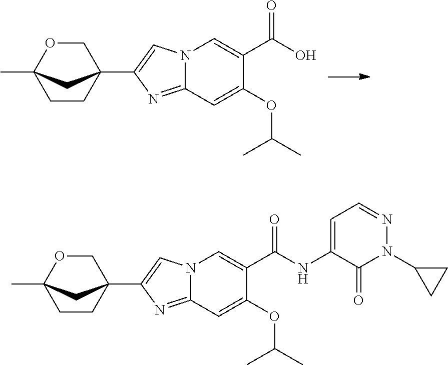

T3P (726 μmol, 432 μL, 50% purity) added to 7-isopropoxy-2-((1S,4R)-1-methyl-2-oxabicyclo[2.2.1]heptan-4-yl)imidazo[1,2-a]pyridine-6-carboxylic acid [preparation 59A](60 mg, 181 μmol) and 4-amino-2-cyclopropyl-pyridazin-3-one (44 mg, 236 μmol, Hydrochloride) in pyridine (1.2 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 μm; Mobile phase A: MeCN; Mobile phase B: H₂O; Modifier: 0.1% TFA) to provide N-(2-cyclopropyl-3-oxo-pyridazin-4-yl)-7-isopropoxy-2-[(1S,4R)-1-methyl-2-oxabicyclo[2.2.1]heptan-4-yl]imidazo[1,2-a]pyridine-6-carboxamide (79.9 mg, 75% yield, Trifluoroacetic acid). LCMS (ESI) m/z 464.0 (M+H)⁺. 1H NMR (500 MHz, DMSO-d6) δ ppm 0.99-1.04 (m, 2H) 1.05-1.10 (m, 2H) 1.41 (s, 3H) 1.58 (d, J=6.10 Hz, 6H) 1.71-1.77 (m, 1H) 1.84 (td, J=12.21, 4.27 Hz, 1H) 1.90-1.97 (m, 2H) 2.01-2.12 (m, 2H) 3.84 (d, J=6.71 Hz, 1H) 3.93 (dd, J=6.41, 3.36 Hz, 1H) 4.13-4.21 (m, 1H) 5.19-5.26 (m, 1H) 7.38 (s, 1H) 7.96 (d, J=4.88 Hz, 1H) 8.08 (s, 1H) 8.13-8.17 (m, 1H) 9.42 (s, 1H) 10.91 (s, 1H).

### Example 75: N-(2-cyclopropyl-3-oxo-2,3-dihydropyridazin-4-yl)-7-isopropoxy-2-((1R,4S)-1-methyl-2-oxabicyclo[2.2.1]heptan-4-yl)imidazo[1,2-a]pyridine-6-carboxamide

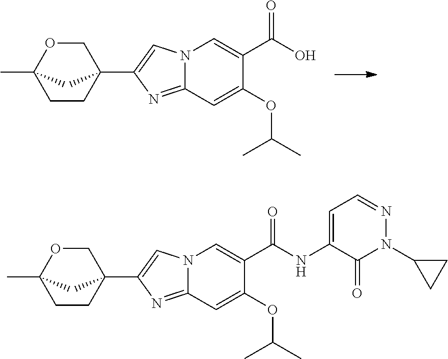

T3P (726 μmol, 432 μL, 50% purity) added to 7-isopropoxy-2-((1R,4S)-1-methyl-2-oxabicyclo[2.2.1]heptan-4-yl)imidazo[1,2-a]pyridine-6-carboxylic acid [preparation 59B](60 mg, 181 μmol) and 4-amino-2-cyclopropyl-pyridazin-3-one (44 mg, 236 μmol, Hydrochloride) in pyridine (1.2 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 μm; Mobile phase A: MeCN; Mobile phase B: H₂O; Modifier: 0.1% TFA) to provide N-(2-cyclopropyl-3-oxo-pyridazin-4-yl)-7-isopropoxy-2-[(1R,4S)-1-methyl-2-oxabicyclo[2.2.1]heptan-4-yl]imidazo[1,2-a]pyridine-6-carboxamide (65.7 mg, 62.0% yield, Trifluoroacetic acid) LCMS (ESI) m/z 464.0 (M+H)⁺. 1H NMR (500 MHz, DMSO-d6) δ ppm 0.99-1.04 (m, 2H) 1.06-1.10 (m, 2H) 1.41 (s, 3H) 1.58 (d, J=6.10 Hz, 6H) 1.68-1.78 (m, 1H) 1.84 (td, J=12.21, 4.88 Hz, 1H) 1.91-1.97 (m, 2H) 2.00-2.12 (m, 2H) 3.84 (d, J=6.10 Hz, 1H) 3.93 (dd, J=6.41, 3.36 Hz, 1H) 4.14-4.21 (m, 1H) 5.21 (dt, J=11.75, 6.03 Hz, 1H) 7.36 (s, 1H) 7.96 (d, J=4.88 Hz, 1H) 8.06 (br s, 1H) 8.16 (d, J=4.88 Hz, 1H) 9.41 (s, 1H) 10.92 (s, 1H).

### Example 76: N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide

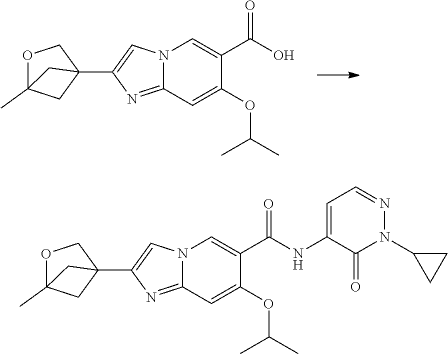

T3P (420.42 μmol, 250 μL, 50% purity) was added to 7-isopropoxy-2-(1-methyl-2-oxabicyclo[2.1.1]hexan-4-yl)imidazo[1,2-a]pyridine-6-carboxylic acid [preparation 4] (35 mg, 105 μmol) and 4-amino-2-cyclopropyl-pyridazin-3-one (19 mg, 105 μmol, HCl) in Pyridine (1.0 mL) at rt. After stirring for 4 h, the mixture was diluted with water and extracted with DCM and then EtOAc, dried over MgSO₄, filtered and concentrated. The crude material was purified by mass-directed reverse-phase HPLC (Column: Sunfire C18 100×19 mm, 5 μm; Mobile phase A: MeCN; Mobile phase B: H₂O; Modifier: 0.1% TFA) to provide N-(2-cyclopropyl-3-oxo-pyridazin-4-yl)-7-isopropoxy-2-(1-methyl-2-oxabicyclo[2.1.1]hexan-4-yl)imidazo[1,2-a]pyridine-6-carboxamide (7.8 mg, 13% yield, Trifluoroacetic acid). LCMS (ESI) m/z 449.9 (M+H)⁺. 1H NMR (500 MHz, DMSO-d6) δ ppm 0.99-1.04 (m, 2H) 1.06-1.10 (m, 2H) 1.45 (s, 3H) 1.57 (d, J=6.10 Hz, 6H) 1.83-1.90 (m, 2H) 2.07-2.14 (m, 2H) 3.93 (s, 2H) 4.17 (tt, J=7.63, 3.66 Hz, 1H) 5.19 (dt, J=11.75, 6.03 Hz, 1H) 7.36 (s, 1H) 7.96 (d, J=4.27 Hz, 1H) 8.05 (br s, 1H) 8.16 (d, J=4.27 Hz, 1H) 9.40 (s, 1H) 10.93 (s, 1H).

### Example 77: N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyrimidine-6-carboxamide

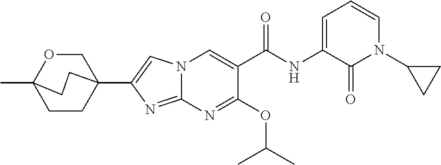 [SMILES: CC(C)Oc1nc2nc(C34CCC(C)(CC3)OC4)cn2cc1C(=O)Nc1cccn(C2CC2)c1=O]

To a solution of compound 7-isopropoxy-2-(1-methyl-2-oxabicyclo[2.2.2]octan-4-yl)imidazo[1,2-a]pyrimidine-6-carboxylic acid [preparation 45] (30.0 mg, 86.9 μmol) and 3-amino-1-cyclopropylpyridin-2(1H)-one (19.6 mg, 130 μmol) in pyridine (4 mL) was added an EtOAc solution of T₃P® (4 mL, 50% w/w). The mixture was stirred at 20° C. for 16 h. The mixture was concentrated in vacuo to give a residue, which was diluted with saturated NaHCO₃ aq. to pH=7. The resulting mixture was extracted with EtOAc (3×50 mL). The combined organic layer was washed with brine (50 mL), dried (Na₂SO₄) and filtered. The filtrate was concentrated and the residue was purified by prep-HPLC (Column: Agela DuraShell C18 150×25 mm×5 μm, water (0.05% NH₃H₂O+10 mM NH₄HCO₃)-ACN as a mobile phase, from 27% to 57%, Gradient Time=10 minutes, Flow Rate (ml/min): 25) to give N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyrimidine-6-carboxamide (26 mg, 60% yield) as a white solid. LCMS (ESI) m/z 478.3 (M+H)⁺; ¹HNMR (500 MHz, CHLOROFORM-d) 6 ppm=10.78 (s, 1H), 9.13 (s, 1H), 8.53-8.50 (m, 1H), 7.14 (s, 1H), 7.07-7.04 (m, 1H), 6.24-6.20 (m, 1H), 5.89-5.84 (m, 1H), 4.12 (s, 2H), 3.49-3.44 (m, 1H), 2.25-2.19 (m, 2H), 2.02-1.95 (m, 4H), 1.79-1.72 (m, 2H), 1.63 (d, J=6.4 Hz, 6H), 1.20-1.15 (m, 5H), 0.94-0.90 (m, 2H).

### Examples 78 and 79: (R)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide and (S)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

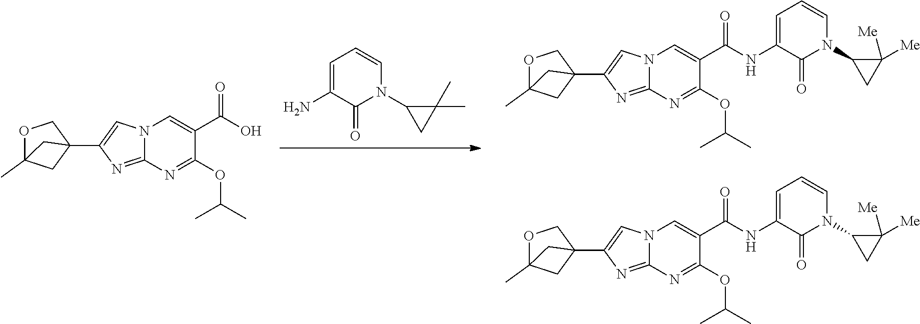

To a solution of 3-amino-1-(2,2-dimethylcyclopropyl)pyridin-2(1H)-one hydrochloride [preparation 46], (60 mg, 279.47 μmol) and 7-isopropoxy-2-(1-methyl-2-oxabicyclo[2.1.1]hexan-4-yl)imidazo[1,2-a]pyrimidine-6-carboxylic acid [preparation 8](90 mg, 284 μmol) in pyridine (1 mL) was added T3P® (1 mL, 50% w/w in EtOAc). The mixture was stirred at 20° C. for 1 h. The reaction mixture was diluted with saturated aq. NaHCO₃ (30 mL) and the mixture was extracted with EtOAc (3×20 mL). The combined organic layers were washed with brine (30 mL), dried (Na₂SO₄) and filtered. The filtrate was concentrated and the residue was purified by prep-TLC (EtOAc) to give racemic N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (60 mg, 45% yield) as an off-white solid. LCMS (ESI) m/z 478.3 (M+H)⁺.

The enantiomers of N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (60 mg, 125.64 μmol) were separated by SFC (Column: DAICEL CHIRALCEL OD (250 mm*30 mm, 10 um); Mobile Phase: from 50% to 50% of 0.1% NH₃·H₂O MeOH; Flow Rate (mL/min): 80; Column temp: 35° C.) to yield Peak 1, Example 78, (R)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide as an off-white solid (27.5 mg, 45% yield, >99% ee, retention time=2.620 min, stereochemistry arbitrarily assigned). LCMS (ESI) m/z 478.3 (M+H)⁺; ¹HNMR (500 MHz, DMSO) δ ppm=10.75 (s, 1H), 9.46 (s, 1H), 8.42 (d, J=7.5 Hz, 1H), 7.71 (s, 1H), 7.32 (d, J=8.5 Hz, 1H), 6.30 (t, J=7.0 Hz, 1H), 5.65-5.55 (m, 1H), 3.87 (s, 2H), 3.20-3.10 (m, 1H), 2.00-1.90 (m, 2H), 1.80-1.70 (m, 2H), 1.54 (d, J=5.5 Hz, 6H), 1.43 (s, 3H), 1.24 (s, 3H), 1.10-1.00 (m, 1H), 0.90-0.80 (m, 1H), 0.76 (s, 3H).

Peak 2, Example 79, (S)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide as an off-white solid (28 mg, 47% yield, >99% ee, retention time=3.215 min, stereochemistry arbitrarily assigned). LCMS (ESI) m/z 478.3 (M+H)⁺; ¹HNMR (500 MHz, DMSO) δ ppm=10.75 (s, 1H), 9.46 (s, 1H), 8.42 (d, J=7.5 Hz, 1H), 7.71 (s, 1H), 7.32 (d, J=8.5 Hz, 1H), 6.30 (t, J=7.0 Hz, 1H), 5.65-5.55 (m, 1H), 3.87 (s, 2H), 3.20-3.10 (m, 1H), 2.00-1.90 (m, 2H), 1.80-1.70 (m, 2H), 1.54 (d, J=5.5 Hz, 6H), 1.43 (s, 3H), 1.24 (s, 3H), 1.10-1.00 (m, 1H), 0.90-0.80 (m, 1H), 0.76 (s, 3H).

### Example 80: 2-(3-cyanobicyclo[1.1.1]pentan-1-yl)-N-(1-cyclopropyl-2-oxo-1,2-dihydropyridin-3-yl)-7-isopropoxyimidazo[1,2-a]pyridine-6-carboxamide

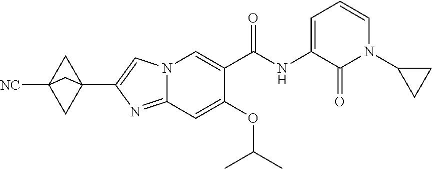 [SMILES: CC(C)Oc1cc2nc(C34CC(C#N)(C3)C4)cn2cc1C(=O)Nc1cccn(C2CC2)c1=O]

To the mixture of 3-amino-1-cyclopropyl-pyridin-2-one (26.4 mg, 141 μmol, Hydrochloride), 2-(3-cyano-1-bicyclo[1.1.1]pentanyl)-7-isopropoxy-imidazo[1,2-a]pyridine-6-carboxylic acid (40.0 mg, 128 μmol), and HATU (53.9 mg, 141 μmol) in DMF (1 mL) was added Hunig's base (66.4 mg, 514 μmol, 89.5 μL). The mixture was stirred at 22° C. overnight. The reaction mixture was partitioned between EtOAc/water. The aqueous layer was extracted with EtOAc (5 mL×3). The combined organic phases were concentrated and purified by reverse phase prep HPLC (SunFire C18 column, 60 mL/min flow rate, MeCN/H₂O/0.1% TFA; Gradient (% organic): 5-50). 2-(3-cyano-1-bicyclo[1.1.1]pentanyl)-N-(1-cyclopropyl-2-oxo-3-pyridyl)-7-isopropoxy-imidazo[1,2-a]pyridine-6-carboxamide (21.7 mg, 48.9 μmol, 38.1% yield) was obtained as a white solid. LCMS m/z=444.3 [M+H]⁺; ¹H NMR (500 MHz, DMSO-d₆) δ: 0.83-0.94 (m, 2H) 1.01-1.08 (m, 2H) 1.53 (d, J=6.10 Hz, 6H) 2.59 (s, 6H) 3.35 (br s, 1H) 5.06 (dt, J=11.75, 6.03 Hz, 1H) 6.24-6.36 (m, 1H) 7.21 (s, 1H) 7.34 (dd, J=6.71, 1.83 Hz, 1H) 7.87 (s, 1H) 8.44 (dd, J=7.32, 1.83 Hz, 1H) 9.24 (s, 1H) 10.72 (s, 1H).

### Example 81: racemic N-(1-trans-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

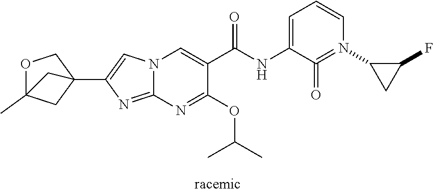

To a mixture of 7-isopropoxy-2-(1-methyl-2-oxabicyclo[2.1.1]hexan-4-yl)imidazo[1,2-a]pyrimidine-6-carboxylic acid [preparation 8] (59.0 mg, 186 μmol), rac-(Trans)-3-amino-1-(2-fluorocyclopropyl)pyridin-2(1H)-one [preparation 61] (41.9 mg, 204.51 μmol, Hydrochloride) in pyridine (1 mL) was added T3P® (592 mg, 930 μmol, 553 μL, 50% w/w) at rt. The vial contained this reaction mixture was capped and stirred at rt for 2 h. The mixture was diluted with EtOAc and water. The aqueous phase was extracted with EtOAc (3×5 mL). The combined organic layers were dried over anhydrous MgSO4 and filtered. The filtrate was evaporated in vacuo to afford a crude residual, which was purified by reverse phase HPLC purification system (C18 column, 5-60% acetonitrile in water with 0.1% TFA) to afford racemic N-(1-trans-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (75.0 mg, 129 μmol, 69.4% yield, Trifluoroacetic acid) as an off white solid. LCMS m/z=468.1 [M+H]⁺; ¹H NMR (400 MHz, MeOH-d₄) δ:1.48-1.58 (m, 1H) 1.53-1.55 (m, 3H) 1.72 (dd, J=6.27, 1.00 Hz, 6H) 1.76-1.85 (m, 1H) 1.97-2.02 (m, 2H) 2.20-2.25 (m, 2H) 3.78-3.88 (m, 1H) 4.06 (s, 2H) 4.82-5.03 (m, 1H) 5.84 (quin, J=6.21 Hz, 1H) 6.41 (t, J=7.15 Hz, 1H) 7.30 (dd, J=7.03, 1.76 Hz, 1H) 7.90-7.97 (m, 1H) 8.57 (dd, J=7.53, 1.76 Hz, 1H) 9.56-9.67 (m, 1H).

### Examples 82 and 83: N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide and N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

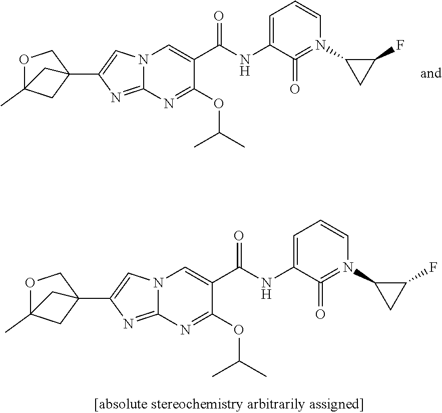

Chiral SFC (Daicel Chiralpak AD-H; 250×30 mm, 5 μm; 30% EtOH+0.1% Et₂NH in CO₂) was used to purify racemic N-(1-trans-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide [Example 81] to afford: Peak 1: Example 82: N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, stereochemistry arbitrarily assigned (9.8 mg, 11.3%, >99% ee); LCMS m/z=467.9 [M+H]⁺; ¹H NMR (400 MHz, MeOH-d₄) δ: 1.49-1.56 (m, 4H) 1.67 (dd, J=6.27, 1.51 Hz, 6H) 1.78-1.84 (s, 1H) 1.86-1.90 (m, 2H) 2.13 (dd, J=4.77, 1.51 Hz, 2H) 3.76-3.81 (m, 1H) 4.02 (s, 2H) 4.96-5.02 (m, 1H) 5.75-5.86 (m, 1H) 6.39 (t, J=7.15 Hz, 1H) 7.26 (d, J=5.02 Hz, 1H) 7.63 (s, 1H) 8.57 (dd, J=7.66, 1.63 Hz, 1H) 9.41 (s, 1H).

Peak 2: Example 83: N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, stereochemistry arbitrarily assigned (9.3 mg, 10.7%, 85% ee); LCMS m/z=467.9 [M+H]⁺; ¹H NMR (400 MHz, MeOH-d₄) δ: 1.48-1.54 (m, 4H) 1.67 (dd, J=6.27, 1.51 Hz, 6H) 1.79-1.84 (s, 1H) 1.86-1.91 (m, 2H) 2.13 (dd, J=4.77, 1.51 Hz, 2H) 3.75-3.80 (m, 1H) 4.03 (s, 2H) 4.95-5.01 (m, 1H) 5.77-5.86 (m, 1H) 6.39 (t, J=7.15 Hz, 1H) 7.25 (d, J=5.02 Hz, 1H) 7.62 (s, 1H) 8.57 (dd, J=7.66, 1.63 Hz, 1H) 9.42 (s, 1H).

### Example 84: N-(1-(2,2-difluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

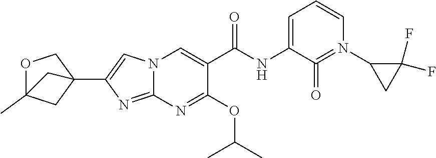

To a vial charged with 7-isopropoxy-2-(1-methyl-2-oxabicyclo[2.1.1]hexan-4-yl)imidazo[1,2-a]pyrimidine-6-carboxylic acid [preparation 8] (43.6 mg, 138 μmol), 3-amino-1-(2,2-difluorocyclopropyl)pyridin-2(1H)-one hydrochloride [preparation 60] (25.6 mg, 138 μmol) and pyridine (1 mL) was added an EtOAc solution of T3P® (437 mg, 687 mol, 409 μL, 50% w/w) at rt. The vial was sealed and maintained at rt for 2 h. The mixture was diluted with EtOAc and water. The aqueous phase was extracted with EtOAc (5 mL×3). The combined organic layers were dried over anhydrous MgSO4 and filtered. The filtrate was evaporated in vacuo to afford a crude residual, which was purified by reverse phase HPLC purification system (C18 column, 10-90% acetonitrile in water with 0.1% TFA) to afford N-[l-[(1S)-2,2-difluorocyclopropyl]-2-oxo-3-pyridyl]-7-isopropoxy-2-(1-methyl-2-oxabicyclo[2.1.1]hexan-4-yl)imidazo[1,2-a]pyrimidine-6-carboxamide (24.1 mg, 40.2 μmol, 29.2% yield, Trifluoroacetic acid) as an off white solid. LCMS m/z=486.2 [M+H]⁺; ¹H NMR (400 MHz, MeOH-d₄) δ:1.49-1.58 (m, 3H) 1.71 (dd, J=6.27, 2.76 Hz, 6H) 1.95-2.04 (m, 2H) 2.05-2.18 (m, 1H) 2.18-2.33 (m, 3H) 4.01-4.11 (m, 3H) 5.83 (spt, J=6.23 Hz, 1H) 6.44 (t, J=7.28 Hz, 1H) 7.36 (dd, J=7.15, 1.63 Hz, 1H) 7.88-7.98 (m, 1H) 8.59 (dd, J=7.53, 1.76 Hz, 1H) 9.56-9.67 (m, 1H).

### Example 85: (rac)-Cis-7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide

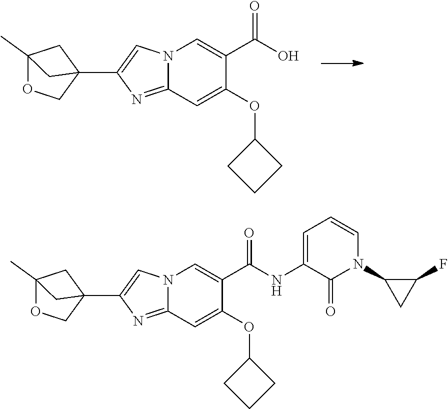

Prepared in a manner similar to 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide, but coupling 7-(cyclobutoxy)-2-(1-methyl-2-oxabicyclo[2.1.1]hexan-4-yl)imidazo[1,2-a]pyridine-6-carboxylic acid [preparation 33] to (rac)-Cis-3-amino-1-(2-fluorocyclopropyl)pyridin-2(1H)-one [preparation 63]. LCMS m/z=479.4 [M+H]⁺; 1H NMR (500 MHz, METHANOL-d4) Shift 9.39 (s, 1H), 8.62 (dd, J=1.68, 7.48 Hz, 1H), 8.03 (s, 1H), 7.39-7.47 (m, 1H), 7.09 (s, 1H), 6.42 (t, J=7.25 Hz, 1H), 5.16-5.25 (m, 1H), 4.89-4.95 (m, 1H), 4.04 (s, 2H), 3.39-3.47 (m, 1H), 2.62-2.74 (m, 4H), 2.17-2.23 (m, 2H), 2.04-2.15 (m, 1H), 1.99 (dd, J=1.68, 4.58 Hz, 2H), 1.83-1.95 (m, 1H), 1.47-1.62 (m, 5H).

### Example 86: (rac)-Cis-2-(2-oxabicyclo[2.1.1]hexan-4-yl)-7-cyclobutoxy-N-(1-(2-fluorocyclopropyl)-2-oxo-1,2-dihydropyridin-3-yl)imidazo[1,2-a]pyrimidine-6-carboxamide

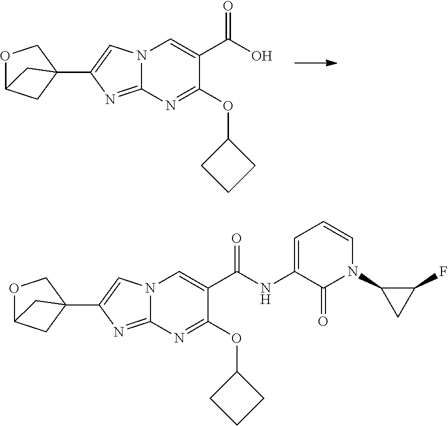

Prepared in a manner similar to 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide, but coupling 2-(2-oxabicyclo[2.1.1]hexan-4-yl)-7-cyclobutoxyimidazo[1,2-a]pyrimidine-6-carboxylic acid [preparation 30] to (rac)-Cis-3-amino-1-(2-fluorocyclopropyl)pyridin-2(1H)-one [preparation 63]. LCMS m/z=466.5 [M+H]⁺; ¹H NMR (500 MHz, METHANOL-d4) δ 9.62 (s, 1H), 8.61 (dd, J=1.68, 7.48 Hz, 1H), 7.94 (s, 1H), 7.45 (br d, J=7.17 Hz, 1H), 6.43 (t, J=7.25 Hz, 1H), 5.64 (quin, J=7.17 Hz, 1H), 4.91-4.94 (m, 1H), 4.70 (s, 1H), 3.99 (s, 2H), 3.40-3.46 (m, 2H), 2.62-2.77 (m, 4H), 2.30-2.36 (m, 2H), 2.01-2.12 (m, 1H), 1.98 (dd, J=1.68, 4.73 Hz, 2H), 1.81-1.93 (m, 1H), 1.50-1.63 (m, 2H).

### Example 87: (rac)-Cis-2-(2-oxabicyclo[2.1.1]hexan-4-yl)-N-(1-(2-fluorocyclopropyl)-2-oxo-1,2-dihydropyridin-3-yl)-7-isopropoxyimidazo[1,2-a]pyrimidine-6-carboxamide

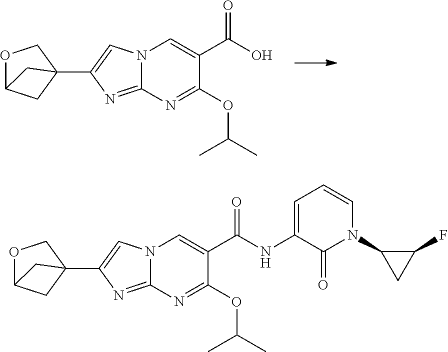 [SMILES: CC(C)Oc1nc2nc(C34COC(C3)C4)cn2cc1C(=O)O ; CC(C)Oc1nc2nc(C34COC(C3)C4)cn2cc1C(=O)Nc1cccn([C@@H]2C[C@@H]2F)c1=O]

Prepared in a manner similar to 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide, but coupling 2-(2-oxabicyclo[2.1.1]hexan-4-yl)-7-isopropoxyimidazo[1,2-a]pyrimidine-6-carboxylic acid [preparation 26] to (rac)-Cis-3-amino-1-(2-fluorocyclopropyl)pyridin-2(1H)-one [preparation 63]. ¹H NMR (500 MHz, METHANOL-d₄) δ 9.61 (s, 1H), 8.61 (dd, J=1.68, 7.32 Hz, 1H), 7.94 (s, 1H), 7.44 (d, J=7.02 Hz, 1H), 6.42 (t, J=7.25 Hz, 1H), 5.82 (quin, J=6.26 Hz, 1H), 4.90-5.07 (m, 2H), 4.00 (s, 2H), 3.39-3.47 (m, 1H), 2.34 (d, J=4.88 Hz, 2H), 1.99 (dd, J=1.68, 4.73 Hz, 2H), 1.70 (dd, J=1.98, 6.26 Hz, 6H), 1.52-1.61 (m, 2H).

### Examples 88 and 89: 2-(2-oxabicyclo[2.1.1]hexan-4-yl)-N-(1-((1R,2S)-2-fluorocyclopropyl)-2-oxo-1,2-dihydropyridin-3-yl)-7-isopropoxyimidazo[1,2-a]pyrimidine-6-carboxamide and 2-(2-oxabicyclo[2.1.1]hexan-4-yl)-N-(1-((1S,2R)-2-fluorocyclopropyl)-2-oxo-1,2-dihydropyridin-3-yl)-7-isopropoxyimidazo[1,2-a]pyrimidine-6-carboxamide

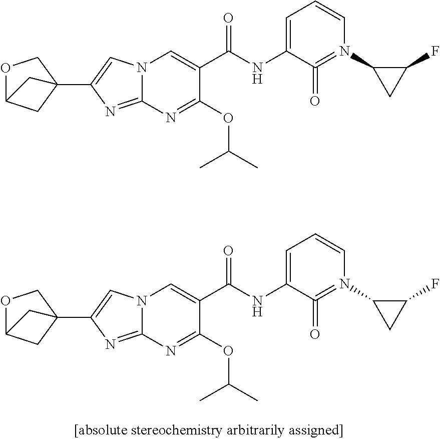

(rac)-Cis-2-(2-oxabicyclo[2.1.1]hexan-4-yl)-N-(1-(2-fluorocyclopropyl)-2-oxo-1,2-dihydropyridin-3-yl)-7-isopropoxyimidazo[1,2-a]pyrimidine-6-carboxamide [Example 87] was subjected to Prep-SFC (Daicel Chiralpak AD-H; 250×30 mm, 5 μm; 40% EtOH+0.1% Et₂NH in CO₂) to afford enantiomerically enriched samples:

Peak 1, Example 88: 2-(2-oxabicyclo[2.1.1]hexan-4-yl)-N-(1-((1R,2S)-2-fluorocyclopropyl)-2-oxo-1,2-dihydropyridin-3-yl)-7-isopropoxyimidazo[1,2-a]pyrimidine-6-carboxamide, stereochemistry arbitrarily assigned, (>99% ee): LCMS m/z=454.4 [M+H]⁺; ¹H NMR (500 MHz, METHANOL-d₄) δ 9.40 (s, 1H), 8.60 (dd, J=1.68, 7.32 Hz, 1H), 7.65 (s, 1H), 7.40 (d, J=6.87 Hz, 1H), 6.40 (t, J=7.25 Hz, 1H), 5.74-5.83 (m, 1H), 4.94-5.06 (m, 1H), 4.90-4.91 (m, 1H), 3.96 (s, 2H), 3.38-3.43 (m, 1H), 2.25 (d, J=5.04 Hz, 2H), 1.87 (dd, J=1.68, 4.73 Hz, 2H), 1.64 (dd, J=2.06, 6.18 Hz, 6H), 1.51-1.60 (m, 2H)

Peak 2, Example 89: 2-(2-oxabicyclo[2.1.1]hexan-4-yl)-N-(1-((1S,2R)-2-fluorocyclopropyl)-2-oxo-1,2-dihydropyridin-3-yl)-7-isopropoxyimidazo[1,2-a]pyrimidine-6-carboxamide, stereochemistry arbitrarily assigned, (94% ee): LCMS m/z=454.4 [M+H]⁺; ¹H NMR (500 MHz, METHANOL-d₄) δ 9.40 (s, 1H), 8.60 (dd, J=1.75, 7.40 Hz, 1H), 7.65 (s, 1H), 7.40 (d, J=6.10 Hz, 1H), 6.40 (t, J=7.25 Hz, 1H), 5.78 (quin, J=6.26 Hz, 1H), 4.91-5.06 (m, 1H), 4.89-4.91 (m, 1H), 3.96 (s, 2H), 3.38-3.43 (m, 1H), 2.25 (d, J=4.88 Hz, 2H), 1.87 (dd, J=1.83, 4.73 Hz, 2H), 1.64 (dd, J=1.98, 6.26 Hz, 6H), 1.51-1.60 (m, 2H).

### Example 90: (rac)-Cis-N-(1-(2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide

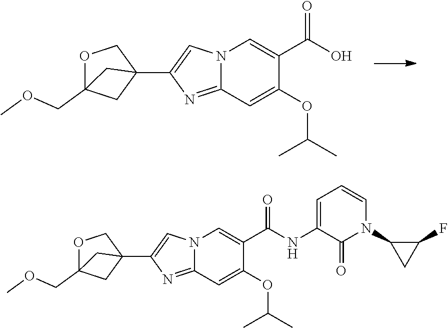

Prepared in a manner similar to 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide, but coupling 7-isopropoxy-2-[1-(methoxymethyl)-2-oxabicyclo[2.1.1]hexan-4-yl]imidazo[1,2-a]pyridine-6-carboxylic acid [Example 11, Step b] to (rac)-Cis-3-amino-1-(2-fluorocyclopropyl)pyridin-2(1H)-one [preparation 63]. LCMS m/z=498.5 [M+H]⁺; ¹H NMR (500 MHz, METHANOL-d4) Shift 9.36-9.42 (m, 1H), 8.59 (dd, J=1.68, 7.48 Hz, 1H), 7.64 (s, 1H), 7.39 (br d, J=6.87 Hz, 1H), 6.39 (t, J=7.25 Hz, 1H), 5.70-5.83 (m, 1H), 5.00-5.07 (m, 1H), 4.89-4.94 (m, 1H), 4.03 (s, 2H), 3.70 (s, 2H), 3.42 (s, 4H), 2.18 (dd, J=1.53, 4.58 Hz, 2H), 1.92 (dd, J=1.60, 4.50 Hz, 2H), 1.63 (dd, J=1.68, 6.26 Hz, 6H), 1.48-1.59 (m, 2H).

### Example 91: (rac)-Cis-8-fluoro-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

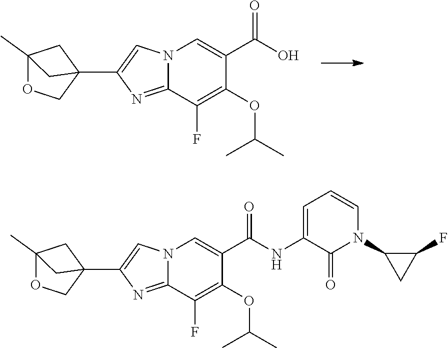

Prepared in a manner similar to 7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide, but coupling 8-fluoro-7-isopropoxy-2-(1-methyl-2-oxabicyclo[2.1.1]hexan-4-yl)imidazo[1,2-a]pyridine-6-carboxylic acid [preparation 73] to (rac)-Cis-3-amino-1-(2-fluorocyclopropyl)pyridin-2(1H)-one [preparation 63]. LCMS m/z=485.4 [M+H]⁺; ¹H NMR (500 MHz, METHANOL-d₄) δ 9.01 (s, 1H), 8.60 (dd, J=1.60, 7.40 Hz, 1H), 7.87 (d, J=2.90 Hz, 1H), 7.41 (d, J=7.00 Hz, 1H), 6.40 (t, J=7.25 Hz, 1H), 4.89-4.96 (m, 2H), 4.03 (s, 2H), 3.37-3.43 (m, 1H), 2.15 (dd, J=1.53, 4.58 Hz, 2H), 1.90 (dd, J=1.60, 4.50 Hz, 2H), 1.54-1.59 (m, 1H), 1.47-1.54 (m, 10H).

### Example 92 and 93: 8-fluoro-N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide and 8-fluoro-N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

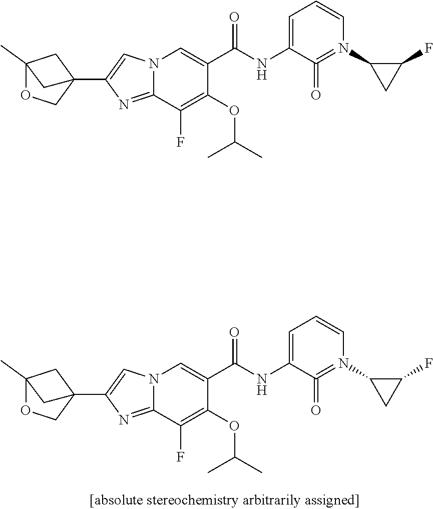

(rac)-Cis-8-fluoro-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide [Example 91] was subjected to Prep-SFC (Daicel Chiralpak AD-H; 250×30 mm, 5 μm; 40% EtOH+0.1% Et₂NH in CO₂) to afford enantiomerically enriched samples:

Peak 1, Example 92: 8-fluoro-N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide, stereochemistry arbitrarily assigned (>99% ee); LCMS m/z=485.4 [M+H]⁺; ¹H NMR (500 MHz, METHANOL-d4) Shift 9.03 (d, J=1.07 Hz, 1H), 8.62 (dd, J=1.68, 7.48 Hz, 1H), 7.89 (d, J=3.05 Hz, 1H), 7.43 (d, J=6.87 Hz, 1H), 6.43 (t, J=7.25 Hz, 1H), 4.94-4.98 (m, 1H), 4.05 (s, 2H), 3.39-3.46 (m, 1H), 2.17 (dd, J=1.68, 4.58 Hz, 2H), 1.88-1.96 (m, 3H), 1.57-1.61 (m, 1H), 1.53-1.56 (m, 7H), 1.52 (s, 3H)

Peak 2, Example 93: 8-fluoro-N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide, stereochemistry arbitrarily assigned (98% ee); LCMS m/z=485.5 [M+H]⁺; ¹H NMR (500 MHz, METHANOL-d4) Shift 9.03 (d, J=1.07 Hz, 1H), 8.62 (dd, J=1.68, 7.48 Hz, 1H), 7.89 (d, J=3.05 Hz, 1H), 7.43 (d, J=5.95 Hz, 1H), 6.43 (t, J=7.17 Hz, 1H), 4.93-4.99 (m, 1H), 4.05 (s, 2H), 3.40-3.45 (m, 1H), 2.17 (dd, J=1.68, 4.58 Hz, 2H), 1.87-1.97 (m, 3H), 1.57-1.62 (m, 1H), 1.55 (t, J=5.04 Hz, 7H), 1.52 (s, 3H).

### Example 94: (rac)-Cis-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

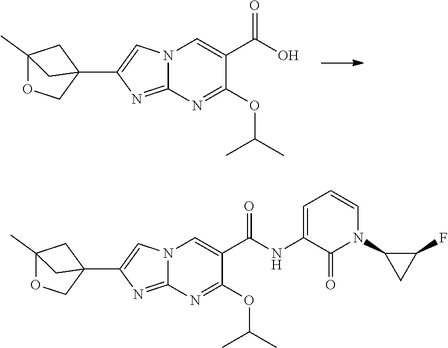

Prepared in a manner similar to N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, but using 7-isopropoxy-2-(1-methyl-2-oxabicyclo[2.1.1]hexan-4-yl)imidazo[1,2-a]pyrimidine-6-carboxylic acid [preparation 8] and (rac)-Cis-3-amino-1-(2-fluorocyclopropyl)pyridin-2(1H)-one [preparation 63]. LCMS m/z=468.4 [M+H]⁺; ¹H NMR (500 MHz, METHANOL-d4) δ 11.50 (s, 1H), 10.29 (s, 1H), 9.28 (dd, J=1.68, 7.32 Hz, 1H), 8.53 (s, 1H), 8.27 (d, J=7.02 Hz, 1H), 7.17 (t, J=7.25 Hz, 1H), 6.42 (quin, J=6.18 Hz, 1H), 5.79-6.00 (m, 1H), 4.69 (s, 2H), 4.26-4.33 (m, 1H), 3.71 (s, 1H), 3.55 (s, 1H), 2.78-2.84 (m, 1H), 2.78-2.84 (m, 2H), 2.57 (dd, J=1.60, 4.35 Hz, 2H), 2.36 (dd, J=3.59, 6.18 Hz, 6H), 2.24 (s, 3H).

### Example 95: (rac)-trans-7-isopropoxy-2-(1-methyl-2-oxabicyclo[2.1.1]hexan-4-yl)-N-(1-(2-methylcyclopropyl)-2-oxo-1,2-dihydropyridin-3-yl)imidazo[1,2-a]pyrimidine-6-carboxamide

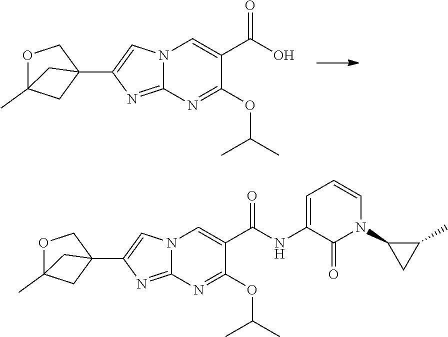

Prepared in a manner similar to N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, but using 7-isopropoxy-2-(1-methyl-2-oxabicyclo[2.1.1]hexan-4-yl)imidazo[1,2-a]pyrimidine-6-carboxylic acid [preparation 8] and trans-racemic 3-amino-1-(2-methylcyclopropyl)pyridin-2(1H)-one hydrochloride [preparation 76]. LCMS m/z=464.5 [M+H]⁺; ¹H NMR (500 MHz, DMSO-d₆) δ 10.63 (s, 1H), 9.63 (s, 1H), 8.40 (dd, J=1.68, 7.32 Hz, 1H), 7.92 (br s, 1H), 7.37 (dd, J=1.60, 6.94 Hz, 1H), 6.31 (t, J=7.17 Hz, 1H), 5.63 (quin, J=6.14 Hz, 1H), 3.92 (s, 2H), 3.18-3.22 (m, 1H), 2.07-2.14 (m, 2H), 1.83 (br d, J=4.27 Hz, 2H), 1.59 (dd, J=2.52, 6.18 Hz, 6H), 1.44 (s, 3H), 1.17-1.26 (m, 4H), 1.07-1.12 (m, 1H), 0.83-0.92 (m, 1H).

### Example 96: 7-((S)-sec-butoxy)-N-(1-cis-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide

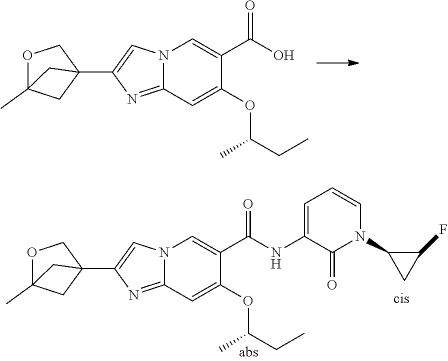

Prepared as a mixture of diastereomers in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but using (S)-7-(sec-butoxy)-2-(1-methyl-2-oxabicyclo[2.1.1]hexan-4-yl)imidazo[1,2-a]pyridine-6-carboxylic acid [preparation 44] and (rac)-Cis-3-amino-1-(2-fluorocyclopropyl)pyridin-2(1H)-one [preparation 63]. LCMS m/z=481.5 [M+H]⁺; ¹H NMR (500 MHz, METHANOL-d4) Shift 9.16 (s, 1H), 8.52-8.68 (m, 1H), 7.73 (s, 1H), 7.40 (br d, J=6.56 Hz, 1H), 7.02 (s, 1H), 6.30-6.46 (m, 1H), 4.71-4.82 (m, 1H), 4.03 (s, 2H), 3.39-3.48 (m, 1H), 2.04-2.26 (m, 3H), 1.83-1.97 (m, 3H), 1.56-1.67 (m, 4H), 1.43-1.56 (m, 5H), 0.98-1.10 (m, 3H).

### Example 97: (rac)-Cis-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

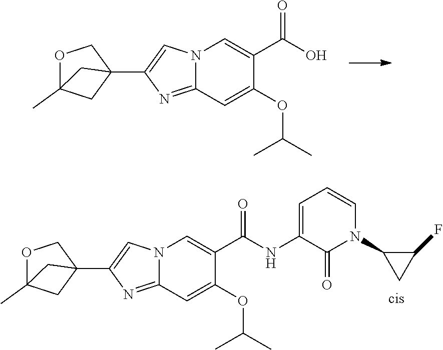

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19] but using 7-isopropoxy-2-(1-methyl-2-oxabicyclo[2.1.1]hexan-4-yl)imidazo[1,2-a]pyridine-6-carboxylic acid [preparation 4] and (rac)-Cis-3-amino-1-(2-fluorocyclopropyl)pyridin-2(1H)-one [preparation 63]. LCMS m/z=467.5 [M+H]⁺; ¹H NMR (500 MHz, METHANOL-d4) Shift 9.13 (s, 1H), 8.59 (dd, J=1.68, 7.48 Hz, 1H), 7.71 (s, 1H), 7.38 (d, J=7.02 Hz, 1H), 7.00 (s, 1H), 6.39 (t, J=7.25 Hz, 1H), 4.96-5.05 (m, 2H), 4.01 (s, 2H), 3.36-3.46 (m, 1H), 2.06-2.15 (m, 2H), 1.83-1.92 (m, 2H), 1.62 (d, J=6.10 Hz, 6H), 1.46-1.59 (m, 6H).

### Example 98: trans-7-isopropoxy-2-((1S,4R)-1-methyl-2-oxabicyclo[2.2.1]heptan-4-yl)-N-(1-(2-methylcyclopropyl)-2-oxo-1,2-dihydropyridin-3-yl)imidazo[1,2-a]pyridine-6-carboxamide

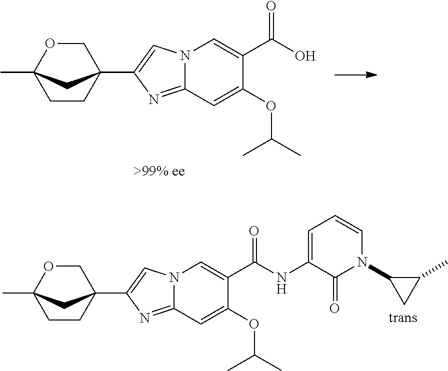

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-isopropoxy-2-((1S,4R)-1-methyl-2-oxabicyclo[2.2.1]heptan-4-yl)imidazo[1,2-a]pyridine-6-carboxylic acid [preparation 59A] and Trans-racemic 3-amino-1-(2-methylcyclopropyl)pyridin-2(1H)-one hydrochloride [preparation 76]. (Product is mixture of two diastereomers). LCMS m/z=477.5 [M+H]⁺; ¹H NMR (500 MHz, DMSO-d₆) δ 10.77 (s, 1H), 9.17 (s, 1H), 8.42 (dd, J=1.60, 7.40 Hz, 1H), 7.80 (s, 1H), 7.30 (dd, J=1.68, 7.02 Hz, 1H), 7.20 (s, 1H), 6.27 (t, J=7.17 Hz, 1H), 4.99-5.07 (m, 1H), 3.93 (dd, J=3.43, 6.33 Hz, 1H), 3.75 (d, J=6.41 Hz, 1H), 3.15-3.22 (m, 1H), 2.69 (s, 7H), 1.52 (dd, J=3.20, 5.95 Hz, 6H), 1.37 (s, 3H), 1.18-1.20 (m, 3H), 1.05-1.11 (m, 1H), 0.82-0.89 (m, 1H).

### Example 99: N-(1-(2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide

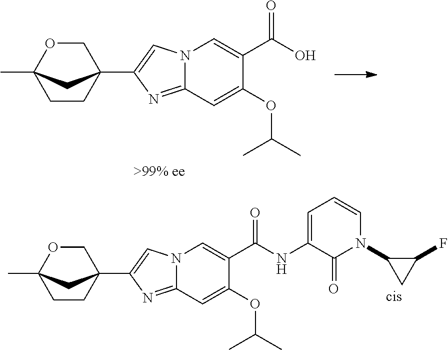

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting 7-isopropoxy-2-((1S,4R)-1-methyl-2-oxabicyclo[2.2.1]heptan-4-yl)imidazo[1,2-a]pyridine-6-carboxylic acid [preparation 59A] and Cis-3-amino-1-(2-fluorocyclopropyl)pyridin-2(1H)-one [preparation 63]. (Product is mixture of two diastereomers). LCMS m/z=481.5 [M+H]⁺; ¹H NMR (500 MHz, METHANOL-d4) δ 9.36 (s, 1H), 8.60 (dd, J=1.83, 7.48 Hz, 1H), 7.99 (s, 1H), 7.43 (d, J=7.02 Hz, 1H), 7.27 (s, 1H), 6.41 (t, J=7.25 Hz, 1H), 5.13-5.22 (m, 1H), 4.00-4.05 (m, 1H), 3.95-3.99 (m, 1H), 3.38-3.43 (m, 1H), 2.12-2.22 (m, 2H), 2.05 (s, 2H), 1.85-1.98 (m, 2H), 1.67 (d, J=6.10 Hz, 6H), 1.51-1.60 (m, 2H), 1.49 (s, 3H).

### Example 100: N-(1-(2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide

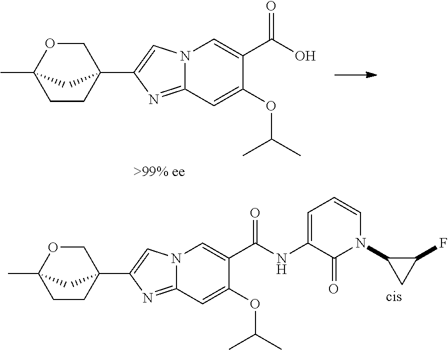

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-isopropoxy-2-((1R,4S)-1-methyl-2-oxabicyclo[2.2.1]heptan-4-yl)imidazo[1,2-a]pyridine-6-carboxylic acid [preparation 59B] and (rac)-Cis-3-amino-1-(2-fluorocyclopropyl)pyridin-2(1H)-one [preparation 63]. (Product is a mixture of two diastereomers) LCMS m/z=481.5 [M+H]⁺; ¹H NMR (500 MHz, METHANOL-d₄) δ 9.36 (s, 1H), 8.60 (dd, J=1.60, 7.40 Hz, 1H), 7.99 (s, 1H), 7.43 (d, J=6.71 Hz, 1H), 7.28 (s, 1H), 6.41 (t, J=7.17 Hz, 1H), 5.12-5.23 (m, 1H), 5.01-5.06 (m, 1H), 4.02 (dd, J=3.05, 6.71 Hz, 1H), 3.95-3.99 (m, 1H), 3.38-3.44 (m, 1H), 2.12-2.24 (m, 2H), 2.05 (s, 2H), 1.83-1.97 (m, 2H), 1.68 (d, J=5.95 Hz, 6H), 1.51-1.60 (m, 2H), 1.49 (s, 3H).

### Example 101: (rac)-Cis-N-(1-(2-fluorocyclopropyl)-2-oxo-1,2-dihydropyridin-3-yl)-7-isopropoxy-2-(1-methyl-2-oxabicyclo[2.2.1]heptan-4-yl)imidazo[1,2-a]pyrimidine-6-carboxamide

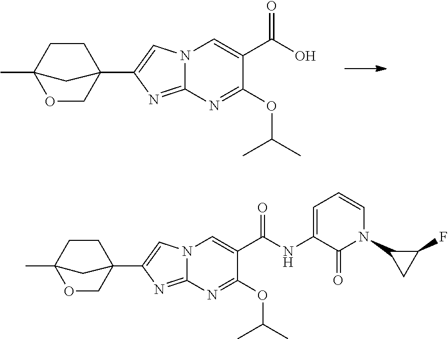

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-isopropoxy-2-(1-methyl-2-oxabicyclo[2.2.1]heptan-4-yl)imidazo[1,2-a]pyrimidine-6-carboxylic acid [preparation 64] and (rac)-Cis-3-amino-1-(2-fluorocyclopropyl)pyridin-2(1H)-one [preparation 63]. (Product is a mixture of 4 diastereomers). LCMS m/z=482.5 [M+H]⁺; ¹H NMR (500 MHz, METHANOL-d4) δ 9.38 (s, 1H), 8.59 (dd, J=1.68, 7.48 Hz, 1H), 7.58 (s, 1H), 7.39 (d, J=6.71 Hz, 1H), 6.39 (t, J=7.25 Hz, 1H), 5.71-5.81 (m, 1H), 4.04 (dd, J=3.36, 6.41 Hz, 1H), 3.91 (d, J=6.56 Hz, 1H), 3.38-3.43 (m, 1H), 2.12-2.21 (m, 1H), 2.01-2.08 (m, 2H), 1.92-1.98 (m, 3H), 1.84-1.91 (m, 1H), 1.77-1.84 (m, 1H), 1.63 (dd, J=1.98, 6.26 Hz, 6H), 1.45 (s, 3H), 1.28 (s, 2H).

### Example 102: (S)—N-(1-cyclopropyl-2-oxo-1,2-dihydropyridin-3-yl)-7-((1,1-difluoropropan-2-yl)oxy)-2-(1-methyl-2-oxabicyclo[2.1.1]hexan-4-yl)imidazo[1,2-a]pyridine-6-carboxamide

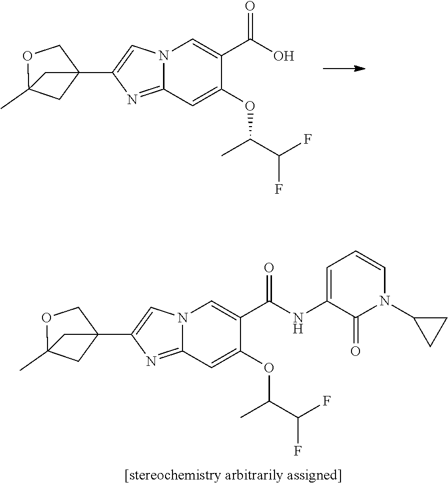 [SMILES: C[C@H](Oc1cc2nc(C34COC(C)(C3)C4)cn2cc1C(=O)O)C(F)F ; CC(Oc1cc2nc(C34COC(C)(C3)C4)cn2cc1C(=O)Nc1cccn(C2CC2)c1=O)C(F)F]

7-[(1S)-2,2-difluoro-1-methyl-ethoxy]-2-(1-methyl-2-oxabicyclo[2.1.1]hexan-4-yl)imidazo[1,2-a]pyridine-6-carboxylic acid [preparation 62A] (15 mg, 42 μmol) was dissolved in DMF (425 μL), HATU (18 mg, 46.8 μmol) and DIPEA (22 μL, 127 mol,) were added. 3-amino-1-cyclopropyl-pyridin-2-one (10 mg, 51 μmol, Hydrochloride) followed. The reaction was then stirred at room temperature overnight. The crude reaction mixture was purified by reverse-phase HPLC (Waters SunFire Prep C18 5 um OBD 19×100 mm gradient 5-45% ACN water with 1% TFA). LCMS m/z=485.3 [M+H]⁺; ¹H NMR (500 MHz, DMSO-d₆) δ 10.48 (s, 1H), 9.33 (s, 1H), 8.41 (dd, J=1.68, 7.32 Hz, 1H), 8.04 (br s, 1H), 7.51 (s, 1H), 7.37 (dd, J=1.68, 7.02 Hz, 1H), 6.40-6.71 (m, 1H), 6.33 (t, J=7.17 Hz, 1H), 5.40 (br s, 1H), 3.92 (s, 2H), 3.51 (dt, J=3.66, 7.63 Hz, 1H), 2.08 (br d, J=3.51 Hz, 2H), 1.78-1.87 (m, 2H), 1.57 (d, J=6.26 Hz, 3H), 1.44 (s, 3H), 1.01-1.09 (m, 2H), 0.86-0.96 (m, 2H).

### Example 103: (rac)-Cis-7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

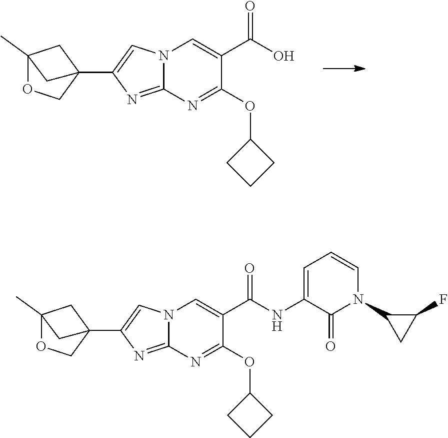

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-cyclobutoxy-2-(1-methyl-2-oxabicyclo[2.1.1]hexan-4-yl)imidazo[1,2-a]pyrimidine-6-carboxylic acid [preparation 56] and (rac)-Cis-3-amino-1-(2-fluorocyclopropyl)pyridin-2(1H)-one [preparation 63]. LCMS m/z=480.5 [M+H]⁺; ¹H NMR (500 MHz, METHANOL-d₄) δ 9.58 (br s, 1H), 8.57-8.65 (m, 1H), 7.83-7.91 (m, 1H), 7.44 (d, J=6.56 Hz, 1H), 6.42 (t, J=7.17 Hz, 1H), 5.56-5.70 (m, 1H), 5.02-5.07 (m, 1H), 4.02 (s, 2H), 2.62-2.78 (m, 4H), 2.18 (br d, J=4.58 Hz, 2H), 2.02-2.12 (m, 1H), 1.96 (br d, J=4.43 Hz, 2H), 1.81-1.92 (m, 1H), 1.44-1.61 (m, 6H).

### Example 104: 7-cyclopropoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide

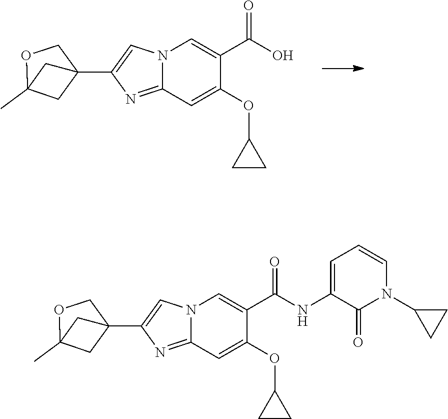 [SMILES: CC12CC(c3cn4cc(C(=O)O)c(OC5CC5)cc4n3)(CO1)C2 ; CC12CC(c3cn4cc(C(=O)Nc5cccn(C6CC6)c5=O)c(OC5CC5)cc4n3)(CO1)C2]

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-cyclopropoxy-2-(1-methyl-2-oxabicyclo[2.1.1]hexan-4-yl)imidazo[1,2-a]pyridine-6-carboxylic acid [preparation 51] and 3-amino-1-cyclopropylpyridin-2(1H)-one.]. LCMS m/z=447.3 [M+H]⁺; ¹H NMR (500 MHz, DMSO-d₆) δ 10.50 (s, 1H), 9.31 (br s, 1H), 8.40 (dd, J=1.68, 7.32 Hz, 1H), 8.03 (br s, 1H), 7.49 (s, 1H), 7.35 (dd, J=1.68, 7.02 Hz, 1H), 6.26-6.36 (m, 1H), 3.89-3.94 (m, 2H), 3.49 (s, 1H), 2.53-2.58 (m, 1H), 2.08 (br d, J=3.36 Hz, 2H), 1.76-1.87 (m, 2H), 1.41-1.47 (m, 3H), 1.12-1.20 (m, 2H), 1.04-1.09 (m, 2H), 0.98-1.03 (m, 2H), 0.89-0.94 (m, 2H).

### Example 105: (rac)-Cis-7-cyclopropoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide

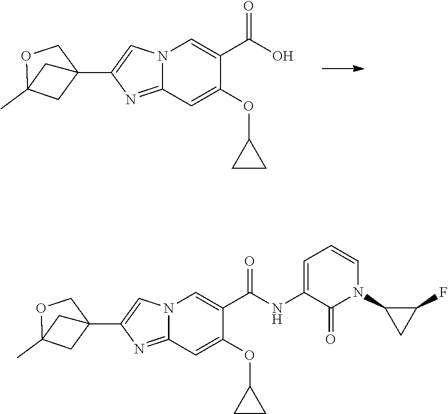 [SMILES: CC12CC(c3cn4cc(C(=O)O)c(OC5CC5)cc4n3)(CO1)C2 ; CC12CC(c3cn4cc(C(=O)Nc5cccn([C@@H]6C[C@@H]6F)c5=O)c(OC5CC5)cc4n3)(CO1)C2]

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-cyclopropoxy-2-(1-methyl-2-oxabicyclo[2.1.1]hexan-4-yl)imidazo[1,2-a]pyridine-6-carboxylic acid [preparation 51] and (rac)-Cis-3-amino-1-(2-fluorocyclopropyl)pyridin-2(1H)-one [preparation 63]. LCMS m/z=465.4 [M+H]⁺; ¹H NMR (500 MHz, METHANOL-d₄) δ 9.38 (s, 1H), 8.58 (dd, J=1.60, 7.40 Hz, 1H), 8.06 (s, 1H), 7.62 (s, 1H), 7.43 (br d, J=7.02 Hz, 1H), 6.42 (t, J=7.25 Hz, 1H), 4.89-4.93 (m, 1H), 4.31-4.43 (m, 1H), 4.05 (s, 2H), 3.41 (br d, J=5.65 Hz, 1H), 2.18-2.25 (m, 2H), 2.00 (dd, J=1.60, 4.50 Hz, 2H), 1.53-1.60 (m, 2H), 1.53 (s, 3H), 1.28-1.32 (m, 2H), 1.06-1.14 (m, 2H).

### Example 106: (rac)-Cis-7-cyclobutoxy-N-(1-(2-fluorocyclopropyl)-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide

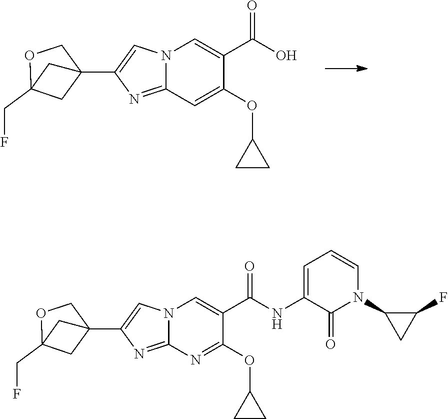 [SMILES: O=C(O)c1cn2cc(C34COC(CF)(C3)C4)nc2cc1OC1CC1 ; O=C(Nc1cccn([C@@H]2C[C@@H]2F)c1=O)c1cn2cc(C34COC(CF)(C3)C4)nc2nc1OC1CC1]

Prepared in a manner similar to (S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide [Example 19], but starting with 7-cyclobutoxy-2-(1-(fluoromethyl)-2-oxabicyclo[2.1.1]hexan-4-yl)imidazo[1,2-a]pyridine-6-carboxylic acid [preparation 74] and (rac)-Cis-3-amino-1-(2-fluorocyclopropyl)pyridin-2(1H)-one [preparation 63]. LCMS m/z=498.4 [M+H]⁺; ¹H NMR (500 MHz, METHANOL-d4) Shift 9.61 (d, J=1.37 Hz, 1H), 8.61 (dd, J=1.68, 7.48 Hz, 1H), 7.91-7.97 (m, 1H), 7.45 (d, J=6.56 Hz, 1H), 6.43 (t, J=7.17 Hz, 1H), 5.59-5.68 (m, 1H), 4.90-4.95 (m, 1H), 4.73 (s, 1H), 4.64 (s, 1H), 4.09 (s, 2H), 3.40-3.47 (m, 1H), 2.60-2.77 (m, 4H), 2.33 (br d, J=4.58 Hz, 2H), 2.01-2.11 (m, 3H), 1.80-1.92 (m, 1H), 1.49-1.64 (m, 2H).

### Examples 107 and 108: (R)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide and (S)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide

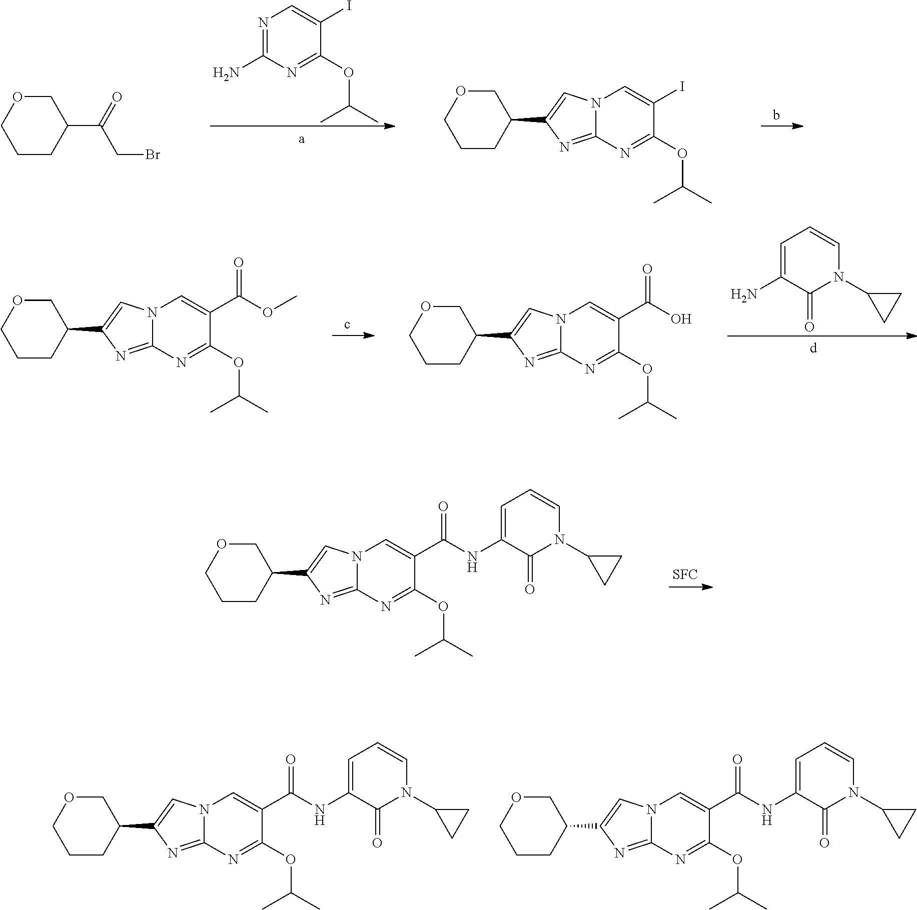

Step a: To a solution of compound 5-iodo-4-isopropoxypyrimidin-2-amine (417 mg, 1.50 mmol, prepared as described in WO2020/150626) and compound 2-bromo-1-(tetrahydro-2H-pyran-3-yl)ethan-1-one (310 mg, 1.50 mmol) in t-BuOH (10 mL) was added NaHCO₃ (252 mg, 2.99 mmol) at 25° C. The mixture was stirred at 100° C. for 16 h. The mixture was concentrated and the residue was purified by silica-gel chromatography (PE:EtOAc=2:3) to give compound 3 (218 mg, 562.99 μmol, 37.61% yield.) as yellow oil. LCMS (ESI) m/z 387.9 (M+H)⁺. ¹H NMR (500 MHz, CDCl₃) δ:8.44 (s, 1H), 7.05 (s, 1H), 5.51-5.45 (m, 1H), 3.93 (d, J=11.5 Hz, 1H), 3.61 (t, J=10.5 Hz, 1H), 3.53-3.48 (m, 1H), 3.03-2.97 (m, 1H), 2.13-2.10 (m, 1H), 1.93-1.87 (m, 1H), 1.78-1.67 (m, 3H), 1.41 (d, J=6.0 Hz, 6H).

Step b: To a solution of 6-iodo-7-isopropoxy-2-(tetrahydro-2H-pyran-3-yl)imidazo[1,2-a]pyrimidine (210 mg, 542 μmol) in MeOH (25 mL) was added Pd(dppf)Cl₂ (39.7 mg, 54.2 mol) and TEA (549 mg, 5.42 mmol). The mixture was degassed with CO (3×) and subsequently stirred at 80° C. under CO (50 psi) for 16 h. Solvent was evaporated and the residue was purified by silica gel chromatography (PE:EA=1:1) to give methyl 7-isopropoxy-2-(tetrahydro-2H-pyran-3-yl)imidazo[1,2-a]pyrimidine-6-carboxylate (120 mg, 69% yield.) as a yellow oil. ¹H NMR (400 MHz, CDCl₃) δ: 8.84 (s, 1H), 7.16 (s, 1H), 5.63-5.57 (m, 1H), 4.15 (dd, J₁=10.8 Hz, J₂=2.8 Hz, 1H), 3.95 (d, J=11.6 Hz, 1H), 3.91 (s, 3H), 3.65 (t, J=10.8 Hz, 1H), 3.56-3.50 (m, 1H), 3.07-3.00 (m, 1H), 2.14 (J₁=12.8 Hz, J₂=3.6 Hz, 1H), 1.98-1.88 (m, 1H), 1.78-1.70 (m, 2H), 1.44 (d, J=6.4 Hz, 6H).

Step c: To a solution of methyl 7-isopropoxy-2-(tetrahydro-2H-pyran-3-yl)imidazo[1,2-a]pyrimidine-6-carboxylate (125 mg, 391 μmol) in MeOH (6 mL) and H₂O (2 mL) was added LiOH·H₂O (49.3 mg, 1.17 mmol) at 25° C. The mixture was stirred at 25° C. for 3 h and then the MeOH was evaporated under vacuum. The resulting mixture was neutralized with conc. HCl to pH=7 and dried by lyophilization to give 7-isopropoxy-2-(tetrahydro-2H-pyran-3-yl)imidazo[1,2-a]pyrimidine-6-carboxylic acid (210 mg) as a white solid, which was used in the next step without further purification. LCMS (ESI) m/z 305.9 (M+H)⁺. ¹H NMR (400 MHz, DMSO) S: 9.49 (s, 1H), 7.80 (s, 1H), 5.43-5.37 (m, 1H), 3.97 (dd, J₁=10.8 Hz, J₂=3.2 Hz, 1H), 3.78-3.76 (m, 1H), 3.13 (s, 2H), 3.04-3.02 (m, 1H), 2.06-2.04 (m, 1H), 1.82-1.75 (m, 1H), 1.66-1.57 (m, 2H), 1.38 (d, J=6.4 Hz, 6H).

Step d: To a solution of 7-isopropoxy-2-(tetrahydro-2H-pyran-3-yl)imidazo[1,2-a]pyrimidine-6-carboxylic acid (109 mg, 357 μmol) in an EtOAc solution of T3P® (2.5 mL, 50% w/w) was added 3-amino-1-cyclopropylpyridin-2(1H)-one (107 mg, 714 μmol) and pyridine (2.5 mL) at 25° C. The reaction was stirred at 25° C. for 2 h. The solution was concentrated and the residue was diluted with saturated aqueous NaHCO₃ (20 mL) to pH=7, extracted with DCM (3×30 mL). The combined organic layers were washed with brine (50 mL), dried (Na₂SO₄) and filtered. The filtrate was concentrated and the residue was purified by base modified reverse phase HPLC to give racemic N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide (100 mg, 229 μmol) as a white solid. LCMS (ESI) m/z 438.2 (M+H)⁺.

Racemic N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide was purified by prep-SFC (Column: DAICEL CHIRALCEL OD-H (250 mm*30 mm, 5 um)); Mobile Phase: from 40% to 40% of 0.1% NH₃H₂O MEOH; Flow Rate (ml/min): 80) to give (R)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide, Example 107 (19.0 mg, 19% yield, >99% ee) as a white solid. Stereochemistry arbitrarily assigned. LCMS (ESI) m/z 438.0 (M+H)⁺. ¹H NMR (400 MHz, CDCl₃) δ: 10.78 (s, 1H), 9.14 (s, 1H), 8.52 (d, J=8.0 Hz, 1H), 7.24 (s, 1H), 7.06 (d, J=8.0 Hz, 1H), 6.22 (t, J=8.0 Hz, 1H), 5.88-5.82 (m, 1H), 4.19-4.17 (m, 1H), 3.96 (d, J=12.0 Hz, 1H), 3.66 (t, J=12.0 Hz, 1H), 3.57-3.51 (m, 1H), 3.50-3.44 (m, 1H), 3.06 (t, J=12.0 Hz, 1H), 2.16 (d, J=12.0 Hz, 1H), 1.98-1.93 (m, 1H), 1.74 (s, 2H), 1.64 (d, J=8.0 Hz, 6H), 1.19-1.15 (m, 2H), 0.93-0.90 (m, 1H).

Peak 2 from the purification was arbitrarily assigned as (S)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide, Example 108 (22 mg, 22% yield, >99% ee) as a white solid. LCMS (ESI) m/z 438.1 (M+H)⁺. ¹H NMR (400 MHz, CDCl₃) δ: 10.76 (s, 1H), 9.11 (s, 1H), 8.50 (d, J=8.0 Hz, 1H), 7.22 (s, 1H), 7.04 (d, J=8.0 Hz, 1H), 6.20 (t, J=8.0 Hz, 1H), 5.85-5.79 (m, 1H), 4.17-4.14 (m, 1H), 3.94 (d, J=12.0 Hz, 1H), 3.64 (t, J=12.0 Hz, 1H), 3.54-3.49 (m, 1H), 3.47-3.41 (m, 1H), 3.6-3.01 (m, 1H), 2.14 (d, J=8.0 Hz, 1H), 1.96-1.89 (m, 1H), 1.77-1.72 (m, 2H), 1.62 (d, J=8.0 Hz, 6H), 1.18-1.13 (m, 2H), 0.92-0.88 (m, 2H).

### Examples 109 and 110: (R)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide and (S)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide

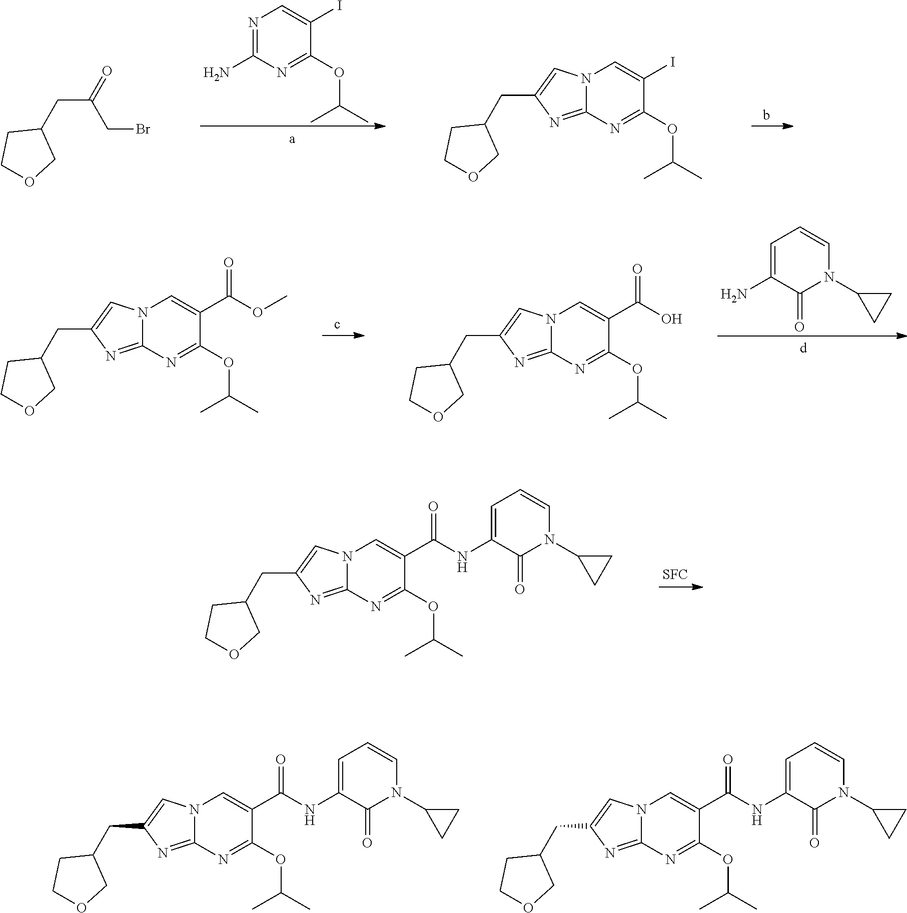

Step a: To a mixture of 1-bromo-3-(tetrahydrofuran-3-yl)propan-2-one (624 mg, 2.24 mmol, prepared as described in WO2020/150626) and 5-iodo-4-isopropoxypyrimidin-2-amine (463 mg, 2.24 mmol, prepared as described in WO2020/150626) in t-BuOH (15 mL) was added NaHCO₃ (376 mg, 4.47 mmol) at 25° C. The mixture was stirred at 90° C. for 16 h and then concentrated in vacuo to give a residue, which was purified by silica gel chromatography (PE/EtOAc=2/3) to give 6-iodo-7-isopropoxy-2-((tetrahydrofuran-3-yl)methyl)imidazo[1,2-a]pyrimidine (164 mg, 19% yield) as a yellow oil. LCMS (ESI) m/z 388.0 (M+H)⁺. ¹H NMR (400 MHz, CHLOROFORM-d) δ ppm=8.45 (s, 1H), 7.03 (s, 1H), 5.49 (td, J=6.1, 12.4 Hz, 1H), 3.89 (dd, J=5.5, 8.2 Hz, 2H), 3.81-3.76 (m, 1H), 3.51 (dd, J=5.6, 8.3 Hz, 1H), 2.86-2.73 (m, 3H), 2.14-2.01 (m, 1H), 1.72-1.64 (m, 1H), 1.42 (d, J=6.1 Hz, 6H).

Step b: To a solution of 6-iodo-7-isopropoxy-2-((tetrahydrofuran-3-yl)methyl)imidazo[1,2-a]pyrimidine (155 mg, 400 μmol) in MeOH (30 mL) was added Pd(dppf)Cl₂ (29.3 mg, 40.0 mol) and TEA (405 mg, 4.00 mmol). The mixture was degassed with CO (3×) and then stirred at 80° C. under CO (50 Psi) for 24 h. The mixture was concentrated and the residue was purified by silica gel chromatography (PE/EA=1/1) to give methyl 7-isopropoxy-2-((tetrahydrofuran-3-yl)methyl)imidazo[1,2-a]pyrimidine-6-carboxylate (75 mg, 59% yield) as a yellow oil. LCMS (ESI) m/z 320.1 (M+H)⁺. ¹H NMR (400 MHz, CHLOROFORM-d) 6 ppm=8.84 (s, 1H), 7.12 (s, 1H), 5.59 (td, J=6.1, 12.4 Hz, 1H), 4.19-4.07 (m, 1H), 3.93-3.88 (m, 5H), 3.78 (q, J=7.6 Hz, 1H), 3.52 (dd, J=5.5, 8.4 Hz, 1H), 2.82 (br s, 1H), 2.11-2.05 (m, 1H), 1.75-1.63 (m, 2H), 1.44 (d, J=6.1 Hz, 6H).

Step c: To a solution of methyl 7-isopropoxy-2-((tetrahydrofuran-3-yl)methyl)imidazo[1,2-a]pyrimidine-6-carboxylate (70 mg, 219 μmol) in MeOH (6 mL) and H₂O (2 mL) was added LiOH·H₂O (27.6 mg, 658 μmol) at 25° C. The reaction was stirred at 25° C. for 2 h and then the MeOH was evaporated in vacuo. The mixture was neutralized with conc. HCl to pH=7 and dried by lyophilization to give 7-isopropoxy-2-((tetrahydrofuran-3-yl)methyl)imidazo[1,2-a]pyrimidine-6-carboxylic acid (154 mg) as a yellow solid which was used in the next step without further purification. LCMS (ESI) m/z 306.1 (M+H)⁺. ¹H NMR: (400 MHz, DMSO-d₆) δ ppm=8.65 (s, 1H), 7.38 (s, 1H), 5.30 (td, J=6.1, 12.2 Hz, 1H), 4.50-4.25 (m, 1H), 3.79-3.59 (m, 3H), 2.65-2.53 (m, 3H), 2.03-1.90 (m, 1H), 1.64-1.52 (m, 1H), 1.30 (d, J=6.4 Hz, 6H).

Step d: To a solution of 7-isopropoxy-2-((tetrahydrofuran-3-yl)methyl)imidazo[1,2-a]pyrimidine-6-carboxylic acid (67 mg, 219 μmol) and 3-amino-1-cyclopropylpyridin-2(1H)-one hydrochloride (50 mg, 268 μmol) in pyridine (2 mL) was added an EtOAc solution of T₃P® (2 mL, 50% w/w). The mixture was stirred at 25° C. for 1 h and then quenched dropwise with saturated aqueous NaHCO₃ (20 mL). The mixture was extracted with EtOAc (3×20 mL). The combined organic layers were washed with brine (20 mL), dried (Na₂SO4), filtered and concentrated. The residue was purified by prep-HPLC (Column: Boston Prime C18 (150 mm*30 mm, 5 um), water (0.05% NH₃H₂O+10 mM NH₄HCO₃)-ACN as a mobile phase, from 33% to 63%, Gradient Time=10 minutes, Flow Rate (ml/min): 25) to give racemic N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide (70 mg, 73% yield) as a white solid. LCMS (ESI) m/z 438.2 (M+H)⁺. ¹H NMR (500 MHz, CHLOROFORM-d) δ ppm=10.78 (s, 1H), 9.13 (s, 1H), 8.52 (dd, J=1.8, 7.4 Hz, 1H), 7.21 (s, 1H), 7.06 (dd, J=1.8, 6.9 Hz, 1H), 6.23 (t, J=7.2 Hz, 1H), 5.90-5.78 (m, 1H), 3.95-3.88 (m, 2H), 3.79 (q, J=7.5 Hz, 1H), 3.54 (dd, J=5.6, 8.5 Hz, 1H), 3.49-3.43 (m, 1H), 2.90-2.75 (m, 3H), 2.15-2.05 (m, 1H), 1.73-1.70 (m, 1H), 1.64 (d, J=6.3 Hz, 6H), 1.18 (q, J=7.0 Hz, 2H), 0.96-0.89 (m, 2H).

Racemic N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide (70 mg, 160.00 mol) was separated by SFC (Column: Phenomenex Lux Cellulose-4 (250*30 mm, 5 um); Mobile Phase: from 45% to 45% of 0.1% NH₃·H₂O MeOH; Flow Rate (mL/min): 80; Column temp: 40° C.) to give as Peak 1: (R)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide, Example 109, stereochemistry arbitrarily assigned (27 mg, 39% yield, >99% ee) as a white solid. LCMS (ESI) m/z 438.2 (M+H)⁺. ¹H NMR (400 MHz, CHLOROFORM-d) δ ppm=10.78 (s, 1H), 9.13 (s, 1H), 8.57-8.49 (m, 1H), 7.21 (s, 1H), 7.06 (d, J=6.8 Hz, 1H), 6.22 (t, J=7.2 Hz, 1H), 5.84 (td, J=6.2, 12.5 Hz, 1H), 3.96-3.87 (m, 2H), 3.79 (q, J=7.5 Hz, 1H), 3.54 (br dd, J=5.4, 8.3 Hz, 1H), 3.51-3.43 (m, 1H), 2.85-2.79 (m, 3H), 2.10 (br dd, J=5.5, 12.3 Hz, 1H), 1.74-1.68 (m, 1H), 1.64 (d, J=6.4 Hz, 6H), 1.24-1.14 (m, 2H), 0.96-0.89 (m, 2H).

Peak 2 from the purification was arbitrarily assigned as (S)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide, Example 110 (34.4 mg, 49% yield, >97% ee) as a white solid. LCMS (ESI) m/z 438.2 (M+H)⁺. ¹H NMR (400 MHz, CHLOROFORM-d) δ ppm=10.78 (s, 1H), 9.13 (s, 1H), 8.52 (dd, J=1.5, 7.3 Hz, 1H), 7.21 (s, 1H), 7.06 (dd, J=1.6, 7.0 Hz, 1H), 6.22 (t, J=7.2 Hz, 1H), 5.84 (td, J=6.2, 12.5 Hz, 1H), 3.96-3.88 (m, 2H), 3.79 (q, J=7.6 Hz, 1H), 3.54 (br dd, J=5.4, 8.3 Hz, 1H), 3.50-3.43 (m, 1H), 2.85-2.78 (m, 3H), 2.10 (br dd, J=5.4, 12.5 Hz, 1H), 1.70 (br dd, J=5.9, 12.7 Hz, 1H), 1.64 (d, J=6.1 Hz, 6H), 1.21-1.15 (m, 2H), 0.95-0.89 (m, 2H).

### Examples 111 and 112: N-(1-cyclopropyl-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide and N-(1-cyclopropyl-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide

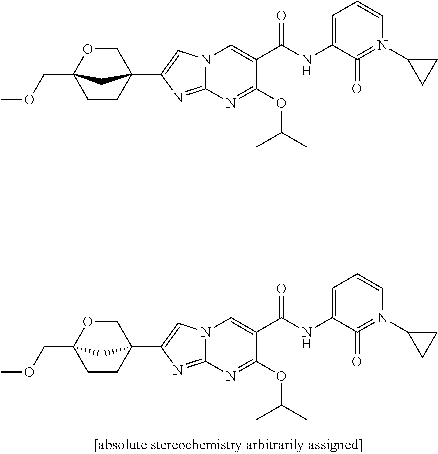

N-(1-Cyclopropyl-2-oxo-1,2-dihydropyridin-3-yl)-7-isopropoxy-2-(1-(methoxymethyl)-2-oxabicyclo[2.2.1]heptan-4-yl)imidazo[1,2-a]pyrimidine-6-carboxamide (Example 53, 53 mg, 0.107 mmol) was purified by SFC: CHIRALPAK IB 30×250 mm, 5 um, mobile phase: 50% MeOH in CO₂ (flow rate: 100 mL/min, ABPR 120 bar, MBPR 40 psi, 40° C.) to provide:

Peak 1: N-(1-cyclopropyl-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide (16.7 mg, 100% ee). [stereochemistry arbitrarily assigned]. LCMS m/z=494.2 [M+H]⁺; 1H NMR (400 MHz, MeOH-d₄) δ 9.39 (s, 1H), 8.58 (dd, 1H, J=1.8, 7.5 Hz), 7.61 (s, 1H), 7.36 (dd, 1H, J=1.8, 7.0 Hz), 6.38 (t, 1H, J=7.3 Hz), 5.9-5.7 (m, 1H), 5.79 (quin, 1H, J=6.3 Hz), 4.06 (dd, 1H, J=3.3, 6.5 Hz), 3.96 (d, 1H, J=6.5 Hz), 3.70 (d, 2H, J=2.0 Hz), 3.47 (td, 1H, J=3.5, 7.7 Hz), 3.44 (s, 3H), 2.3-2.0 (m, 3H), 2.0-1.8 (m, 3H), 1.66 (d, 6H, J=6.3 Hz), 1.2-1.1 (m, 2H), 1.0-0.9 (m, 2H)

Peak 2: N-(1-cyclopropyl-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide (16.9 mg, >98% ee). (stereochemistry arbitrarily assigned). LCMS m/z=494.2 [M+H]⁺; 1H NMR (400 MHz, MeOH-d₄) δ 9.40 (s, 1H), 8.59 (dd, 1H, J=1.8, 7.5 Hz), 7.62 (s, 1H), 7.37 (dd, 1H, J=1.8, 7.0 Hz), 6.39 (t, 1H, J=7.2 Hz), 5.9-5.7 (m, 1H), 4.2-3.9 (m, 2H), 3.70 (d, 2H, J=1.8 Hz), 3.6-3.5 (m, 1H), 3.44 (s, 3H), 2.3-2.0 (m, 3H), 2.0-1.8 (m, 3H), 1.66 (d, 6H, J=6.0 Hz), 1.3-1.1 (m, 2H), 1.1-0.9 (m, 2H).

### Examples 113 and 114: 7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide and 7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

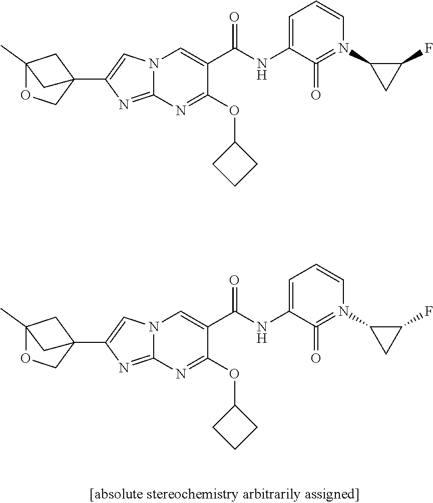

Cis-7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (Example 103) was further purified by SFC: CHIRALPAK AD-H 30×250 mm, 5 um Method: 40% IPA w/0.1% DEA in CO₂ (flow rate: 100 mL/min, ABPR 120 bar, MBPR 60 psi, column temp 40° C.) to give:

Peak 1: 7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (100% ee), 4.4 mg. (stereochemistry arbitrarily assigned). LCMS m/z=480.2 [M+H]⁺; 1H NMR (500 MHz, MeOH-d₄) δ: 9.40 (s, 1H), 8.61 (dd, J=1.68, 7.48 Hz, 1H), 7.62 (s, 1H), 7.41 (d, J=6.10 Hz, 1H), 6.41 (t, J=7.25 Hz, 1H), 5.64-5.54 (m, 1H), 5.08-5.01 (m, 1H), 4.00 (s, 2H), 3.43 (br d, J=6.56 Hz, 1H), 2.64 (q, J=8.04 Hz, 4H), 2.68-2.57 (m, 1H), 2.11 (dd, J=1.68, 4.58 Hz, 2H), 2.06-1.96 (m, 1H), 1.90-1.78 (m, 3H), 1.62-1.51 (m, 2H), 1.50 (s, 3H)

Peak 2: 7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (>93% ee), 4.5 mg. (stereochemistry arbitrarily assigned). LCMS m/z=480.2 [M+H]⁺; 1H NMR (500 MHz, MeOH-d₄) δ: 9.40 (s, 1H), 8.61 (dd, J=1.75, 7.40 Hz, 1H), 7.62 (s, 1H), 7.41 (d, J=7.63 Hz, 1H), 6.41 (t, J=7.25 Hz, 1H), 5.63-5.54 (m, 1H), 5.07-5.00 (m, 1H), 4.00 (s, 2H), 3.42 (br s, 1H), 3.43-3.40 (m, 1H), 2.64 (q, J=8.04 Hz, 4H), 2.11 (dd, J=1.53, 4.58 Hz, 2H), 2.06-1.96 (m, 1H), 1.89-1.79 (m, 3H), 1.61-1.51 (m, 2H), 1.50 (s, 3H).

### Example 115: N-(1-cyclopropyl-2-oxo-1,2-dihydropyridin-3-yl)-7-isopropoxy-2-(4-methyl-2-oxabicyclo[2.1.1]hexan-1-yl)imidazo[1,2-a]pyrimidine-6-carboxamide

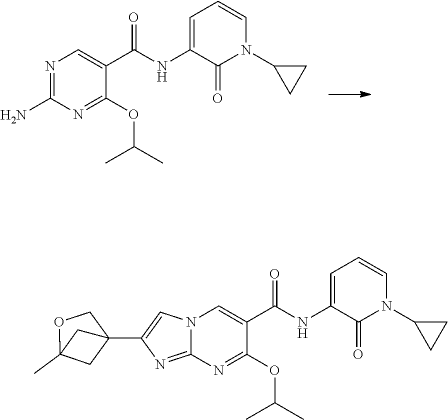 [SMILES: CC(C)Oc1nc(N)ncc1C(=O)Nc1cccn(C2CC2)c1=O ; CC(C)Oc1nc2nc(C34COC(C)(C3)C4)cn2cc1C(=O)Nc1cccn(C2CC2)c1=O]

A mixture of 2-amino-N-(1-cyclopropyl-2-oxo-1,2-dihydropyridin-3-yl)-4-isopropoxypyrimidine-5-carboxamide (Preparation 39, 60 mg, 182.18 μmol), 2-bromo-1-(4-methyl-2-oxabicyclo[2.1.1]hexan-1-yl)ethan-1-one (Preparation 88, 51.88 mg, 236.83 μmol) and aq. NaHCO₃ (450 mM, 546.53 μmol, 21.26 μL) in nBuOH (1.21 mL) was stirred at 90° C. for 16 h. The mixture was filtered and concentrated in vacuo. The residue was purified by HPLC (column: XSelect CSH Prep C18 5 um OBD 19×100 mm; Mobile phase A: MeCN; Mobile phase B: H₂O, Modifier: 0.1% NH₄OH) to provide N-(1-cyclopropyl-2-oxo-1,2-dihydropyridin-3-yl)-7-isopropoxy-2-(4-methyl-2-oxabicyclo[2.1.1]hexan-1-yl)imidazo[1,2-a]pyrimidine-6-carboxamide (9 mg, 11.0% yield). LCMS m/z=450.3 [M+H]⁺; 1H NMR (500 MHz, DMSO-d₆) δ ppm 10.71 (s, 1H), 9.53 (s, 1H), 8.44 (dd, J=7.32, 1.83 Hz, 1H), 7.76 (s, 1H), 7.35 (dd, J=6.71, 1.83 Hz, 1H), 6.32 (t, J=7.02 Hz, 1H), 5.62 (quin, J=6.26 Hz, 1H), 3.64 (s, 2H), 3.56-3.49 (m, 1H), 2.00 (dd, J=4.27, 1.22 Hz, 2H), 1.72 (dd, J=4.27, 1.83 Hz, 2H), 1.56 (d, J=6.10 Hz, 6H), 1.39 (s, 3H), 1.09-1.04 (m, 2H), 0.96-0.91 (m, 2H).

### Example 116: N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide

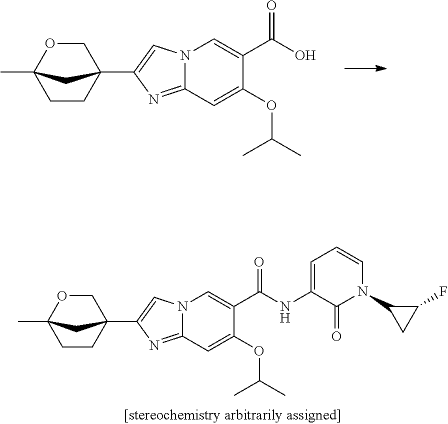

To a solution of 7-isopropoxy-2-((1S,4R)-1-methyl-2-oxabicyclo[2.2.1]heptan-4-yl)imidazo[1,2-a]pyridine-6-carboxylic acid (Preparation 59A, 30 mg, 90.81 μmol) in pyridine (1 mL) was added 3-amino-1-((1R,2R)-2-fluorocyclopropyl)pyridin-2(1H)-one hydrochloride (Preparation 84A, 18.32 mg, 108.97 μmol) and T3P® (1 mL) and the reaction was stirred at 25° C. for 16 h. The mixture was concentrated in vacuo and the residue was diluted with saturated NaHCO₃ aq. to pH=8. This mixture was extracted with EtOAc (50 mL×3), the combined organic layer was washed with brine (50 mL), dried over Na₂SO₄ and filtered. The filtrate was concentrated in vacuo and the residue was purified by HPLC (Column: Phenomenex Synergi C18 150×30 mm×4 um; Mobile Phase: from 49% to 69% of water (0.05% (NH₄HCO₃)-MeCN)) to give N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide (14.6 mg, 30% yield) as white solid. LCMS m/z=481.3 [M+H]⁺; 1HNMR (400 MHz, MeOH-d₄) δ ppm: 9.11 (s, 1H), 8.55 (dd, J=1.6, 7.5 Hz, 1H), 7.67 (s, 1H), 7.22 (dd, J=1.6, 7.2 Hz, 1H), 6.98 (s, 1H), 6.36 (t, J=7.2 Hz, 1H), 5.07-4.99 (m, 1H), 4.81 (d, J=3.6 Hz, 1H), 4.04 (dd, J=3.2, 6.4 Hz, 1H), 3.92 (d, J=6.4 Hz, 1H), 3.86-3.72 ((, 1H), 2.20-2.03 (m, 2H), 1.96 (d, J 2.4 Hz, 2H), 1.90-1.70 (i, 3H), 1.63 (d, J=6.0 Hz, 6H), 1.54-1.48 (m, 1H), 1.46 (s, 3H).

### Examples 117 to 136

The compounds in the following table were prepared from the appropriate 7-alkoxyimidazo[1,2-a]pyridine-6-carboxylic acid and one of the following amino pyridin-2(1H)-ones, following a similar procedure to that described in Example 116.

| | |
|---|---|
| Amine 1: | 3-amino-1-((1S,2S)-2-fluorocyclopropyl)pyridin-2(1H)-one |
| | hydrochloride (Preparation 85A) |
| Amine 2: | 3-amino-1-((1R,2R)-2-fluorocyclopropyl)pyridin-2(1H)-one |
| | hydrochloride (Preparation 84A) |
| Amine 3: | 3-amino-1-((1R,2S)-2-fluorocyclopropyl)pyridin-2(1H)-one |
| | (Preparation 86A) |
| Amine 4: | 3-amino-1-((1S,2R)-2-fluorocyclopropyl)pyridin-2(1H)-one |
| | (Preparation 87A) |
| Amine 5: | 3-amino-1-((1R,2S)-2-methylcyclopropyl)pyridin-2(1H)-one |
| | hydrochloride (Preparation 82) |
| Amine 6: | 3-amino-1-((1S,2R)-2-methylcyclopropyl)pyridin-2(1H)-one |
| | hydrochloride (Preparation 83) |
| Amine 7: | 3-amino-1-((1S,2R)-2-fluorocyclopropyl)pyridin-2(1H)-one |
| | hydrochloride (Preparation 87B) |

| Example No | Name/Structure/Starting Materials (SM)/Data |
|---|---|
| 117 | N-(1-((1S,2S)-2-fluorocyclopropyl)-2-oxo-1,2-dihydropyridin-3-yl)-7- |
| | isopropoxy-2-((1S,4R)-1-methyl-2-oxabicyclo[2.2.1]heptan-4- |
| | yl)imidazo[1,2-a]pyridine-6-carboxamide |
| | 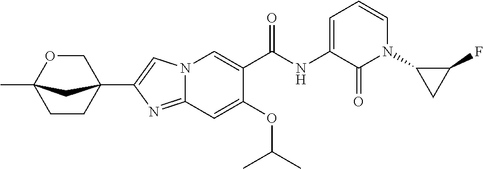 [SMILES: CC(C)Oc1cc2nc([C@@]34CC[C@@](C)(C3)OC4)cn2cc1C(=O)Nc1cccn([C@H]2C[C@@H]2F)c1=O] |
| | [stereochemistry arbitrarily assigned] |
| | SM: 7-isopropoxy-2-((1S,4R)-1-methyl-2-oxabicyclo[2.2.1]heptan-4- |
| | yl)imidazo[1,2-a]pyridine-6-carboxylic acid (Preparation 59A) and |
| | Amine 1 |
| | white solid, 12.4 mg, 28% yield. LCMS m/z = 481.3 [M + H]⁺; 1H NMR |
| | (400 MHz, MeOH-d₄) δ: 9.11 (s, 1H), 8.55 (dd, J = 1.6, 7.6 Hz, 1H), |
| | 7.67 (s, 1H), 7.22 (dd, J = 1.6, 7.2 Hz, 1H), 6.98 (s, 1H), 6.36 (t, J = 7.2 |
| | Hz, 1H), 5.03-4.97 (m, 1H), 4.81 (d, J = 3.6 Hz, 1H), 4.04 (dd, J = 3.6, |
| | 6.4 Hz, 1H), 3.92 (d, J = 6.4 Hz, 1H), 3.87-3.74 (m, 1H), 2.18-2.04 (m, |
| | 2H), 1.96 (d, J = 2.4 Hz, 2H), 1.88-1.69 (m, 3H), 1.63 (d, J = 6.0 Hz, |
| | 6H), 1.54-1.47 (m, 1H), 1.46 (s, 3H). |
| 118* | N-(1-((1R,2R)-2-fluorocyclopropyl)-2-oxo-1,2-dihydropyridin-3-yl)-7- |
| | isopropoxy-2-((1R,4S)-1-methyl-2-oxabicyclo[2.2.1]heptan-4- |
| | yl)imidazo[1,2-a]pyridine-6-carboxamide |
| | 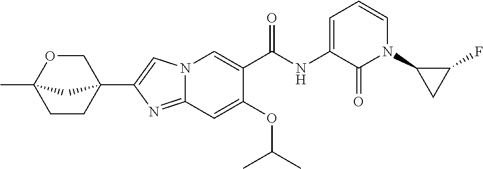 [SMILES: CC(C)Oc1cc2nc([C@]34CC[C@](C)(C3)OC4)cn2cc1C(=O)Nc1cccn([C@@H]2C[C@H]2F)c1=O] |
| | [stereochemistry arbitrarily assigned] |
| | SM: 7-isopropoxy-2-((1R,4S)-1-methyl-2-oxabicyclo[2.2.1]heptan-4- |
| | yl)imidazo[1,2-a]pyridine-6-carboxylic acid (Preparation 59B) and |
| | Amine 2 |
| | white solid, 26.4 mg, 67.5% yield. LCMS m/z = 481.2 [M + H]⁺; 1H |
| | NMR (500 MHz, MeOH-d₄) δ: 9.11 (s, 1H), 8.55 (dd, J = 7.5, 1.5 Hz, |
| | 1H), 7.67 (s, 1H), 7.24-7.21 (m, 1H), 6.98 (s, 1H), 6.36 (t, J = 7.0 Hz, |
| | 1H), 5.02-4.97 (m, 1H), 4.85-4.82 (m, 1H), 4.06-4.03 (m, 1H), 3.92 (d, J = |
| | 6.5 Hz, 1H), 3.83-3.76 (m, 1H), 2.20-2.13 (m, 1H), 2.10-2.03 (m, 1H), |
| | 1.99-1.93 (m, 2H), 1.91-1.84 (m, 1H), 1.84-1.71 (m, 2H), 1.63 (dd, J = |
| | 6.0, 2.0 Hz, 6H), 1.51-1.49 (m, 1H), 1.46 (s, 3H). |
| 119 | N-(1-((1S,2S)-2-fluorocyclopropyl)-2-oxo-1,2-dihydropyridin-3-yl)-7- |
| | isopropoxy-2-((1R,4S)-1-methyl-2-oxabicyclo[2.2.1]heptan-4- |
| | yl)imidazo[1,2-a]pyridine-6-carboxamide |
| | 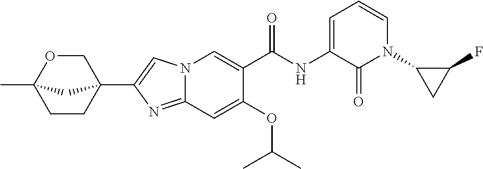 [SMILES: CC(C)Oc1cc2nc([C@]34CC[C@](C)(C3)OC4)cn2cc1C(=O)Nc1cccn([C@H]2C[C@@H]2F)c1=O] |
| | [stereochemistry arbitrarily assigned] |
| | SM: 7-isopropoxy-2-((1R,4S)-1-methyl-2-oxabicyclo[2.2.1]heptan-4- |
| | yl)imidazo[1,2-a]pyridine-6-carboxylic acid (Preparation 59B) and |
| | Amine 1 |
| | white solid, 23.8 mg, 60.8% yield. LCMS m/z = 481.1 [M + H]⁺; 1H |
| | NMR (500 MHz, MeOH-d₄) δ: 9.11 (s, 1H), 8.55 (d, J = 7.5 Hz, 1H), |
| | 7.67 (s, 1H), 7.24-7.21 (m, 1H), 6.98 (s, 1H), 6.36 (t, J = 7.0 Hz, 1H), |
| | 5.02-4.99 (m, 1H), 4.98-4.94 (m, 1H), 4.06-4.03 (m, 1H), 3.92 (d, J = |
| | 6.5 Hz, 1H), 3.83-3.76 (m, 1H), 2.20-2.13 (m, 1H), 2.10-2.05 (m, 1H), |
| | 1.99-1.93 (m, 2H), 1.91-1.85 (m, 1H), 1.84-1.71 (m, 2H), 1.63 (dd, J = |
| | 6.0, 1.0 Hz, 6H), 1.51-1.48 (m, 1H), 1.46 (s, 3H). |
| 120 | N-(1-((1R,2R)-2-fluorocyclopropyl)-2-oxo-1,2-dihydropyridin-3-yl)-7- |
| | isopropoxy-2-((1R,4S)-1-methyl-2-oxabicyclo[2.2.1]heptan-4- |
| | yl)imidazo[1,2-a]pyrimidine-6-carboxamide |
| | 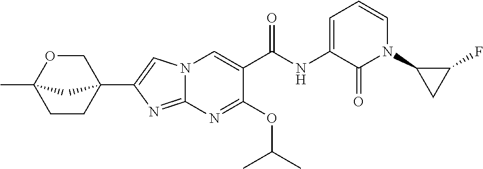 [SMILES: CC(C)Oc1nc2nc([C@]34CC[C@](C)(C3)OC4)cn2cc1C(=O)Nc1cccn([C@@H]2C[C@H]2F)c1=O] |
| | [stereochemistry arbitrarily assigned] |
| | SM: 7-isopropoxy-2-((1R,4S)-1-methyl-2-oxabicyclo[2.2.1]heptan-4- |
| | yl)imidazo[1,2-a]pyrimidine-6-carboxylic acid (Preparation 92) and |
| | Amine 2 |
| | white solid, 15.1 mg, 35% yield. LMCS m/z = 482.2 [M + H]⁺; 1H NMR |
| | (400 MHz, MeOH-d₄) δ: 9.35 (s, 1H), 8.51 (dd, J = 7.5, 1.5 Hz, 1H), |
| | 7.57 (s, 1H), 7.21 (d, J = 7.0 Hz, 1H), 6.35-6.31 (m, 1H), 5.80-5.71 |
| | (m, 1H), 4.04 (dd, J = 6.0, 4.0 Hz, 1H), 3.90 (dd, J = 6.0, 4.0 Hz, 1H), |
| | 3.84-3.76 (m, 1H), 2.19-2.04 (m, 3H), 1.92 (s, 2H), 1.84-1.74 (m, |
| | 3H), 1.64 (d, J = 6.4 Hz, 6 H), 1.52-1.47 (m, 1H), 1.45 (s, 3H). |
| 121 | N-(1-((1S,2S)-2-fluorocyclopropyl)-2-oxo-1,2-dihydropyridin-3-yl)-7- |
| | isopropoxy-2-((1R,4S)-1-methyl-2-oxabicyclo[2.2.1]heptan-4- |
| | yl)imidazo[1,2-a]pyrimidine-6-carboxamide |
| | 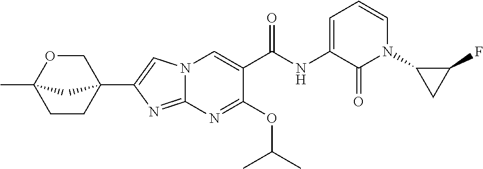 [SMILES: CC(C)Oc1nc2nc([C@]34CC[C@](C)(C3)OC4)cn2cc1C(=O)Nc1cccn([C@H]2C[C@@H]2F)c1=O] |
| | [stereochemistry arbitrarily assigned] |
| | SM: 7-isopropoxy-2-((1R,4S)-1-methyl-2-oxabicyclo[2.2.1]heptan-4- |
| | yl)imidazo[1,2-a]pyrimidine-6-carboxylic acid (Preparation 92) and |
| | Amine 1 |
| | white solid, 11.1 mg, 25% yield. LCMS m/z = 482.3 [M + H]⁺; 1H NMR |
| | (400 MHz, MeOH-d₄) δ: 9.34 (s, 1H), 8.50 (dd, J = 7.5, 1.5 Hz, 1H), |
| | 7.57 (s, 1H), 7.21 (d, J = 7.0 Hz, 1H), 6.35-6.31 (m, 1H), 5.80-5.70 |
| | (m, 1H), 4.03 (dd, J = 6.0, 4.0 Hz, 1H), 3.90 (dd, J = 6.0, 4.0 Hz, 1H), |
| | 3.84-3.76 (m, 1H), 2.19-1.97 (m, 3H), 1.94 (s, 2H), 1.85-1.71 (m, |
| | 3H), 1.64 (d, J = 6.4 Hz, 6H), 1.53-1.48 (m, 1H), 1.45 (s, 3H). |
| 122 | N-(1-((1R,2R)-2-fluorocyclopropyl)-2-oxo-1,2-dihydropyridin-3-yl)-7- |
| | isopropoxy-2-((1S,4R)-1-methyl-2-oxabicyclo[2.2.1]heptan-4- |
| | yl)imidazo[1,2-a]pyrimidine-6-carboxamide |
| | 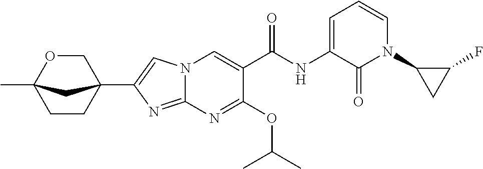 [SMILES: CC(C)Oc1nc2nc([C@@]34CC[C@@](C)(C3)OC4)cn2cc1C(=O)Nc1cccn([C@@H]2C[C@H]2F)c1=O] |
| | [stereochemistry arbitrarily assigned] |
| | SM: 7-isopropoxy-2-((1S,4R)-1-methyl-2-oxabicyclo[2.2.1]heptan-4- |
| | yl)imidazo[1,2-a]pyrimidine-6-carboxylic acid (Preparation 91) and |
| | Amine 2 |
| | white solid, 5.9 mg, 13% yield. LCMS m/z = 482.3 [M + H]⁺; 1H NMR |
| | (400 MHz, MeOH-d₄) δ: 9.37 (s, 1H), 8.54 (dd, J = 7.5, 1.5 Hz, 1H), 7.58 |
| | (s, 1H), 7.24 (d, J = 7.0 Hz, 1H), 6.38-6.34 (m, 1H), 5.80-5.74 (m, |
| | 1H), 4.04 (dd, J = 6.0, 4.0 Hz, 1H), 3.91 (dd, J = 6.0, 4.0 Hz, 1H), 3.85- |
| | 3.77 (m, 1H), 2.09-2.04 (m, 3H), 1.95 (s, 2H), 1.87-1.71 (m, 3H), 1.64 |
| | (d, J = 6.4 Hz, 6H), 1.54-1.49 (m, 1H), 1.46 (s, 3H). |
| 123 | N-(1-((1S,2S)-2-fluorocyclopropyl)-2-oxo-1,2-dihydropyridin-3-yl)-7- |
| | isopropoxy-2-((1S,4R)-1-methyl-2-oxabicyclo[2.2.1]heptan-4- |
| | yl)imidazo[1,2-a]pyrimidine-6-carboxamide |
| | 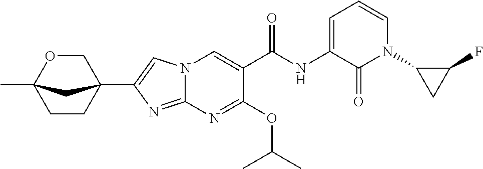 [SMILES: CC(C)Oc1nc2nc([C@@]34CC[C@@](C)(C3)OC4)cn2cc1C(=O)Nc1cccn([C@H]2C[C@@H]2F)c1=O] |
| | [stereochemistry arbitrarily assigned] |
| | SM: 7-isopropoxy-2-((1S,4R)-1-methyl-2-oxabicyclo[2.2.1]heptan-4- |
| | yl)imidazo[1,2-a]pyrimidine-6-carboxylic acid (Preparation 91) and |
| | Amine 1 |
| | white solid, 6.0 mg, 14% yield. LCMS m/z = 482.3 [M + H]⁺; 1H NMR |
| | (400 MHz, MeOH-d₄) δ: 9.37 (s, 1H), 8.54 (dd, J = 7.5, 1.5 Hz, 1H), |
| | 7.58 (s, 1H), 7.24 (d, J = 7.0 Hz, 1H), 6.38-6.34 (m, 1H), 5.80-5.74 |
| | (m, 1H), 4.04 (dd, J = 6.0, 4.0 Hz, 1H), 3.91 (dd, J = 6.0, 4.0 Hz, 1H), |
| | 3.85-3.77 (m, 1H), 2.09-2.04 (m, 3H), 1.95 (s, 2H), 1.87-1.71 (m, |
| | 3H), 1.64 (d, J = 6.4 Hz, 6H), 1.54-1.49 (m, 1H), 1.46 (s, 3H). |
| 124 | 8-fluoro-N-(1-((1R,2S)-2-fluorocyclopropyl)-2-oxo-1,2-dihydropyridin- |
| | 3-yl)-7-isopropoxy-2-((1S,4R)-1-methyl-2-oxabicyclo[2.2.1]heptan-4- |
| | yl)imidazo[1,2-a]pyridine-6-carboxamide |
| | 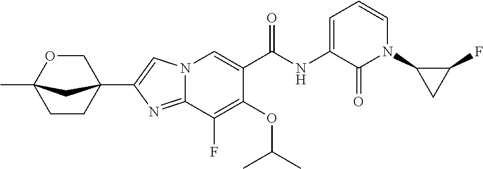 [SMILES: CC(C)Oc1c(C(=O)Nc2cccn([C@@H]3C[C@@H]3F)c2=O)cn2cc([C@@]34CC[C@@](C)(C3)OC4)nc2c1F] |
| | [stereochemistry arbitrarily assigned] |
| | SM: 8-fluoro-7-isopropoxy-2-((1S,4R)-1-methyl-2- |
| | oxabicyclo[2.2.1]heptan-4-yl)imidazo[1,2-a]pyridine-6-carboxylic acid |
| | (Preparation 93) and Amine 3 |
| | white solid, 23.8 mg, 55% yield. LMCS m/z = 499.2 [M + H]⁺; 1H NMR |
| | (500 MHz, MeOH-d₄) δ: 8.98 (d, J = 1.0 Hz, 1H), 8.58 (dd, J = 7.5, |
| | 1.5 Hz, 1H), 7.83 (d, J = 3.0 Hz, 1H), 7.40 (d, J = 7.0 Hz, 1H), 6.41- |
| | 6.38 (m, 1H), 5.05-5.02 (m, 1H), 4.93-4.92 (m, 1H), 4.05 (dd, J = 6.0, |
| | 4.0 Hz, 1H), 3.94 (dd, J = 6.5, 4.0 Hz, 1H), 3.42-3.38 (m, 1H), 2.20- |
| | 2.07 (m, 2H), 1.99 (s, 2H), 1.89-1.87 (m, 2H), 1.60-1.52 (m, 2H), 1.52- |
| | 1.49 (m, 6H), 1.46 (s, 3H). |
| 125 | 8-fluoro-N-(1-((1S,2R)-2-fluorocyclopropyl)-2-oxo-1,2-dihydropyridin- |
| | 3-yl)-7-isopropoxy-2-((1S,4R)-1-methyl-2-oxabicyclo[2.2.1]heptan-4- |
| | yl)imidazo[1,2-a]pyridine-6-carboxamide |
| | 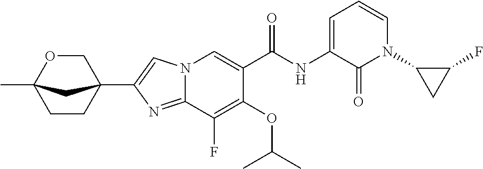 [SMILES: CC(C)Oc1c(C(=O)Nc2cccn([C@H]3C[C@H]3F)c2=O)cn2cc([C@@]34CC[C@@](C)(C3)OC4)nc2c1F] |
| | [stereochemistry arbitrarily assigned] |
| | SM: 8-fluoro-7-isopropoxy-2-((1S,4R)-1-methyl-2- |
| | oxabicyclo[2.2.1]heptan-4-yl)imidazo[1,2-a]pyridine-6-carboxylic acid |
| | (Preparation 93) and Amine 4 |
| | white solid, 20.4 mg, 48% yield. LCMS m/z = 499.2 [M + H]⁺. 1H NMR |
| | (500 MHz, MeOH-d₄) δ: 8.99 (d, J = 1.0 Hz, 1H), 8.58 (dd, J = 7.5, |
| | 1.5 Hz, 1H), 7.83 (d, J = 3.0 Hz, 1H), 7.40 (d, J = 7.0 Hz, 1H), 6.41- |
| | 6.38 (m, 1H), 5.05-5.02 (m, 1H), 4.93-4.92 (m, 1H), 4.05 (dd, J = 6.0, |
| | 4.0 Hz, 1H), 3.94 (dd, J = 6.5, 4.0 Hz, 1H), 3.42-3.38 (m, 1H), 2.20- |
| | 2.07 (m, 2H), 1.99 (s, 2H), 1.89-1.87 (m, 2H), 1.60-1.52 (m, 2H), 1.52- |
| | 1.49 (m, 6H), 1.46 (s, 3H). |
| 126 | 8-fluoro-N-(1-((1R,2S)-2-fluorocyclopropyl)-2-oxo-1,2-dihydropyridin- |
| | 3-yl)-7-isopropoxy-2-((1R,4S)-1-methyl-2-oxabicyclo[2.2.1]heptan-4- |
| | yl)imidazo[1,2-a]pyridine-6-carboxamide |
| | 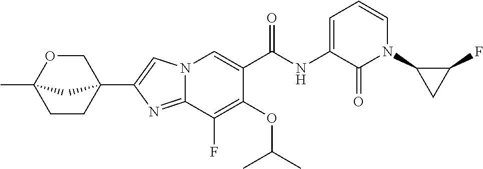 [SMILES: CC(C)Oc1c(C(=O)Nc2cccn([C@@H]3C[C@@H]3F)c2=O)cn2cc([C@]34CC[C@](C)(C3)OC4)nc2c1F] |
| | [stereochemistry arbitrarily assigned] |
| | SM: 8-fluoro-7-isopropoxy-2-((1R,4S)-1-methyl-2- |
| | oxabicyclo[2.2.1]heptan-4-yl)imidazo[1,2-a]pyridine-6-carboxylic acid |
| | (Preparation 94) and Amine 3 |
| | white solid, 16.2 mg, 38% yield. LCMS m/z = 499.2 [M + H]⁺. 1H NMR |
| | (500 MHz, MeOH-d₄) δ: 8.98 (s, 1H), 8.57 (dd, J = 7.5, 1.5 Hz, 1H), |
| | 7.82 (d, J = 3.0 Hz, 1H), 7.39 (d, J = 7.0 Hz, 1H), 6.40-6.38 (m, 1H), |
| | 5.05-5.02 (m, 1H), 4.93-4.92 (m, 1H), 4.05 (dd, J = 6.0, 4.0 Hz, 1H), |
| | 3.94 (dd, J = 6.5, 4.0 Hz, 1H), 3.42-3.38 (m, 1H), 2.20-2.07 (m, 2H), |
| | 1.99 (s, 2H), 1.89-1.87 (m, 2H), 1.60-1.52 (m, 2H), 1.52-1.49 (m, |
| | 6H), 1.46 (s, 3H). |
| 127 | 8-fluoro-N-(1-((1S,2R)-2-fluorocyclopropyl)-2-oxo-1,2-dihydropyridin- |
| | 3-yl)-7-isopropoxy-2-((1R,4S)-1-methyl-2-oxabicyclo[2.2.1]heptan-4- |
| | yl)imidazo[1,2-a]pyridine-6-carboxamide |
| | 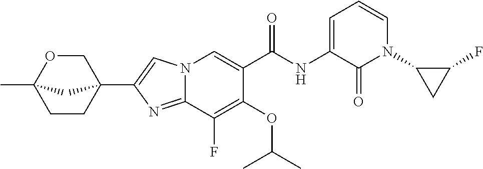 [SMILES: CC(C)Oc1c(C(=O)Nc2cccn([C@H]3C[C@H]3F)c2=O)cn2cc([C@]34CC[C@](C)(C3)OC4)nc2c1F] |
| | [stereochemistry arbitrarily assigned] |
| | SM: 8-fluoro-7-isopropoxy-2-((1R,4S)-1-methyl-2- |
| | oxabicyclo[2.2.1]heptan-4-yl)imidazo[1,2-a]pyridine-6-carboxylic acid |
| | (Preparation 94) and Amine 4 |
| | white solid, 19.1 mg, 45% yield. LCMS m/z = 499.2 [M + H]⁺. 1H NMR |
| | (500 MHz, MeOH-d₄) δ: 8.99 (s, 1H), 8.59 (dd, J = 7.5, 1.5 Hz, 1H), |
| | 7.83 (d, J = 3.0 Hz, 1H), 7.40 (d, J = 7.0 Hz, 1H), 6.41-6.38 (m, 1H), |
| | 5.05-5.02 (m, 1H), 4.93-4.92 (m, 1H), 4.05 (dd, J = 6.0, 4.0 Hz, 1H), |
| | 3.94 (dd, J = 6.5, 4.0 Hz, 1H), 3.42-3.38 (m, 1H), 2.20-2.07 (m, 2H), |
| | 1.99 (s, 2H), 1.89-1.87 (m, 2H), 1.60-1.52 (m, 2H), 1.52-1.49 (m, |
| | 6H), 1.46 (s, 3H). |
| 128 | 7-cyclobutoxy-N-(1-((1S,2R)-2-fluorocyclopropyl)-2-oxo-1,2- |
| | dihydropyridin-3-yl)-2-(1-(methoxymethyl)-2-oxabicyclo[2.1.1]hexan- |
| | 4-yl)imidazo[1,2-a]pyrimidine-6-carboxamide |
| | 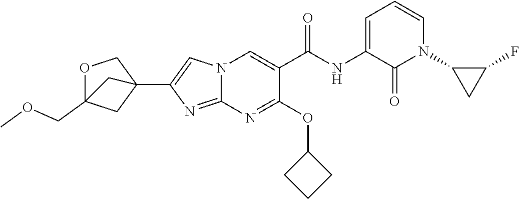 [SMILES: COCC12CC(c3cn4cc(C(=O)Nc5cccn([C@H]6C[C@H]6F)c5=O)c(OC5CCC5)nc4n3)(CO1)C2] |
| | SM: 7-cyclobutoxy-2-(1-(methoxymethyl)-2-oxabicyclo[2.1.1]hexan-4- |
| | yl)imidazo[1,2-a]pyrimidine-6-carboxylic acid (Preparation 90) and |
| | Amine 7 |
| | white solid, 13.1 mg, 31% yield. LCMS m/z = 510.2 [M + H]⁺; 1H NMR |
| | (500 MHz, MeOH-d₄) δ: 9.32 (s, 1H), 8.52 (d, J = 9.0 Hz, 1H), 7.59 (s, |
| | 1H), 7.35 (d, J = 4.0 Hz, 1H), 6.35-6.33 (m, 1H), 5.54-5.50 (m, 1H), |
| | 5.05-4.91 (m, 1H), 4.01 (s, 2H), 3.70 (s, 2H), 3.42 (s, 3H), 3.41-3.40 |
| | (m, 1H), 2.64-2.60 (m, 4H), 2.17-2.16 (m, 2H), 2.01-1.99 (m, 1H), |
| | 1.91-1.89 (m, 2H), 1.56-1.53 (m, 1H), 1.56-1.51 (m, 2H). |
| 129** | N-(1-((1R,2S)-2-fluorocyclopropyl)-2-oxo-1,2-dihydropyridin-3-yl)-7- |
| | isopropoxy-2-((1R,4S)-1-(methoxymethyl)-2-oxabicyclo[2.2.1]heptan- |
| | 4-yl)imidazo[1,2-a]pyrimidine-6-carboxamide |
| | 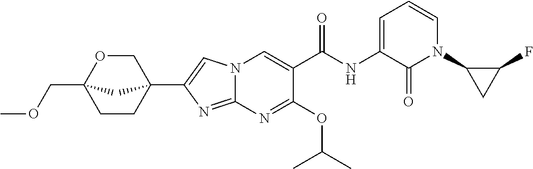 [SMILES: COC[C@]12CC[C@](c3cn4cc(C(=O)Nc5cccn([C@@H]6C[C@@H]6F)c5=O)c(OC(C)C)nc4n3)(CO1)C2] |
| | [stereochemistry arbitrarily assigned] |
| | SM: 7-isopropoxy-2-((1R,4S)-1-(methoxymethyl)-2- |
| | oxabicyclo[2.2.1]heptan-4-yl)imidazo[1,2-a]pyrimidine-6-carboxylic |
| | acid (Preparation 96) and Amine 3 |
| | (36.9 mg, 65% yield) as white solid. LCMS m/z = 512.2 [M + H]⁺; 1H |
| | NMR (400 MHz, MeOH-d₄) δ: 9.38 (s, 1H), 8.59 (dd, J = 7.6, 1.6 Hz, |
| | 1H), 7.60 (s, 1H), 7.39 (d, J = 6.4 Hz, 1H), 6.39 (t, J = 7.2 Hz, 1H), |
| | 5.81-5.73 (m, 1H), 5.08-5.02 (m, 1H), 4.06-4.02 (m, 1H), 3.96-3.93 (m, |
| | 1H), 3.71-3.65 (m, 2H), 3.42-3.38 (m, 4H), 2.18-2.12 (m, 1H), 2.10-2.03 |
| | (m, 2H), 1.96-1.89 (m, 2H), 1.89-1.84 (m, 1H), 1.64 (d, J = 6.4 Hz, 6H), |
| | 1.59-1.50 (m, 2H). |
| 130** | N-(1-((1S,2R)-2-fluorocyclopropyl)-2-oxo-1,2-dihydropyridin-3-yl)-7- |
| | isopropoxy-2-((1R,4S)-1-(methoxymethyl)-2-oxabicyclo[2.2.1]heptan- |
| | 4-yl)imidazo[1,2-a]pyrimidine-6-carboxamide |
| | 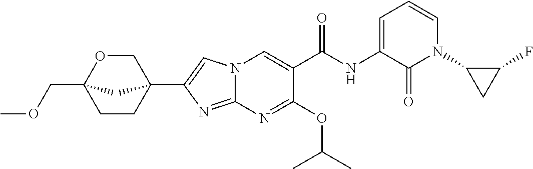 [SMILES: COC[C@]12CC[C@](c3cn4cc(C(=O)Nc5cccn([C@H]6C[C@H]6F)c5=O)c(OC(C)C)nc4n3)(CO1)C2] |
| | [stereochemistry arbitrarily assigned] |
| | SM: 7-isopropoxy-2-((1R,4S)-1-(methoxymethyl)-2- |
| | oxabicyclo[2.2.1]heptan-4-yl)imidazo[1,2-a]pyrimidine-6-carboxylic |
| | acid (Preparation 96) and Amine 4 |
| | white solid, 51.5 mg, 91% yield. LCMS m/z = 512.2 [M + H]⁺; 1H NMR |
| | (400 MHz, MeOH-d₄) δ: 9.38 (s, 1H), 8.58 (dd, J = 7.6, 1.6 Hz, 1H), |
| | 7.59 (s, 1H), 7.39 (d, J = 6.8 Hz, 1H), 6.39 (t, J = 7.2 Hz, 1H), 5.80-5.72 |
| | (m, 1H), 5.08-5.02 (m, 1H), 4.06-4.02 (m, 1H), 3.95-3.93 (m, 1H), 3.71- |
| | 3.65 (m, 2H), 3.44-3.41 (m, 4H), 2.19-2.12 (m, 1H), 2.10-2.03 (m, 2H), |
| | 1.96-1.82 (m, 3H), 1.64 (d, J = 6.4 Hz, 6H), 1.59-1.50 (m, 2H). |
| 131*** | N-(1-((1R,2S)-2-fluorocyclopropyl)-2-oxo-1,2-dihydropyridin-3-yl)-7- |
| | isopropoxy-2-((1S,4R)-1-(methoxymethyl)-2-oxabicyclo[2.2.1]heptan- |
| | 4-yl)imidazo[1,2-a]pyrimidine-6-carboxamide |
| | 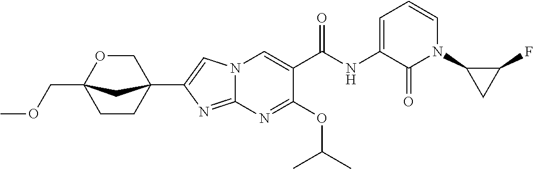 [SMILES: COC[C@@]12CC[C@@](c3cn4cc(C(=O)Nc5cccn([C@@H]6C[C@@H]6F)c5=O)c(OC(C)C)nc4n3)(CO1)C2] |
| | [stereochemistry arbitrarily assigned] |
| | SM: 7-isopropoxy-2-((1S,4R)-1-(methoxymethyl)-2- |
| | oxabicyclo[2.2.1]heptan-4-yl)imidazo[1,2-a]pyrimidine-6-carboxylic |
| | acid (Preparation 95) and Amine 3 |
| | White solid (28.0 mg, 49.5% yield). LCMS m/z = 512.2 [M + H]⁺; 1H |
| | NMR (400 MHz, MeOH-d₄) δ: 9.38 (s, 1H), 8.59 (d, J = 7.2 Hz, 1H), |
| | 7.60 (s, 1H), 7.39 (d, J = 6.4 Hz, 1H), 6.39 (t, J = 6.8 Hz, 1H), 5.80-5.70 |
| | (m, 1H), 5.10-5.00 (m, 1H), 4.00-3.90 (m, 2H), 3.68 (s, 2H), 3.42 (s, |
| | 3H), 3.30-3.20 (m, 1H), 2.20-2.10 (m, 3H), 2.00-1.90 (m, 3H), 3.00-2.90 |
| | (m, 1H), 2.60-2.50 (m, 3H), 2.40-2.30 (m, 3H), 1.64 (d, J = 6.00 Hz, 6H), |
| | 1.60-1.59 (m, 2H). |
| 132*** | N-(1-((1S,2R)-2-fluorocyclopropyl)-2-oxo-1,2-dihydropyridin-3-yl)-7- |
| | isopropoxy-2-((1S,4R)-1-(methoxymethyl)-2-oxabicyclo[2.2.1]heptan- |
| | 4-yl)imidazo[1,2-a]pyrimidine-6-carboxamide |
| | 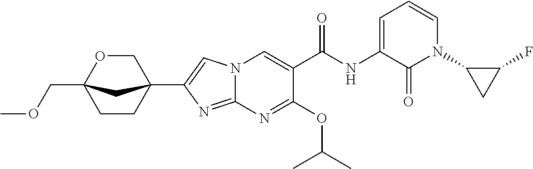 [SMILES: COC[C@@]12CC[C@@](c3cn4cc(C(=O)Nc5cccn([C@H]6C[C@H]6F)c5=O)c(OC(C)C)nc4n3)(CO1)C2] |
| | [stereochemistry arbitrarily assigned] |
| | SM: 7-isopropoxy-2-((1S,4R)-1-(methoxymethyl)-2- |
| | oxabicyclo[2.2.1]heptan-4-yl)imidazo[1,2-a]pyrimidine-6-carboxylic |
| | acid (Preparation 95) and Amine 4 |
| | White solid, 29.2 mg, 51.6% yield. LCMS m/z = 512.2 [M + H]⁺; 1H |
| | NMR (400 MHz, MeOH-d₄) δ: 9.38 (s, 1H), 8.59 (d, J = 7.2 Hz, 1H), |
| | 7.60 (s, 1H), 7.39 (d, J = 6.4 Hz, 1H), 6.39 (t, J = 6.8 Hz, 1H), 5.80-5.70 |
| | (m, 1H), 5.10-5.00 (m, 1H), 4.00-3.90 (m, 2H), 3.68 (s, 2H), 3.42 (s, |
| | 3H), 3.30-3.20 (m, 1H), 2.20-2.10 (m, 3H), 2.00-1.90 (m, 3H), 3.00-2.90 |
| | (m, 1H), 2.60-2.50 (m, 3H), 2.40-2.30 (m, 3H), 1.64 (d, J = 6.0 Hz, 6H), |
| | 1.60-1.59 (m, 2H). |
| 133* | 7-isopropoxy-2-(1-methyl-2-oxabicyclo[2.1.1]hexan-4-yl)-N-(1- |
| | ((1R,2S)-2-methylcyclopropyl)-2-oxo-1,2-dihydropyridin-3- |
| | yl)imidazo[1,2-a]pyridine-6-carboxamide |
| | 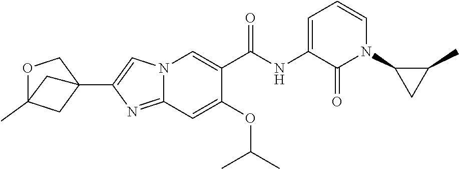 |
| | SM: 7-isopropoxy-2-(1-methyl-2-oxabicyclo[2.1.1]hexan-4- |
| | yl)imidazo[1,2-a]pyridine-6-carboxylic acid (Preparation 4) and Amine |
| | 5 |
| | white solid, 26 mg, 45% yield. LCMS m/z = 463.1 [M + H]⁺; 1H NMR |
| | (400 MHz, MeOH-d₄) δ: 9.12 (s, 1H), 8.59 (dd, J = 7.6, 1.6 Hz, 1H), |
| | 7.70 (s, 1H), 7.32 (d, J = 6.4 Hz, 1H), 7.00 (s, 1H), 6.38 (t, J = 6.8 Hz, |
| | 1H), 5.10-5.00 (m, 1H), 4.01 (s, 2H), 3.50-3.40 (m, 1H), 2.15-2.10 (m, |
| | 2H), 1.90-1.85 (m, 2H), 1.60-1.55 (m, 6H), 1.50 (s, 3H), 1.45-1.35 (m, |
| | 1H), 1.30-1.20 (m, 2H), 0.84 (d, J = 6.4 Hz, 3H). |
| 134 | 7-isopropoxy-2-(1-methyl-2-oxabicyclo[2.1.1]hexan-4-yl)-N-(1- |
| | ((1S,2R)-2-methylcyclopropyl)-2-oxo-1,2-dihydropyridin-3- |
| | yl)imidazo[1,2-a]pyridine-6-carboxamide |
| | 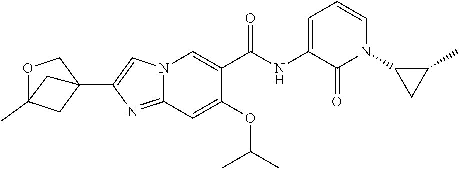 |
| | SM: 7-isopropoxy-2-(1-methyl-2-oxabicyclo[2.1.1]hexan-4- |
| | yl)imidazo[1,2-a]pyridine-6-carboxylic acid (Preparation 4) and Amine |
| | 6 |
| | white solid, 25.1 mg, 43% yield. LCMS m/z = 463.1 [M + H]⁺; 1H NMR |
| | (400 MHz, MeOH-d₄) δ: 9.11 (s, 1H), 8.59 (dd, J = 7.6, 1.6 Hz, 1H), |
| | 7.70 (s, 1H), 7.32 (d, J = 6.4 Hz, 1H), 7.00 (s, 1H), 6.38 (t, J = 6.8 Hz, |
| | 1H), 5.10-5.00 (m, 1H), 4.01 (s, 2H), 3.50-3.40 (m, 1H), 2.15-2.10 (m, |
| | 2H), 1.90-1.85 (m, 2H), 1.60-1.55 (m, 6H), 1.50 (s, 3H), 1.45-1.35 (m, |
| | 1H), 1.30-1.20 (m, 2H), 0.84 (d, J = 6.4 Hz, 3H). |
| 135**** | 7-isopropoxy-2-(1-methyl-2-oxabicyclo[2.1.1]hexan-4-yl)-N-(1- |
| | ((1R,2S)-2-methylcyclopropyl)-2-oxo-1,2-dihydropyridin-3- |
| | yl)imidazo[1,2-a]pyrimidine-6-carboxamide |
| | 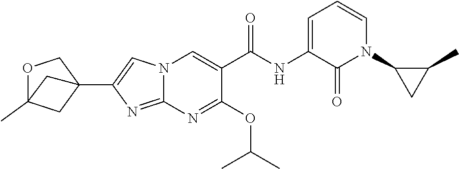 |
| | SM: 7-isopropoxy-2-(1-methyl-2-oxabicyclo[2.1.1]hexan-4- |
| | yl)imidazo[1,2-a]pyrimidine-6-carboxylic acid (Preparation 8) and |
| | Amine 5 |
| | (43.2 mg, 58% yield) as white solid. LCMS m/z = 464.2 [M + H]⁺; 1H |
| | NMR (500 MHz, MeOH-d₄) δ: 9.38 (s, 1H), 8.59 (dd, J = 7.5, 1.5 Hz, |
| | 1H), 7.62 (s, 1H), 7.34 (dd, J = 7.0, 1.5 Hz, 1H), 6.38 (t, J = 7.0 Hz, 1H), |
| | 5.81-5.74 (m, 1H), 4.00 (s, 2H), 3.48-3.43 (m, 1H), 2.14-2.08 (m, 2H), |
| | 1.89-1.84 (m, 2H), 1.64 (dd, J = 6.0, 3.0 Hz, 6H), 1.50 (s, 3H), 1.48-1.43 |
| | (m, 1H), 1.31-1.25 (m, 1H), 0.88-0.86 (m, 1H), 0.84 (d, J = 6.5 Hz, 3H). |
| 136 | 7-isopropoxy-2-(1-methyl-2-oxabicyclo[2.1.1]hexan-4-yl)-N-(1- |
| | ((1S,2R)-2-methylcyclopropyl)-2-oxo-1,2-dihydropyridin-3- |
| | yl)imidazo[1,2-a]pyrimidine-6-carboxamide |
| | 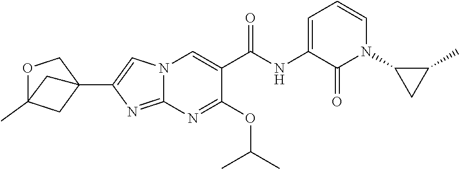 |
| | SM: 7-isopropoxy-2-(1-methyl-2-oxabicyclo[2.1.1]hexan-4- |
| | yl)imidazo[1,2-a]pyrimidine-6-carboxylic acid (Preparation 8) and |
| | Amine 6 |
| | white solid, 37.8 mg, 50.4% yield. LCMS m/z = 464.2 [M + H]⁺; ¹H |
| | NMR: (500 MHz, MeOH-d₄) δ: 9.38 (s, 1H), 8.59 (dd, J = 7.5, 1.5 Hz, |
| | 1H), 7.62 (s, 1H), 7.34 (dd, J = 7.0, 1.5 Hz, 1H), 6.38 (t, J = 7.0 Hz, 1H), |
| | 5.81-5.74 (m, 1H), 4.00 (s, 2H), 3.48-3.43 (m, 1H), 2.14-2.08 (m, 2H), |
| | 1.89-1.84 (m, 2H), 1.64 (dd, J = 6.0, 3.0 Hz, 6H), 1.50 (s, 3H), 1.48-1.43 |
| | (m, 1H), 1.31-1.25 (m, 1H), 0.88-0.86 (m, 1H), 0.84 (d, J = 6.5 Hz, 3H). |

*prep-HPLC (Column: Welch Xtimate C18 150 × 25 mm 5 × um; mobile phase: water (10 mm NH₄CO₃)-
MeCN; gradient 42%-72% over 10 mins. Flow Rate 25 mL/min
**prep-HPLC (Column: Boston Prime C18 150 × 30 mm × 5 μm; mobile phase: water (NH₃•H₂O+NH₄HCO₃)-
MeCN; gradient: 30-60% over 10 mins at 25 mL/min
***the crude was purified by prep TLC (PE/EtOAc = 1/1)
****prep-HPLC (Column: AgNO₃_silica 150 × 25 mm × 15 um; Condition: Heptane-EtOH
(0.1% NH₃H₂O); Begin B: 5; End B: 95; Gradient Time (min): 9; Flow Rate 25 mL/min

### Example 137 and 138: 7-isopropoxy-N-(1-((1R,2R)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide and 7-isopropoxy-N-(1-((1S,2S)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide

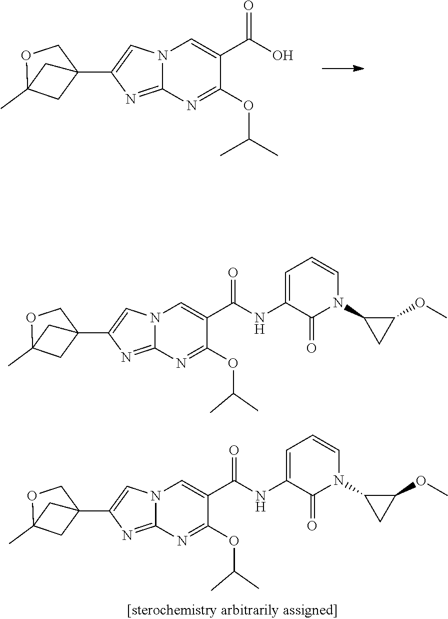

To a solution of 7-isopropoxy-2-(1-methyl-2-oxabicyclo[2.1.1]hexan-4-yl)imidazo[1,2-a]pyrimidine-6-carboxylic acid (Preparation 8, 50 mg, 157.56 μmol) in pyridine (2 mL) was added trans-3-amino-1-(2-methoxycyclopropyl)pyridin-2(1H)-one hydrochloride (Preparation 78, 34.07 mg, 189.07 μmol) and T3P® (2 mL) and the reaction stirred at 25° C. for 16 h. The mixture was concentrated and the residue was diluted with saturated aq. NaHCO₃ to pH=8. This mixture was extracted with EtOAc (50 mL×3), the combined organic layer was washed with brine (50 mL), dried over Na₂SO₄ and filtered. The filtrate was concentrated in vacuo and the residue was purified by TLC (PE/EtOAc=1/1) to give trans-7-isopropoxy-N-(1-(2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide (30 mg, 40% yield) as yellow oil. LCMS m/z=480.0 [M+H]⁺

Trans-7-isopropoxy-N-(1-(2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide was further purified by prep. SFC: ChiralCel OD-3 150×4.6 mm I.D., 5 um Mobile phase: A: CO₂ B: MeOH (0.05% DEA) Isocratic: 40% B Flow rate: 2.5 mL/min Column temp:40° C. to give

Peak 1: 7-isopropoxy-N-(1-((1R,2R)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide [stereochemistry arbitrarily assigned](16 mg, 46% yield). LCMS m/z=480.2 [M+H]⁺; 1HNMR: (500 MHz, MeOH-d₄) δ ppm: 9.38 (s, 1H), 8.50 (d, J=7.5 Hz, 1H), 7.61 (s, 1H), 7.23 (d, J=7.0 Hz, 1H), 6.44-6.19 (m, 1H), 5.79-5.74 (m, 1H), 4.89 (s, 1H), 4.00 (s, 2H), 3.61 (s, 3H), 3.49-3.42 (m, 2H), 2.11 (d, J=4.5 Hz, 2H), 1.86 (d, J=4.5 Hz, 2H), 1.64 (d, J=6.5 Hz, 6H), 1.50 (s, 3H), 1.46-1.42 (m, 1H), 1.37-1.33 (m, 1H).

and Peak 2: 7-isopropoxy-N-(1-((1S,2S)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide [stereochemistry arbitrarily assigned](16.9 mg, 48% yield). LCMS m/z=480.2 [M+H]⁺; 1HNMR (500 MHz, MeOH-d₄) δ ppm 9.38 (s, 1H), 8.50 (d, J=7.5 Hz, 1H), 7.61 (s, 1H), 7.23 (d, J=7.0 Hz, 1H), 6.33 (t, J=7.0 Hz, 1H), 5.79-5.74 (m, 1H), 4.00 (s, 2H), 3.61 (s, 3H), 3.49-3.43 (m, 2H), 2.11 (d, J=4.5 Hz, 2H), 1.86 (d, J=4.5 Hz, 2H), 1.64 (d, J=6.5 Hz, 6H), 1.50 (s, 3H), 1.44 (d, J=4.5 Hz, 1H), 1.38-1.33 (m, 1H).

### Example 139 and 140: 7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide and 7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide

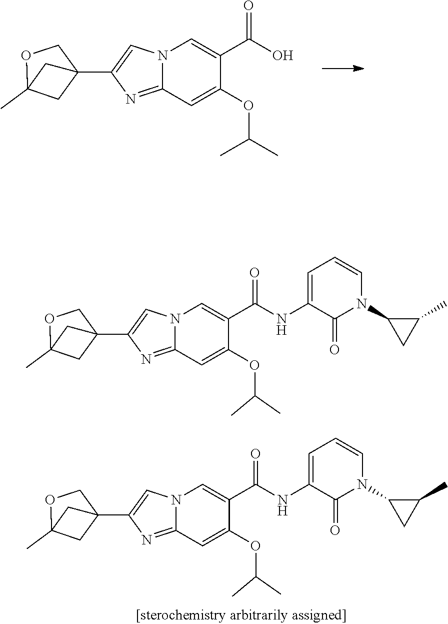

Trans-7-isopropoxy-2-(1-methyl-2-oxabicyclo[2.1.1]hexan-4-yl)-N-(1-(2-methylcyclopropyl)-2-oxo-1,2-dihydropyridin-3-yl)imidazo [1,2-a]pyridine-6-carboxamide was obtained as a yellow oil, 40 mg, 54.7%, from trans-3-amino-1-(2-methylcyclopropyl)pyridin-2(1H)-one hydrochloride (Preparation 76) and 7-isopropoxy-2-(1-methyl-2-oxabicyclo[2.1.1]hexan-4-yl)imidazo[1,2-a]pyridine-6-carboxylic acid (Preparation 4) following the method described in Example 137 and 138.

The compound was further purified by SFC, Column: Chiralpak AD-3 (250 mm×30 mm×10 um) Mobile phase: A: CO₂ B: NH₃ (0.05% DEA), Gradient: from 5% to 40% of B in 2 min Flow rate: 4 mL/min, Column temp.: 35° C. to give:

Peak 1: 7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide (20.1 mg, 50.25% yield) as a white solid. LCMS m/z=463.2 [M+H]⁺; 1H NMR (500 MHz, CDCl₃) δ: 10.79 (s, 1H), 8.97 (s, 1H), 8.51 (d, J=7.5 Hz, 1H), 7.34 (s, 1H), 7.02 (d, J=7.0 Hz, 1H), 6.20 (t, J=7.0 Hz, 1H), 4.90-4.80 (m, 1H), 4.07 (s, 2H), 3.20-3.10 (m, 1H), 2.10-2.00 (m, 2H), 2.00-1.90 (m, 2H), 1.70-1.60 (m, 9H), 1.27 (d, J=5.5 Hz, 3H), 1.30-1.20 (m, 1H), 1.10-1.00 (m, 1H), 0.90-0.80 (m, 1H) and

Peak 2: 7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide, (18.2 mg, 45.50% yield) as white solid. LCMS m/z=463.2 [M+H]⁺; 1H NMR (500 MHz, CDCl₃) δ: 10.79 (s, 1H), 8.97 (s, 1H), 8.51 (d, J=7.5 Hz, 1H), 7.34 (s, 1H), 7.02 (d, J=7.0 Hz, 1H), 6.20 (t, J=7.0 Hz, 1H), 4.90-4.80 (m, 1H), 4.07 (s, 2H), 3.20-3.10 (m, 1H), 2.10-2.00 (m, 2H), 2.00-1.90 (m, 2H), 1.70-1.60 (m, 9H), 1.27 (d, J=5.5 Hz, 3H), 1.30-1.20 (m, 1H), 1.10-1.00 (m, 1H), 0.90-0.80 (m, 1H).

### Examples 141 and 142: 7-isopropoxy-2-((1R,4S)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide and 7-isopropoxy-2-((1R,4S)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide

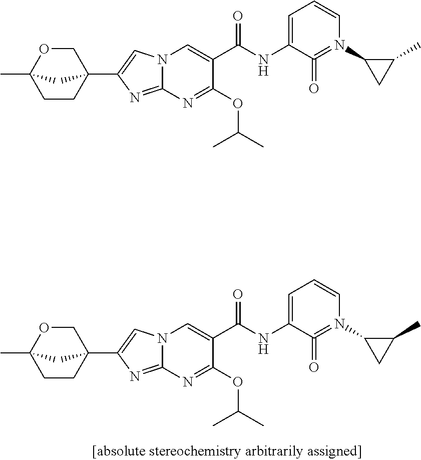

Trans-7-isopropoxy-2-((1R,4S)-1-methyl-2-oxabicyclo [2.2.1]heptan-4-yl)-N-(1-(2-methylcyclopropyl)-2-oxo-1,2-dihydropyridin-3-yl)imidazo[1,2-a]pyrimidine-6-carboxamide was obtained as a white solid, 65 mg. 90% yield, from 7-isopropoxy-2-((1R,4S)-1-methyl-2-oxabicyclo[2.2.1]heptan-4-yl)imidazo[1,2-a]pyrimidine-6-carboxylic acid (Preparation 92) and trans-3-amino-1-(2-methylcyclopropyl)pyridin-2(1H)-one hydrochloride (Preparation 76), following the procedure described in Example 137 and 138.

This compound was further purified by SFC: ChiralPak AD-3 150×4.6 mm I.D., 3 um; Mobile phase: A: CO₂ B: IPA (0.05% DEA); Gradient: from 5% to 40% of B in 5.5 min; Flow rate: 2.5 mL/min Column temp.:40° C.; to give:

Peak 1: 7-isopropoxy-2-((1R,4S)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide (stereochemistry arbitrarily assigned) (28.7 mg, 44% yield) as a white solid. LCMS m/z=478.3 [M+H]⁺; 1HNMR (400 MHz, CDCl₃) δ ppm: 10.81 (s, 1H), 9.13 (s, 1H), 8.50-8.47 (m, 1H), 7.22 (s, 1H), 7.04-7.01 (m, 1H), 6.22-6.18 (m, 1H), 5.89-5.82 (m, 1H), 4.14-4.11 (m, 1H), 3.96 (d, J=6.4 Hz, 1H), 3.16-3.12 (m, 1H), 2.22-2.14 (m, 1H), 2.10-2.05 (m, 1H), 2.03-2.00 (m, 1H), 1.92 (d, J=9.2 Hz, 1H), 1.87-1.81 (m, 2H), 1.64 (d, J=6.4 Hz, 6H), 1.48 (s, 3H), 1.29 (d, J=4.8 Hz, 3H), 1.25-1.20 (m, 1H), 1.09-1.04 (m, 1H), 0.97-0.92 (m, 1H)

Peak 2: 7-isopropoxy-2-((1R,4S)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide (stereochemistry arbitrarily assigned) (32 mg, 49% yield) as white solid. LCMS m/z=478.3 [M+H]⁺;

1HNMR (400 MHz, CDCl₃) δ:10.82 (s, 1H), 9.14 (s, 1H), 8.50-8.47 (m, 1H), 7.22 (s, 1H), 7.04-7.01 (m, 1H), 6.22-6.18 (m, 1H), 5.89-5.82 (m, 1H), 4.14-4.11 (m, 1H), 3.96 (d, J=6.4 Hz, 1H), 3.16-3.12 (m, 1H), 2.21-2.14 (m, 1H), 2.11-2.07 (m, 1H), 2.03-2.00 (m, 1H), 1.94-1.91 (m, 1H), 1.88-1.81 (m, 2H), 1.64 (d, J=6.0 Hz, 6H), 1.49 (s, 3H), 1.29 (d, J=5.2 Hz, 3H), 1.27-1.21 (m, 1H), 1.10-1.04 (m, 1H), 0.97-0.92 (m, 1H).

### Example 143: N-(1-((1R,2R)-2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

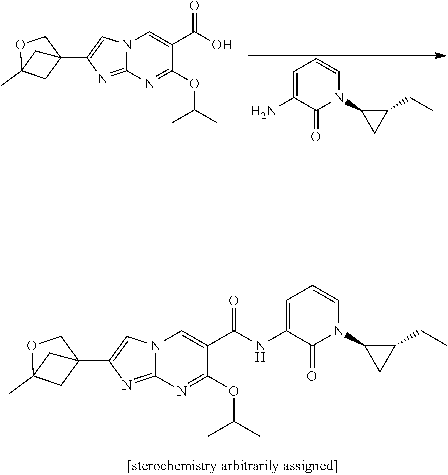 [SMILES: CC(C)Oc1nc2nc(C34COC(C)(C3)C4)cn2cc1C(=O)O ; CC[C@@H]1C[C@H]1n1cccc(N)c1=O ; CC[C@@H]1C[C@H]1n1cccc(NC(=O)c2cn3cc(C45COC(C)(C4)C5)nc3nc2OC(C)C)c1=O]

To a solution of 7-isopropoxy-2-(1-methyl-2-oxabicyclo[2.1.1]hexan-4-yl)imidazo[1,2-a]pyrimidine-6-carboxylic acid (Preparation 8, 100.0 mg, 0.315 mmol) and trans-3-amino-1-(2-ethylcyclopropyl)pyridin-2(1H)-one hydrochloride (Preparation 77, 112.3 mg, 0.63 mmol) in pyridine (5 mL) was added T3P® (5 mL) and the reaction stirred at 20° C. for 2 h. The reaction mixture was concentrated in vacuo, the residue was diluted with water (10 mL), then aqueous NaHCO₃ (10 mL) and extracted with EtOAc (20 mL×3). The combined organic layer was washed with brine (30 mL), dried over Na₂SO₄, filtered and the filtrate concentrated in vacuo. The residue was purified by prep-HPLC (Column: Phenomenex Synergi C18 150*30 mm*4 um; Mobile Phase: from 49% to 69% of water (0.05% (NH₄HCO₃)-MeCN) to give trans-N-(1-(−2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (80 mg, 52% yield) as a white solid. LCMS m/z=478.3 [M+H]⁺

trans-N-(1-(−2-Ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide was further purified by SFC: Column: Phenomenex-Cellulose-2 (250 mm*30 mm, 5 um); Mobile phase: A: CO₂ B: IPA (0.05% DEA); Isocratic: 60% B; Flow rate: 2.8 mL/min; Column temp.: 35° C.; to give:

Peak 1: N-(1-((1R,2R)-2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide [stereochemistry was arbitrarily assigned] (30.20 mg, 38% yield) as a white solid. 1H NMR (400 MHz, MeOH-d₄) δ ppm: 9.37 (s, 1H), 8.51 (d, J=7.2 Hz, 1H), 7.61 (s, 1H), 7.28 (d, J=6.8 Hz, 1H), 6.32 (t, J=7.2 Hz, 1H), 5.79-5.72 (m, 1H), 4.00 (s, 2H), 3.18 (d, J=7.2 Hz, 1H), 2.11 (d, J=4.4 Hz, 2H), 1.86 (d, J=4.4 Hz, 2H), 1.64 (d, J=6.0 Hz, 6H), 1.58-1.54 (m, 1H), 1.52-1.50 (m, 3H), 1.47-1.43 (m, 1H), 1.29-1.23 (m, 1H), 1.15-1.12 (m, 3H), 1.10-1.09 (m, 1H), 1.00-0.95 (m, 1H).

Further elution provided N-(1-((1S,2S)-2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (22.5 mg, 28% yield) as white solid.

### Example 144: N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

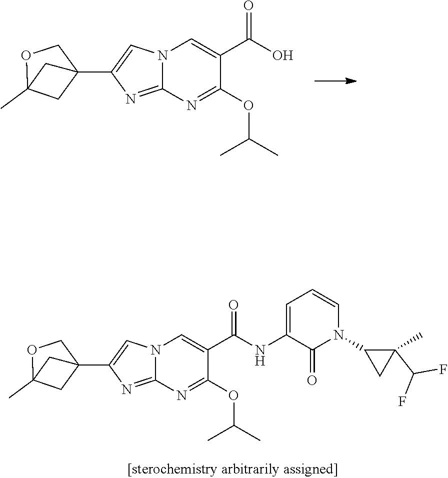

Cis-N-(1-(2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide was obtained as a white solid (50 mg, 62% yield) from 7-isopropoxy-2-(1-methyl-2-oxabicyclo[2.1.1]hexan-4-yl)imidazo[1,2-a]pyrimidine-6-carboxylic acid (Preparation 8) and cis-3-amino-1-(2-(difluoromethyl)-2-methylcyclopropyl)pyridin-2(1H)-one hydrochloride (Preparation 80) following a similar procedure to that described in Example 143.

This compound (50 mg, 97.36 μmol) was further purified by prep. SFC: Column: Chiralcel OD-3 100×4.6 mm I.D., 3 um Mobile phase: A: CO₂ B: MeOH (0.05% DEA) Isocratic: 40% B Flow rate: 2.8 mL/min Column temp: 35° C. to give:

Peak 1: N-(1-((1R,2R)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (stereochemistry arbitrarily assigned, 22.8 mg, 46% yield) as a white solid and

Peak 2: N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (stereochemistry arbitrarily assigned, 20.3 mg, 41% yield) as a white solid. LCMS m/z=514.1 [M+H]⁺; 1H NMR (400 MHz, DMSO-d₆) δ:10.69 (s, 1H), 9.43 (s, 1H), 8.40 (d, J=6.0 Hz, 1H), 7.68 (s, 1H), 7.30 (d, J=5.6 Hz, 1H), 6.32 (t, J=7.2 Hz, 1H), 6.02-5.73 (m, 1H), 5.58-5.52 (m, 1H), 3.84 (s, 2H), 3.62 (t, J=6.8 Hz, 1H), 1.96 (d, J=4.4 Hz, 2H), 1.72-1.71 (m, 2H), 1.50 (dd, J=1.2, 6.0 Hz, 6H), 1.39 (s, 5H), 0.81 (s, 3H).

### Example 145: N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide

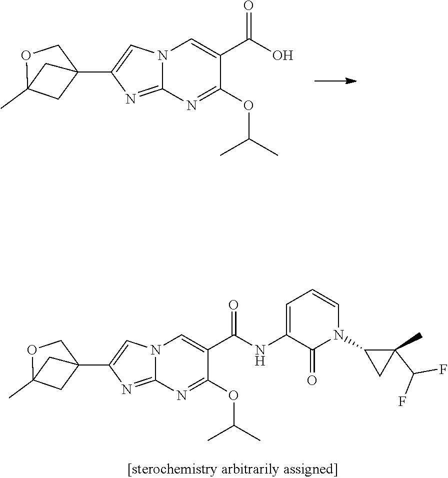

Trans-N-(1-(2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide was obtained as a yellow oil (50 mg, 62% yield) from 7-isopropoxy-2-(1-methyl-2-oxabicyclo[2.1.1]hexan-4-yl)imidazo[1,2-a]pyrimidine-6-carboxylic acid (Preparation 8) and trans-3-amino-1-(2-(difluoromethyl)-2-methylcyclopropyl)pyridin-2(1H)-one hydrochloride (Preparation 81) following a similar procedure to that described in Example 143.

The compound was purified by prep. SFC: Chiralpak AD-3 50×4.6 mm I.D., 3 um Mobile phase: A: CO₂ B: EtOH (0.05% DEA) Gradient: from 5% to 40% of B in 2 min Flow rate: 4 mL/min Column temp.: 35° C. to give:

Peak 1: N-(1-((1R,2R)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (stereochemistry arbitrarily assigned)(3.2 mg, 19% yield) as white solid and

Peak 2: N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide (stereochemistry arbitrarily assigned)(11.6 mg, 39% yield) as a white solid. LCMS m/z=514.1 [M+H]⁺; 1H NMR (500 MHz, DMSO-d₆) δ ppm: 10.71 (s, 1H), 9.46 (s, 1H), 8.40 (d, J=7.5 Hz, 1H), 7.71 (s, 1H), 7.36 (d, J=7.0 Hz, 1H), 6.31 (t, J=7.5 Hz, 1H), 5.61-5.36 (m, 2H), 3.87 (s, 2H), 3.47 (s, 1H), 1.99 (d, J=4.0 Hz, 2H), 1.75 (d, J=3.0 Hz, 2H), 1.72-1.70 (m, 1H), 1.54 (d, J=6.0 Hz, 6H), 1.43 (s, 3H), 1.31 (s, 3H), 1.23 (s, 1H).

### Examples 146 and 147: 8-fluoro-7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide and 8-fluoro-7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide

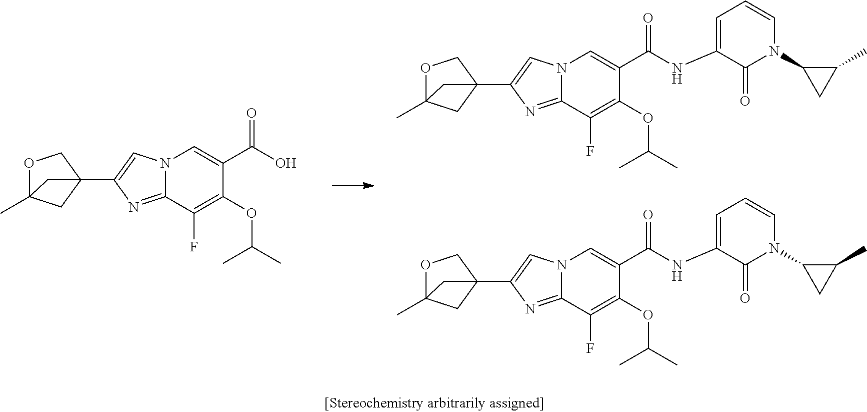

8-Fluoro-7-isopropoxy-2-(1-methyl-2-oxabicyclo [2.1.1]hexan-4-yl)-N-(1-((1S,2S)-2-methylcyclopropyl)-2-oxo-1,2-dihydropyridin-3-yl)imidazo[1,2-a]pyridine-6-carboxamide and 8-fluoro-7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide were obtained as white solids, from 8-fluoro-7-isopropoxy-2-(1-methyl-2-oxabicyclo[2.1.1]hexan-4-yl)imidazo[1,2-a]pyridine-6-carboxylic acid (Preparation 73) and trans-3-amino-1-(2-methylcyclopropyl)pyridin-2(1H)-one hydrochloride (Preparation 76), following the procedure described in Example 143.

LCMS m/z=481.3 [M+H]⁺; 1H NMR (500 MHz, MeOH-d₄) δ ppm: 8.99 (d, J=1.0 Hz, 1H), 8.53 (d, J=7.5 Hz, 1H), 7.86 (d, J=3.0 Hz, 1H), 7.32 (d, J=8.5 Hz, 1H), 6.34 (t, J=7.5 Hz, 1H), 4.95-4.92 (m, 1H), 4.03 (s, 2H), 3.13-3.10 (m, 1H), 2.15-2.14 (m, 2H), 1.90-1.89 (m, 2H), 1.54-1.50 (m, 6H), 1.50 (s, 3H), 1.27 (s, 3H), 1.15-1.11 (m, 1H), 1.02-0.99 (m, 1H), 0.96-0.93 (m, 1H).

LCMS m/z=481.3 [M+H]⁺; 1H NMR: (500 MHz, MeOH-d₄) δ ppm: 8.99 (d, J=1.0 Hz, 1H), 8.53 (d, J=7.5 Hz, 1H), 7.86 (d, J=3.0 Hz, 1H), 7.32 (d, J=8.5 Hz, 1H), 6.34 (t, J=7.5 Hz, 1H), 4.95-4.92 (m, 1H), 4.03 (s, 2H), 3.13-3.10 (m, 1H), 2.15-2.14 (m, 2H), 1.90-1.89 (m, 2H), 1.54-1.52 (m, 6H), 1.50 (s, 3H), 1.27 (s, 3H), 1.26-1.24 (m, 1H), 1.15-1.11 (m, 1H), 0.95-0.94 (m, 1H).

### Examples 148 and 149: 7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide and 7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide

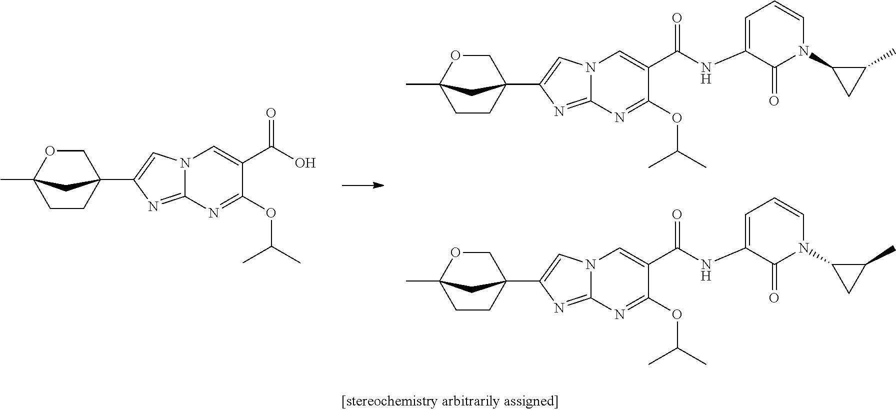

Trans-7-isopropoxy-2-((1S,4R)-1-methyl-2-oxabicyclo[2.2.1]heptan-4-yl)-N-(1-(2-methylcyclopropyl)-2-oxo-1,2-dihydropyridin-3-yl)imidazo[1,2-a]pyrimidine-6-carboxamide was obtained as a white solid, 75 mg. 97% yield, from 7-isopropoxy-2-((1S,4R)-1-methyl-2-oxabicyclo[2.2.1]heptan-4-yl)imidazo[1,2-a]pyrimidine-6-carboxylic acid (Preparation 91) and trans-3-amino-1-(2-methylcyclopropyl)pyridin-2(1H)-one hydrochloride (Preparation 76), following a similar procedure to that described in Example 143.

This compound was further purified by SFC: Phenomenex-Cellulose-2 (250 mm*30 mm, 5 um); Mobile phase: A: CO₂ B: IPA (0.05% DEA); Isocratic: 60% B; Flow rate: 2.8 mL/min; Column temp.: 35° C. to give:

Peak 1: 7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide (stereochemistry arbitrarily assigned) (30.40 mg) as a white solid. LCMS m/z=478.3 [M+H]⁺; 1H NMR: (400 MHz, MeOH-d₄) δ ppm: 9.36 (s, 1H), 8.52 (d, J=7.2 Hz, 1H), 7.58 (s, 1H), 7.29 (d, J=6.8 Hz, 1H), 6.33 (t, J=7.2 Hz, 1H), 5.79-5.73 (m, 1H), 4.04 (d, J=6.4 Hz, 1H), 3.90 (d, J=6.4 Hz, 1H), 3.14-3.11 (m, 1H), 2.20-2.13 (m, 1H), 2.08-2.02 (m, 1H), 1.95 (s, 2H), 1.88-1.79 (m, 2H), 1.64 (d, J=6.0 Hz, 6H), 1.46 (s, 3H), 1.28 (s, 4H), 1.16-1.11 (m, 1H), 0.95-0.90 (m, 1H).

Peak 2: 7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide (stereochemistry arbitrarily assigned) (27.50 mg) as white solid. LCMS m/z=478.3 [M+H]⁺; 1H NMR (400 MHz, MeOH-d₄) δ ppm: 9.36 (s, 1H), 8.52 (d, J=7.2 Hz, 1H), 7.58 (s, 1H), 7.29 (d, J=6.8 Hz, 1H), 6.33 (t, J=7.2 Hz, 1H), 5.79-5.72 (m, 1H), 4.04 (d, J=6.4 Hz, 1H), 3.90 (d, J=6.4 Hz, 1H), 3.15-3.11 (m, 1H), 2.20-2.12 (m, 1H), 2.08-2.03 (m, 1H), 1.95 (s, 2H), 1.90-1.79 (m, 2H), 1.64 (d, J=6.0 Hz, 6H), 1.46 (s, 3H), 1.28 (s, 4H), 1.16-1.11 (m, 1H), 0.95-0.92 (m, 1H).

### Examples 150 and 151: 7-isopropoxy-2-((1S,4R)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide and 7-isopropoxy-2-((1S,4R)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide

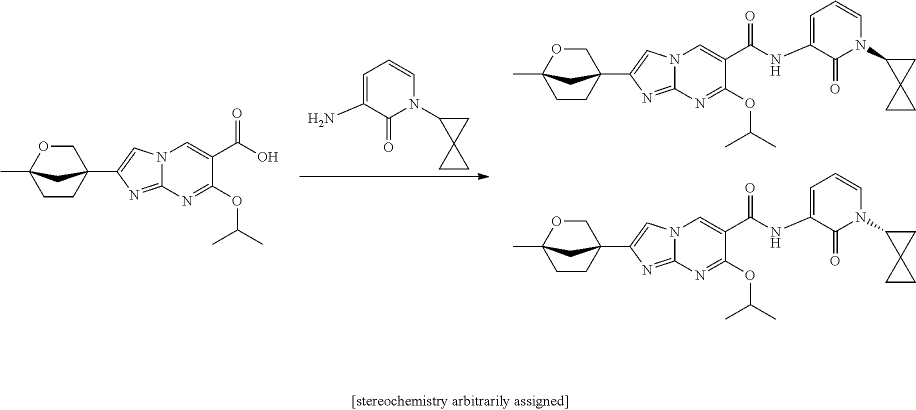 [SMILES: CC(C)Oc1nc2nc([C@@]34CC[C@@](C)(C3)OC4)cn2cc1C(=O)O ; Nc1cccn(C2CC23CC3)c1=O ; CC(C)Oc1nc2nc([C@@]34CC[C@@](C)(C3)OC4)cn2cc1C(=O)Nc1cccn([C@@H]2CC23CC3)c1=O ; CC(C)Oc1nc2nc([C@@]34CC[C@@](C)(C3)OC4)cn2cc1C(=O)Nc1cccn([C@H]2CC23CC3)c1=O]

To a solution of 7-isopropoxy-2-((1S,4R)-1-methyl-2-oxabicyclo[2.2.1]heptan-4-yl)imidazo[1,2-a]pyrimidine-6-carboxylic acid (Preparation 91, 50 mg, 150.9 μmol) in T3P® (1.5 mL) was added 3-amino-1-(spiro[2.2]pentan-1-yl)pyridin-2(1H)-one (Preparation 79, 27 mg, 153.2 μmol) and pyridine (1.5 mL) and the reaction was stirred at 20° C. for 3 h. The reaction was evaporated under vacuum, the residue was diluted with aqueous NaHCO₃ (30 mL) to pH=7 and extracted with EtOAc (30 mL×3). The combined organic layers were washed with brine (50 mL) and dried over Na₂SO₄. The filtrate was concentrated in vacuo and the residue was purified by prep-HPLC (Column: Welch Xtimate C18 150*25 mm*5 um)); Mobile Phase: from 42% to 72% of water (10 mM NH₄HCO₃)-MeCN at 25 mL/min to give racemic 7-isopropoxy-2-((1S,4R)-1-methyl-2-oxabicyclo[2.2.1]heptan-4-yl)-N-(2-oxo-1-(spiro[2.2]pentan-1-yl)-1,2-dihydropyridin-3-yl)imidazo[1,2-a]pyrimidine-6-carboxamide (50 mg, 77% yield) as a white solid.

This was further purified by SFC: DAICEL CHIRALCEL OD-H (250 mm*30 mm, 5 um)); Mobile Phase: from 50% to 50% of 0.1% NH₃H₂O EtOH at 80 mL/min to give:

Peak 1: 7-isopropoxy-2-((1S,4R)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide (stereochemistry arbitrarily assigned)(19.5 mg, 39% yield) as a white solid. LCMS m/z=490.1 [M+H]⁺; 1H NMR: (500 MHz, DMSO-d₆) δ:10.72 (s, 1H), 9.45 (s, 1H), 8.41 (dd, J=7.5 Hz, J=2.0 Hz, 1H), 7.69 (s, 1H), 7.39 (dd, J=7.0 Hz, J=2.0 Hz, 1H), 6.33 (t, J=7.0 Hz, 1H), 5.63-5.58 (m, 1H), 3.93 (dd, J=6.0 Hz, J=3.0 Hz, 2H), 3.75 (d, J=6.5 Hz, 1H), 2.08-1.92 (m, 2H), 1.83-1.77 (m, 4H), 1.56 (d, J=6.5 Hz, 1H), 1.52 (dd, J=6.0 Hz, J=2.5 Hz, 6H), 1.48 (d, J=6.0 Hz, 1H), 1.38 (s, 3H), 1.09-0.85 (m, 4H).

Peak 2: 7-isopropoxy-2-((1S,4R)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide (stereochemistry arbitrarily assigned) (21.50 mg, 43% yield). LCMS m/z=490.3 [M+H]⁺; 1H NMR: (500 MHz, DMSO-d₆) δ:10.71 (s, 1H), 9.45 (s, 1H), 8.41 (dd, J=7.5 Hz, J=1.5 Hz, 1H), 7.69 (s, 1H), 7.39 (dd, J=6.5 Hz, J=1.5 Hz, 1H), 6.33 (t, J=7.5 Hz, 1H), 5.63-5.58 (m, 1H), 3.93 (dd, J=6.0 Hz, J=3.0 Hz, 2H), 3.75 (d, J=6.0 Hz, 1H), 2.05-1.92 (m, 2H), 1.81-1.70 (m, 4H), 1.55 (d, J=6.5 Hz, 1H), 1.52 (dd, J=6.0 Hz, J=2.5 Hz, 6H), 1.49-1.47 (m, 1H), 1.38 (s, 3H), 1.11-0.85 (m, 4H).

### Examples 152 and 153: 7-isopropoxy-2-((1R,4S)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide and 7-isopropoxy-2-((1R,4S)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide

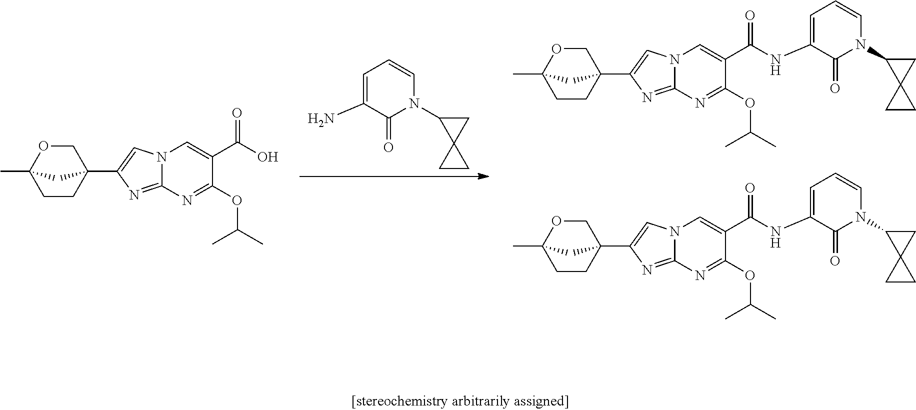 [SMILES: CC(C)Oc1nc2nc([C@]34CC[C@](C)(C3)OC4)cn2cc1C(=O)O ; Nc1cccn(C2CC23CC3)c1=O ; CC(C)Oc1nc2nc([C@]34CC[C@](C)(C3)OC4)cn2cc1C(=O)Nc1cccn([C@@H]2CC23CC3)c1=O ; CC(C)Oc1nc2nc([C@]34CC[C@](C)(C3)OC4)cn2cc1C(=O)Nc1cccn([C@H]2CC23CC3)c1=O]

Racemic 7-isopropoxy-2-((1R,4S)-1-methyl-2-oxabicyclo[2.2.1]heptan-4-yl)-N-(2-oxo-1-(-spiro[2.2]pentan-1-yl)-1,2-dihydropyridin-3-yl)imidazo[1,2-a]pyrimidine-6-carboxamide was obtained from 3-amino-1-(spiro[2.2]pentan-1-yl)pyridin-2(1H)-one (Preparation 79) and 7-isopropoxy-2-((1R,4S)-1-methyl-2-oxabicyclo[2.2.1]heptan-4-yl)imidazo[1,2-a]pyrimidine-6-carboxylic acid (Preparation 92) following the procedure described in Examples 149 and 150.

This compound (35.0 mg, 71.49 μmol) was purified by SFC: Chiralpak IG-3 100 um, 4.6 mm I.D. 3 um; Mobile phase: 40% of EtOH (0.05% DEA) in CO₂; Flow rate: 2.8 mL/min; Column temp.: 35° C.; ABPR: 1500 psi) to give: Peak 1: 7-isopropoxy-2-((1R,4S)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide (stereochemistry arbitrarily assigned)(16.8 mg) as a white solid. LCMS m/z=490.3 [M+H]⁺; 1HNMR (500 MHz, CDCl₃) δ ppm: 10.82 (d, J=3.5 Hz, 1H), 9.14 (d, J=7.0 Hz, 1H), 8.53-8.48 (m, 1H), 7.22 (s, 1H), 7.11-7.09 (m, 1H), 6.26-6.21 (m, 1H), 5.89-5.82 (m, 1H), 4.16-4.12 (m, 1H), 4.05-4.03 (m, 1H), 3.98-3.96 (m, 1H), 2.25-2.11 (m, 2H), 2.09-1.94 (m, 2H), 1.90-1.81 (m, 2H), 1.63-1.58 (m, 6H), 1.49 (d, J=4.0 Hz, 3H), 1.34-1.32 (m, 2H), 1.26-1.19 (m, 2H), 1.03-0.92 (m, 2H)

Peak 2: 7-isopropoxy-2-((1R,4S)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide (13 mg) (stereochemistry arbitrarily assigned) as white solid. LCMS m/z=490.3 [M+H]⁺; 1HNMR: (500 MHz, CDCl₃) δ ppm: 10.83 (s, 1H), 9.14 (d, J=6.0 Hz, 1H), 8.53-8.48 (m, 1H), 7.22 (s, 1H), 7.11-7.09 (m, 1H), 6.26-6.21 (m, 1H), 5.89-5.83 (m, 1H), 4.14-4.12 (m, 1H), 4.05-4.03 (m, 1H), 3.98-3.96 (m, 1H), 2.21-2.12 (m, 2H), 2.07-1.97 (m, 2H), 1.88-1.82 (m, 2H), 1.63-1.59 (m, 6H), 1.49 (d, J=4.0 Hz, 3H), 1.34-1.32 (m, 2H), 1.26-1.20 (m, 2H), 1.03-0.93 (m, 2H).

### Example 154 and 155: N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide and N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

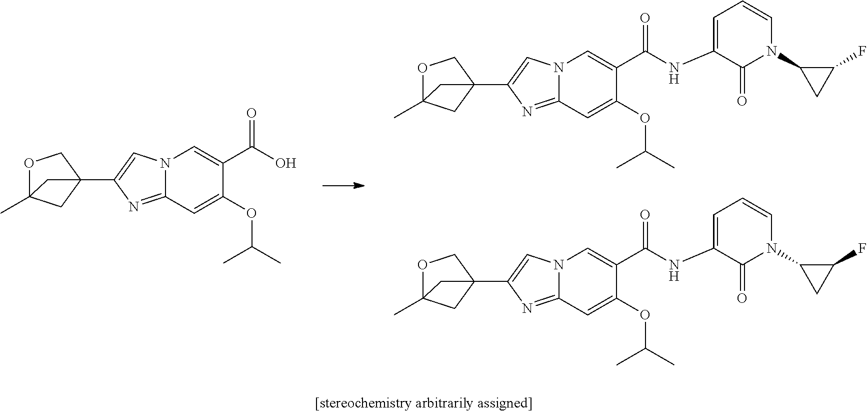 [SMILES: CC(C)Oc1cc2nc(C34COC(C)(C3)C4)cn2cc1C(=O)O ; CC(C)Oc1cc2nc(C34COC(C)(C3)C4)cn2cc1C(=O)Nc1cccn([C@@H]2C[C@H]2F)c1=O ; CC(C)Oc1cc2nc(C34COC(C)(C3)C4)cn2cc1C(=O)Nc1cccn([C@H]2C[C@@H]2F)c1=O]

Trans-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide was obtained (50 mg), from 7-isopropoxy-2-(1-methyl-2-oxabicyclo[2.1.1]hexan-4-yl)imidazo[1,2-a]pyridine-6-carboxylic acid (Preparation 4) and trans-3-amino-1-(2-fluorocyclopropyl)pyridin-2(1H)-one (Preparation 61) following a similar procedure to that described in Example 149 and 150.

This was further purified by SFC (Column: DAICEL CHIRALCEL IC (250 mm, 30 mm, 10 um); Mobile Phase: from 50% to 50% of 0.1% NH₃·H₂O EtOH; Flow Rate (mL/min): 80; Column temp: 35° C.) to give:

Peak 1: N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide (stereochemistry arbitrarily assigned)(23.4 mg, 47% yield). LCMS m/z=467.1 [M+H]⁺; 1H NMR (400 MHz, MeOH-d₄) δ ppm: 9.12 (s, 1H), 8.55 (d, J=7.6 Hz, 1H), 7.70 (s, 1H), 7.22 (d, J=7.6 Hz, 1H), 7.00 (s, 1H), 6.36 (t, J=7.2 Hz, 1H), 5.10-5.00 (m, 1H), 4.80-4.70 (m, 1H), 4.01 (s, 2H), 3.80-3.70 (m, 1H), 2.10-2.00 (m, 2H), 1.90-1.80 (m, 2H), 1.78-1.70 (m, 1H), 1.63 (d, J=6.4 Hz, 6H), 1.50-1.40 (m, 4H).

Peak 2: N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide (stereochemistry arbitrarily assigned)(24.8 mg, 50% yield) as an off-white solid. 1H NMR (400 MHz, MeOH-d₄) δ ppm: 9.12 (s, 1H), 8.55 (d, J=7.6 Hz, 1H), 7.70 (s, 1H), 7.22 (d, J=7.6 Hz, 1H), 7.00 (s, 1H), 6.36 (t, J=7.2 Hz, 1H), 5.10-5.00 (m, 1H), 4.80-4.70 (m, 1H), 4.01 (s, 2H), 3.80-3.70 (m, 1H), 2.10-2.00 (m, 2H), 1.90-1.80 (m, 2H), 1.78-1.70 (m, 1H), 1.63 (d, J=6.4 Hz, 6H), 1.50-1.40 (m, 4H).

### Example 156 and 157: 7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyrimidine-6-carboxamide and 7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyrimidine-6-carboxamide

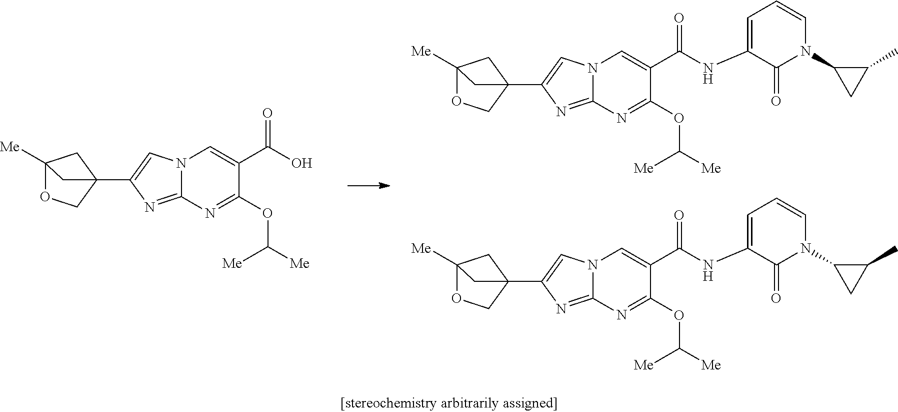 [SMILES: CC(C)Oc1nc2nc(C34COC(C)(C3)C4)cn2cc1C(=O)O ; CC(C)Oc1nc2nc(C34COC(C)(C3)C4)cn2cc1C(=O)Nc1cccn([C@@H]2C[C@H]2C)c1=O ; CC(C)Oc1nc2nc(C34COC(C)(C3)C4)cn2cc1C(=O)Nc1cccn([C@H]2C[C@@H]2C)c1=O]

DIPEA (244.36 mg, 1.89 mmol) and HATU (252.29 mg, 0.662 mmol) were added to a solution of 7-isopropoxy-2-(1-methyl-2-oxabicyclo[2.1.1]hexan-4-yl)imidazo[1,2-a]pyrimidine-6-carboxylic acid (Preparation 8, 200 mg, 0.63 mmol) in DMF (3 mL). Trans-racemic 3-amino-1-(2-methylcyclopropyl)pyridin-2(1H)-one hydrochloride Preparation 76, 126.47 mg, 0.63 mmol) was added and the reaction stirred at rt overnight. The resulting mixture was concentrated under reduced pressure and the residue purified by RPHPLC using a gradient of 5-60% MeCN in water to give trans N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide. LCMS m/z=464.5 [M+H]⁺; 1H NMR (500 MHz, DMSO-d₆) δ: 10.63 (s, 1H), 9.63 (s, 1H), 8.40 (dd, J=1.68, 7.32 Hz, 1H), 7.96-7.86 (m, 1H), 7.37 (dd, J=1.60, 6.94 Hz, 1H), 6.31 (t, J=7.17 Hz, 1H), 5.70-5.55 (m, 1H), 3.92 (s, 2H), 3.26-3.16 (m, 1H), 2.19-2.06 (m, 2H), 1.83 (br d, J=4.27 Hz, 2H), 1.59 (dd, J=2.52, 6.18 Hz, 6H), 1.44 (s, 3H), 1.27-1.16 (m, 4H), 1.13-1.05 (m, 1H), 0.93-0.81 (m, 1H)

This was further purified by SFC CHIRALPAK AD-H 30×250 mm, 5 um, Method: 30% MeOH w/ 0.1% DEA in CO₂ (flow rate: 100 mL/min, column temp 40° C.) to provide:

Peak 1: 7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyrimidine-6-carboxamide (stereochemistry arbitrarily assigned) 12 mg. LCMS m/z=464.3 [M+H]⁺; 1H NMR (400 MHz, MeOH-d₄) δ 9.40 (s, 1H), 8.56 (dd, J=1.76, 7.28 Hz, 1H), 7.63 (s, 1H), 7.33 (dd, J=1.88, 6.90 Hz, 1H), 6.37 (t, J=7.28 Hz, 1H), 5.79 (quin, J=6.27 Hz, 1H), 4.02 (s, 2H), 3.16-3.15 (m, 1H), 2.17-2.09 (m, 2H), 1.88 (dd, J=1.63, 4.64 Hz, 2H), 1.66 (d, J=6.27 Hz, 6H), 1.52 (s, 3H), 1.30 (d, J=1.51 Hz, 4H), 1.20-1.10 (m, 1H), 1.01-0.91 (m, 1H) and

Peak 2: 7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyrimidine-6-carboxamide (stereochemistry arbitrarily assigned) 12.6 mg. LCMS m/z=464.3 [M+H]⁺; 1H NMR (400 MHz, MeOH-d₄) δ: 9.40 (s, 1H), 8.56 (dd, J=1.76, 7.28 Hz, 1H), 7.63 (s, 1H), 7.33 (dd, J=1.76, 7.03 Hz, 1H), 6.37 (t, J=7.28 Hz, 1H), 5.79 (quin, J=6.27 Hz, 1H), 4.03 (s, 2H), 3.16-3.15 (m, 1H), 2.13 (dd, J=1.63, 4.64 Hz, 2H), 1.88 (dd, J=1.63, 4.64 Hz, 2H), 1.66 (d, J=6.27 Hz, 6H), 1.52 (s, 3H), 1.33-1.27 (m, 4H), 1.20-1.12 (m, 1H), 1.00-0.89 (m, J=5.80 Hz, 1H).

### Examples 158 and 159: N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide and N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

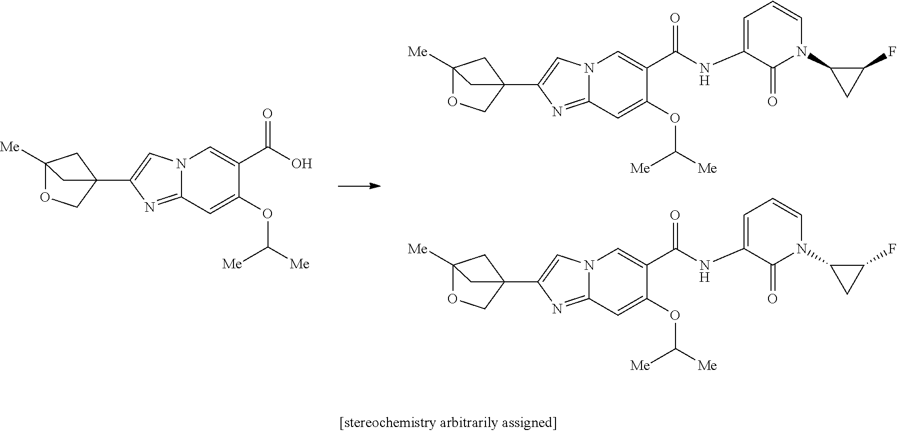 [SMILES: CC(C)Oc1cc2nc(C34COC(C)(C3)C4)cn2cc1C(=O)O ; CC(C)Oc1cc2nc(C34COC(C)(C3)C4)cn2cc1C(=O)Nc1cccn([C@@H]2C[C@@H]2F)c1=O ; CC(C)Oc1cc2nc(C34COC(C)(C3)C4)cn2cc1C(=O)Nc1cccn([C@H]2C[C@H]2F)c1=O]

Cis-3-amino-1-(2-fluorocyclopropyl)pyridin-2(1H)-one hydrochloride (Preparation 63, 15 mg, 73.3 μmol) was dissolved in DMF (1 mL), HATU (30.74 mg, 80.63 μmol) and DIPEA (28.42 mg, 219.9 μmol) were added and the solution stirred for 1 min. 7-isopropoxy-2-(1-methyl-2-oxabicyclo[2.1.1]hexan-4-yl)imidazo[1,2-a]pyrimidine-6-carboxylic acid (Preparation 84, 24.22 mg, 73.3 μmol) was added and the reaction stirred at rt overnight. The reaction was partitioned between water and EtOAc, the layers separated and the organic phase purified directly by silica gel column chromatography (10% MeOH/EtOAc) to give N-(1-((cis)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, 17 mg. LCMS m/z=468.4 [M+H]⁺; 1H NMR (500 MHz, MeOH-d₄) δ:11.50 (s, 1H), 10.29 (s, 1H), 9.28 (dd, J=1.68, 7.32 Hz, 1H), 8.53 (s, 1H), 8.27 (d, J=7.02 Hz, 1H), 7.17 (t, J=7.25 Hz, 1H), 6.42 (quin, J=6.18 Hz, 1H), 5.99-5.79 (m, 1H), 4.69 (s, 2H), 4.36-4.26 (m, 2H), 3.71 (s, 1H), 3.55 (s, 1H), 2.84-2.78 (m, 2H), 2.72 (s, 1H), 2.57 (dd, J=1.60, 4.35 Hz, 2H), 2.51-2.40 (m, 1H), 2.36 (dd, J=3.59, 6.18 Hz, 7H), 2.24 (s, 4H)

N-(1-((cis)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide was further purified by SFC: CHIRALPAK IA 30×250 mm, 5 um, Method: 40% EtOH w/ 0.1% DEA in CO₂ (flow rate: 100 mL/min, ABPR 120 bar, MBPR 40 psi, column temp 40° C.) to provide

Peak 1: N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, 2.8 mg (100% ee) (stereochemistry arbitrarily assigned). LCMS m/z=468.3 [M+H]⁺; 1H NMR (400 MHz, MeOH-d₄) δ 9.39 (s, 1H), 8.59 (dd, J=1.63, 7.40 Hz, 1H), 7.61 (s, 1H), 7.39 (d, J=6.02 Hz, 1H), 6.39 (t, J=7.28 Hz, 1H), 5.82-5.73 (m, 1H), 5.08-5.02 (m, 1H), 4.00 (s, 2H), 3.44-3.39 (m, 1H), 2.11 (dd, J=1.76, 4.77 Hz, 2H), 1.86 (dd, J=1.76, 4.52 Hz, 2H), 1.63 (dd, J=1.51, 6.27 Hz, 6H), 1.60-1.50 (m, 2H), 1.49 (s, 3H)

and Peak 2: N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide, 1.9 mg, (>90% ee). (stereochemistry arbitrarily assigned). LCMS m/z=468.1 [M+H]⁺; 1H NMR (400 MHz, MeOH-d₄) δ 9.39 (s, 1H), 8.59 (dd, J=1.76, 7.53 Hz, 1H), 7.61 (s, 1H), 7.39 (d, J=5.77 Hz, 1H), 6.39 (t, J=7.28 Hz, 1H), 5.77 (quin, J=6.27 Hz, 1H), 5.10-5.01 (m, 1H), 4.00 (s, 2H), 3.44-3.38 (m, 1H), 2.14-2.07 (m, 2H), 1.86 (dd, J=1.63, 4.64 Hz, 2H), 1.63 (dd, J=1.51, 6.27 Hz, 6H), 1.60-1.50 (m, 2H), 1.49 (s, 3H).

### Examples 160 and 161: 7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide and 7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide

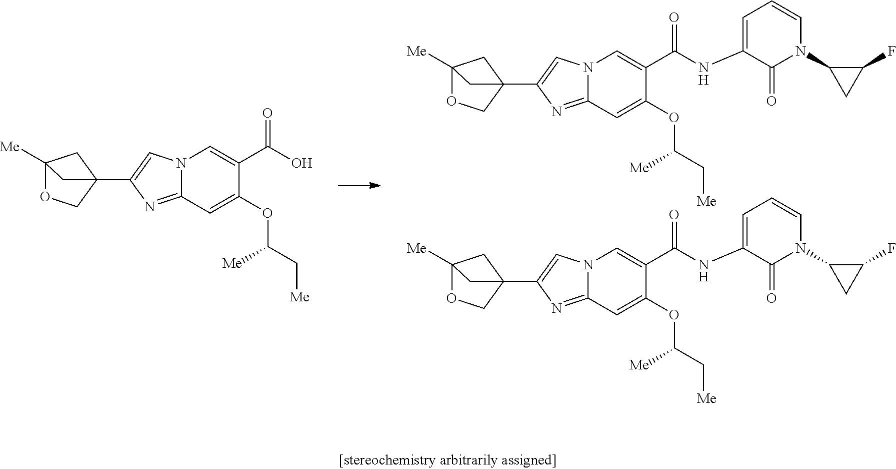

Cis-3-amino-1-(2-fluorocyclopropyl)pyridin-2(1H)-one hydrochloride (Preparation 63, 15 mg, 73.3 μmol) was dissolved in DMF (1 mL), HATU (30.74 mg, 80.63 μmol) and DIPEA (28.42 mg, 219.9 μmol) were added and the solution stirred for 1 min. (S)-7-(sec-Butoxy)-2-(1-methyl-2-oxabicyclo[2.1.1]hexan-4-yl)imidazo[1,2-a]pyridine-6-carboxylic acid (Preparation 44, 24.22 mg, 73.3 μmol) was added and the reaction was stirred at rt overnight. The reaction was partitioned between water and EtOAc, the layers separated and the organic phase purified directly by silica gel column chromatography (10% MeOH/EtOAc) to give cis 7-((S)-sec-butoxy)-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide, 18 mg, 50%. LCMS m/z=481.5 [M+H]⁺; 1H NMR (500 MHz, MeOH-d₄) δ: 9.16 (s, 1H), 8.68-8.52 (m, 1H), 7.73 (s, 1H), 7.40 (br d, J=6.56 Hz, 1H), 7.02 (s, 1H), 6.46-6.30 (m, 1H), 4.82-4.71 (m, 1H), 4.03 (s, 2H), 3.48-3.39 (m, 1H), 2.26-2.04 (m, 3H), 1.97-1.83 (m, 3H), 1.67-1.56 (m, 4H), 1.56-1.43 (m, 5H), 1.10-0.98 (m, 3H)

Cis 7-((S)-sec-butoxy)-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide was purified by SFC: CHIRALPAK IB 30×250 mm, 5 um Method: 40% MeOH w/ 0.1% DEA in C₀₂ (flow rate: 100 mL/min, column temp 40 deg ° C.) to obtain

Peak 1: 7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide (>91% ee), 4 mg. (Stereochemistry was arbitrarily assigned). LCMS m/z=481.2 [M+H]⁺; ¹H NMR (500 MHz, MeOH-d₄) δ: 9.17 (s, 1H), 8.63 (dd, J=1.75, 7.40 Hz, 1H), 7.73 (s, 1H), 7.41 (d, J=5.95 Hz, 1H), 7.02 (s, 1H), 6.41 (t, J=7.25 Hz, 1H), 5.08-5.02 (m, 1H), 4.83-4.76 (m, 1H), 4.03 (s, 2H), 3.45-3.40 (m, 1H), 2.27-2.17 (m, 1H), 2.15-2.10 (m, 2H), 1.98-1.88 (m, 3H), 1.60 (d, J=6.10 Hz, 4H), 1.57-1.54 (m, 1H), 1.52 (s, 3H), 1.07 (t, J=7.40 Hz, 3H)

And Peak 2: 7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide (>88% ee), 4.4 mg. (Stereochemistry was arbitrarily assigned). LCMS m/z=481.2 [M+H]+ 1H NMR (500 MHz, MeOH-d₄) δ: 9.16 (s, 1H), 8.62 (dd, J=1.60, 7.40 Hz, 1H), 7.73 (s, 1H), 7.40 (d, J=6.87 Hz, 1H), 7.02 (s, 1H), 6.41 (t, J=7.25 Hz, 1H), 5.10-4.98 (m, 1H), 4.82-4.73 (m, 1H), 4.03 (s, 2H), 3.45-3.39 (m, 1H), 2.27-2.17 (m, 1H), 2.13 (dd, J=1.45, 4.50 Hz, 2H), 1.90 (dd, J=1.60, 4.50 Hz, 3H), 1.62-1.56 (m, 4H), 1.55-1.49 (m, 4H), 1.06 (t, J=7.40 Hz, 3H).

### Examples 162 and 163: N-(1-((1R,2S)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide and N-(1-((1S,2R)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide

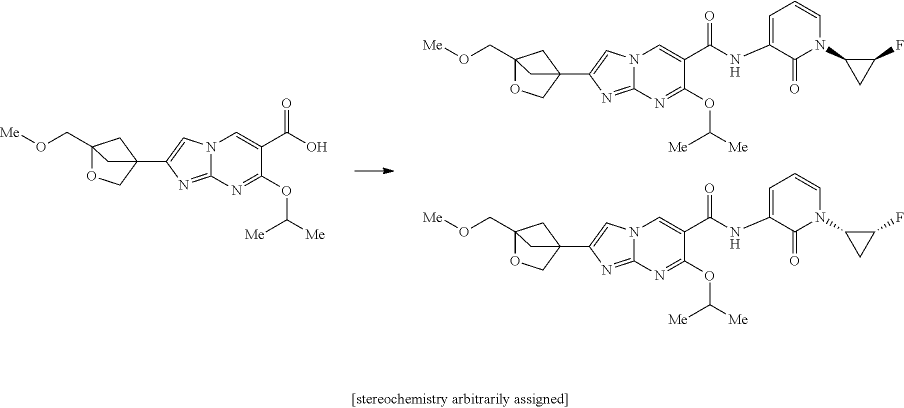 [SMILES: COCC12CC(c3cn4cc(C(=O)O)c(OC(C)C)nc4n3)(CO1)C2 ; COCC12CC(c3cn4cc(C(=O)Nc5cccn([C@@H]6C[C@@H]6F)c5=O)c(OC(C)C)nc4n3)(CO1)C2 ; COCC12CC(c3cn4cc(C(=O)Nc5cccn([C@H]6C[C@H]6F)c5=O)c(OC(C)C)nc4n3)(CO1)C2]

N-(1-((1R,2S)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide and N-(1-((1S,2R)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide were obtained from 7-isopropoxy-2-(1-(methoxymethyl)-2-oxabicyclo[2.1.1]hexan-4-yl)imidazo[1,2-a]pyrimidine-6-carboxylic acid (Preparation 29) and cis-3-amino-1-(2-fluorocyclopropyl)pyridin-2(1H)-one hydrochloride (Preparation 63), following a similar procedure to that described in Example 160 and 161.

Peak 1: N-(1-((1R,2S)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide (100% ee), 2.9 mg (stereochemistry arbitrarily assigned). LCMS m/z=498.1 [M+H]⁺; 1H NMR (500 MHz, MeOH-d₄) δ: 9.42 (s, 1H), 8.62 (dd, J=1.83, 7.48 Hz, 1H), 7.66 (s, 1H), 7.42 (d, J=5.95 Hz, 1H), 6.42 (t, J=7.17 Hz, 1H), 5.80 (quin, J=6.26 Hz, 1H), 5.09-5.01 (m, 1H), 4.05 (s, 2H), 3.73 (s, 2H), 3.47-3.42 (m, 5H), 2.21 (dd, J=1.68, 4.58 Hz, 2H), 1.94 (dd, J=1.68, 4.58 Hz, 2H), 1.66 (dd, J=2.06, 6.18 Hz, 6H), 1.62-1.53 (m, 2H)

Peak 2: N-(1-((1S,2R)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide (89% ee), 3.3 mg (stereochemistry arbitrarily assigned). LCMS m/z=498.1 [M+H]⁺; 1H NMR (500 MHz, MeOH-d₄) δ: 9.42 (s, 1H), 8.62 (dd, J=1.68, 7.48 Hz, 1H), 7.66 (s, 1H), 7.42 (d, J=6.87 Hz, 1H), 6.42 (t, J=7.25 Hz, 1H), 5.75-5.85 (m, 1H), 5.01-5.08 (m, 1H), 4.05 (s, 2H), 3.73 (s, 2H), 3.44 (s, 4H), 2.21 (dd, J=1.60, 4.65 Hz, 2H), 1.94 (dd, J=1.68, 4.58 Hz, 2H), 1.66 (dd, J=2.14, 6.26 Hz, 6H), 1.53-1.62 (m, 2H).

### Examples 164 and 165: N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide and N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide

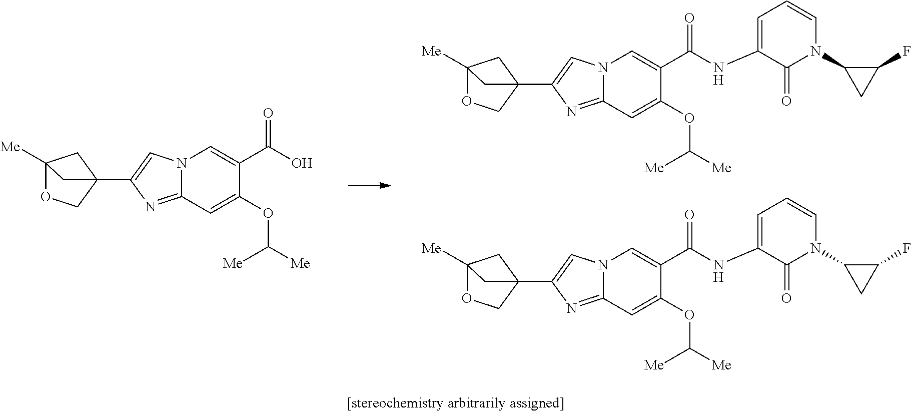 [SMILES: CC(C)Oc1cc2nc(C34COC(C)(C3)C4)cn2cc1C(=O)O ; CC(C)Oc1cc2nc(C34COC(C)(C3)C4)cn2cc1C(=O)Nc1cccn([C@@H]2C[C@@H]2F)c1=O ; CC(C)Oc1cc2nc(C34COC(C)(C3)C4)cn2cc1C(=O)Nc1cccn([C@H]2C[C@H]2F)c1=O]

Cis-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide was obtained from 7-isopropoxy-2-(1-methyl-2-oxabicyclo[2.1.1]hexan-4-yl)imidazo[1,2-a]pyridine-6-carboxylic acid (Preparation 4) and cis-3-amino-1-(2-fluorocyclopropyl)pyridin-2(1H)-one hydrochloride (Preparation 63), following a similar procedure to that described in Example 160 and 161.

This was purified by SFC: CHIRALPAK IB 30×250 mm, 5 um Method: 30% MeOH w/0.1% DEA in CO₂ (flow rate: 100 mL/min, ABPR 120 bar, MBPR 40 psi, column temp 40° C.) to give

Peak 1: N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide (100% ee), 0.5 mg. (stereochemisry arbitrarily assigned). 1H NMR (500 MHz, MeOH-d₄) δ: 9.16 (s, 1H), 8.62 (d, J=7.48 Hz, 1H), 7.73 (s, 1H), 7.41 (d, J=7.78 Hz, 1H), 7.02 (s, 1H), 6.42 (t, J=7.25 Hz, 1H), 5.04 (br dd, J=6.33, 12.13 Hz, 2H), 4.62 (s, 1H), 4.03 (s, 2H), 3.39 (br s, 2H), 2.13 (d, J=6.10 Hz, 2H), 1.90 (d, J=6.10 Hz, 2H), 1.64 (d, J=6.10 Hz, 6H), 1.60-1.53 (m, 2H), 1.52 (s, 3H)

Peak 2: N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide (>93% ee), 0.2 mg. (stereochemisry arbitrarily assigned). 1H NMR (500 MHz, MeOH-d₄) δ: 9.13 (s, 1H), 8.60 (dd, J=1.60, 7.55 Hz, 1H), 7.71 (s, 1H), 7.38 (d, J=6.41 Hz, 1H), 7.00 (s, 1H), 6.39 (t, J=7.25 Hz, 1H), 5.01 (dt, J=5.72, 11.79 Hz, 2H), 4.01 (s, 2H), 3.43-3.38 (m, 3H), 2.11 (dd, J=1.60, 4.65 Hz, 2H), 1.88 (dd, J=1.68, 4.58 Hz, 2H), 1.62 (d, J=5.95 Hz, 6H), 1.59-1.51 (m, 2H), 1.50 (s, 3H), 1.46-1.39 (m, 2H).

### Examples 166 and 167: 2-(2-oxabicyclo[2.1.1]hexan-4-yl)-7-cyclobutoxy-N-(1-((1R,2S)-2-fluorocyclopropyl)-2-oxo-1,2-dihydropyridin-3-yl)imidazo[1,2-a]pyrimidine-6-carboxamide and 2-(2-oxabicyclo[2.1.1]hexan-4-yl)-7-cyclobutoxy-N-(1-((1S,2R)-2-fluorocyclopropyl)-2-oxo-1,2-dihydropyridin-3-yl)imidazo[1,2-a]pyrimidine-6-carboxamide

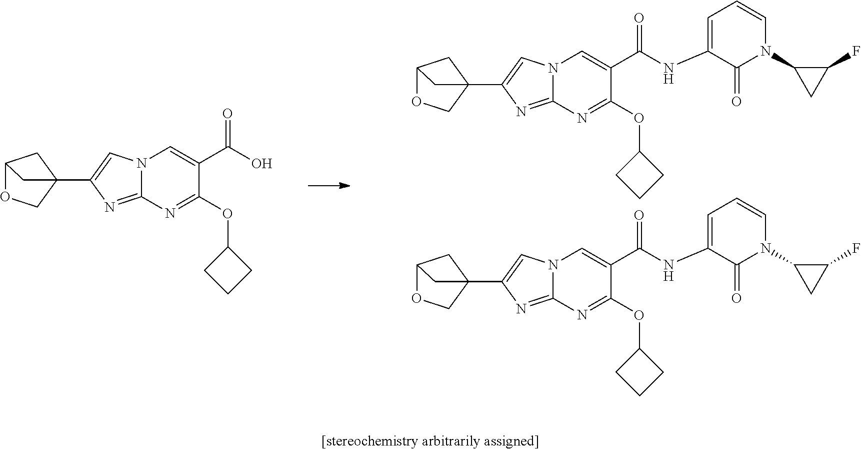

2-(2-Oxabicyclo[2.1.1]hexan-4-yl)-7-cyclobutoxyimidazo[1,2-a]pyrimidine-6-carboxylic acid (Preparation 30, 23.11 mg, 73.3 μmol) was dissolved in DMF (1 mL), HATU (30.74 mg, 80.63 μmol) and DIPEA (28.42 mg, 219.9 μmol) were added and the reaction stirred for 1 min. Cis-3-amino-1-(2-fluorocyclopropyl)pyridin-2(1H)-one hydrochloride (Preparation 63, 15 mg, 73.3 μmol) was then added and the mixture stirred at rt overnight.

The mixture was concentrated in vacuo and the residue purified by RPHPLC (SunFire C18 column, 60 mL/min, 5-60% MeCN/(0.1% TFA (aq)) to give cis-2-(2-oxabicyclo[2.1.1]hexan-4-yl)-7-cyclobutoxy-N-(1-(2-fluorocyclopropyl)-2-oxo-1,2-dihydropyridin-3-yl)imidazo[1,2-a]pyrimidine-6-carboxamide, 87% yield. LCMS m/z=466.5 [M+H]⁺

The compound was further purified by SFC: CHIRALPAK IB 30×250 mm, 5 um Method: 50% MeOH w/ 0.1% DEA in CO₂ (flow rate: 100 mL/min, ABPR 120 bar, MBPR 40 psi, column temp 40° C.) to provide

Peak 1: 2-(2-oxabicyclo[2.1.1]hexan-4-yl)-7-cyclobutoxy-N-(1-((1R,2S)-2-fluorocyclopropyl)-2-oxo-1,2-dihydropyridin-3-yl)imidazo[1,2-a]pyrimidine-6-carboxamide (100% ee), 3.5 mg. (stereochemistry arbitrarily assigned). LCMS m/z=466.1 [M+H]⁺; 1H NMR (500 MHz, MeOH-d₄) δ: 9.43 (s, 1H), 8.63 (dd, J=1.83, 7.48 Hz, 1H), 7.67 (s, 1H), 7.43 (d, J=6.56 Hz, 1H), 6.43 (t, J=7.17 Hz, 1H), 5.64-5.57 (m, 1H), 5.08-5.02 (m, 1H), 4.68-4.65 (m, 1H), 3.97 (s, 2H), 3.44 (br d, J=1.37 Hz, 1H), 2.70-2.61 (m, 4H), 2.27 (d, J=5.04 Hz, 2H), 2.03 (br d, J=6.10 Hz, 1H), 1.92-1.80 (m, 3H), 1.63-1.52 (m, 2H) and

Peak 2: 2-(2-oxabicyclo[2.1.1]hexan-4-yl)-7-cyclobutoxy-N-(1-((1S,2R)-2-fluorocyclopropyl)-2-oxo-1,2-dihydropyridin-3-yl)imidazo[1,2-a]pyrimidine-6-carboxamide (96% ee), 4.9 mg. (stereochemistry arbitrarily assigned). LCMS m/z=466.1 [M+H]⁺; 1H NMR (500 MHz, MeOH-d₄) δ: 9.43 (s, 1H), 8.63 (dd, J=1.75, 7.40 Hz, 1H), 7.67 (s, 1H), 7.43 (d, J=7.78 Hz, 1H), 6.43 (t, J=7.17 Hz, 1H), 5.61 (t, J=7.02 Hz, 1H), 5.09-5.04 (m, 1H), 4.67 (s, 1H), 3.97 (s, 2H), 3.46-3.42 (i, 1H), 2.72-2.60 (m, 4H), 2.27 (d, J=5.04 Hz, 2H), 2.08-1.99 (m, 1H), 1.91-1.74 (m, 3H), 1.64-1.53 (in, 2H).

### Examples 168 to 175

The following compounds were obtained from the appropriate carboxylic acid and cis-3-amino-1-(2-fluorocyclopropyl)pyridin-2(1H)-one hydrochloride (Preparation 63), following a similar method to that described in Examples 166 and 167.

| Example | |
|---|---|
| No | Name/Structure/Starting material (SM)/Data |
| 168 | N-(1-((1R,2S)-2-fluorocyclopropyl)-2-oxo-1,2-dihydropyridin-3-yl)-7- |
| and | isopropoxy-2-((1S,4R)-1-methyl-2-oxabicyclo[2.2.1]heptan-4- |
| 169 | yl)imidazo[1,2-a]pyridine-6-carboxamide and N-(1-((1S,2R)-2- |
| | fluorocyclopropyl)-2-oxo-1,2-dihydropyridin-3-yl)-7-isopropoxy-2- |
| | ((1S,4R)-1-methyl-2-oxabicyclo[2.2.1]heptan-4-yl)imidazo[1,2-a]pyridine- |
| | 6-carboxamide |
| | 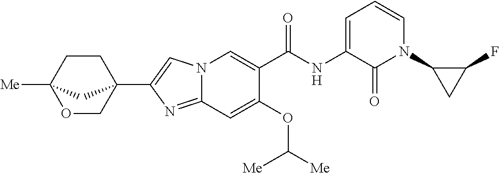 [SMILES: CC(C)Oc1cc2nc([C@@]34CC[C@@](C)(C3)OC4)cn2cc1C(=O)Nc1cccn([C@@H]2C[C@@H]2F)c1=O] |
| | 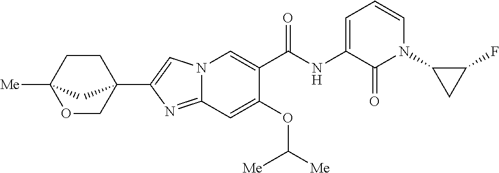 [SMILES: CC(C)Oc1cc2nc([C@@]34CC[C@@](C)(C3)OC4)cn2cc1C(=O)Nc1cccn([C@H]2C[C@H]2F)c1=O] |
| | [stereochemistry arbitrarily assigned] |
| | SM: 7-isopropoxy-2-((1S,4R)-1-methyl-2-oxabicyclo[2.2.1]heptan-4- |
| | yl]imidazo[1,2-a]pyridine-6-carboxylic acid (Preparation 59A) |
| | Peak 1: Example 168, (100% ee), 3.7 mg, (stereochemistry arbitrarily |
| | assigned) LCMS m/z = 481.2 [M + H]⁺; 1H NMR (500 MHz, MeOH-d₄) δ: |
| | 9.15 (s, 1H), 8.62 (dd, J = 1.75, 7.40 Hz, 1H), 7.70 (s, 1H), 7.41 (d, J = 5.95 |
| | Hz, 1H), 7.01 (s, 1H), 6.41 (t, J = 7.17 Hz, 1H), 5.08-4.98 (m, 2H), 4.07 (dd, |
| | J = 3.36, 6.56 Hz, 1H), 3.94 (d, J = 6.41 Hz, 1H), 3.45-3.39 (m, 1H), 2.25-2.14 |
| | (m, 1H), 2.13-2.06 (m, 1H), 2.03-1.95 (m, 2H), 1.89 (dq, J = 5.42, 14.27 Hz, |
| | 2H), 1.64 (d, J = 5.95 Hz, 6H), 1.61-1.57 (m, 1H), 1.56-1.52 (m, 1H), 1.48 (s, |
| | 3H) |
| | Peak 2: Example 169, 2.3 mg (stereochemistry arbitrarily assigned). LCMS |
| | m/z = 481.2 [M + H]⁺; 1H NMR (500 MHz, MeOH-d₄) δ: 9.15 (s, 1H), 8.62 |
| | (dd, J = 1.83, 7.48 Hz, 1H), 7.70 (s, 1H), 7.41 (d, J = 7.48 Hz, 1H), 7.01 (s, |
| | 1H), 6.41 (t, J = 7.25 Hz, 1H), 5.08-4.99 (m, 3H), 4.07 (dd, J = 3.43, 6.48 Hz, |
| | 1H), 3.94 (d, J = 6.56 Hz, 1H), 3.41-3.39 (m, 1H), 2.24-2.14 (m, 1H), 2.14- |
| | 2.04 (m, 2H), 2.02-1.95 (m, 2H), 1.94-1.80 (m, 2H), 1.64 (d, J = 6.10 Hz, |
| | 6H), 1.60-1.52 (m, 2H), 1.48 (s, 3H) |
| 170 | N-(1-((1R,2S)-2-fluorocyclopropyl)-2-oxo-1,2-dihydropyridin-3-yl)-7- |
| and | isopropoxy-2-((1R,4S)-1-methyl-2-oxabicyclo[2.2.1]heptan-4- |
| 171 | yl)imidazo[1,2-a]pyridine-6-carboxamide and N-(1-((1S,2R)-2- |
| | fluorocyclopropyl)-2-oxo-1,2-dihydropyridin-3-yl)-7-isopropoxy-2- |
| | ((1R,4S)-1-methyl-2-oxabicyclo[2.2.1]heptan-4-yl)imidazo[1,2-a]pyridine- |
| | 6-carboxamide |
| | 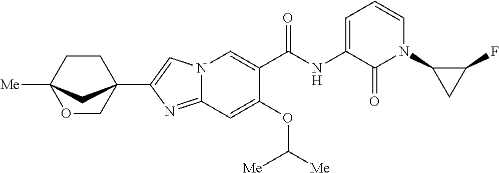 [SMILES: CC(C)Oc1cc2nc([C@]34CC[C@](C)(C3)OC4)cn2cc1C(=O)Nc1cccn([C@@H]2C[C@@H]2F)c1=O] |
| | 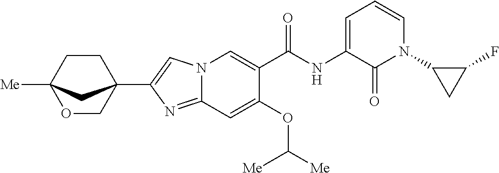 |
| | [stereochemistry arbitrarily assigned] |
| | SM: 7-isopropoxy-2-((1R,4S)-1-methyl-2-oxabicyclo[2.2.1]heptan-4- |
| | yl)imidazo[1,2-a]pyridine-6-carboxylic acid (Preparation 59B) |
| | Peak 1: Example 170, (100% ee) (stereochemistry arbitrarily assigned). 4.3 |
| | mg, LCMS m/z = 481.2 [M + H]⁺; 1H NMR (500 MHz, MeOH-d₄) δ: 9.15 (s, |
| | 1H), 8.62 (dd, J = 1.68, 7.48 Hz, 1H), 7.70 (s, 1H), 7.41 (d, J = 5.95 Hz, 1H), |
| | 7.01 (s, 1H), 6.41 (t, J = 7.25 Hz, 1H), 5.03 (td, J = 5.07, 11.52 Hz, 2H), 4.07 |
| | (dd, J = 3.36, 6.41 Hz, 1H), 3.94 (d, J = 6.56 Hz, 1H), 3.45-3.40 (m, 1H), 2.23- |
| | 2.15 (m, 1H), 2.13-2.06 (m, 1H), 2.00-1.97 (m, 2H), 1.94-1.79 (m, 2H), 1.64 |
| | (d, J = 6.10 Hz, 6H), 1.61-1.52 (m, 2H), 1.48 (s, 3H) |
| | And Peak 2: Example 171, (95% ee) (stereochemistry arbitrarily assigned). |
| | 6.9 mg. LCMS m/z = 481.2 [M + H]⁺; 1H NMR (500 MHz, MeOH-d₄) δ: |
| | 9.15 (s, 1H), 8.62 (dd, J = 1.68, 7.48 Hz, 1H), 7.70 (s, 1H), 7.41 (d, J = 7.02 |
| | Hz, 1H), 7.01 (s, 1H), 6.41 (t, J = 7.25 Hz, 1H), 5.09-4.99 (m, 2H), 4.07 (dd, |
| | J = 3.36, 6.56 Hz, 1H), 3.94 (d, J = 6.56 Hz, 1H), 3.45-3.40 (m, 1H), 2.23-2.15 |
| | (m, 1H), 2.13-2.06 (m, 1H), 2.02-1.95 (m, 2H), 1.94-1.81 (m, 2H), 1.64 (d, |
| | J = 6.10 Hz, 6H), 1.61-1.52 (m, 2H), 1.48 (s, 3H) |
| 172 | 7-cyclobutoxy-N-(1-((1R,2S)-2-fluorocyclopropyl)-2-oxo-1,2- |
| and | dihydropyridin-3-yl)-2-(1-methyl-2-oxabicyclo[2.1.1]hexan-4- |
| 173 | yl)imidazo[1,2-a]pyridine-6-carboxamide and 7-cyclobutoxy-N-(1-((1S,2R)- |
| | 2-fluorocyclopropyl)-2-oxo-1,2-dihydropyridin-3-yl)-2-(1-methyl-2- |
| | oxabicyclo[2.1.1]hexan-4-yl)imidazo[1,2-a]pyridine-6-carboxamide |
| | 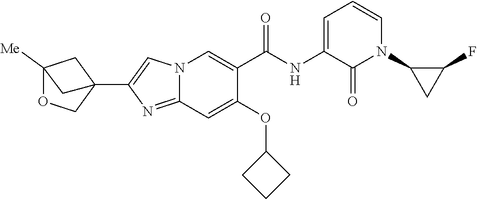 [SMILES: CC12CC(c3cn4cc(C(=O)Nc5cccn([C@@H]6C[C@@H]6F)c5=O)c(OC5CCC5)cc4n3)(CO1)C2] |
| | 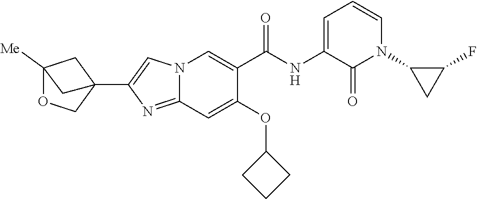 [SMILES: CC12CC(c3cn4cc(C(=O)Nc5cccn([C@H]6C[C@H]6F)c5=O)c(OC5CCC5)cc4n3)(CO1)C2] |
| | [stereochemistry arbitrarily assigned] |
| | SM: 7-cyclobutoxy-2-(1-methyl-2-oxabicyclo[2.1.1]hexan-4-yl)imidazo[1,2- |
| | a]pyridine-6-carboxylic acid (Preparation 33) |
| | Peak 1: Example 172 (100% ee). (Stereochemistry arbitrarily assigned), 3.4 |
| | mg. LCMS m/z = 479.1 [M + H]⁺; 1H NMR (500 MHz, MeOH-d₄) 9.16 (s, |
| | 1H), 8.63 (dd, J = 1.68, 7.48 Hz, 1H), 7.73 (s, 1H), 7.42 (d, J = 5.80 Hz, 1H), |
| | 6.83 (s, 1H), 6.42 (t, J = 7.17 Hz, 1H), 5.10-5.03 (m, 2H), 4.03 (s, 2H), 3.45- |
| | 3.41 (m, 1H), 2.71-2.62 (m, 4H), 2.17-2.10 (m, 2H), 2.09-2.00 (m, 1H), |
| | 1.94-1.86 (m, 3H), 1.62-1.54 (m, 2H), 1.52 (s, 3H) |
| | And Peak 2: Example 173 (>90% ee), (stereochemistry arbitrarily assigned), |
| | 5 mg. LCMS m/z = 479.1 [M + H]⁺; 1H NMR (500 MHz, MeOH-d₄) 9.16 (s, |
| | 1H), 8.63 (dd, J = 1.68, 7.48 Hz, 1H), 7.73 (s, 1H), 7.42 (d, J = 6.10 Hz, 1H), |
| | 6.83 (s, 1H), 6.42 (t, J = 7.25 Hz, 1H), 5.10-5.02 (m, 2H), 4.03 (s, 2H), 3.46- |
| | 3.41 (m, 1H), 2.72-2.59 (m, 4H), 2.15-2.10 (m, 2H), 2.10-1.99 (m, 1H), |
| | 1.93-1.86 (m, 3H), 1.63-1.53 (m, 2H), 1.52 (s, 3H). |
| 174 | 7-cyclopropoxy-N-(1-((1R,2S)-2-fluorocyclopropyl)-2-oxo-1,2- |
| and | dihydropyridin-3-yl)-2-(1-methyl-2-oxabicyclo[2.1.1]hexan-4- |
| 175 | yl)imidazo[1,2-a]pyridine-6-carboxamide and 7-cyclopropoxy-N-(1- |
| | ((1S,2R)-2-fluorocyclopropyl)-2-oxo-1,2-dihydropyridin-3-yl)-2-(1-methyl- |
| | 2-oxabicyclo[2.1.1]hexan-4-yl)imidazo[1,2-a]pyridine-6-carboxamide |
| | 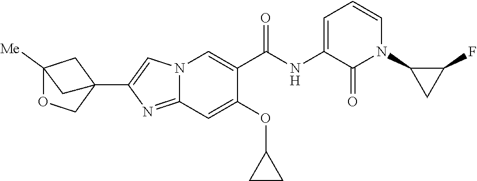 [SMILES: CC12CC(c3cn4cc(C(=O)Nc5cccn([C@@H]6C[C@@H]6F)c5=O)c(OC5CC5)cc4n3)(CO1)C2] |
| | 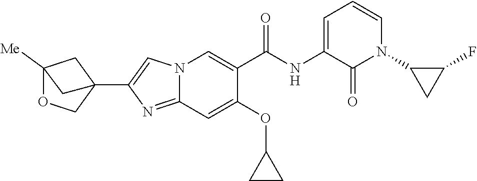 |
| | [stereochemistry arbitrarily assigned] |
| | SM: 7-cyclopropoxy-2-(1-methyl-2-oxabicyclo[2.1.1]hexan-4- |
| | yl)imidazo[1,2-a]pyridine-6-carboxylic acid (Preparation 51) |
| | Peak 1: Example 174, 1 mg. (stereochemistry arbitrarily assigned). LCMS |
| | m/z = 465.2 [M + H]⁺; 1H NMR (500 MHz, MeOH-d₄) δ 9.16 (s, 1H), 8.60 |
| | (dd, J = 1.75, 7.40 Hz, 1H), 7.76 (s, 1H), 7.41 (d, J = 5.65 Hz, 1H), 7.38 (s, |
| | 1H), 6.42 (t, J = 7.25 Hz, 1H), 5.09-5.01 (m, 1H), 4.23-4.18 (m, 1H), 4.04 (s, |
| | 2H), 3.45-3.40 (m, 2H), 2.17-2.11 (m, 2H), 1.91 (dd, J = 1.68, 4.58 Hz, 2H), |
| | 1.62-1.50 (m, 6H), 1.27-1.20 (m, 2H), 1.09-1.02 (m, 2H) |
| | Peak 2: Example 175 (stereochemistry arbitrarily assigned). 1.5 mg. LCMS |
| | m/z = 465.2 [M + H]⁺; 1H NMR (500 MHz, MeOH-d₄) δ 9.16 (s, 1H), 8.60 |
| | (dd, J = 1.75, 7.40 Hz, 1H), 7.76 (s, 1H), 7.41 (d, J = 7.02 Hz, 1H), 7.38 (s, |
| | 1H), 6.42 (t, J = 7.25 Hz, 1H), 5.09-4.97 (m, 1H), 4.25-4.17 (m, 1H), 4.04 (s, |
| | 2H), 3.45-3.40 (m, 1H), 2.14 (dd, J = 1.68, 4.58 Hz, 2H), 1.91 (dd, J = 1.75, |
| | 4.50 Hz, 2H), 1.61-1.54 (m, 2H), 1.53 (s, 3H), 1.26-1.20 (m, 2H), 1.10-1.01 |
| | (m, 2H) |

### Examples 176, 177, 178 and 179

### N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide, N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide, N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide and N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide

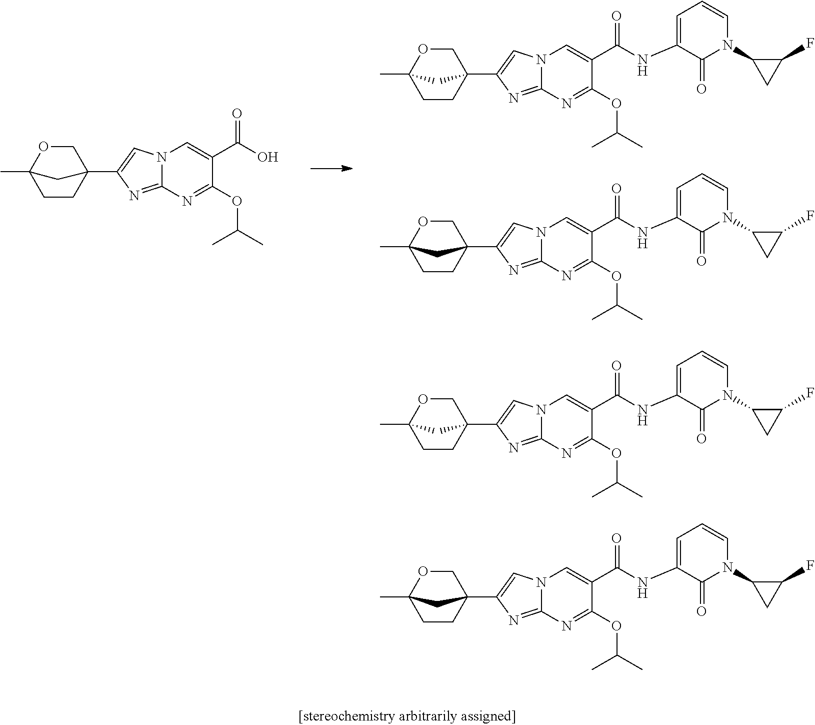 [SMILES: CC(C)Oc1nc2nc(C34CCC(C)(C3)OC4)cn2cc1C(=O)O ; CC(C)Oc1nc2nc([C@@]34CC[C@@](C)(C3)OC4)cn2cc1C(=O)Nc1cccn([C@H]2C[C@H]2F)c1=O ; CC(C)Oc1nc2nc([C@]34CC[C@](C)(C3)OC4)cn2cc1C(=O)Nc1cccn([C@@H]2C[C@@H]2F)c1=O ; CC(C)Oc1nc2nc([C@]34CC[C@](C)(C3)OC4)cn2cc1C(=O)Nc1cccn([C@H]2C[C@H]2F)c1=O ; CC(C)Oc1nc2nc([C@@]34CC[C@@](C)(C3)OC4)cn2cc1C(=O)Nc1cccn([C@@H]2C[C@@H]2F)c1=O]

Cis-N-(1-(2-fluorocyclopropyl)-2-oxo-1,2-dihydropyridin-3-yl)-7-isopropoxy-2-(1-methyl-2-oxabicyclo[2.2.1]heptan-4-yl)imidazo[1,2-a]pyrimidine-6-carboxamide was obtained, 44% yield from 7-isopropoxy-2-(1-methyl-2-oxabicyclo[2.2.1]heptan-4-yl)imidazo[1,2-a]pyrimidine-6-carboxylic acid (Preparation 64) and cis-3-amino-1-(2-fluorocyclopropyl)pyridin-2(1H)-one hydrochloride (Preparation 63), following the procedure described in Example 166 and 167. LCMS m/z=482.5 [M+H]⁺

Cis-N-(1-(2-fluorocyclopropyl)-2-oxo-1,2-dihydropyridin-3-yl)-7-isopropoxy-2-(1-methyl-2-oxabicyclo[2.2.1]heptan-4-yl)imidazo[1,2-a]pyrimidine-6-carboxamide was purified by SFC: CHIRALPAK IB 30×250 mm, 5 um Method: 30% EtOH w/ 0.1% DEA in CO₂ (flow rate: 100 mL/min, ABPR 120 bar, MBPR 40 psi, column temp 40° C.) to afford:

Peak 1: [Example 176] N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide (>99% ee), 1 mg. (stereochemistry arbitrarily assigned). LCMS m/z=482.2 [M+H]⁺; 1H NMR (500 MHz, MeOH-d₄) δ: 9.39 (s, 1H), 8.60 (d, J=9.00 Hz, 1H), 7.62-7.53 (m, 1H), 7.59 (s, 1H), 7.40 (d, J=5.80 Hz, 1H), 6.43-6.35 (m, 1H), 5.82-5.72 (m, 1H), 5.04 (s, 2H), 4.10-3.99 (m, 1H), 3.91 (d, J=6.87 Hz, 1H), 3.43-3.41 (m, 1H), 2.16 (s, 1H), 2.06 (s, 1H), 1.95 (s, 2H), 1.91-1.80 (m, 2H), 1.64 (dd, J=2.44, 6.26 Hz, 6H), 1.59-1.50 (m, 1H), 1.59-1.49 (m, 1H), 1.46 (s, 3H)

Peak 2: [Example 177] N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide (>92% ee) 1.1 mg. (stereochemistry arbitrarily assigned). LCMS m/z=482.2 [M+H]⁺; 1H NMR (500 MHz, MeOH-d₄) δ 9.39 (s, 1H), 8.57-8.65 (m, 1H), 7.59 (s, 1H), 7.40 (d, J=5.95 Hz, 1H), 6.40 (t, J=7.25 Hz, 1H), 5.77 (t, J=6.26 Hz, 1H), 5.07-4.97 (m, 2H), 4.04 (dd, J=3.36, 6.56 Hz, 1H), 3.91 (d, J=6.56 Hz, 1H), 3.43-3.38 (m, 2H), 2.16 (s, 1H), 2.05 (br s, 1H), 1.98-1.92 (m, 2H), 1.89-1.73 (m, 2H), 1.69-1.51 (m, 8H), 1.46 (s, 3H)

Peak 3: [Example 178] N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide (>96% ee), 1.1 mg (stereochemistry arbitrarily assigned). LCMS m/z=482.2 [M+H]⁺; 1H NMR (500 MHz, MeOH-d₄) δ: 9.39 (s, 1H), 8.60 (dd, J=1.68, 7.48 Hz, 1H), 7.59 (s, 1H), 7.40 (d, J=8.09 Hz, 1H), 6.40 (t, J=7.25 Hz, 1H), 5.85-5.68 (m, 1H), 5.04 (br d, J=3.51 Hz, 1H), 4.04 (dd, J=3.43, 6.48 Hz, 1H), 3.91 (d, J=6.56 Hz, 1H), 3.41 (br d, J=5.65 Hz, 1H), 2.20-2.12 (m, 1H), 2.09-2.00 (m, 1H), 2.10-1.99 (m, 1H), 1.97-1.76 (m, 4H), 1.64 (dd, J=1.83, 6.26 Hz, 6H), 1.60-1.51 (m, 2H), 1.46-1.44 (m, 1H), 1.48-1.43 (m, 1H)

Peak 4: [Example 179] N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide (>90% ee) 1.2 mg. (stereochemistry arbitrarily assigned). LCMS m/z=482.2 [M+H]⁺; 1H NMR (500 MHz, MeOH-d₄) δ 9.39 (s, 1H), 8.63-8.56 (m, 1H), 7.59 (s, 1H), 7.40 (d, J=6.56 Hz, 1H), 6.40 (t, J=7.25 Hz, 1H), 5.83-5.69 (m, 1H), 5.04-4.94 (m, 1H), 4.04 (dd, J=3.59, 6.33 Hz, 1H), 3.91 (d, J=6.26 Hz, 1H), 3.41 (br s, 1H), 2.25-2.08 (m, 1H), 2.10-1.99 (m, 1H), 1.97-1.91 (m, 2H), 1.91-1.90 (m, 1H), 1.88-1.70 (m, 2H), 1.64 (dd, J=2.29, 6.26 Hz, 6H), 1.59-1.50 (m, 2H), 1.46 (s, 3H)

### Example 180: 7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyridine-6-carboxamide trifluoroacetate

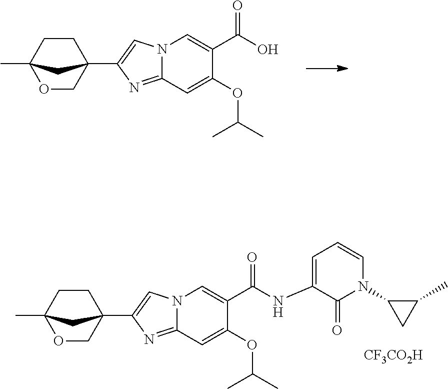

7-Isopropoxy-2-((1R,4S)-1-methyl-2-oxabicyclo[2.2.1]heptan-4-yl)imidazo[1,2-a]pyridine-6-carboxylic acid (Preparation 59B, 52.05 mg, 157.56 μmol) was dissolved in DMF (1 mL). HATU (60.07 mg, 157.56 μmol) and DIPEA (61.09 mg, 472.68 μmol) were added followed by 3-amino-1-((1S,2R)-2-methylcyclopropyl)pyridin-2(1H)-one hydrochloride (Preparation 83, 25.87 mg, 157.56 μmol) and the reaction stirred at rt overnight. The reaction was concentrated in vacuo, the residue partitioned between EtOAc and water, the layers separated and the organic phase concentrated in vacuo. The crude was purified by RPHPLC using a gradient of 5-50% MeCN-water, modified by TFA to afford the title compound. LCMS m/z=477.4 [M+H]⁺; 1H NMR (600 MHz, DMSO-d₆) δ:10.62 (br s, 1H), 9.39 (s, 1H), 8.45 (d, J=7.27 Hz, 1H), 8.11 (s, 1H), 7.41-7.33 (m, 2H), 6.34 (t, J=6.90 Hz, 1H), 5.18 (br s, 1H), 3.93-3.88 (m, 1H), 3.84 (d, J=6.54 Hz, 1H), 2.13-2.02 (m, 2H), 1.99-1.89 (m, 2H), 1.83 (dt, J=4.36, 11.99 Hz, 1H), 1.77-1.68 (m, 1H), 1.58-1.57 (m, 1H), 1.56 (d, J=5.81 Hz, 7H), 1.40 (s, 4H), 1.38-1.29 (m, 1H), 1.16 (q, J=7.75 Hz, 1H), 0.88-0.83 (m, 1H), 0.74 (br d, J=6.54 Hz, 3H).

### Examples 181 to 190

The compounds in the following table were prepared from the appropriate carboxylic acid and aminopyridin-2(1H)-one, following a similar procedure to that described in Example 180.

| Amine | Name |
|---|---|
| Amine 5: | 3-amino-1-((1R,2S)-2-methylcyclopropyl)pyridin-2(1H)-one |
| | hydrochloride (Preparation 82) |
| Amine 6: | 3-amino-1-((1S,2R)-2-methylcyclopropyl)pyridin-2(1H)-one |
| | hydrochloride (Preparation 83) |
| Amine 7: | 3-amino-1-((1S,2R)-2-fluorocyclopropyl)pyridin-2(1H)-one |
| | hydrochloride (Preparation 87B) |

| Example No | Name/Structure/Starting Materials/Data |
|---|---|
| 181 | 7-isopropoxy-2-((1R,4S)-1-methyl-2-oxabicyclo[2.2.1]heptan-4-yl)-N- |
| | (1-((1R,2S)-2-methylcyclopropyl)-2-oxo-1,2-dihydropyridin-3- |
| | yl)imidazo[1,2-a]pyridine-6-carboxamide trifluoroacetate |
| | 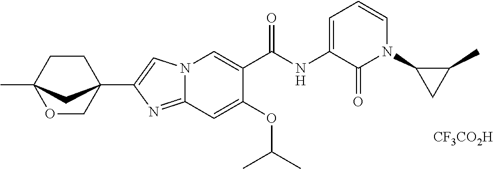 |
| | SM: 7-isopropoxy-2-((1R,4S)-1-methyl-2-oxabicyclo[2.2.1]heptan-4- |
| | yl)imidazo[1,2-a]pyridine-6-carboxylic acid (Preparation 59B) and |
| | Amine 5 |
| | LMCS m/z = 477.4 [M + H]⁺; 1H NMR (600 MHz, DMSO-d₆) δ: 10.62 |
| | (br s, 1H), 9.38 (s, 1H), 8.45 (d, J = 7.27 Hz, 1H), 8.10 (s, 1H), 7.39 (br d, |
| | J = 6.54 Hz, 1H), 7.36 (br s, 1H), 6.33 (t, J = 7.27 Hz, 1H), 5.18 (br d, |
| | J = 5.09 Hz, 1H), 3.95-3.88 (m, 1H), 3.84 (d, J = 6.54 Hz, 1H), 2.15-2.00 |
| | (m, 3H), 1.98-1.91 (m, 3H), 1.83 (dt, J = 4.72, 12.17 Hz, 1H), 1.77-1.69 |
| | (m, 1H), 1.56 (d, J = 6.54 Hz, 7H), 1.48-1.42 (m, 1H), 1.40 (s, 4H), 1.38- |
| | 1.31 (m, 1H), 1.19-1.13 (m, 1H), 0.90-0.83 (m, 1H), 0.74 (d, J = 5.81 Hz, |
| | 3H). |
| 182 | 7-isopropoxy-2-((1S,4R)-1-methyl-2-oxabicyclo[2.2.1]heptan-4-yl)-N- |
| | (1-((1S,2R)-2-methylcyclopropyl)-2-oxo-1,2-dihydropyridin-3- |
| | yl)imidazo[1,2-a]pyridine-6-carboxamide trifluoroacetate |
| | 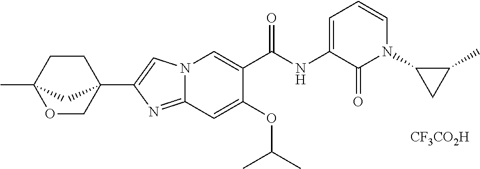 |
| | SM: 7-isopropoxy-2-((1S,4R)-1-methyl-2-oxabicyclo[2.2.1]heptan-4- |
| | yl)imidazo[1,2-a]pyridine-6-carboxylic acid (Preparation 59A) and |
| | Amine 6 |
| | LCMS m/z = 477.4 [M + H]⁺; 1H NMR (600 MHz, DMSO-d₆) δ: 10.62 |
| | (br s, 1H), 9.39 (s, 1H), 8.45 (d, J = 7.27 Hz, 1H), 8.11 (s, 1H), 7.39 (br d, |
| | J = 7.27 Hz, 1H), 7.36 (br s, 1H), 6.34 (t, J = 7.27 Hz, 1H), 5.18 (br s, 1H), |
| | 3.92-3.86 (m, 1H), 3.84 (d, J = 6.54 Hz, 1H), 2.12-2.01 (m, 2H), 1.98- |
| | 1.92 (m, 2H), 1.84 (dt, J = 5.09, 11.99 Hz, 1H), 1.78-1.70 (m, 1H), 1.56 |
| | (d, J = 5.81 Hz, 7H), 1.42-1.32 (m, 5H), 1.16 (q, J = 7.99 Hz, 1H), 0.90- |
| | 0.83 (m, 1H), 0.74 (d, J = 5.81 Hz, 3H). |
| 183 | 7-isopropoxy-2-((1S,4R)-1-methyl-2-oxabicyclo[2.2.1]heptan-4-yl)-N- |
| | (1-((1R,2S)-2-methylcyclopropyl)-2-oxo-1,2-dihydropyridin-3- |
| | yl)imidazo[1,2-a]pyridine-6-carboxamide trifluoroacetate |
| | 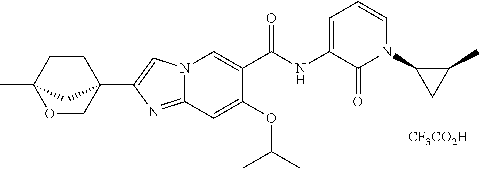 |
| | SM: 7-isopropoxy-2-((1S,4R)-1-methyl-2-oxabicyclo[2.2.1]heptan-4- |
| | yl)imidazo[1,2-a]pyridine-6-carboxylic acid (Preparation 59A) and |
| | Amine 5 |
| | 1H NMR (600 MHz, DMSO-d₆) δ 10.74 (s, 1H), 9.15 (s, 1H), 8.46 (d, J = 7.27 |
| | Hz, 1H), 7.79 (s, 1H), 7.33 (br d, J = 6.54 Hz, 1H), 7.17 (br s, 1H), 6.30 (t, |
| | J = 6.90 Hz, 1H), 5.01 (td, J = 6.09, 11.81 Hz, 1H), 3.93 (dd, J = 3.27, 6.18 Hz, |
| | 1H), 3.75 (d, J = 6.54 Hz, 1H), 3.37-3.26 (m, 1H), 2.10-2.01 (m, 1H), 1.92 (br t, |
| | J = 8.36 Hz, 1H), 1.84-1.73 (m, 3H), 1.72-1.65 (m, 1H), 1.51 (br d, J = 5.81 Hz, |
| | 7H), 1.42-1.32 (m, 4H), 1.14 (q, J = 7.99 Hz, 1H), 0.85 (q, J = 5.57 Hz, 1H), 0.74 |
| | (d, J = 6.54 Hz, 3H). |
| 184* | 7-(((S)-1,1-difluoropropan-2-yl)oxy)-N-(1-((1S,2R)-2- |
| | fluorocyclopropyl)-2-oxo-1,2-dihydropyridin-3-yl)-2-(1-methyl-2- |
| | oxabicyclo[2.1.1]hexan-4-yl)imidazo[1,2-a]pyridine-6-carboxamide |
| | 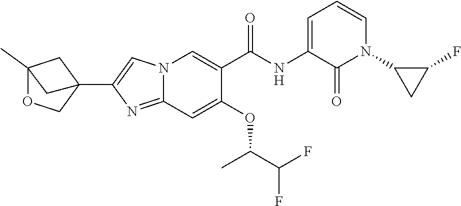 |
| | [stereochemistry arbitrarily assigned] |
| | SM: (S)-7-((1,1-difluoropropan-2-yl)oxy)-2-(1-methyl-2- |
| | oxabicyclo[2.1.1]hexan-4-yl)imidazo[1,2-a]pyridine-6-carboxylic acid |
| | (Preparation 62A) and Amine 7. |
| | LCMS m/z = 502.8 [M + H]⁺; 1H NMR (600 MHz, DMSO-d₆) δ: 10.49 |
| | (s, 1H), 9.19 (s, 1H), 8.44 (d, J = 7.99 Hz, 1H), 7.86 (s, 1H), 7.44 (d, |
| | J = 7.27 Hz, 1H), 7.37 (br s, 1H), 6.58-6.45 (m, 1H), 6.36 (t, J = 6.90 Hz, |
| | 1H), 5.22 (br s, 1H), 5.11-4.94 (m, 1H), 3.88 (s, 2H), 2.00 (dd, J = 1.45, |
| | 4.36 Hz, 2H), 1.75 (dd, J = 1.45, 4.36 Hz, 2H), 1.66-1.56 (m, 2H), 1.54 |
| | (d, J = 5.81 Hz, 3H), 1.49-1.44 (m, 1H), 1.42 (s, 3H) |
| 185* | 7-(((R)-1,1-difluoropropan-2-yl)oxy)-N-((1S,2R)-2- |
| | fluorocyclopropyl)-2-oxo-1,2-dihydropyridin-3-yl)-2-(1-methyl-2- |
| | oxabicyclo[2.1.1]hexan-4-yl)imidazo[1,2-a]pyridine-6-carboxamide |
| | 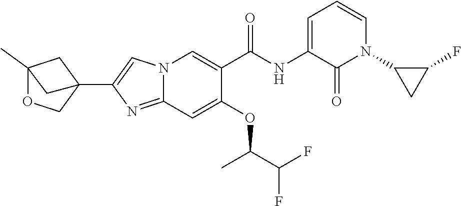 |
| | [stereochemistry arbitrarily assigned] |
| | SM: (R)-7-((1,1-difluoropropan-2-yl)oxy)-2-(1-methyl-2- |
| | oxabicyclo[2.1.1]hexan-4-yl)imidazo[1,2-a]pyridine-6-carboxylic acid |
| | (Preparation 62 B) and Amine 7. |
| | LCMS m/z = 502.8 [M + H]⁺; 1H NMR (600 MHz, DMSO-d₆) δ: 10.48 |
| | (s, 1H), 9.19 (s, 1H), 8.46-8.41 (m, 1H), 7.86 (s, 1H), 7.43 (d, J = 6.54 |
| | Hz, 1H), 7.37 (br s, 1H), 6.58-6.44 (m, 1H), 6.40-6.34 (m, 1H), 5.21 (br |
| | s, 1H), 5.11-4.96 (m, 1H), 3.88 (s, 2H), 2.00 (dd, J = 1.45, 4.36 Hz, 2H), |
| | 1.75 (dd, J = 1.45, 4.36 Hz, 2H), 1.65-1.56 (m, 1H), 1.53 (d, J = 6.54 Hz, |
| | 4H), 1.50-1.43 (m, 1H), 1.42 (s, 3H) |
| 186 | 7-isopropoxy-2-((1R,4S)-1-methyl-2-oxabicyclo[2.2.1]heptan-4-yl)-N- |
| | (1-((1S,2R)-2-methylcyclopropyl)-2-oxo-1,2-dihydropyridin-3- |
| | yl)imidazo[1,2-a]pyrimidine-6-carboxamide trifluoroacetate |
| | 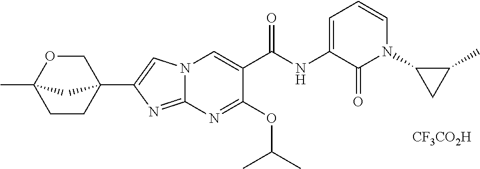 |
| | [stereochemistry arbitrarily assigned] |
| | SM: 7-Isopropoxy-2-((1R,4S)-1-methyl-2-oxabicyclo[2.2.1]heptan-4- |
| | yl)imidazo[1,2-a]pyrimidine-6-carboxylic acid (Preparation 92) and |
| | Amine 6 |
| | LCMS m/z = 482.2 [M + H]⁺. 1H NMR (500 MHz, MeOH-d₄) δ 10.59 (s, |
| | 1H), 9.59 (s, 1H), 8.60 (dd, J = 1.60, 7.40 Hz, 1H), 7.89 (s, 1H), 7.44 (d, |
| | J = 6.10 Hz, 1H), 6.42 (t, J = 7.25 Hz, 1H), 5.81 (spt, J = 6.21 Hz, 1H), 5.09- |
| | 4.91 (m, 1H), 4.02 (dd, J = 3.13, 6.64 Hz, 1H), 3.98-3.93 (m, 1H), 3.48- |
| | 3.39 (m, 1H), 2.24-2.09 (m, 2H), 2.04 (s, 2H), 1.97-1.82 (m, 2H), 1.70 |
| | (dd, J = 1.37, 6.26 Hz, 6H), 1.61-1.51 (m, 2H), 1.48 (s, 3H) |
| 187 | 7-isopropoxy-2-((1R,4S)-1-methyl-2-oxabicyclo[2.2.1]heptan-4-yl)-N- |
| | (1-((1S,2R)-2-methylcyclopropyl)-2-oxo-1,2-dihydropyridin-3- |
| | yl)imidazo[1,2-a]pyrimidine-6-carboxamide trifluoroacetate |
| | 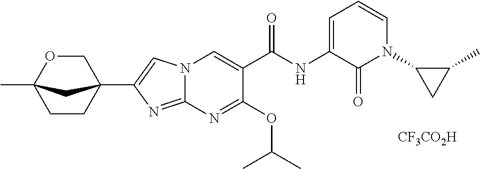 |
| | [stereochemistry arbitrarily assigned] |
| | SM: 7-isopropoxy-2-((1S,4R)-1-methyl-2-oxabicyclo[2.2.1]heptan-4- |
| | yl)imidazo[1,2-a]pyrimidine-6-carboxylic acid (Preparation 91) and |
| | Amine 6 LCMS m/z = 482.2 [M + H]⁺; 1H NMR (500 MHz, MeOH-d₄) |
| | δ: 10.59 (s, 1H), 9.59 (s, 1H), 8.61 (dd, J = 1.37, 7.48 Hz, 1H), 7.88 (s, |
| | 1H), 7.44 (d, J = 6.56 Hz, 1H), 6.42 (t, J = 7.25 Hz, 1H), 5.86-5.74 (m, |
| | 1H), 5.08-4.97 (m, 1H), 4.02 (dd, J = 3.20, 6.71 Hz, 1H), 3.96 (d, J = 6.71 |
| | Hz, 1H), 3.47-3.40 (m, 1H), 2.23-2.10 (m, 2H), 2.03 (s, 2H), 1.97-1.82 |
| | (m, 2H), 1.69 (dd, J = 2.14, 6.26 Hz, 7H), 1.61-1.52 (m, 2H), 1.48 (s, 3H) |
| 188 | 7-cyclobutoxy-2-(1-methyl-2-oxabicyclo[2.1.1]hexan-4-yl)-N-(1- |
| | ((1S,2R)-2-methylcyclopropyl)-2-oxo-1,2-dihydropyridin-3- |
| | yl)imidazo[1,2-a]pyrimidine-6-carboxamide trifluoroacetate |
| | 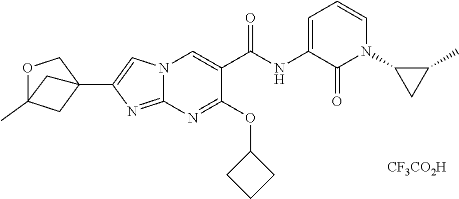 |
| | SM: 7-cyclobutoxy-2-(1-methyl-2-oxabicyclo[2.1.1]hexan-4- |
| | yl)imidazo[1,2-a]pyrimidine-6-carboxylic acid (Preparation 56) and |
| | Amine 6. |
| | LCMS m/z = 476.3 [M + H]⁺; 1H NMR (600 MHz, DMSO-d₆) δ: 10.62 |
| | (br s, 1H), 9.58 (s, 1H), 8.46 (br d, J = 7.27 Hz, 1H), 7.88 (br s, 1H), 7.39 |
| | (d, J = 6.54 Hz, 1H), 6.34 (t, J = 7.27 Hz, 1H), 5.47 (quin, J = 7.08 Hz, 1H), |
| | 3.90 (s, 2H), 2.64-2.52 (m, 4H), 2.08 (br s, 2H), 2.00-1.92 (m, 1H), |
| | 1.85-1.72 (m, 3H), 1.43 (s, 3H), 1.41-1.32 (m, 1H), 1.16 (q, J = 7.99 Hz, |
| | 1H), 0.90-0.85 (m, 1H), 0.75 (br d, J = 5.81 Hz, 3H) |
| 189 | 7-cyclobutoxy-2-(1-methyl-2-oxabicyclo[2.1.1]hexan-4-yl)-N-(1- |
| | ((1R,2S)-2-methylcyclopropyl)-2-oxo-1,2-dihydropyridin-3- |
| | yl)imidazo[1,2-a]pyrimidine-6-carboxamide trifluoroacetate |
| | 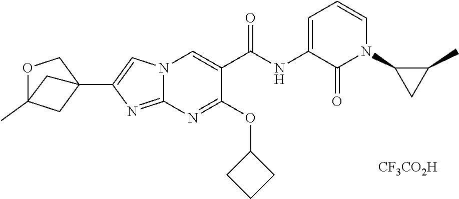 |
| | SM: 7-cyclobutoxy-2-(1-methyl-2-oxabicyclo[2.1.1]hexan-4- |
| | yl)imidazo[1,2-a]pyrimidine-6-carboxylic acid (Preparation 56) and |
| | Amine 5. |
| | LCMS m/z = 476.3 [M + H]⁺; 1H NMR (600 MHz, DMSO-d₆) δ: 10.62 |
| | (br s, 1H), 9.58 (s, 1H), 8.46 (br d, J = 7.27 Hz, 1H), 7.88 (br s, 1H), 7.39 |
| | (d, J = 6.54 Hz, 1H), 6.34 (t, J = 7.27 Hz, 1H), 5.47 (quin, J = 7.27 Hz, 1H), |
| | 3.90 (s, 2H), 2.62-2.51 (m, 5H), 2.08 (br s, 2H), 2.01-1.92 (m, 1H), |
| | 1.84-1.72 (m, 4H), 1.43 (s, 4H), 1.40-1.32 (m, 1H), 1.16 (q, J = 7.75 Hz, |
| | 1H), 0.91-0.84 (m, 1H), 0.75 (br d, J = 5.81 Hz, 4H). |
| 190 | 7-cyclobutoxy-2-(1-methyl-2-oxabicyclo[2.1.1]hexan-4-yl)-N-(1- |
| | ((1S,2R)-2-methylcyclopropyl)-2-oxo-1,2-dihydropyridin-3- |
| | yl)imidazo[1,2-a]pyridine-6-carboxamide trifluoroacetate |
| | 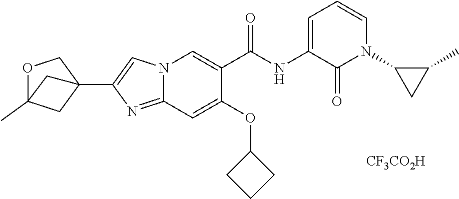 |
| | SM: 7-cyclobutoxy-2-(1-methyl-2-oxabicyclo[2.1.1]hexan-4- |
| | yl)imidazo[1,2-a]pyridine-6-carboxylic acid (Preparation 33) and |
| | Amine 6. |
| | LCMS m/z = 475.3 [M + H]⁺; 1H NMR (600 MHz, DMSO-d₆) δ: 10.64 |
| | (br s, 1H), 9.40 (s, 1H), 8.47 (br d, J = 7.27 Hz, 1H), 8.12 (s, 1H), 7.39 (br |
| | d, J = 7.27 Hz, 1H), 7.11 (br s, 1H), 6.34 (t, J = 7.27 Hz, 1H), 5.24-5.14 |
| | (m, 1H), 3.93 (s, 2H), 2.64-2.52 (m, 4H), 2.13 (d, J = 3.63 Hz, 2H), 2.01- |
| | 1.93 (m, 1H), 1.86 (d, J = 4.36 Hz, 2H), 1.79 (sxt, J = 9.30 Hz, 1H), 1.45 |
| | (s, 3H), 1.40-1.32 (m, 1H), 1.16 (q, J = 7.27 Hz, 1H), 0.87 (br d, J = 3.63 |
| | Hz, 1H), 0.74 (br d, J = 5.81 Hz, 3H). |

*compound was purified by purified by RPHPLC (PSR 310) ammonium hydroxide conditions 5-55%
MeCN-water.

### Examples 191 and 192: 7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyridine-6-carboxamide and 7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyridine-6-carboxamide

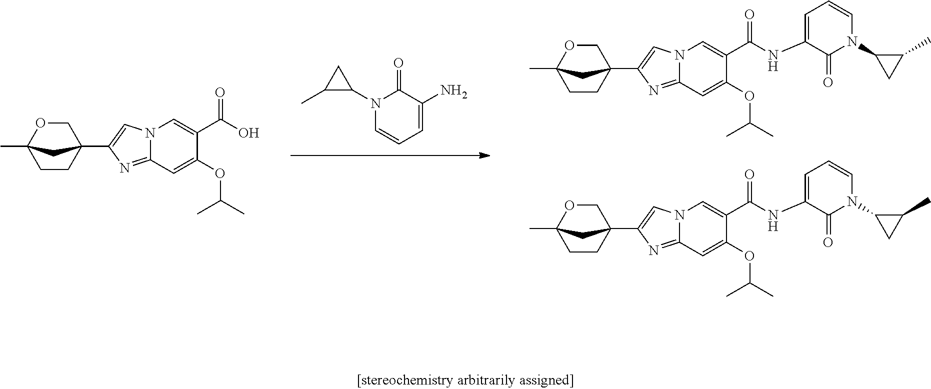

7-Isopropoxy-2-((1S,4R)-1-methyl-2-oxabicyclo[2.2.1]heptan-4-yl)imidazo[1,2-a]pyridine-6-carboxylic acid (Preparation 59A, 208.22 mg, 630.24 μmol) was dissolved in DMF (3 mL), DIPEA (329.33 μL, 1.89 mmol) and HATU (252.29 mg, 661.75 μmol) were added and the solution stirred for 1 min. Trans-rac-3-Amino-1-(2-methylcyclopropyl)pyridin-2(1H)-one hydrochloride (126.47 mg, 630.24 μmol, Preparation 76) was added and the mixture stirred at rt overnight. The mixture was concentrated in vacuo and the residue purified by RPHPLC using a gradient of 5-60% MeCN in water to give 7-isopropoxy-2-((1S,4R)-1-methyl-2-oxabicyclo[2.2.1]heptan-4-yl)-N-(1-(2-methylcyclopropyl)-2-oxo-1,2-dihydropyridin-3-yl)imidazo[1,2-a]pyridine-6-carboxamide.

The compound was further purified chiral SFC: CHIRALPAK AD-H 30×250 mm, 5 um Method: 40% MeOH w/ 0.1% DEA in CO₂ (flow rate: 100 mL/min, ABPR 120 bar, MBPR 40 psi, column temp 40 deg C.) to provide Peak 1: [Example 191] 7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyridine-6-carboxamide (100% ee), 77.8 mg. (Stereochemistry arbitrarily assigned). 1H NMR (500 MHz, MeOH-d₄) 9.34 (s, 1H), 8.55 (dd, J=1.68, 7.48 Hz, 1H), 7.97 (s, 1H), 7.34 (dd, J=1.68, 7.02 Hz, 1H), 7.25 (s, 1H), 6.36 (t, J=7.25 Hz, 1H), 5.17 (q, J=12.20 Hz, 1H), 4.04-4.00 (m, 1H), 3.98-3.94 (m, 1H), 3.15-3.10 (m, 1H), 2.23-2.12 (m, 2H), 2.04 (s, 2H), 1.97-1.82 (m, 2H), 1.68 (d, J=6.10 Hz, 6H), 1.48 (s, 3H), 1.32-1.24 (m, 5H), 1.18-1.11 (m, 1H)

Peak 2: [Example 192] 7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyridine-6-carboxamide (100% ee), 72.6 mg. 1H NMR (500 MHz, MeOH-d₄) 9.30 (s, 1H), 8.55 (dd, J=1.60, 7.40 Hz, 1H), 7.92 (s, 1H), 7.33 (dd, J=1.53, 7.02 Hz, 1H), 7.21 (s, 1H), 6.36 (t, J=7.25 Hz, 1H), 5.14 (td, J=5.99, 12.13 Hz, 1H), 4.03 (dd, J=3.20, 6.56 Hz, 1H), 3.96 (d, J=6.71 Hz, 1H), 3.13 (br t, J=7.10 Hz, 1H), 2.23-2.10 (m, 2H), 2.03 (s, 2H), 1.97-1.81 (m, 2H), 1.67 (d, J=6.10 Hz, 6H), 1.48 (s, 3H), 1.32-1.24 (m, 6H), 1.17-1.10 (m, 1H), 0.99-0.91 (m, 1H)

Assays

Compounds of the invention were assessed for their ability to inhibit IRAK4 activity. The inhibitory properties of the compounds of the disclosure described herein can be evidenced by testing in any one of the following assays.

1. Biochemical Assay

The 2-hour 1 mM ATP Biochemical Assay employed a MesoScale Detection (MSD) format. The kinase reaction was based on the IRAK4 phosphorylation of a biotin labeled peptide (IRAK1 activation loop sequence 360-389).

The kinase reaction in 30 μl was carried out in wells of a 384 well polypropylene assay plate, with 0.1 nM IRAK4, 1.6 μM of biotinylated peptide substrate and 1 mM ATP in 50 mM Hepes, pH 7.5, 60 mM NaCl, 5 mM MgCl₂, 0.25 mM MnCl₂, 2 mM DTT, 0.01% BSA, 0.01% BSA, and 1% DMSO (from compound DMSO stocks), for 2 hour at room temperature. The activity was quenched with 11 μl of 70 mM EDTA, pH 8.

To detect the phosphorylated biotinylated peptide substrate, 30 μl of the quenched reaction mixture was added to equivalent wells of a 384 well streptavidin coated MesoScale plate (Meso Scale Discovery #L21SA-1). After a 1 hour incubation of the plate for 1 hour at room temperature with gentle mixing, the plate wells were washed 3 times with 50 mM Tris, pH 7.5, 150 mM NaCl, 0.02% Tween-20.

A 25 μl volume of 1:500 anti-P-Threonine Rabbit polyclonal Antibody plus 1:500 Goat-anti-Rabbit Sulfo Tag Antibody (Meso Scale Discovery R32AB-1) in 50 mM Tris, pH 7.5, 150 mM NaCl, 0.02% Tween-20 plus 2% BSA was then added to each well. After a 1-hour incubation of the plate for 1 hour at room temperature with gentle mixing, the plate wells were washed, 3 times with 50 mM Tris, pH 7.5, 150 mM NaCl, 0.02% Tween-20. A 40 μl volume of 2×MSD Read Buffer (Meso Scale Discovery R92TC-1) was added to each well, and the plate was read immediately in an MSD Plate Reader (Meso Scale Discovery).

2. MDR1-MDCK assay procedure
Human MDR1 transfected MDCK cells (NIH cell line in-licensed from Absorption Systems) were used in the assay.
The compounds were tested at 1 μM concentration prepared in transport buffer (Hank's balanced salt solution with HEPES)
MDR1-MDCK cell were cultured for 7 days in 96 well transwell insert plates (Corning). Insert plates were washed before the assay and TEER (Trans epithelial electric resistance) was measured.
These plates were loaded with test compound solution 85 μL for A-B transport and 260 μL for B-A transport in the respective donor compartment. The volume of receiver buffer (Transport buffer supplemented with 1% BSA) in the respective receiver compartment was 250 and 75 μL.
10 μL samples was taken from donor compartment (T=0 timepoint)
Assay plates were incubated for 120 minutes.
At 120 minutes (T=120 timepoint) samples from respective donor (10 uL) and receiver (50 μL) compartments was taken.
After addition of 40 μL transport buffer with BSA to donor samples, crash solution (Acetonitrile with internal standard, 110 μL) was added to all samples.
After centrifugation 50 μL supernatant was transferred to separate plate and mixed with 50 μL water.
Samples were analyzed using LC-MS/MS coupled with high throughput injection system.
Analyte/internal standard area ratios were used for apparent permeability (Pₐₚₚ), efflux ratio and mass recovery estimation based on equations below.

Pₐₚₚ=(dCᵣ/dt)×Vᵣ/(A×C_{E})

Mass balance=100×((Vᵣ×Cᵣ^{final})+(V_{d}×C_{d}^{final}))/(V_{d}×C_{E})
Where:
dCᵣ/dt is the cumulative concentration in the receiver compartment versus time in μM s⁻¹
Vᵣ is the volume of the receiver compartment in cm³
V_{d} is the volume of the donor compartment in cm³
A is the area of the insert (0.143 cm² for 96-well insert)
C_{E} is the estimated experimental concentration (Time=0) of the dosing solution
Cᵣ^{final} is the concentration of the receiver at the end of the incubation period
C_{d}^{final} is the concentration of the donor at the end of the incubation period.

3. Solubility Assay

Sample Receipt and Preparation:
Samples received as 10 mM DMSO stock solutions for solubility analysis by Chemiluminescent Nitrogen Detection (CLND).
   Frozen on dry ice in 96 well plates
   Prior to setup: thaw, centrifuge, and sonicated in a water bath to facilitate dissolution.

Buffer Preparation:
Potassium Phosphate Buffer, pH 6.8
   0.2M potassium phosphate, monobasic solution was prepared by dissolving 27.22 g/L of monobasic potassium phosphate in water
   62.5 mL of the 0.2M monobasic potassium phosphate solution was transferred to a 250 mL volumetric flask
   28 mL of 0.2N NaOH was added to the 250 mL volumetric flask
   Water was added to bring to volume
   Final pH was measured

Kinetic Solubility Assay Setup:
Dilute the 10 mM DMSO stock solution 50-fold in buffer (2% DMSO) in 1 well of a Millipore solubility filter plate
   0.45 μm polycarbonate filter membrane
Seal the filter plate with heat sealing film
Incubate on a rotary shaker
   24 hours at ambient temperature
After incubation remove seal and vacuum filter, collecting filtrate
Seal collection plate containing filtrates for analysis.

Kinetic Solubility Assay:
Inject filtrates into the nitrogen detector for quantification on Analiza's Automated Discovery Workstation (ADW).
Solubility results generated in μg/mL

4. Kpuu Assay

Generic Study Protocol for in vivo PK Studies (non-GLP)

In Vivo

For the brain-to-plasma partition coefficient (Kp) evaluation, a dosing solution was intravenously infused into animals at a constant flow rate for 4 to 24 h. Blood samples were serially collected during infusion, and CSF and brain samples were harvested at the end of infusion.

For characterization of PK properties, a dosing solution was administered to animals via oral gavage or parenteral routes. Blood samples were collected after administration. Other biological samples, including tissue, bile, urine, and feces, can be collected during or at the end of the study if necessary.

All the animal experiments were conducted in accordance with the internally approved animal protocols.

Bioanalysis

Tissue samples were typically homogenized in phosphate buffer saline (PBS) using a bead ruptor.

CSF samples were typically diluted with 8% BSA in PBS to prevent from non-specific binding. Artificial CSF (aCSF) is used as the surrogate matrix.

Dosing solutions were spiked into plasma for analysis when needed.

Calibration curves were prepared by spiking the analyte(s) into blank matrices, which were processed together with plasma, tissue homogenate and/or CSF samples by protein precipitation using a proper organic solvent (e.g. acetonitrile and methanol) containing generic analogue internal standards (e.g. verapamil, chrysin and glyburide). Matrix matching was used when analyzing multiple matrices in the same run. Samples above the upper limit of quantitation (ULOQ) needed to be diluted into the calibration range using either a pre-extraction or post-extraction dilution approach.

Processed samples were analyzed by LC-MS/MS using a proper method performing within the acceptable sensitivity, selectivity, precision and accuracy. For an analytical run to be accepted, over 75% of the calibration standards in the dual calibration curves needed to be within 20% of the nominal concentrations.

Compound- or study-specific bioanalytical methods that deviate from the typical procedure might be used when necessary, which will be documented in a study specific protocol included in the data upload.

PK

Plasma concentrations were analyzed by non-compartmental analysis (NCA) using a “Linear up log down” fitting to generate basic PK parameters that include but are not limited to volume of distribution (Vd), maximal concentration (Cmax), time to reach maximal concentration (Tmax), area under the curve (AUC), half-life (t1/2), clearance (CL) and bioavailability (F). The PK parameters were normalized to the adjusted dose when dosing solution analysis was conducted.

Brain concentrations were compared against plasma concentrations at the corresponding timepoint for the calculation of partition coefficient (Kp).

Unbound drug partition coefficient (Kpuu), defined as the ratio of unbound drug partition across the blood-brain barrier, was calculated using the equation below:

$$K p u u = \frac{{C}_{b} \times {F}_{u b}}{{C}_{p} \times {F}_{u p}}$$

C_{b}: measured total drug concentration in brain
F_{ub}: unbound drug fraction in brain
Cₚ: measured total drug concentration in plasma
Fᵤₚ: unbound drug fraction in plasma

Compound- or study-specific PK analysis that deviates from the typical procedure might be used when necessary, which will be documented in a study specific protocol included in the data upload.

Determination of Fraction Unbound (Fu):

The unbound fraction of the test compound was determined based on the protocols described below.

1) Dilute initial 10 mM test article to 125 M by adding 5 L to a total volume of 395 L solvent solution (100% acetonitrile) in a 1 mL 96-well plate (Waters 186002481 Milford, MA). Ensure that the compounds are in solution.

2) Thaw frozen (rat, human, mouse, dog, and/or monkey) plasma (BIOIVT, Westbury, NY) in and warm the PBS buffer in a warm (37° C.) water bath.
Dilute the 125 uM test article solutions by adding 8 L to a final volume of 992 L of plasma to make a final concentration of 1 M in a 2 mL 96 well plate (Costar 3961). Mix thoroughly.
This spiked plasma solution is shown in FIG. 1.

3) Prepare a chilled ‘crash’ solution of internal standard in a solvent solution.
Pipette 200 L of 25 ng/mL solution of internal standard, CPDPX (8-Cyclopentyl-1,3-dipropylxanthine, Sigma-Aldrich, C101) in 1:1 acetonitrile/methanol solvent solution into a 1 mL 96-well plate.
Chill on ice or refrigerate at 4° C.
This solution becomes the ‘Crash’ plate in FIG. 1.

4) From remaining spiked plasma, remove 50 μL (T=0 h) of each plasma sample and place into a crash plate which contains 200 μL. To matrix match, add 50 μL of blank buffer to the crashed sample (similar to PPB samples). Maintain remaining spiked plasma at 37° C. for 4 h time point

5) Transfer 500 μL of the warmed PBS buffer to the white side of the RED device (Thermo Scientific, Rockford IL, baseplate cat #89811, insert cat #89810) and 300 μL of the spiked plasma to the corresponding red ring side of the RED device.

6) Cover all the RED device plates with a lid and transfer them to a 37° C. incubator with 5% C₀₂ environment and shake at 200 rpm for 4 hours.

7) Reaction Termination after 4 hours:
Add 50 μL of sample (plasma or buffer sample) and 50 μL of the opposite blank matrix (add blank buffer to the plasma samples and blank plasma to the buffer samples) to crash plates (same as above) to the crash plate containing 200 μL. Mix crash plate thoroughly.
From remaining spiked plasma, remove 50 μL (T=4 h) of each plasma sample and place into a crash plate. To matrix match, add 50 μL of blank buffer to the crashed sample (similar to the protein binding samples).
Centrifuge crash plate at 3900 rpm for 10 minutes at 4° C. (Eppendorf Centrifuge 5810R, Hamburg, Germany)

8) Sample preparation of LC/MS/MS:
Transfer 30 μL of supernatant from the crash plates to 384-well plates containing 120 μL of 0.1 formic acid in 90:10 water:acetonitrile using the PPB 96 to 384 pretty method on the Tecan. Inject into the LC/MS.
Volumes and diluent composition may be adjusted based on instrument (LC-MS/MS) sensitivity and test article sensitivity, solubility, and polarity to ensure adequate signal and retention of test articles within the linear limitation of the instrument.

9) Standard Curve
Prepare standard curve of pooled test articles treated in a similar fashion as the reaction samples using plasma and buffer.

10) Data processing and analysis

Multiquant will be chosen application used to process the data for PPB.

Equations:

Equation 1. Calculation of % Free (% PPBunb)

% Free=(PAR of buffer side/PAR of plasma side)*100
PAR—Peak area ratio (PAR)
Fu % Free. 100
Fu=fraction unbound

Equation 2. Final calculation using dilution factor (D)

This dilution factor formula is used only if tissue or plasma is diluted.

### Data for Examples

| | IRAK4 MSD | | pH 6.8 | |
|---|---|---|---|---|
| Example | BIOCHEMICAL | MDR1-MDCK | Solubility | Brain |
| number | IC50 (nM) | Efflux Ratio | (ug/mL) | Kpu, u |
| 1 | 0.2 | 1.7 | 56.0 | |
| 2 | 0.4 | 3.3 | 65.7 | |
| 3 | 0.2 | 2.9 | 36.0 | 0.25 |
| 4 | 0.2 | 7.1 | 57.4 | 0.26 |
| 5 | 0.1 | 6.1 | 32.9 | 0.38 |
| 6 | 0.1 | 7.9 | 58.0 | 0.27 |
| 7 | 0.3 | 5.0 | 79.2 | 0.66 |
| 8 | 0.3 | 5.4 | 75.9 | 0.49 |
| 9 | 0.4 | 9.7 | 70.6 | 0.18 |
| 10 | 0.8 | 9.4 | 79.3 | 0.22 |
| 11 | 0.3 | 13.8 | 62.4 | 0.21 |
| 12 | 0.3 | 4.3 | 22.3 | 0.33 |
| 13 | 0.3 | 3.8 | 20.3 | |
| 14 | 1.3 | 4.8 | 75.7 | |
| 15 | 0.4 | 8.1 | 78.6 | 0.41 |
| 16 | 0.3 | 2.8 | 5.2 | |
| 17 | 0.5 | 3.9 | 8.5 | |
| 18 | 0.2 | 8.5 | 61.0 | 0.70 |
| 19 | 0.5 | 6.0 | 47.0 | 0.49 |
| 20 | 0.7 | 2.8 | 68.1 | |
| 21 | 0.9 | 3.2 | 61.3 | |
| 22 | 0.3 | 4.3 | 69.0 | |
| 23 | 0.5 | 2.8 | 61.9 | |
| 24 | 2.6 | 6.2 | 52.3 | |
| 25 | 0.3 | 3.2 | 66.7 | 0.36 |
| 26 | 0.7 | 4.1 | 65.3 | |
| 27 | 0.6 | 4.0 | 65.7 | 0.42 |
| 28 | 0.2 | 18.2 | 62.6 | |
| 29 | 0.6 | 13.6 | 60.2 | |
| 30 | 0.2 | 11.1 | 46.5 | |
| 31 | 0.3 | 2.7 | 24.2 | |
| 32 | 0.5 | 5.4 | 72.4 | |
| 33 | 1.4 | 8.4 | 10.1 | 0.45 |
| 34 | 1.0 | 11.1 | 39.2 | |
| 35 | 0.3 | 11.8 | 77.0 | 0.56 |
| 36 | 1.7 | 6.4 | 20.8 | |
| 37 | 3.1 | 10.2 | 64.4 | |
| 38 | 0.3 | 7.0 | | |
| 39 | 0.2 | 6.8 | 68.9 | |
| 40 | 0.4 | 7.5 | 70.2 | |
| 41 | 0.6 | 9.1 | | |
| 42 | 0.3 | 10.8 | 72.5 | |
| 43 | 1.3 | 10.3 | | |
| 44 | 0.6 | 2.8 | | |
| 45 | 0.2 | 5.5 | 6.8 | |
| 46 | 0.2 | 5.0 | 9.0 | 1.81 |
| 47 | 0.2 | 5.1 | 9.0 | |
| 48 | 0.8 | 3.2 | 63.2 | |
| 49 | 0.4 | 3.6 | 56.1 | 0.66 |
| 50 | 0.5 | 2.9 | | 0.66 |
| 51 | 1.8 | 3.1 | | |
| 52 | 0.3 | 15.9 | | |
| 53 | 1.1 | 7.6 | | |
| 54 | 0.5 | 18.1 | 28.3 | 0.44 |
| 55 | 1.1 | 4.7 | | |
| 56 | 0.6 | 4.1 | 67.5 | |
| 57 | 23.0 | 7.9 | | |
| 58 | 0.4 | 9.0 | 72.1 | |
| 59 | 9.0 | 9.4 | | |
| 60 | 0.2 | 9.0 | 55.5 | |
| 61 | 2.0 | 2.8 | 65.8 | 0.45 |
| 62 | 0.4 | 3.6 | 58.2 | |
| 63 | 0.8 | 5.5 | 67.1 | 0.48 |
| 64 | 0.5 | 5.0 | 49.6 | |
| 65 | 1.5 | 6.6 | 61.2 | |
| 66 | 0.3 | 7.2 | 74.1 | 0.86 |
| 67 | 1.3 | 2.8 | 48.8 | 0.96 |
| 68 | 4.3 | 2.2 | 53.6 | |
| 69 | 0.3 | 4.7 | 6.1 | |
| 70 | 1.2 | 3.9 | 9.9 | |
| 71 | 1.5 | 2.1 | 8.9 | |
| 72 | 4.2 | 1.7 | 1.5 | |
| 73 | 14.0 | 1.0 | | |
| 74 | 1.2 | 1.0 | 47.5 | |
| 75 | 1.6 | 1.4 | 40.4 | |
| 76 | 1.6 | 3.6 | 50.9 | |
| 77 | 0.3 | 2.7 | 7.2 | 0.61 |
| 78 | 11.0 | 2.1 | | |
| 79 | 0.7 | 2.9 | 45.7 | 0.89 |
| 80 | 0.3 | 4.9 | 9.5 | 0.90 |
| 81 | 1.3 | 6.4 | | |
| 82 | 0.5 | 6.9 | 64.0 | |
| 83 | 0.6 | 5.6 | 65.1 | |
| 84 | 0.6 | 7.4 | 37.7 | 0.67 |
| 85 | 0.6 | 7.7 | 44.5 | |
| 86 | 0.8 | 0.9 | 44.9 | |
| 87 | 0.6 | 23.2 | 48.4 | |
| 88 | 22.0 | 24.4 | 20.7 | |
| 89 | 0.2 | 20.0 | 23.9 | |
| 90 | 0.3 | 28.3 | 74.8 | |
| 91 | 0.8 | 5.1 | | |
| 92 | 61.0 | | | |
| 93 | 0.6 | 4.9 | 62.8 | |
| 94 | 0.3 | 15.7 | 65.2 | |
| 95 | 1.2 | 3.1 | 35.3 | |
| 96 | 0.2 | 10.4 | 75.7 | |
| 97 | 0.3 | 12.6 | 59.5 | |
| 98 | 0.7 | 2.1 | 89.7 | |
| 99 | 0.4 | | 62.0 | |
| 100 | 0.6 | 12.6 | 27.6 | |
| 101 | 0.4 | 9.6 | | |
| 102 | 0.3 | 10.8 | 69.3 | |
| 103 | 0.4 | 12.8 | 58.8 | |
| 104 | 1.7 | 4.3 | 74.5 | |
| 105 | 0.7 | 15.8 | 38.7 | |
| 106 | 0.5 | 12.8 | 44.4 | |
| 107 | 0.3 | 3.0 | | |
| 108 | 0.1 | 4.0 | | 0.52 |
| 109 | 0.5 | 7.1 | 50.2 | 0.25 |
| 110 | 0.5 | 7.0 | | |
| 111 | 0.2 | 12.4 | 71.2 | |
| 112 | 0.3 | 9.0 | 73.3 | 0.51 |
| 113 | 12.6 | 8.7 | 59.5 | |
| 114 | 0.3 | 10.4 | 68.6 | |
| 115 | 0.9 | 4.0 | 20.9 | |
| 116 | 0.6 | 5.3 | 55.6 | |
| 117 | 0.7 | 3.9 | 23.1 | |
| 118 | 0.9 | 5.1 | 56.2 | |
| 119 | 0.7 | 4.2 | 79.0 | 0.73 |
| 120 | 0.7 | | 76.7 | |
| 121 | 1.1 | | 33.9 | |
| 122 | 0.5 | | 48.8 | |
| 123 | 1.0 | | 73.0 | |
| 124 | 45.5 | | | |
| 125 | 0.5 | | 39.9 | |
| 126 | 66.5 | 4.4 | | |
| 127 | 0.7 | 3.4 | 32.6 | |
| 128 | 0.3 | 20.8 | 72.5 | |
| 129 | 3.4 | 31.1 | | |
| 130 | 0.2 | 26.1 | 63.6 | 0.08 |
| 131 | 11.4 | 29.6 | | |
| 132 | 0.2 | 28.0 | 70.5 | 0.14 |
| 133 | 21.4 | 4.3 | | |
| 134 | 0.5 | 4.4 | 38.6 | |
| 135 | 4.2 | 5.3 | | |
| 136 | 4.0 | 5.2 | 50.4 | 0.40 |
| 137 | 1.4 | 4.8 | | |
| 138 | 6.3 | 3.8 | | |
| 139 | 0.5 | 3.0 | 38.4 | |
| 140 | 1.1 | | | |
| 141 | 0.4 | | 43.2 | |
| 142 | 1.2 | | 37.0 | |
| 143 | 6.1 | | | |
| 144 | 0.8 | | 34.4 | |
| 145 | 1.7 | 3.6 | | |
| 146 | 0.9 | 1.7 | 29.7 | |
| 147 | 15.9 | 1.5 | | |
| 148 | 1.2 | 2.6 | | |
| 149 | 0.4 | 2.8 | 34.5 | 1.34 |
| 150 | 2.7 | 1.0 | | |
| 151 | 1.1 | 1.2 | | |
| 152 | 0.8 | 1.4 | 24.9 | |
| 153 | 1.7 | 1.5 | | |
| 154 | 1.0 | 5.0 | 37.2 | |
| 155 | 0.9 | 6.2 | 35.8 | 0.36 |
| 156 | 0.4 | 2.5 | 62.8 | |
| 157 | 1.0 | 3.2 | | |
| 158 | 4.4 | 16.0 | | |
| 159 | 0.2 | 12.7 | 68.2 | 0.28 |
| 160 | 3.1 | | | |
| 161 | 0.1 | 8.6 | 54.6 | |
| 162 | 7.2 | 38.2 | | |
| 163 | 0.2 | 34.0 | 66.0 | |
| 164 | 8.6 | 18.7 | | |
| 165 | 0.1 | 16.9 | | |
| 166 | 41.0 | 2.6 | | |
| 167 | 0.2 | 2.3 | 19.2 | |
| 168 | 7.5 | | | |
| 169 | 0.3 | 10.4 | | |
| 170 | 9.4 | | | |
| 171 | 0.2 | 9.8 | 47.9 | |
| 172 | 27.0 | | | |
| 173 | 0.2 | 10.9 | 22.6 | |
| 174 | 37.5 | | | |
| 175 | 0.2 | 8.9 | 43.0 | |
| 176 | 0.3 | 8.1 | | |
| 177 | 9.2 | 7.9 | | |
| 178 | 0.5 | 6.4 | | |
| 179 | 4.7 | | | |
| 180 | 0.2 | 2.6 | 23.0 | 1.58 |
| 181 | 24.9 | 5.3 | | |
| 182 | 0.2 | 3.9 | 31.8 | |
| 183 | 15.4 | 2.9 | | |
| 184 | 0.2 | 19.2 | 73.4 | |
| 185 | 0.1 | 21.8 | 73.2 | |
| 186 | 0.2 | 10.8 | 64.7 | 0.32 |
| 187 | 0.2 | 10.9 | 55.1 | 0.19 |
| 188 | 0.8 | 4.4 | 49.7 | |
| 189 | 34.6 | 3.4 | | |
| 190 | 0.9 | 4.1 | 54.2 | |
| 191 | 0.4 | 1.5 | 48.5 | |

| | | | |
|---|---|---|---|
| 192 | 0.7 | 1.7 | 60.6 |

### Comparator Compounds

### Comparator 1A: N-(6-(difluoromethyl)pyridin-2-yl)-7-isopropoxy-2-(1-methyl-2-oxabicyclo[2.1.1]hexan-4-yl)imidazo[1,2-a]pyridine-6-carboxamide

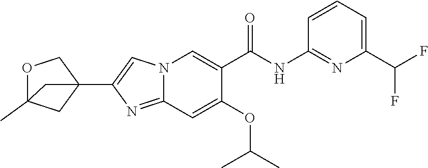 [SMILES: CC(C)Oc1cc2nc(C34COC(C)(C3)C4)cn2cc1C(=O)Nc1cccc(C(F)F)n1]

### Comparator 2A: N-(6-(difluoromethyl)pyridin-2-yl)-7-isopropoxy-2-(1-methyl-2-oxabicyclo[2.1.1]hexan-4-yl)imidazo[1,2-a]pyrimidine-6-carboxamide

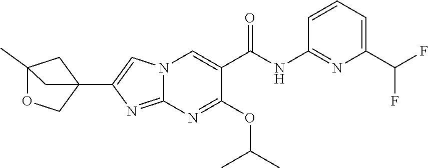 [SMILES: CC(C)Oc1nc2nc(C34COC(C)(C3)C4)cn2cc1C(=O)Nc1cccc(C(F)F)n1]

### Comparator 3A: N-(6-(difluoromethyl)pyridin-2-yl)-7-isopropoxy-2-((1R,4S)-1-methyl-2-oxabicyclo[2.2.1]heptan-4-yl)imidazo[1,2-a]pyridine-6-carboxamide (stereochemistry arbitrarily assigned)

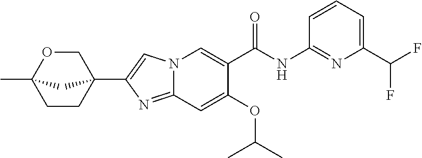 [SMILES: CC(C)Oc1cc2nc([C@]34CC[C@](C)(C3)OC4)cn2cc1C(=O)Nc1cccc(C(F)F)n1]

### Comparator 4A: N-(6-(difluoromethyl)pyridin-2-yl)-7-isopropoxy-2-((1S,4R)-1-methyl-2-oxabicyclo[2.2.1]heptan-4-yl)imidazo[1,2-a]pyrimidine-6-carboxamide (stereochemistry arbitrarily assigned)

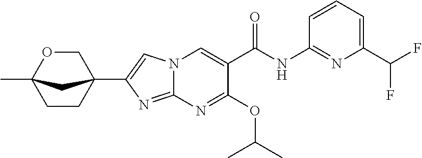 [SMILES: CC(C)Oc1nc2nc([C@@]34CC[C@@](C)(C3)OC4)cn2cc1C(=O)Nc1cccc(C(F)F)n1]

### Comparator 5A: N-(6-(difluoromethyl)pyridin-2-yl)-7-isopropoxy-2-(1-methyl-2-oxabicyclo[2.2.2]octan-4-yl)imidazo[1,2-a]pyrimidine-6-carboxamide

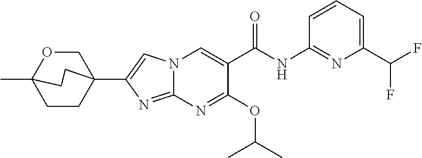 [SMILES: CC(C)Oc1nc2nc(C34CCC(C)(CC3)OC4)cn2cc1C(=O)Nc1cccc(C(F)F)n1]

### Comparator 1B: 7-isopropoxy-2-(1-methyl-2-oxabicyclo[2.1.1]hexan-4-yl)-N-(6-methylpyrazolo[1,5-a]pyrimidin-3-yl)imidazo[1,2-a]pyridine-6-carboxamide

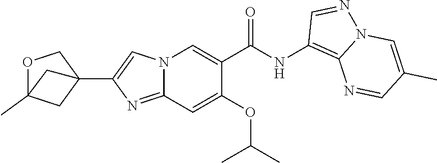

### Comparator 2B: 7-isopropoxy-2-(1-methyl-2-oxabicyclo[2.1.1]hexan-4-yl)-N-(6-methylpyrazolo[1,5-a]pyrimidin-3-yl)imidazo[1,2-a]pyrimidine-6-carboxamide

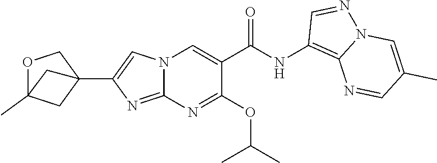

### Comparator 3B: 7-isopropoxy-2-((1S,4R)-1-methyl-2-oxabicyclo[2.2.1]heptan-4-yl)-N-(6-methylpyrazolo[1,5-a]pyrimidin-3-yl)imidazo[1,2-a]pyridine-6-carboxamide (stereochemistry arbitrarily assigned)

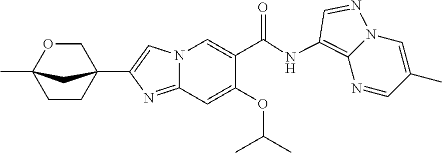

To the mixture of 7-isopropoxy-2-((1S,4R)-1-methyl-2-oxabicyclo[2.2.1]heptan-4-yl)imidazo[1,2-a]pyridine-6-carboxylic acid [preparation 59A] (33.0 mg, 100 μmol), 3-amino-1-cyclopropyl-pyridin-2-one HCl salt (15.2 mg, 81.3 μmol), HATU (28.4 mg, 74.6 mol) in DMF (0.8 mL) was added Hunig's base (47 μL, 271 μmol). The mixture was stirred at rt overnight. The reaction mixture partitioned between EtOAc and water. The aqueous layer was extracted with EtOAc. The combined organic phases were concentrated and purified by normal phase silica gel column (12 g, eluting with EtOAc 100%-EtOAc/EtOH 7/1) to get 7-isopropoxy-2-((1S,4R)-1-methyl-2-oxabicyclo[2.2.1]heptan-4-yl)-N-(6-methylpyrazolo[1,5-a]pyrimidin-3-yl)imidazo[1,2-a]pyridine-6-carboxamide as an off-white powder after lyophilization (25 mg, 80% yield). LCMS (ESI) m/z 461.0 (M+H)⁺. 1H NMR (METHANOL-d4, 500 MHz) δ 9.16 (s, 1H), 8.61 (dd, 1H, J=1.5, 7.6 Hz), 7.73 (s, 1H), 7.36 (dd, 1H, J=1.8, 6.7 Hz), 6.84 (s, 1H), 6.40 (t, 1H, J=7.0 Hz), 5.07 (quin, 1H, J=7.0 Hz), 4.61 (s, 1H), 4.03 (s, 2H), 3.4-3.5 (m, 1H), 2.6-2.8 (m, 4H), 2.13 (dd, 2H, J=1.5, 4.6 Hz), 2.05 (br d, 1H, J=2.4 Hz), 1.8-1.9 (m, 3H), 1.52 (s, 3H), 1.1-1.2 (m, 2H), 0.9-1.0 (m, 2H).

### Comparator 4B: 7-isopropoxy-2-((1S,4R)-1-methyl-2-oxabicyclo[2.2.1]heptan-4-yl)-N-(6-methylpyrazolo[1,5-a]pyrimidin-3-yl)imidazo[1,2-a]pyrimidine-6-carboxamide (stereochemistry arbitrarily assigned)

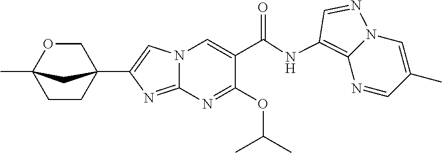

### Comparator 5B: 7-isopropoxy-2-(1-methyl-2-oxabicyclo[2.2.2]octan-4-yl)-N-(6-methylpyrazolo[1,5-a]pyrimidin-3-yl)imidazo[1,2-a]pyridine-6-carboxamide

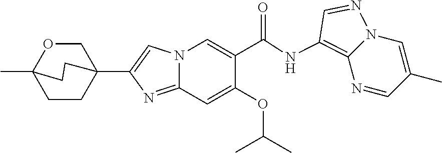

### Comparator 6B: 7-isopropoxy-2-(1-methyl-2-oxabicyclo[2.2.2]octan-4-yl)-N-(6-methylpyrazolo[1,5-a]pyrimidin-3-yl)imidazo[1,2-a]pyrimidine-6-carboxamide

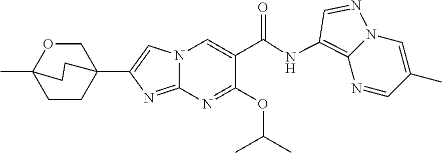

### Comparator 1C: 7-isopropoxy-N-(1-methyl-1H-pyrazol-3-yl)-2-(1-methyl-2-oxabicyclo[2.1.1]hexan-4-yl)imidazo[1,2-a]pyridine-6-carboxamide

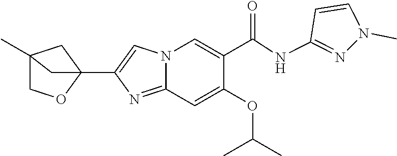

### Comparator 2C: 7-isopropoxy-N-(1-methyl-1H-pyrazol-3-yl)-2-(1-methyl-2-oxabicyclo[2.1.1]hexan-4-yl)imidazo[1,2-a]pyrimidine-6-carboxamide

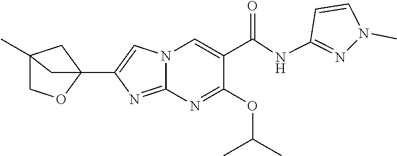

### Comparator 3C: 7-isopropoxy-N-(1-methyl-1H-pyrazol-3-yl)-2-((1S,4R)-1-methyl-2-oxabicyclo[2.2.1]heptan-4-yl)imidazo[1,2-a]pyridine-6-carboxamide

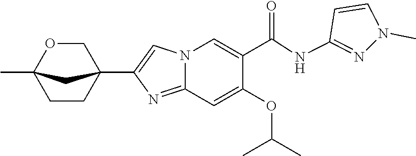

### Comparator 4C: 7-isopropoxy-N-(1-methyl-1H-pyrazol-3-yl)-2-((1S,4R)-1-methyl-2-oxabicyclo[2.2.1]heptan-4-yl)imidazo[1,2-a]pyrimidine-6-carboxamide (stereochemistry arbitrarily assigned)

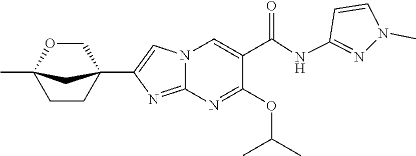

To a solution of 7-isopropoxy-2-(1-methyl-2-oxabicyclo[2.2.1]heptan-4-yl)imidazo[1,2-a]pyrimidine-6-carboxylic acid [preparation 64] (200 mg, 0.603 mmol) and compound 1-methyl-1H-pyrazol-3-amine (117 mg, 1.21 mmol) in pyridine (2 mL) was added T3P® (2 mL, 50% w/w in EtOAc). The mixture was stirred at 20° C. for 2 h. The mixture was diluted with saturated aqueous NaHCO₃ (50 mL) and the mixture was extracted with EtOAc (3×50 mL). The combined organic layers were washed with brine (50 mL), (Na₂SO₄) and filtered. The filtrate was concentrated and the residue was purified by prep-HPLC (Column: Welch Xtimate C18 150×25 mm×5 μm; Condition: water (10 mm NH₄HCO₃)-ACN; Begin B: 42; End B: 72; Gradient Time (min): 10; 100% B Hold Time (min): 2; Flow Rate (mL/min): 25) to give racemic 7-isopropoxy-N-(1-methyl-1H-pyrazol-3-yl)-2-(1-methyl-2-oxabicyclo[2.2.1]heptan-4-yl)imidazo[1,2-a]pyrimidine-6-carboxamide (100 mg, 40% yield) as a white solid. LCMS (ESI) m/z 411.0 (M+H)⁺.

The racemate was separated by SFC (Column: DAICEL CHIRALPAK AD (250 mm*30 mm, 10 um); Mobile Phase: from 50% to 50% of 0.1% NH₃H₂O ETOH; Flow Rate (ml/min): 70; Column temp: 40° C.) to give 7-isopropoxy-N-(1-methyl-1H-pyrazol-3-yl)-2-((1S,4R)-1-methyl-2-oxabicyclo[2.2.1]heptan-4-yl)imidazo[1,2-a]pyrimidine-6-carboxamide (stereochemistry arbitrarily assigned), Comparator 4C (30.1 mg, 30.10% yield, >99% ee, peak 1) as a white solid. LCMS (ESI) m/z 411.3 (M+H)⁺. ¹HNMR (500 MHz, CHLOROFORM-d) δ ppm=10.09 (1H, s), 9.18 (s, 1H), 7.31 (d, J=2.5 Hz, 1H), 7.23 (s, 1H), 6.76 (d, J=2.5 Hz, 1H), 5.90-5.80 (m, 1H), 4.20-4.10 (m, 1H), 4.00-3.90 (m, 1H), 3.85 (s, 3H), 2.20-2.10 (m, 1H), 2.10-2.05 (m, 1H), 2.05-2.00 (m, 1H), 2.00-1.95 (m, 1H), 1.90-1.80 (m, 2H), 1.58 (d, J=6.0 Hz, 6H), 1.48 (s, 3H). The enantiomer, 7-isopropoxy-N-(1-methyl-1H-pyrazol-3-yl)-2-((1R,4S)-1-methyl-2-oxabicyclo[2.2.1]heptan-4-yl)imidazo[1,2-a]pyrimidine-6-carboxamide (30 mg, 30.00% yield, 96% ee, peak 2) as a white solid. LCMS (ESI) m/z 411.3 (M+H)⁺. ¹HNMR (500 MHz, CHLOROFORM-d) δ ppm=10.09 (1H, s), 9.18 (s, 1H), 7.31 (d, J=2.5 Hz, 1H), 7.23 (s, 1H), 6.76 (d, J=2.5 Hz, 1H), 5.90-5.80 (m, 1H), 4.20-4.10 (m, 1H), 4.00-3.90 (m, 1H), 3.85 (s, 3H), 2.20-2.10 (m, 1H), 2.10-2.05 (m, 1H), 2.05-2.00 (m, 1H), 2.00-1.95 (m, 1H), 1.90-1.80 (m, 2H), 1.58 (d, J=6.0 Hz, 6H), 1.48 (s, 3H).

### Comparator 5C: 7-isopropoxy-N-(1-methyl-1H-pyrazol-3-yl)-2-(1-methyl-2-oxabicyclo[2.2.2]octan-4-yl)imidazo[1,2-a]pyridine-6-carboxamide

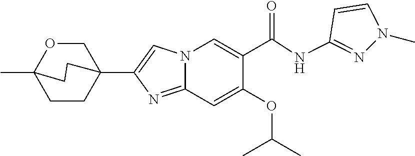

### Comparator 6C: 7-isopropoxy-N-(1-methyl-1H-pyrazol-3-yl)-2-(1-methyl-2-oxabicyclo[2.2.2]octan-4-yl)imidazo[1,2-a]pyrimidine-6-carboxamide

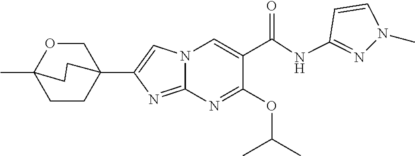

### Comparator 1D: 7-isopropoxy-N-(1-methyl-2-oxo-1,2-dihydropyridin-3-yl)-2-(1-methyl-2-oxabicyclo[2.1.1]hexan-4-yl)imidazo[1,2-a]pyridine-6-carboxamide

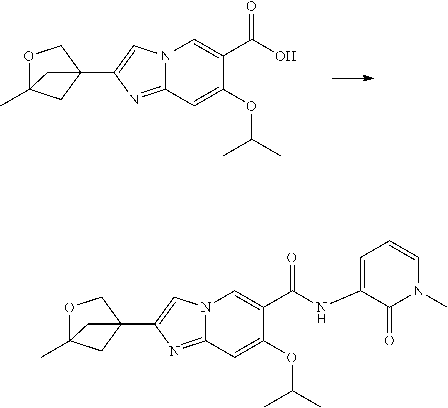

T3P (502 mg, 790 μmol, 470 μL, 50% purity) added to 7-isopropoxy-2-(1-methyl-2-oxabicyclo[2.1.1]hexan-4-yl)imidazo[1,2-a]pyridine-6-carboxylic acid [preparation 4] (50 mg, 158 μmol) and 3-amino-1-methyl-pyridin-2-one (33 mg, 205 μmol, Hydrochloride) in Pyridine (0.9 mL) at rt. After stirring overnight, the mixture was diluted with water and extracted with DCM and then EtOAc, dried over MgSO₄, filtered and concentrated. The crude material was purified by mass-directed reverse-phase HPLC (column: XSelect CSH Prep C18 5 um OBD 19×100 mm; Mobile phase A: MeCN; Mobile phase B: H₂O, Modifier: 0.1% NH₄OH) to provide 7-isopropoxy-2-(1-methyl-2-oxabicyclo[2.1.1]hexan-4-yl)-N-(1-methyl-2-oxo-3-pyridyl)imidazo[1,2-a]pyridine-6-carboxamide (22.9 mg, 54.2 μmol, 34% yield). LCMS (ESI) m/z 422.9 (M+H)⁺. 1H NMR (500 MHz, DMSO-d6) δ ppm 1.43 (s, 3H) 1.52 (d, J=6.10 Hz, 6H) 1.77 (dd, J=4.27, 1.83 Hz, 2H) 2.01 (br d, J=3.05 Hz, 2H) 3.57 (s, 3H) 3.89 (s, 2H) 5.00-5.12 (m, 1H) 6.28-6.40 (m, 1H) 7.21 (s, 1H) 7.48 (dd, J=6.71, 1.83 Hz, 1H) 7.86 (br s, 1H) 8.47 (dd, J=7.94, 1.83 Hz, 1H) 9.22 (s, 1H) 10.76 (s, 1H).

### Comparator 2D: 7-isopropoxy-N-(1-methyl-2-oxo-1,2-dihydropyridin-3-yl)-2-(1-methyl-2-oxabicyclo[2.1.1]hexan-4-yl)imidazo[1,2-a]pyrimidine-6-carboxamide

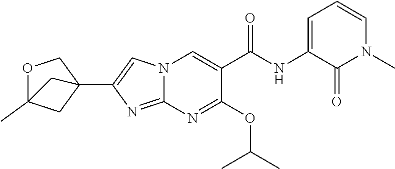

### Comparator 3D: 7-isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide (stereochemistry arbitrarily assigned)

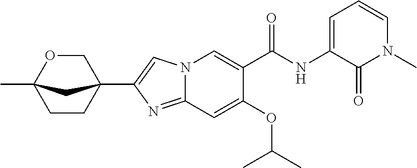

### Comparator 4D: 7-isopropoxy-N-(1-methyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide

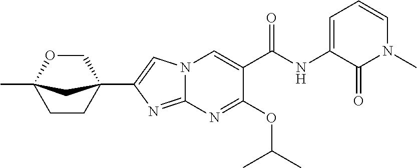

### Comparator 5D: 7-isopropoxy-N-(1-methyl-2-oxo-1,2-dihydropyridin-3-yl)-2-(1-methyl-2-oxabicyclo[2.2.2]octan-4-yl)imidazo[1,2-a]pyridine-6-carboxamide

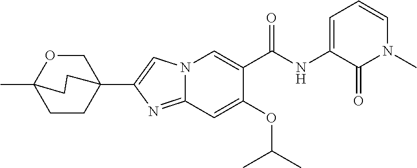

### Comparator 6D: 7-isopropoxy-N-(-methyl-2-oxo-1,2-dihydropyridin-3-yl)-2-(1-methyl-2-oxabicyclo[2.2.2]octan-4-yl)imidazo[1,2-a]pyrimidine-6-carboxamide

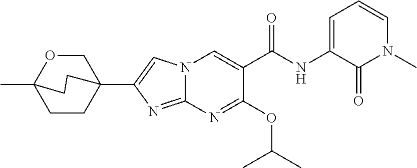

Data for Comparator Compounds:

| | IRAK4 MSD | | pH 6.8 | |
|---|---|---|---|---|
| Comparator | BIOCHEMICAL | MDR1-MDCK | Solubility | Brain |
| Number | IC50 (nM) | Efflux Ratio | (ug/mL) | Kpu, u |
| 1A | 0.2 | 0.9 | 2.1 | 0.4 |
| 2A | 0.2 | 1.1 | 2.4 | 0.4 |
| 1C | 4.8 | 6.4 | 57.8 | 0.5 |
| 2C | 5.0 | 7.4 | 54.5 | 0.3 |
| 2B | 0.8 | 5.8 | 4.3 | 0.3 |
| 1B | 0.2 | 4.3 | 0.3 | 0.3 |
| 3A | 0.1 | 0.7 | 0.8 | |
| 4A | 0.2 | 0.7 | 2.0 | 0.4 |
| 3C | 2.3 | 6.3 | 28.5 | 0.3 |
| 6C | 0.9 | 1.8 | 48.5 | 0.2 |
| 6B | 0.2 | 1.5 | 7.2 | 0.0 |
| 5A | 0.1 | 1.0 | 1.5 | |
| 5C | 1.7 | 2.2 | 39.6 | 0.4 |
| 5B | 1.8 | 1.4 | 5.2 | |
| 5D | 0.2 | 2.0 | 45.6 | 0.6 |
| 2D | 1.7 | 5.7 | 65.3 | |
| 3D | 0.6 | 3.1 | 49.3 | 1.1 |
| 4B | 0.2 | 2.3 | 2.0 | |
| 4C | 2.5 | 4.6 | 51.3 | |
| 4D | 0.5 | 5.6 | 58.8 | 0.2 |
| 6D | 0.5 | 3.0 | 11.2 | 0.4 |
| 3B | 0.1 | 2.0 | 0.7 | |
| 1D | 0.6 | 8.0 | 63.2 | 0.9 |

1. A compound represented by Formula (I): 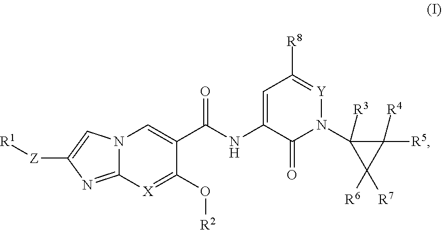
or a pharmaceutically acceptable salt thereof, wherein:
X is CH, CF or N;
Y is CH or N;
Z is ring A or —CH₂-ring A-*, wherein -* indicates the point of connection to R¹;
Ring A is 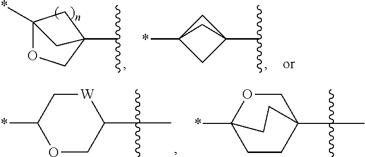
wherein n is 1 or 2; W is absent, CH₂ or O, and [symbol] indicates the point of connection to R¹;
R¹ is H, —CN, C₁₋₃alkoxy or C₁₋₃alkyl optionally substituted with 1 to 3 substituents independently selected from halo and C₁-C₃alkoxy;
R² is C₃₋₆cycloalkyl or C₁₋₄alkyl, wherein the C₃₋₆cycloalkyl or C₁₋₄alkyl is optionally substituted with 1 to 3 halo; and
R³, R⁴, R⁵, R⁶ and R⁷ are each independently selected from H, halo, CN, C₁₋₄alkyl, C₁₋₄haloalkyl, C₁₋₄alkoxy, and C₁₋₄alkoxyC₁₋₄alkyl, or any two of R³, R⁴, R⁵, R⁶ and R⁷ together with the carbon atoms from which they are attached form a C₃₋₆cycloalkyl or a 4 to 6 membered heterocyclyl containing one or two heteroatoms independently selected 0, N, and S; and
R⁸ is H or halo.

2. The compound of claim 1, or a pharmaceutically acceptable salt thereof, wherein X is CH.

3. The compound of claim 1, or a pharmaceutically acceptable salt thereof, wherein X is N.

4. The compound of any one of claims 1 to 3, or a pharmaceutically acceptable salt thereof, wherein Y is CH.

5. The compound of any one of claims 1 to 3, or a pharmaceutically acceptable salt thereof, wherein Y is N.

6. The compound of any one of claims 1 to 5, or a pharmaceutically acceptable salt thereof, wherein Z is ring A and ring A is

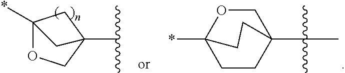

7. The compound of any one of claims 1 to 5, or a pharmaceutically acceptable salt thereof, wherein Z is ring A and ring A is

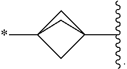 [SMILES: *C12CC(*)(C1)C2]

8. The compound of any one of claims 1 to 5, or a pharmaceutically acceptable salt thereof, wherein ring A is

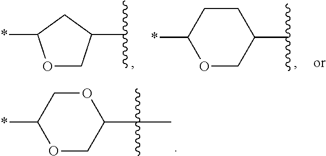

9. The compound of claim 1, wherein the compound is represented by Formula (II), (III), (IV) or (V): 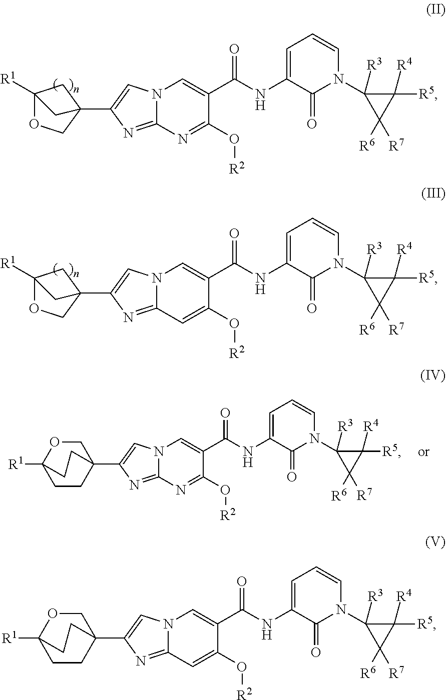
or a pharmaceutically acceptable salt thereof.

10. The compound of claim 1, wherein the compound is represented by Formula (IIA), (IIB), (IIIA), or (IIIB): 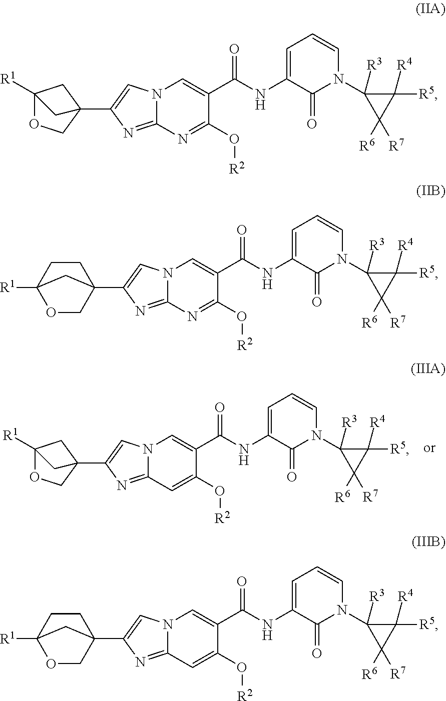
or a pharmaceutically acceptable salt thereof.

11. The compound of any one of claims 1 to 10, or a pharmaceutically acceptable salt thereof, wherein R¹ is H or C₁₋₃alkyl optionally substituted with 1 to 3 substituents independently selected from halo or C₁-C₃alkoxy.

12. The compound of claim 11, or a pharmaceutically acceptable salt thereof, wherein R¹ is C₁₋₃alkyl.

13. The compound of claim 11, or a pharmaceutically acceptable salt thereof, wherein R¹ is C₁₋₃alkyl optionally substituted with 1 to 3 halo.

14. The compound of any one of claims 1 to 10, or a pharmaceutically acceptable salt thereof, wherein R¹ is H, —CH₃, —CH₂F, —CH₂CH₃, —CH₂OCH₃, —OCH₃, or —CN.

15. The compound of any one of claims 1 to 10, or a pharmaceutically acceptable salt thereof, wherein R¹ is —CH₃.

16. The compound of any one of claims 1 to 10, or a pharmaceutically acceptable salt thereof, wherein R¹ is —CH₃ or —CH₂F.

17. The compound of any one of claims 1 to 16, or a pharmaceutically acceptable salt thereof, wherein R² is C₃₋₄alkyl or C₃₋₄cycloalkyl, wherein the C₃₋₄alkyl is optionally substituted with 1 to 3 fluoro,

18. The compound of claim 17, or a pharmaceutically acceptable salt thereof, wherein R² is C₃₋₄alkyl.

19. The compound of any one of claims 1 to 16, or a pharmaceutically acceptable salt thereof, wherein R² is —CH(CH₃)₂, —CH(CH₃)CH₂CH₃, —CH(CH₃)CH₂F, —CH(CH₃)CHF₂, cyclopropyl, or cyclobutyl,

20. The compound of claim 19, or a pharmaceutically acceptable salt thereof, wherein R² is —CH(CH₃)₂.

21. The compound of claim 19, or a pharmaceutically acceptable salt thereof, wherein R² is —CH(CH₃)₂ or —CH(CH₃)CH₂CH₃.

22. The compound of any one of claims 1 to 10, or a pharmaceutically acceptable salt thereof, wherein:
R¹ is H or C₁₋₃alkyl optionally substituted with 1 to 3 substituents independently selected from halo or C₁-C₃alkoxy; and
R² is C₃₋₄alkyl.

23. The compound of any one of claims 1 to 10, or a pharmaceutically acceptable salt thereof, wherein:
R¹ is H or C₁₋₃alkyl optionally substituted with 1 to 3 halo; and
R² is C₃₋₄alkyl.

24. The compound of any one of claims 1 to 23, or a pharmaceutically acceptable salt thereof, wherein R³, R⁴, R⁵, R⁶ and R⁷ are each independently selected from H, halo and C₁₋₃alkyl.

25. The compound of any one of claims 1 to 23, or a pharmaceutically acceptable salt thereof, wherein R³, R⁴, R⁵, R⁶ and R⁷ are each independently selected from H, halo, C₁₋₃alkyl, C₁₋₃haloalkyl and C₁₋₃alkoxy, or any two of R³, R⁴, R⁵, R⁶ and R⁷ together with the carbon atoms from which they are attached form a C₃₋₆cycloalkyl.

26. The compound of any one of claims 1 to 23, or a pharmaceutically acceptable salt thereof, wherein R³, R⁴, R⁵, R⁶ and R⁷ are each independently selected from H, F, and —CH₃.

27. The compound of any one of claims 1 to 23, or a pharmaceutically acceptable salt thereof, wherein R³, R⁴, R⁵, R⁶ and R⁷ are each independently selected from H, F, —CH₃, —CH₂CH₃, —CHF₂, and —OCH₃, or any two of R³, R⁴, R⁵, R⁶ and R⁷ together with the carbon atoms from which they are attached form a cyclopropyl.

28. The compound of claim 26, or a pharmaceutically acceptable salt thereof, wherein R³, R⁴, R⁵, R⁶ and R⁷ are all H.

29. The compound of claim 26, wherein R³, R⁵, R⁶ and R⁷ are all H, and wherein R⁴ is F or —CH₃.

30. The compound of claim 1, wherein the compound is represented by the following formula: 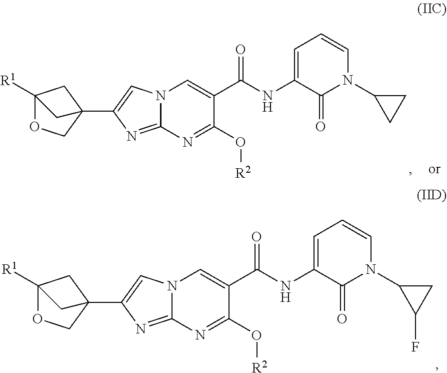
or a pharmaceutically acceptable salt thereof, wherein R¹ is C₁₋₃alkyl and R² is C₃₋₄alkyl.

31. The compound of claim 1, wherein the compound is represented by the following formula: 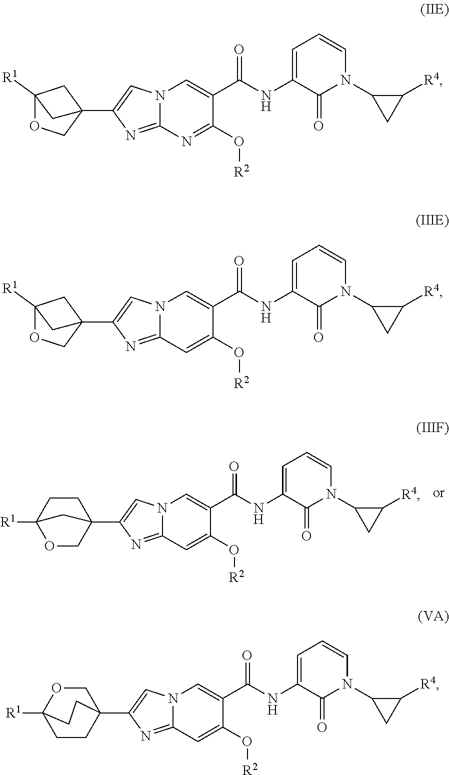
or a pharmaceutically acceptable salt thereof, wherein R¹ is C₁₋₃alkyl optionally substituted with 1 to 3 halo; R² is C₃₋₄alkyl; and R⁴ is H, halo or C₁₋₃alkyl.

32. The compound of claim 31, or a pharmaceutically acceptable salt thereof, wherein R¹ is —CH₃ or —CH₂F; R² is —CH(CH₃)₂ or —CH(CH₃)CH₂CH₃; and R⁴ is H, F or —CH₃.

33. The compound of claim 31 or 32, or a pharmaceutically acceptable salt thereof, wherein when R⁴ is not H, R⁴ and the pyridone group are in a cis orientation.

34. A compound according to claim 1, wherein said compound is selected from:
7-cyclobutoxy-N-(1-cyclopropyl-2-oxo-1,2-dihydropyridin-3-yl)-2-(1-methyl-2-oxabicyclo[2.2.2]octan-4-yl)imidazo[1,2-a]pyridine-6-carboxamide;
N-(1-cyclopropyl-2-oxo-1,2-dihydropyridin-3-yl)-2-(1-ethyl-2-oxabicyclo[2.1.1]hexan-4-yl)-7-isopropoxyimidazo[1,2-a]pyridine-6-carboxamide;
N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyridine-6-carboxamide;
N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyridine-6-carboxamide;
N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyridine-6-carboxamide;
N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyridine-6-carboxamide;
N-(1-cyclopropyl-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide;
N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyridine-6-carboxamide;
N-(1-cyclopropyl-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide;
N-(1-cyclopropyl-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide;
(R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide;
(S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyridine-6-carboxamide;
7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide;
7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide;
N-(1-cyclopropyl-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
(S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide;
7-(sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide;
7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide;
7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide;
7-isopropoxy-2-(1-methyl-2-oxabicyclo[2.1.1]hexan-4-yl)-N-(1-(1-methylcyclopropyl)-2-oxo-1,2-dihydropyridin-3-yl)imidazo[1,2-a]pyrimidine-6-carboxamide;
7-cyclobutoxy-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide;
(S)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide;
(R)—N-(1-cyclopropyl-2-oxo-1,2-dihydropyridin-3-yl)-7-((1-fluoropropan-2-yl)oxy)-2-(1-methyl-2-oxabicyclo[2.1.1]hexan-4-yl)imidazo[1,2-a]pyridine-6-carboxamide;
N-(1-cyclopropyl-2-oxo-1,2-dihydropyridin-3-yl)-7-(((S)-1-fluoropropan-2-yl)oxy)-2-(1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-(1-methyl-2-oxabicyclo[2.2.1]heptan-4-yl)imidazo[1,2-a]pyridine-6-carboxamide;
7-cyclobutoxy-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide;
2-(2-oxabicyclo[2.1.1]hexan-4-yl)-N-(1-cyclopropyl-2-oxo-1,2-dihydropyridin-3-yl)-7-isopropoxyimidazo[1,2-a]pyridine-6-carboxamide;
N-(1-cyclopropyl-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide;
2-(2-oxabicyclo[2.1.1]hexan-4-yl)-7-cyclobutoxy-N-(1-cyclopropyl-2-oxo-1,2-dihydropyridin-3-yl)imidazo[1,2-a]pyrimidine-6-carboxamide;
2-(2-oxabicyclo[2.1.1]hexan-4-yl)-7-cyclobutoxy-N-(1-cyclopropyl-2-oxo-1,2-dihydropyridin-3-yl)imidazo[1,2-a]pyridine-6-carboxamide;
2-(2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide;
2-((1S,4R)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide;
2-((1R,4S)-2-oxabicyclo[2.2.1]heptan-4-yl)-N-(1-cyclopropyl-2-oxo-1,2-dihydropyridin-3-yl)-7-isopropoxyimidazo[1,2-a]pyridine-6-carboxamide;
7-((R)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-((S)-sec-butoxy)-N-(1-(cis-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-7-ethoxy-2-(1-methyl-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-2-(1-ethyl-2-oxabicyclo[2.2.1]heptan-4-yl)-7-isopropoxyimidazo[1,2-a]pyrimidine-6-carboxamide;
N-(1-cyclopropyl-2-oxo-1,2-dihydropyridin-3-yl)-2-(1-(fluoromethyl)-2-oxabicyclo[2.2.1]heptan-4-yl)-7-isopropoxyimidazo[1,2-a]pyrimidine-6-carboxamide;
N-(1-cyclopropyl-2-oxo-1,2-dihydropyridin-3-yl)-2-((1S,4R)-1-(fluoromethyl)-2-oxabicyclo[2.2.1]heptan-4-yl)-7-isopropoxyimidazo[1,2-a]pyrimidine-6-carboxamide;
N-(1-cyclopropyl-2-oxo-1,2-dihydropyridin-3-yl)-2-((1R,4S)-1-(fluoromethyl)-2-oxabicyclo[2.2.1]heptan-4-yl)-7-isopropoxyimidazo[1,2-a]pyrimidine-6-carboxamide;
7-((R)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide;
7-((S)-sec-butoxy)-N-(1-cyclopropyl-2-oxo-1,2-dihydropyridin-3-yl)-2-((1S,4R)-1-methyl-2-oxabicyclo[2.2.1]heptan-4-yl)imidazo[1,2-a]pyrimidine-6-carboxamide;
7-((S)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide;
7-((R)-sec-butoxy)-N-(1-cyclopropyl-2-oxo-1,2-dihydropyridin-3-yl)-2-((1R,4S)-1-methyl-2-oxabicyclo[2.2.1]heptan-4-yl)imidazo[1,2-a]pyrimidine-6-carboxamide;
N-(1-((1R,2S)-2-fluorocyclopropyl)-2-oxo-1,2-dihydropyridin-3-yl)-2-(1-(fluoromethyl)-2-oxabicyclo[2.1.1]hexan-4-yl)-7-isopropoxyimidazo[1,2-a]pyrimidine-6-carboxamide;
N-(1-cyclopropyl-2-oxo-1,2-dihydropyridin-3-yl)-7-isopropoxy-2-(1-(methoxymethyl)-2-oxabicyclo[2.2.1]heptan-4-yl)imidazo[1,2-a]pyrimidine-6-carboxamide;
2-(2-oxabicyclo[2.1.1]hexan-4-yl)-N-(1-cyclopropyl-2-oxo-1,2-dihydropyridin-3-yl)-7-isopropoxyimidazo[1,2-a]pyrimidine-6-carboxamide;
7-((R)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide;
7-((S)-sec-butoxy)-N-(1-cyclopropyl-2-oxo-1,2-dihydropyridin-3-yl)-2-(1-(fluoromethyl)-2-oxabicyclo[2.2.1]heptan-4-yl)imidazo[1,2-a]pyrimidine-6-carboxamide;
7-((R)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-((R)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-8-fluoro-7-isopropoxy-2-(1-methyl-2-oxabicyclo[2.1.1]hexan-4-yl)imidazo[1,2-a]pyridine-6-carboxamide;
N-(1-cyclopropyl-2-oxo-1,2-dihydropyridin-3-yl)-7-isopropoxy-2-(3-methoxybicyclo[1.1.1]pentan-1-yl)imidazo[1,2-a]pyridine-6-carboxamide;
(R)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide;
(S)-7-(sec-butoxy)-N-(1-cyclopropyl-2-oxo-1,2-dihydropyridin-3-yl)-2-(1-methyl-2-oxabicyclo[2.1.1]hexan-4-yl)imidazo[1,2-a]pyrimidine-6-carboxamide;
(R)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide;
(S)-7-(sec-butoxy)-N-(1-cyclopropyl-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-8-fluoro-7-isopropoxy-2-((1S,4R)-1-methyl-2-oxabicyclo[2.2.1]heptan-4-yl)imidazo[1,2-a]pyridine-6-carboxamide;
N-(1-cyclopropyl-2-oxo-1,2-dihydropyridin-3-yl)-8-fluoro-7-isopropoxy-2-((1R,4S)-1-methyl-2-oxabicyclo[2.2.1]heptan-4-yl)imidazo[1,2-a]pyridine-6-carboxamide;
(R)—N-(1-cyclopropyl-2-oxo-1,2-dihydropyridin-3-yl)-2-(1,4-dioxan-2-yl)-7-isopropoxyimidazo[1,2-a]pyridine-6-carboxamide;
(S)—N-(1-cyclopropyl-2-oxo-1,2-dihydropyridin-3-yl)-2-(1,4-dioxan-2-yl)-7-isopropoxyimidazo[1,2-a]pyridine-6-carboxamide;
N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide;
7-cyclobutoxy-N-(2-cyclopropyl-3-oxo-2,3-dihydropyridazin-4-yl)-2-(1-methyl-2-oxabicyclo[2.1.1]hexan-4-yl)imidazo[1,2-a]pyrimidine-6-carboxamide;
N-(2-cyclopropyl-3-oxo-2,3-dihydropyridazin-4-yl)-8-fluoro-7-isopropoxy-2-(1-methyl-2-oxabicyclo[2.1.1]hexan-4-yl)imidazo[1,2-a]pyridine-6-carboxamide;
N-(2-cyclopropyl-3-oxo-2,3-dihydropyridazin-4-yl)-7-isopropoxy-2-((1S,4R)-1-methyl-2-oxabicyclo[2.2.1]heptan-4-yl)imidazo[1,2-a]pyridine-6-carboxamide;
N-(2-cyclopropyl-3-oxo-2,3-dihydropyridazin-4-yl)-7-isopropoxy-2-((1R,4S)-1-methyl-2-oxabicyclo[2.2.1]heptan-4-yl)imidazo[1,2-a]pyridine-6-carboxamide;
N-(2-cyclopropyl-3-oxo-2,3-dihydropyridazin-4-yl)-7-isopropoxy-2-(1-methyl-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-7-isopropoxy-2-(1-methyl-2-oxabicyclo[2.2.2]octan-4-yl)imidazo[1,2-a]pyrimidine-6-carboxamide;
(R)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
(S)—N-(1-(2,2-dimethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
2-(3-cyanobicyclo[1.1.1]pentan-1-yl)-N-(1-cyclopropyl-2-oxo-1,2-dihydropyridin-3-yl)-7-isopropoxyimidazo[1,2-a]pyridine-6-carboxamide;
N-(1-trans-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
N-(1-(2,2-difluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
2-(2-oxabicyclo[2.1.1]hexan-4-yl)-7-cyclobutoxy-N-(1-(2-fluorocyclopropyl)-2-oxo-1,2-dihydropyridin-3-yl)imidazo[1,2-a]pyrimidine-6-carboxamide;
2-(2-oxabicyclo[2.1.1]hexan-4-yl)-N-(1-(2-fluorocyclopropyl)-2-oxo-1,2-dihydropyridin-3-yl)-7-isopropoxyimidazo[1,2-a]pyrimidine-6-carboxamide;
2-(2-oxabicyclo[2.1.1]hexan-4-yl)-N-(1-((1R,2S)-2-fluorocyclopropyl)-2-oxo-1,2-dihydropyridin-3-yl)-7-isopropoxyimidazo[1,2-a]pyrimidine-6-carboxamide;
2-(2-oxabicyclo[2.1.1]hexan-4-yl)-N-(1-((1S,2R)-2-fluorocyclopropyl)-2-oxo-1,2-dihydropyridin-3-yl)-7-isopropoxyimidazo[1,2-a]pyrimidine-6-carboxamide;
N-(1-(2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide;
8-fluoro-N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
8-fluoro-N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
8-fluoro-N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
7-isopropoxy-2-(1-methyl-2-oxabicyclo[2.1.1]hexan-4-yl)-N-(1-(2-methylcyclopropyl)-2-oxo-1,2-dihydropyridin-3-yl)imidazo[1,2-a]pyrimidine-6-carboxamide;
7-((S)-sec-butoxy)-N-(1-cis-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
N-(1-(2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
7-isopropoxy-2-((1S,4R)-1-methyl-2-oxabicyclo[2.2.1]heptan-4-yl)-N-(1-(2-methylcyclopropyl)-2-oxo-1,2-dihydropyridin-3-yl)imidazo[1,2-a]pyridine-6-carboxamide;
N-(1-(2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-(2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
N-(1-(2-fluorocyclopropyl)-2-oxo-1,2-dihydropyridin-3-yl)-7-isopropoxy-2-(1-methyl-2-oxabicyclo[2.2.1]heptan-4-yl)imidazo[1,2-a]pyrimidine-6-carboxamide;
(S)—N-(1-cyclopropyl-2-oxo-1,2-dihydropyridin-3-yl)-7-((1,1-difluoropropan-2-yl)oxy)-2-(1-methyl-2-oxabicyclo[2.1.1]hexan-4-yl)imidazo[1,2-a]pyridine-6-carboxamide;
7-cyclobutoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-cyclopropoxy-N-(1-cyclopropyl-2-oxo-1,2-dihydropyridin-3-yl)-2-(1-methyl-2-oxabicyclo[2.1.1]hexan-4-yl)imidazo[1,2-a]pyridine-6-carboxamide;
7-cyclopropoxy-N-(1-(2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-cyclobutoxy-N-(1-(2-fluorocyclopropyl)-2-oxo-1,2-dihydropyridin-3-yl)-2-(1-(fluoromethyl)-2-oxabicyclo[2.1.1]hexan-4-yl)imidazo[1,2-a]pyrimidine-6-carboxamide;
(R)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide;
(S)—N-(1-cyclopropyl-2-oxo-1,2-dihydropyridin-3-yl)-7-isopropoxy-2-(tetrahydro-2H-pyran-3-yl)imidazo[1,2-a]pyrimidine-6-carboxamide;
(R)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide;
(S)—N-(1-cyclopropyl-2-oxo-1,2-dihydropyridin-3-yl)-7-isopropoxy-2-((tetrahydrofuran-3-yl)methyl)imidazo[1,2-a]pyrimidine-6-carboxamide;
N-(1-cyclopropyl-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide;
N-(1-cyclopropyl-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide;
7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
N-(1-cyclopropyl-2-oxo-1,2-dihydropyridin-3-yl)-7-isopropoxy-2-(4-methyl-2-oxabicyclo[2.1.1]hexan-1-yl)imidazo[1,2-a]pyrimidine-6-carboxamide;
N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-((1S,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-((1R,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
N-(1-((1S,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
N-(1-((1R,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide;
N-(1-((1S,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide;
N-(1-((1R,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide;
N-(1-((1S,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide;
8-fluoro-N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
8-fluoro-N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
8-fluoro-N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
8-fluoro-N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
7-cyclobutoxy-N-(1-((1S,2R)-2-fluorocyclopropyl)-2-oxo-1,2-dihydropyridin-3-yl)-2-(1-(methoxymethyl)-2-oxabicyclo[2.1.1]hexan-4-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide;
N-(1-((1S,2R)-2-fluorocyclopropyl)-2-oxo-1,2-dihydropyridin-3-yl)-7-isopropoxy-2-((1R,4S)-1-(methoxymethyl)-2-oxabicyclo[2.2.1]heptan-4-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide;
N-(1-((1S,2R)-2-fluorocyclopropyl)-2-oxo-1,2-dihydropyridin-3-yl)-7-isopropoxy-2-((1S,4R)-1-(methoxymethyl)-2-oxabicyclo[2.2.1]heptan-4-yl)imidazo[1,2-a]pyrimidine-6-carboxamide;
7-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyridine-6-carboxamide;
7-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyridine-6-carboxamide;
7-isopropoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyrimidine-6-carboxamide;
7-isopropoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyrimidine-6-carboxamide;
7-isopropoxy-N-(1-((1R,2R)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-isopropoxy-N-(1-((1S,2S)-2-methoxycyclopropyl)-2-oxo-1,2-dihydropyridin-3-yl)-2-(1-methyl-2-oxabicyclo[2.1.1]hexan-4-yl)imidazo[1,2-a]pyrimidine-6-carboxamide;
7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide;
7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide;
7-isopropoxy-2-((1R,4S)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide;
7-isopropoxy-2-((1R,4S)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide;
N-(1-((1R,2R)-2-ethylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
N-(1-((1S,2S)-2-(difluoromethyl)-2-methylcyclopropyl)-2-oxo-1,2-dihydropyridin-3-yl)-7-isopropoxy-2-(1-methyl-2-oxabicyclo[2.1.1]hexan-4-yl)imidazo[1,2-a]pyrimidine-6-carboxamide;
8-fluoro-7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyridine-6-carboxamide;
8-fluoro-7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyridine-6-carboxamide;
7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyrimidine-6-carboxamide;
7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyrimidine-6-carboxamide;
7-isopropoxy-2-((1S,4R)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide;
7-isopropoxy-2-((1S,4R)-1-methyl-2-oxabicyclo[2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide;
7-isopropoxy-2-((1R,4S)-1-methyl-2-oxabicyclo[2.2.1]heptan-4-yl)-N-(2-oxo-1-((S)-spiro[2.2]pentan-1-yl)-1,2-dihydropyridin-3-yl)imidazo[1,2-a]pyrimidine-6-carboxamide;
7-isopropoxy-2-((1R,4S)-1-methyl-2-oxabicyclo [2.2.1]heptan-4-yl)-N-(2-oxo-1-((R)-spiro[2.2]pentan-1-yl)-1,2-dihydropyridin-3-yl)imidazo[1,2-a]pyrimidine-6-carboxamide;
N-(1-((1R,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
N-(1-((1S,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
7-isopropoxy-2-(1-methyl-2-oxabicyclo[2.1.1]hexan-4-yl)-N-(1-((1R,2R)-2-methylcyclopropyl)-2-oxo-1,2-dihydropyridin-3-yl)imidazo[1,2-a]pyrimidine-6-carboxamide;
7-isopropoxy-2-(1-methyl-2-oxabicyclo[2.1.1]hexan-4-yl)-N-(1-((1S,2S)-2-methylcyclopropyl)-2-oxo-1,2-dihydropyridin-3-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
7-((S)-sec-butoxy)-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-((S)-sec-butoxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
N-(1-((1R,2S)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide;
N-(1-((1S,2R)-2-fluorocyclopropyl)-2-oxo-1,2-dihydropyridin-3-yl)-7-isopropoxy-2-(1-(methoxymethyl)-2-oxabicyclo[2.1.1]hexan-4-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
N-(1-((1S,2R)-2-fluorocyclopropyl)-2-oxo-1,2-dihydropyridin-3-yl)-7-isopropoxy-2-(1-methyl-2-oxabicyclo[2.1.1]hexan-4-yl)imidazo[1,2-a]pyridine-6-carboxamide;
2-(2-oxabicyclo[2.1.1]hexan-4-yl)-7-cyclobutoxy-N-(1-((1R,2S)-2-fluorocyclopropyl)-2-oxo-1,2-dihydropyridin-3-yl)imidazo[1,2-a]pyrimidine-6-carboxamide;
2-(2-oxabicyclo[2.1.1]hexan-4-yl)-7-cyclobutoxy-N-(1-((1S,2R)-2-fluorocyclopropyl)-2-oxo-1,2-dihydropyridin-3-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyridine-6-carboxamide;
N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyridine-6-carboxamide;
7-cyclobutoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-cyclobutoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-cyclopropoxy-N-(1-((1R,2S)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-cyclopropoxy-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
N-(1-((1R,2S)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide;
N-(1-((1S,2R)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide;
N-(1-((1S,2R)-2-fluorocyclopropyl)-2-oxo-1,2-dihydropyridin-3-yl)-7-isopropoxy-2-((1R,4S)-1-methyl-2-oxabicyclo[2.2.1]heptan-4-yl)imidazo[1,2-a]pyrimidine-6-carboxamide;
N-(1-((1R,2S)-2-fluorocyclopropyl)-2-oxo-1,2-dihydropyridin-3-yl)-7-isopropoxy-2-((1S,4R)-1-methyl-2-oxabicyclo[2.2.1]heptan-4-yl)imidazo[1,2-a]pyrimidine-6-carboxamide;
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyridine-6-carboxamide trifluoroacetate;
7-isopropoxy-2-((1R,4S)-1-methyl-2-oxabicyclo[2.2.1]heptan-4-yl)-N-(1-((1R,2S)-2-methylcyclopropyl)-2-oxo-1,2-dihydropyridin-3-yl)imidazo[1,2-a]pyridine-6-carboxamide trifluoroacetate;
7-isopropoxy-2-((1S,4R)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyridine-6-carboxamide trifluoroacetate;
7-isopropoxy-2-((1S,4R)-1-methyl-2-oxabicyclo[2.2.1]heptan-4-yl)-N-(1-((1R,2S)-2-methylcyclopropyl)-2-oxo-1,2-dihydropyridin-3-yl)imidazo[1,2-a]pyridine-6-carboxamide trifluoroacetate;
7-(((S)-1,1-difluoropropan-2-yl)oxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-(((R)-1,1-difluoropropan-2-yl)oxy)-N-(1-((1S,2R)-2-fluorocyclopropyl)-2-oxo-1,2-dihydropyridin-3-yl)-2-(1-methyl-2-oxabicyclo[2.1.1]hexan-4-yl)imidazo[1,2-a]pyridine-6-carboxamide;
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate;
7-isopropoxy-2-((1R,4S)-1-methyl-2-oxabicyclo[2.2.1]heptan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate;
7-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyrimidine-6-carboxamide trifluoroacetate;
7-cyclobutoxy-2-(1-methyl-2-oxabicyclo[2.1.1]hexan-4-yl)-N-(1-((1R,2S)-2-methylcyclopropyl)-2-oxo-1,2-dihydropyridin-3-yl)imidazo[1,2-a]pyrimidine-6-carboxamide trifluoroacetate;
7-cyclobutoxy-2-(1-methyl-2-oxabicyclo[2.1.1]hexan-4-yl)-N-(1-((1S,2R)-2-methylcyclopropyl)-2-oxo-1,2-dihydropyridin-3-yl)imidazo[1,2-a]pyridine-6-carboxamide trifluoroacetate;
7-isopropoxy-2-((1S,4R)-1-methyl-2-oxabicyclo[2.2.1]heptan-4-yl)-N-(1-((1R,2R)-2-methylcyclopropyl)-2-oxo-1,2-dihydropyridin-3-yl)imidazo[1,2-a]pyridine-6-carboxamide; and
7-isopropoxy-2-((1S,4R)-1-methyl-2-oxabicyclo[2.2.1]heptan-4-yl)-N-(1-((1S,2S)-2-methylcyclopropyl)-2-oxo-1,2-dihydropyridin-3-yl)imidazo[1,2-a]pyridine-6-carboxamide;
or a pharmaceutically acceptable salt thereof.

35. A pharmaceutical composition comprising a compound of any one of claims 1 to 34, or a pharmaceutically acceptable salt thereof, and a pharmaceutically acceptable carrier.

36. The pharmaceutical composition of claim 35, further comprising one or more additional pharmaceutical agents.

37. A method of treating an IRAK4 mediated disease in a subject comprising administering to the subject a compound or a pharmaceutically acceptable salt thereof of any one of claims 1 to 34 or a pharmaceutical composition of any one of claim 35 or 36.

38. The method of claim 37, wherein the IRAK4 mediated disease is selected from the group consisting from ophthalmology, uveitis, rheumatoid arthritis, psoriatic arthritis, osteoarthritis, lupus, systemic lupus erythematosus, lupus nephritis, neuropsychiatric lupus, ankylosing spondylitis, osteoporosis, systemic sclerosis, multiple sclerosis, neuromyelitis optica, psoriasis, type I diabetes, type II diabetes, inflammatory bowel disease, Cronh's disease, ulcerative colitis, hyperimmunoglobulinemia D, periodic fever syndrome, Cryopyrin-associated periodic syndromes, Schnitzler's syndrome, systemic juvenile idiopathic arthritis, adult's onset Still's disease, gout, pseudogout, SAPHO syndrome, Castleman's disease, sepsis, stroke, atherosclerosis, celiac disease, deficiency of IL-1 receptor antagonist, Alzheimer's disease, Parkinson's disease, and cancer.

39. The method of claim 38, wherein the cancer is selected from the group consisting of lymphoma, leukemia, and Myelodysplastic Syndrome.

40. The method of claim 39, wherein the leukemia is acute myelogenous leukemia (AML) or chronic lymphocytic leukemia (CLL), and the lymphoma is non-Hodgkin's lymphoma (NHL), small lymphocytic lymphoma (SLL), macroglobulinemia/lymphoplasmacytic lymphoma (WM/LPL), or DLBC lymphomas.

41. The method of claim 37, wherein the IRAK4 mediated disease is selected from the group consisting from is selected from an autoimmune disease, an inflammatory disease, bone diseases, metabolic diseases, neurological and neurodegenerative diseases and/or disorders, cardiovascular diseases, allergies, asthma, hormone-related diseases, ischemic stroke, cerebral ischemia, hypoxia, traumatic brain injury, chronic traumatic encephalopathy, epilepsy, Parkinson's disease, and amyotrophic lateral sclerosis.
